(19) United States
(12) Patent Application Publication
(10) Pub. No.: US 2013/0338142 A1
(43) Pub. Date: Dec. 19, 2013

(54) IMIDAZOPYRAZINE SYK INHIBITORS

(71) Applicant: Gilead Connecticut, Inc., Foster City, CA (US)

(72) Inventors: Peter A. BLOMGREN, North Branford, CT (US); Kevin S. CURRIE, North Branford, CT (US); Randall HALCOMB, Foster City, CA (US); Jeffrey E. KROPF, Branford, CT (US); Seung H. LEE, Branford, CT (US); Jiayao LI, Foster City, CA (US); Jennifer R. LO, Branford, CT (US); Scott A. MITCHELL, East Haven, CT (US); Aaron SCHMITT, Hamden, CT (US); Qiaoyin WU, Foster City, CA (US); Jin-Min XIONG, Branford, CT (US); Jianjun XU, Madison, CT (US); Zhongdong ZHAO, Guilford, CT (US); Jayaraman CHANDRASEKHAR, Brandford, CT (US); Eric LANSDON, San Jose, CA (US)

(21) Appl. No.: 13/918,848

(22) Filed: Jun. 14, 2013

Related U.S. Application Data
(60) Provisional application No. 61/659,936, filed on Jun. 14, 2012.

(51) Int. Cl. C07D 487/04; C07D 498/10; C07D 513/04; C07D 498/04

(52) U.S. Cl. CPC C07D 487/04; C07D 498/04; C07D 498/10; C07D 513/04
USPC 514/211.05; 544/73; 514/230.5; 540/490; 540/552; 514/211.09; 544/350; 514/249; 544/105; 544/71; 544/70; 540/544; 514/211.15; 544/74; 544/48; 514/224.2; 544/117; 514/233.2

(57) ABSTRACT

Certain imidazopyrazines and pharmaceutical compositions thereof are provided herein. Methods of treating patients suffering from certain diseases and disorders responsive to the inhibition of Syk activity, which comprises administering to such patients an amount of an imidazopyrazine compound effective to reduce signs or symptoms of the disease or disorder are provided.

### CROSS REFERENCE TO RELATED APPLICATIONS

This application claims the benefit of U.S. provisional patent application Ser. No. 61/659,936, filed Jun. 14, 2012, the disclosure of which is hereby incorporated herein by reference in its entirety.

### FIELD

The present disclosure relates generally to imidazopyrazine compounds, and more specifically to certain imidazopyrazine compounds, compositions, and methods of their manufacture and use.

### BACKGROUND

Protein kinases, the largest family of human enzymes, encompass well over 500 proteins. Spleen Tyrosine Kinase (Syk) is a member of the Syk family of tyrosine kinases, and is a regulator of early B-cell development as well as mature B-cell activation, signaling, and survival.

Syk is a non-receptor tyrosine kinase that plays critical roles in immunoreceptor- and integrin-mediated signaling in a variety of cell types, including B-cells, macrophages, monocytes, mast cells, eosinophils, basophils, neutrophils, dendritic cells, natural killer cells, platelets, and osteoclasts. Immunoreceptors as described herein include classical immunoreceptors and immunoreceptor-like molecules. Classical immunoreceptors include B-cell and T-cell antigen receptors as well as various immunoglobulin receptors (Fc receptors). Immunoreceptor-like molecules are either structurally related to immunoreceptors or participate in similar signal transduction pathways and are primarily involved in non-adaptive immune functions, including neutrophil activation, natural killer cell recognition, and osteoclast activity. Integrins are cell surface receptors that play key roles in the control of leukocyte adhesion and activation in both innate and adaptive immunity.

Ligand binding leads to activation of both immunoreceptors and integrins, which results in Src family kinases being activated, and phosphorylation of immunoreceptor tyrosine-based activation motifs (ITAMs) in the cytoplasmic face of receptor-associated transmembrane adaptors. Syk binds to the phosphorylated ITAM motifs of the adaptors, leading to activation of Syk and subsequent phosphorylation and activation of downstream signaling pathways.

Syk is essential for B-cell activation through B-cell receptor (BCR) signaling. Syk becomes activated upon binding to phosphoryated BCR and thus initiates the early signaling events following BCR activation. B-cell signaling through BCR can lead to a wide range of biological outputs, which in turn depend on the developmental stage of the B-cell. The magnitude and duration of BCR signals must be precisely regulated. Aberrant BCR-mediated signaling can cause disregulated B-cell activation and/or the formation of pathogenic auto-antibodies leading to multiple autoimmune and/or inflammatory diseases. Mice lacking Syk show impaired maturation of B-cells, diminished immunoglobulin production, compromised T-cell-independent immune responses and marked attenuation of the sustained calcium sign upon BCR stimulation.

A large body of evidence supports the role of B-cells and the humoral immune system in the pathogenesis of autoimmune and/or inflammatory diseases. Protein-based therapeutics (such as Rituxan) developed to deplete B-cells represent an approach to the treatment of a number of autoimmune and inflammatory diseases. Auto-antibodies and their resulting immune complexes are known to play pathogenic roles in autoimmune disease and/or inflammatory disease. The pathogenic response to these antibodies is dependent on signaling through Fe Receptors, which is, in turn, dependent upon Syk. Because of Syk's role in B-cell activation, as well as FeR dependent signaling, inhibitors of Syk can be useful as inhibitors of B-cell mediated pathogenic activity, including autoantibody production. Therefore, inhibition of Syk enzymatic activity in cells is proposed as a treatment for autoimmune disease through its effects on autoantibody production.

Syk also plays a key role in FCεR1 mediated mast cell degranulation and eosinophil activation. Thus, Syk is implicated in allergic disorders including asthma. Syk binds to the phosphorylated gamma chain of FCεR1 via its SH2 domains and is essential for downstream signaling. Syk deficient mast cells demonstrate defective degranulation, arachidonic acid and cytokine secretion. This also has been shown for pharmacologic agents that inhibit Syk activity in mast cells. Treatment with Syk antisense oligonucleotides inhibits antigen-induced infiltration of eosinophils and neutrophils in an animal model of asthma. Syk deficient eosinophils also show impaired activation in response to FCεR1 stimulation. Therefore, small molecule inhibitors of Syk will be useful for treatment of allergy-induced inflammatory diseases including asthma.

Syk is also expressed in mast cells and monocytes and has been shown to be important for the function of these cells. For example, Syk deficiency in mice is associated with impaired IgE-mediated mast cell activation, which is marked diminution of TNF-alpha and other inflammatory cytokine release. Syk kinase inhibitors have also been shown to inhibit mast cell degranulation in cell based assays. Additionally, Syk inhibitors have been shown to inhibit antigen-induced passive cutaneous anaphylaxis, bronchoconstriction and bronchial edema in rats.

Thus, the inhibition of Syk activity can be useful for the treatment of allergic disorders, autoimmune diseases and inflammatory diseases such as: SLE, rheumatoid arthritis, multiple vasculitides, idiopathic thrombocytopenic purpura (ITP), myasthenia gravis, allergic rhinitis, chronic obstructive pulmonary disease (COPD), adult respiratory distress syndrome (ARDs) and asthma. In addition, Syk has been reported to play an important role in ligand-independent tonic signaling through the B-cell receptor, known to be an important survival signal in B-cells. Thus, inhibition of Syk activity may be useful in treating certain types of cancer, including B-cell lymphoma and leukemia.

### BRIEF SUMMARY

Imidazopyrazine compounds useful for inhibiting Syk activity are described herein. Compositions and kits that include the compounds are also provided, as are methods of using and making the compounds. The imidazopyrazine compounds provided herein may find use in treating diseases or conditions such as cancer, an allergic disorder, an inflammatory disease, an autoimmune disease, and/or an acute inflammatory reaction.

In one aspect, provided is a compound having the structure of formula (I):

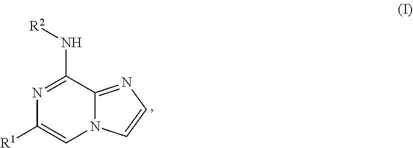

or a pharmaceutically acceptable salt, ester, prodrug, or solvate thereof, wherein:

R¹ is

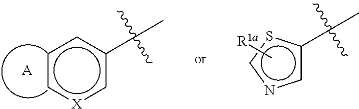

wherein:
A is selected from the group consisting of unsubstituted morpholinyl, substituted morpholinyl, unsubstituted homomorpholinyl, substituted homomorpholinyl, unsubstituted thiomorpholinyl, substituted thiomorpholinyl, unsubstituted thiomorpholinyl S-oxide, substituted thiomorpholinyl S-oxide, unsubstituted thiomorpholinyl sulfone, substituted thiomorpholinyl sulfone, unsubstituted piperidinyl, and substituted piperidinyl; and
X is N or CR^{x}, wherein R^{x} is hydrogen or C₁₋₆ alkyl;
R^{1a} is selected from the group consisting of unsubstituted alkyl, substituted alkyl, unsubstituted cycloalkyl, substituted cycloalkyl, unsubstituted heterocycloalkyl, and substituted heterocycloalkyl;
R² is

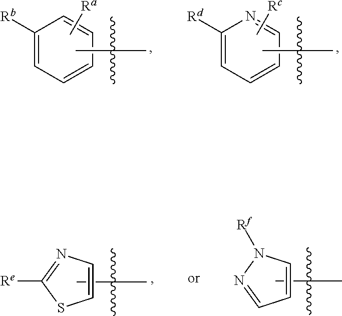

wherein:
R^{a} is selected from the group consisting of hydrogen, halo, and unsubstituted alkoxy;
R^{b} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted heterocycloalkenyl, substituted heterocycloalkenyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy;
   or R^{a} and R^{b} are taken together with the carbons to which they are attached to form a heterocyclyl ring or heteroaryl ring containing 1-3 heteroatoms selected from the group consisting of N and O;
R^{c} is hydrogen;
R^{d} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted heterocycloalkenyl, substituted heterocycloalkenyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy;
R^{e} is selected from the group consisting of hydrogen, unsubstituted alkyl, substituted alkyl, unsubstituted heterocycloalkyl, and substituted heterocycloalkyl; and
   R^{f} is selected from the group consisting of hydrogen, unsubstituted alkyl, substituted alkyl, unsubstituted heterocycloalkyl, and substituted heterocycloalkyl,
      provided that the compound is other than Compound No. 1x to 89x.

In another aspect, provided is a compound having the structure of formula (Ia):

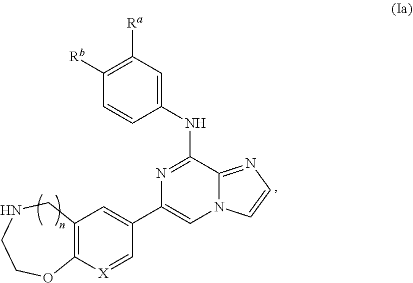

or a pharmaceutically acceptable salt, ester, prodrug, or solvate thereof, wherein:

X is N or CH;

n is 0 or 1;

R^{a} is unsubstituted alkoxy; and

R^{b} is selected from the group consisting of unsubstituted morpholinyl, substituted morpholinyl, unsubstituted piperazinyl, and substituted piperazinyl,

provided that the compound is other than Compound No. 14x, 34x, 77x, 78x or 79x.

In yet another aspect, provided is a compound having the structure of formula (Ib):

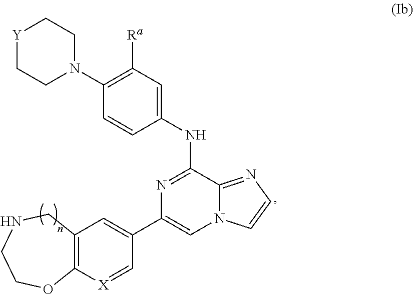

or a pharmaceutically acceptable salt, ester, prodrug, or solvate thereof, wherein:

X is N or CH;

n is 0 or 1;

R^{a} is unsubstituted alkoxy; and

Y is O or NR^{2a}, wherein R^{2a} is selected from the group consisting of unsubstituted alkyl, substituted alkyl, unsubstituted heterocycloalkyl, and substituted heterocycloalkyl,

provided that the compound is other than Compound No. 14x, 34x, 77x, 78x or 79x.

In yet another aspect, provided is a compound having the structure of formula (Ic):

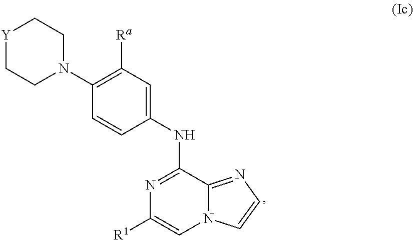

or a pharmaceutically acceptable salt, ester, prodrug, or solvate thereof, wherein:

R¹ is

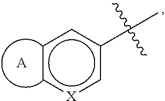

wherein A is selected from the group consisting of unsubstituted morpholinyl, substituted morpholinyl, unsubstituted oxazepanyl, and substituted oxazepanyl;

X is N or CR^{x}, wherein IV is hydrogen or C₁₋₆ alkyl;

R^{a} is unsubstituted alkoxy; and

Y is O or NR^{2a}, wherein R^{2a} is selected from the group consisting of unsubstituted alkyl, substituted alkyl, unsubstituted heterocycloalkyl, and substituted heterocycloalkyl,

provided that the compound is other than Compound No. 14x, 34x, 77x, 78x or 79x.

In yet another aspect, provided is a compound having the structure of formula (Id):

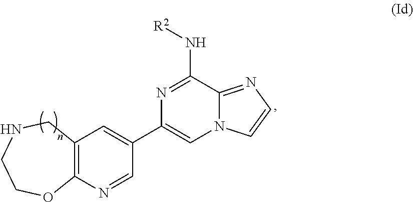

or a pharmaceutically acceptable salt, ester, prodrug, or solvate thereof, wherein:

n is 0 or 1;

R² is selected from the group consisting of unsubstituted phenyl, substituted phenyl, unsubstituted pyridinyl, substituted pyridinyl, unsubstituted pyrazolyl, substituted pyrazolyl, unsubstituted thiazolyl, and substituted thiazolyl,

provided that the compound is other than Compound No. 28x or 37x.

In yet another aspect, provided is a compound having the structure of formula (Ie):

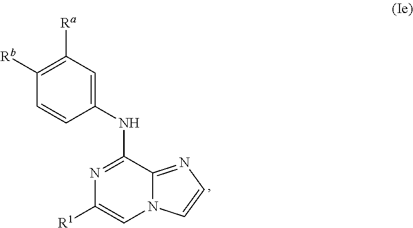

or a pharmaceutically acceptable salt, ester, prodrug, or solvate thereof, wherein:

R¹ is substituted thiazolyl;

R^{a} is selected from the group consisting of hydrogen, halo and unsubstituted alkoxy; and

R^{b} is selected from the group consisting of unsubstituted alkyl, substituted alkyl, unsubstituted alkoxy, substituted alkoxy, unsubstituted sulfonyl, substituted sulfonyl, unsubstituted morpholinyl, substituted morpholinyl, unsubstituted homomorpholinyl, substituted homomorpholinyl, unsubstituted piperazinyl, substituted piperazinyl, unsubstituted piperidinyl, substituted piperidinyl, unsubstituted pyrrolidinyl, substituted pyrrolidinyl, unsubstituted azetidinyl, and substituted azetidinyl.

In yet another aspect, provided is a compound selected from Compound No. 1-9, 12-13, 15-40, 43-45, 47-72, 76-78, 80-96, 98-109, and 111, or a pharmaceutically acceptable salt, ester, prodrug, or solvate thereof. In some embodiments, the compound is selected from Compound No. 1, 3, 4, 6, 8, 24 and 70, or a pharmaceutically acceptable salt thereof. In another embodiment, the compound is Compound No. 1, 3, 4, 24, or a pharmaceutically acceptable salt thereof. In one embodiment, the compound is Compound No. 1, 3 or 4, or a pharmaceutically acceptable salt thereof.

Also provided is a pharmaceutical composition, comprising a compound of formula I, Ia, Ib, Ic, Id or Ie, or a pharmaceutically acceptable salt, ester, prodrug, or solvate thereof, together with at least one pharmaceutically acceptable vehicle chosen from carriers, adjuvants, and excipients.

Also provided is a method for treating a patient having a disease responsive to inhibition of Syk activity, comprising administering to the patient an effective amount of a compound of formula I, Ia, Ib, Ic, Id or Ie, or a pharmaceutically acceptable salt, ester, prodrug, or solvate thereof.

Also provided is a method for treating a patient having a disease chosen from cancer, an allergic disorder, an inflammatory disease, an autoimmune disease, and an acute inflammatory reaction, comprising administering to the patient an effective amount of a compound of formula I, Ia, Ib, Ic, Id or Ie, or a pharmaceutically acceptable salt, ester, prodrug, or solvate thereof. In certain embodiments, the disease is chosen from the group consisting of B-cell lymphoma, Hodgkin's lymphoma, non-Hodgkin's lymphoma, hairy cell leukemia, multiple myeloma, chronic myelogenous leukemia, acute myelogenous leukemia, chronic lymphocytic leukemia, and acute lymphocytic leukemia. In other embodiments, the disease is selected from the group consisting of rheumatoid arthritis, allergic rhinitis, chronic obstructive pulmonary disease (COPD), adult respiratory distress syndrome (ARDS), multiple sclerosis, inflammatory bowel disease, Crohn's disease, ulcerative colitis, systemic lupus erythematosus, ovarian cancer, and polycystic kidney disease.

In some embodiments, the compound is administered intravenously, intramuscularly, parenterally, nasally, or orally. In one embodiment, the compound is administered orally.

Also provided is a method for treating a patient having polycystic kidney disease comprising administering to the patient an effective amount of a compound of formula I, Ia, Id or Ie, or a pharmaceutically acceptable salt, ester, prodrug, or solvate thereof.

Also provided is a method for increasing sensitivity of cancer cells to chemotherapy, comprising administering to a patient undergoing chemotherapy with a chemotherapeutic agent an amount a compound of formula I, Ia, Ib, Ic, Id or Ie, or a pharmaceutically acceptable salt, ester, prodrug, or solvate thereof, sufficient to increase the sensitivity of cancer cells to the chemotherapeutic agent.

Also provided is a method for inhibiting ATP hydrolysis, the method comprising contacting cells expressing Syk with a compound of formula I, Ia, Ib, Ic, Id or Ie, or a pharmaceutically acceptable salt, ester, prodrug, or solvate thereof, in an amount sufficient to detectably decrease the level of ATP hydrolysis in vitro.

Also provided is a method for determining the presence of Syk in a sample, comprising contacting the sample with a compound of formula I, Ia, Ib, Ic, Id or Ie, or a pharmaceutically acceptable salt, ester, prodrug, or solvate thereof under conditions that permit detection of Syk activity, detecting a level of Syk activity in the sample, and therefrom determining the presence or absence of Syk in the sample.

Also provided is a method for inhibiting B-cell activity, comprising contacting cells expressing Syk with a compound of formula I, Ia, Ib, Ic, Id or Ie, or a pharmaceutically acceptable salt, ester, prodrug, or solvate thereof in an amount sufficient to detectably decrease B-cell activity in vitro.

Also provided is a use of a compound of formula I, Ia, Ib, Ic, Id or Ie, or a pharmaceutically acceptable salt, ester, prodrug, or solvate thereof, in the manufacture of a medicament for the treatment of a disease responsive to inhibition of Syk activity.

Also provided are kits that include a compound of formula I, Ia, Ib, Ic, Id or Ie, or a pharmaceutically acceptable salt, ester, prodrug, or solvate thereof. In one embodiment, the kit further includes instructions for use. In one aspect, a kit includes a compound of formula I, Ia, Ib, Ic, Id or Ie, or a pharmaceutically acceptable salt, ester, prodrug, or solvate thereof, and instructions for use of the compounds in the treatment of the diseases described above.

Also provided are articles of manufacture that include a compound of formula I, Ia, Ib, Ic, Id or Ie, or a pharmaceutically acceptable salt, ester, prodrug, or solvate thereof. In one embodiment, the container may be a vial, jar, ampoule, preloaded syringe, or an intravenous bag.

### DETAILED DESCRIPTION

The following description sets forth exemplary methods and parameters. It should be recognized, however, that such description is not intended as a limitation on the scope of the present disclosure but is instead provided as a description of exemplary embodiments.

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

As used in the present specification, the following words, phrases and symbols are generally intended to have the meanings as set forth below, except to the extent that the context in which they are used indicates otherwise. The following abbreviations and terms have the indicated meanings throughout.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “unsubstituted alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms. In some embodiments, alkyl as used in compounds of formula I, Ia, Ib, Ic, Id and Ie has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). For example C₁₋₆ alkyl encompasses both straight and branched chain alkyl of from 1 to 6 carbon atoms. Examples of alkyl groups include methyl, ethyl, propyl, isopropyl, n-butyl, sec-butyl, test-butyl, pentyl, 2-pentyl, isopentyl, neopentyl, hexyl, 2-hexyl, 3-hexyl, 3-methylpentyl. “Alkylene” is a subset of alkyl, referring to the same residues as alkyl, but having two points of attachment. Alkylene groups may, in some embodiments, have from 2 to 20 carbon atoms (i.e., C₂₋₂₀ alkylene), 2 to 8 carbon atoms (i.e., C₂₋₈ alkylene), 2 to 6 carbon atoms (i.e., C₂₋₆ alkylene), or 2 to 4 atoms (i.e., C₂₋₄ alkylene). For example, C₀ alkylene indicates a covalent bond and C₁ alkylene is a methylene group. When an alkyl residue having a specific number of carbons is named, all geometric isomers having that number of carbons may be encompassed; thus, for example, “butyl” can include n-butyl, sec-butyl, isobutyl and t-butyl; “propyl” can include n-propyl and isopropyl. In some embodiments, “lower alkyl” refers to alkyl groups having 1 to 4 carbons (i.e., C₁₋₄ alkyl).

“Alkenyl” indicates an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Alkenyl groups may include, for example, ethenyl; propenyls such as prop-1-en-1-yl, prop-1-en-2-yl, prop-2-en-1-yl (allyl), prop-2-en-2-yl; butenyls such as hut-1-en-1-yl, but-1-en-2-yl, 2-methylprop-1-en-1-yl, but-2-en-1-yl, but-2-en-1-yl, but-2-en-2-yl, buta-1,3-dien-2-yl. In some embodiments, an alkenyl group has from 2 to 20 carbon atoms (i.e., C₂₋₂₀ alkenyl), or 2 to 6 carbon atoms (i.e., C₂₋₆ alkenyl).

“Cycloalkyl” refers to a saturated hydrocarbon ring group, having the specified number of carbon atoms. In some embodiments, cycloalkyl as used in compounds of formula I, Ia, Ib, Ic, Id and Ie has from 3 to 20 ring carbon atoms (i.e., C₃₋₂₀ cycloalkyl), or 3 to 12 ring carbon atoms (i.e., C₃₋₁₂ cycloalkyl), or 3 to 8 ring carbon atoms (i.e., C₃₋₈ cycloalkyl). Examples of cycloalkyl groups include cyclopropyl, cyclobutyl, cyclopentyl, and cyclohexyl. In certain embodiments, cycloalkyl may also include bridged and caged saturated ring groups such as norbornane.

“Cycloalkenyl” refers to an unsaturated hydrocarbon ring group having at least one carbon-carbon double bond within the ring. An example of a cycloalkenyl group is cyclohexene, “Heterocycloalkenyl” refers to an unsaturated hydrocarbon ring group having at least one carbon-carbon double bond within the ring, with one or more heteroatoms selected from nitrogen, oxygen, and sulfur within the ring. An example of a heterocycloalkenyl group is dihydropyran,

By “alkoxy” is meant an alkyl group of the indicated number of carbon atoms attached through an oxygen bridge. Alkoxy groups include, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In some embodiments, alkoxy as used in compounds of formula I, Ia, Ib, Ic, Id and Ie has from 1 to 20 carbon atoms (i.e., C₁₋₂₀ alkoxy), 1 to 8 carbon atoms (i.e., C₁₋₈ alkoxy), 1 to 6 carbon atoms (i.e., C₁₋₆ alkoxy), or 1 to 4 carbon atoms (i.e., C₁₋₄ alkoxy) attached through the oxygen bridge. In certain embodiments, “lower alkoxy” refers to alkoxy groups having 1 to 4 carbons.

“Aminocarbonyl” encompasses a group of the formula —C(O)NRR. In some embodiments, each R is independently chosen from hydrogen and the optional substituents for “substituted amino” described below.

“Acyl” refers to the groups (alkyl)-C(O); (cycloalkyl)-C(O); (aryl)-C(O); (heteroaryl)-C(O); and (heterocycloalkyl)-C(O), wherein the group is attached to the parent structure through the carbonyl carbon, and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃C(O), attached to the parent structure through the carbonyl carbon.

By “alkoxycarbonyl” is meant an ester group of the formula (alkoxy)-C(O) attached through the carbonyl carbon, wherein the alkoxy group has the indicated number of carbon atoms. In some embodiments, a C₁₋₆ alkoxycarbonyl group is an alkoxy group having from 1 to 6 carbon atoms attached through its oxygen to a carbonyl carbon.

By “amino” is meant the group —NH₂.

“Aryl” refers to an aromatic carbocyclic group having a single ring (e.g., phenyl), multiple rings (e.g., biphenyl), or multiple fused rings (e.g., naphthyl). In some embodiments, aryl includes 5- and 6-membered carbocyclic aromatic rings. In other embodiments, aryl includes bicyclic ring systems wherein at least one ring is carbocyclic and aromatic, for example, naphthalene, indane, and tetralin. In yet other embodiments, aryl includes bicyclic ring systems wherein at least one ring is carbocyclic and aromatic, for example, fluorene. In certain embodiments, aryl as used in compounds of formula I, Ia, Ib, Ic, Id and Ie has 3 to 20 ring carbon atoms (i.e., C₃₋₂₀ aryl), 3 to 12 carbon ring atoms (i.e., C₃₋₁₂ aryl), or 3 to 8 carbon ring atoms (i.e., C₃₋₈ aryl). Aryl, however, does not encompass or overlap in any way with heteroaryl, separately defined below. In certain embodiments, if one or more aryl groups are fused with a heteroaryl ring, the resulting ring system is heteroaryl.

The term “aryloxy” refers to the group —O-aryl.

The term “halogen” or “halo” includes fluoro, chloro, bromo, and iodo, and the term “halogen” includes fluorine, chlorine, bromine, and iodine. “Haloalkyl” refers to unbranched or branched chain alkyl group as defined above, wherein one or more hydrogen atoms are substituted by a halogen. For example, where a residue is substituted with more than one halogen, it may be referred to by using a prefix corresponding to the number of halogen moieties attached. For example, dihaloaryl, dihaloalkyl, and trihaloaryl refer to aryl and alkyl substituted with two (“di”) or three (“tri”) halo groups, which may be, but are not necessarily, the same halogen; thus, for example, 4-chloro-3-fluorophenyl is within the scope of dihaloaryl. An alkyl group in which each H is replaced with a halo group is referred to as a “perhaloalkyl.” One example of a perhaloalkyl group is trifluoromethyl (—CF₃). Similarly, “haloalkoxy” refers to an alkoxy group in which one or more hydrogen atoms are substituted by a halogen in the hydrocarbon making up the alkyl moiety of the alkoxy group. Examples of a haloalkoxy group include difluoromethoxy (—OCHF₂) or trifluoromethoxy (—OCF₃).

“Heteroaryl” refers to an aromatic group with a single ring, multiple rings, or multiple fused rings, with one or more heteroatoms selected from nitrogen, oxygen, and sulfur within at least one ring. In some embodiments, heteroaryl is an aromatic, monocyclic or bicyclic ring containing one or more heteroatoms chosen from nitrogen and oxygen with the remaining ring atoms being carbon. In certain embodiments, heteroaryl as used in compounds of formula I, Ia, Ib, Ic, Id and Ie has 3 to 20 ring atoms (i.e., C₃₋₂₀ heteroaryl), 3 to 12 ring atoms (i.e., C₃₋₁₂ heteroaryl), or 3 to 8 ring atoms (i.e., C₃₋₈ heteroaryl). In other embodiments, heteroaryl as used in compounds of formula I, Ia, Ib, Ic, Id and Ie has 1 to 5 heteroatoms, 1 to 4 heteroatoms, 1 to 3 heteroatoms, 1 or 2 heteroatoms, or 1 heteroatom chosen from nitrogen and oxygen in at least one ring. Examples of heteroaryl groups include, but are not limited to, 2-pyridyl, 3-pyridyl, 4-pyridyl, 2,3-pyrazinyl, 3,4-pyrazinyl, 3,5-pyrimidinyl, 2,3-pyrazolinyl, 2,4-isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, and pyrazolyl. Heteroaryl does not encompass or overlap with aryl as defined above.

The term “heteroaryloxy” refers to the group —O-heteroaryl

“Heterocycle”, “heterocyclic”, or “heterocyclyl” refers to a saturated or an unsaturated non-aromatic group having a single ring or multiple condensed rings, and at least one heteroatom independently selected from nitrogen, oxygen and sulfur. In some embodiments, heterocycle groups have 1 to 20 ring atoms (i.e., C₁₋₂₀ heterocycle), 1 to 12 ring atoms (i.e., C₁₋₁₂ heterocycle), or 1 to 8 ring atoms (i.e., C₁₋₈ heterocycle). In other embodiments, heterocycle groups have 1 to 5 heteroatoms, 1 to 4 heteroatoms, 1 to 3 heteroatoms, 1 or 2 heteroatoms, or 1 heteroatom selected form nitrogen, sulfur or oxygen in at least one ring. In certain embodiments, a heterocycle that has more than one ring may be fused, spiro or bridged, or any combination thereof.

By “heterocycloalkyl” refers to a cyclic alkyl group containing at least two carbon atoms, and at least one heteroatom independently selected from nitrogen, oxygen and sulfur. In some embodiments, the heterocycloalkyl of formula I, Ia, Ib, Ic, Id or Ie has 3 to 20 ring atoms (i.e., C₃₋₂₀ heterocycloalkyl), 3 to 12 ring atoms (i.e., C₃₋₁₂ heterocycloalkyl), or 3 to 8 ring atoms (i.e., C₃₋₈ heterocycloalkyl). In other embodiments, heterocycloalkyl groups have 1 to 5 heteroatoms, 1 to 4 heteroatoms, 1 to 3 heteroatoms, 1 or 2 heteroatoms, or 1 heteroatom selected form nitrogen, sulfur or oxygen in at least one ring. Examples of heterocycloalkyl groups may include 1-pyrrolidinyl, 2-pyrrolidinyl, 1-piperidinyl, 2-piperidinyl, 3-piperidinyl, 4-piperidinyl, 1-piperazinyl, 2-piperazinyl, 2-oxetanyl, 3-oxetanyl, 1,3-dioxolanyl, 1-azetidinyl, and 2-azetidinyl. Morpholinyl (also referred to as morpholino) groups are also contemplated, including 2-morpholinyl, 3-morpholinyl, 4-morpholinyl, homomorpholinyl, 1-thiomorpholinyl, 2-thiomorpholinyl, 3-thiomorpholinyl, 4-thiomorpholinyl, 1-thiomorpholinyl S-oxide, 2-thiomorpholinyl S-oxide, 3-thiomorpholinyl S-oxide, 4-thiomorpholinyl S-oxide, 1-thiomorpholinyl sulfone, 2-thiomorpholinyl sulfone, 3-thiomorpholinyl sulfone, and 4-thiomorpholinyl sulfone. Substituted heterocycloalkyl also includes ring systems substituted with one or more oxo moieties, such as mopholinyl-3-one, morpholinyl-N-oxide, 1-oxo-1-thiomorpholinyl and 1,1-dioxo-1-thiomorpholinyl. For example, in one embodiment, the heterocycloalkyl is selected from unsubstituted morpholinyl, substituted morpholinyl, unsubstituted homomorpholinyl, substituted homomorpholinyl, unsubstituted piperazinyl, substituted piperazinyl, unsubstituted piperidinyl, substituted piperidinyl, unsubstituted pyrrolidinyl, substituted pyrrolidinyl, unsubstituted azetidinyl, and substituted azetidinyl. In some embodiments, heterocycloalkyl may have more than one ring, where additional rings may be fused, spiro or bridged, or any combination thereof. Examples of a fused heterocycloalkyl group include hexahydro-1H-[1,4]oxazino[3,4-c][1,4]oxazine, octahydropyrazino[2,1-c][1.4]oxazine and hexahydro-1H-furo[3,4-c]pyrrole. Examples of a spiro heterocycloalkyl group include 1-oxa-3,7-diazaspiro[4.5]decane and 1-oxa-6-azaspiro[3.4]octanyl.

The term “heterocycloalkyloxy” refers to the group —O-heterocylcoalkyl.

The term “nitro” refers to the group

The term “oxime” refers to the group —CR^{s}(═N)OR^{t}, wherein R^{s} is null, hydrogen or alkyl, and R^{t} refers to hydrogen or alkyl. For example, when Rₐ is null, the carbon atom of the C(═N) moiety is part of the parent structure, and the oxime group is attached by the double bond of the nitrogen atom to the parent structure (e.g., piperidin-4-one O-methyl oxime). When R^{s} is hydrogen or alkyl, the oxime group is attached by the carbon atom of the C(═N) moiety to the parent structure (e.g., ethanone O-methyl oxime).

The term “phosphono” refers to the group —PO₃H₂.

“Thiocarbonyl” refers to the group —C(O)SH.

The term “optionally substituted thiocarbonyl” includes the following groups: —C(O)S—(optionally substituted C₁₋₅ alkyl), —C(O)S-(optionally substituted aryl), —C(O)S—(optionally substituted heteroaryl), and C(O)S-(optionally substituted heterocycloalkyl).

The term “sulfanyl” includes the groups: —S-(optionally substituted C₁₋₆ alkyl), —S-(optionally substituted aryl), —S-(optionally substituted heteroaryl), and —S-(optionally substituted heterocycloalkyl). Hence, sulfanyl includes the group C₁₋₆ alkylsulfanyl.

The term “sulfinyl” includes the groups: —S(O)—H, —S(O)-(optionally substituted C₁₋₆ alkyl), —S(O)-optionally substituted aryl), —S(O)-optionally substituted heteroaryl), —S(O)-(optionally substituted heterocycloalkyl); and —S(O)-(optionally substituted amino).

The term “sulfonyl” includes the groups: —S(O₂)—H, —S(O₂)-(optionally substituted C₁₋₅ alkyl), —S(O₂)-optionally substituted aryl), —S(O₂)-optionally substituted heteroaryl), —S(O₂)— (optionally substituted heterocycloalkyl), —S(O₂)-(optionally substituted alkoxy), —S(O₂)-optionally substituted aryloxy), —S(O₂)-optionally substituted heteroaryloxy), —S(O₂)-(optionally substituted heterocyclyloxy); and —S(O₂)-(optionally substituted amino).

The term “substituted”, as used herein, means that any one or more hydrogens on the designated atom or group is replaced with a selection from the indicated group, provided that the designated atom's normal valence is not exceeded. When a substituent is oxo (i.e., ═O), then two hydrogens on the atom are replaced. Combinations of substituents and/or variables are permissible if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure.

“Substituted alkyl” refers to an alkyl group having one or more substitutents including, but not limited to, groups such as optionally substituted alkenyl, optionally substituted alkynyl, optionally substituted cycloalkyl, optionally substituted heterocycloalkyl, optionally substituted aryl, optionally substituted heteroaryl, optionally substituted alkoxy, optionally substituted cycloalkyloxy, optionally substituted heterocycloalkyloxy, optionally substituted amino, optionally substituted sulfonyl, oxime, cyano, oxo, halo, hydroxyl, nitro, carboxyl, and thiol. In some embodiments, a substituted alkyl may have 1 to 5 substituents, 1 to 3 substituents, 1 to 2 substituents, or 1 substituent.

“Substituted cycloalkyl” refers to a cycloalkyl group having one or more substitutents including, but not limited to, groups such as optionally substituted alkenyl, optionally substituted alkynyl, optionally substituted alkyl, optionally substituted cycloalkyl, optionally substituted heterocycloalkyl, optionally substituted aryl, optionally substituted heteroaryl, optionally substituted alkoxy, optionally substituted cycloalkyloxy, optionally substituted heterocycloalkyloxy, optionally substituted amino, optionally substituted sulfonyl, oxime, cyano, oxo, halo, hydroxyl, nitro, carboxyl, and thiol. In some embodiments, a substituted cycloalkyl may have 1 to 5 substituents, 1 to 3 substituents, 1 to 2 substituents, or 1 substituent.

“Substituted aryl” refers to an aryl group having one or more substitutents including, but not limited to, groups such as optionally substituted alkenyl, optionally substituted alkynyl, optionally substituted alkyl, optionally substituted cycloalkyl, optionally substituted heterocycloalkyl, optionally substituted aryl, optionally substituted heteroaryl, optionally substituted alkoxy, optionally substituted cycloalkyloxy, optionally substituted heterocycloalkyloxy, optionally substituted amino, optionally substituted sulfonyl, oxime, cyano, oxo, halo, hydroxyl, nitro, carboxyl, and thiol. In some embodiments, a substituted aryl may have 1 to 5 substituents, 1 to 3 substituents, 1 to 2 substituents, or 1 substituent.

“Substituted heteroaryl” refers to an aryl group having one or more substitutents including, but not limited to, groups such as optionally substituted alkenyl, optionally substituted alkynyl, optionally substituted alkyl, optionally substituted cycloalkyl, optionally substituted heterocycloalkyl, optionally substituted aryl, optionally substituted heteroaryl, optionally substituted alkoxy, optionally substituted cycloalkyloxy, optionally substituted heterocycloalkyloxy, optionally substituted amino, optionally substituted sulfonyl, oxime, cyano, oxo, halo, hydroxyl, nitro, carboxyl, and thiol. In some embodiments, a substituted heteroaryl may have 1 to 5 substituents, 1 to 3 substituents, 1 to 2 substituents, or 1 substituent.

The term “substituted acyl” refers to the groups (substituted alkyl)-C(O)-(substituted cycloalkyl)-C(O); (substituted aryl)-C(O); (substituted hetero aryl)-C(O); and (substituted heterocycloalkyl)-C(O), wherein the group is attached to the parent structure through the carbonyl carbon, and wherein substituted alkyl, cycloalkyl, aryl, heteroaryl, and heterocycloalkyl are as described herein.

The term “substituted alkoxy” refers to alkoxy wherein the alkyl constituent is substituted, including for example, —O-(substituted alkyl), wherein “substituted alkyl” is as described herein.

The term “substituted alkoxycarbonyl” refers to the group (substituted alkyl)-O—C(O), wherein the group is attached to the parent structure through the carbonyl carbon, and wherein “substituted alkyl” is as described herein.

The term “substituted aryloxy” refers to aryloxy wherein the aryl constituent is substituted, including for example, —O-(substituted aryl), wherein “substituted aryl” is as described herein.

The term “substituted heteroaryloxy” refers to heteroaryloxy wherein the aryl constituent is substituted, including for example, —O-(substituted heteroaryl) wherein “substituted heteroaryl” is as described herein.

The term “substituted cycloalkyloxy” refers to cycloalkyloxy wherein the cycloalkyl constituent is substituted, including for example, —O-(substituted cycloalkyl), wherein “substituted cycloalkyl” is as described herein.

The term “substituted heterocycloalkyloxy” refers to heterocycloalkyloxy wherein the alkyl constituent is substituted, including for example, —O-(substituted heterocycloalkyl) wherein “substituted heterocycloalkyl” is as described herein.

The term “substituted amino” refers to the group —NHR or —NRR where each R is independently chosen from, for example, hydroxy, optionally substituted C₁₋₆ alkyl, optionally substituted cycloalkyl, optionally substituted acyl, optionally substituted aminocarbonyl, optionally substituted aryl, optionally substituted heteroaryl, optionally substituted heterocycloalkyl, alkoxycarbonyl, sulfinyl and sulfonyl, provided that only one R may be hydroxyl.

In one aspect, provided is a compound having the structure of formula (I):

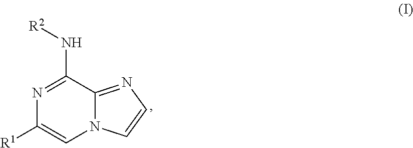

or a pharmaceutically acceptable salt, ester, prodrug, or solvate thereof, wherein:

R¹ is

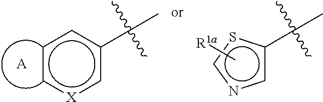

wherein:
A is selected from the group consisting of unsubstituted morpholinyl, substituted morpholinyl, unsubstituted homomorpholinyl, substituted homomorpholinyl, unsubstituted thiomorpholinyl, substituted thiomorpholinyl, unsubstituted thiomorpholinyl S-oxide, substituted thiomorpholinyl S-oxide, unsubstituted thiomorpholinyl sulfone, substituted thiomorpholinyl sulfone, unsubstituted piperidinyl and substituted piperidinyl; and
X is N or CR^{x}, wherein R^{x} is hydrogen or C₁₋₆ alkyl;
R^{1a} is selected from the group consisting of unsubstituted alkyl, substituted alkyl, unsubstituted cycloalkyl, substituted cycloalkyl, unsubstituted heterocycloalkyl, and substituted heterocycloalkyl;

R² is

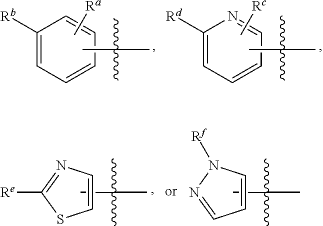

wherein:
R^{a} is selected from the group consisting of hydrogen, halo, and unsubstituted alkoxy;
R^{b} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted heterocycloalkenyl, substituted heterocycloalkenyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy;
   or R^{a} and R^{b} are taken together with the carbons to which they are attached to form a heterocyclyl ring or heteroaryl ring containing 1-3 heteroatoms selected from the group consisting of N and 0;
R^{c} is hydrogen;
R^{d} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted heterocycloalkenyl, substituted heterocycloalkenyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy;
R^{e} is selected from the group consisting of hydrogen, unsubstituted alkyl, substituted alkyl, unsubstituted heterocycloalkyl, and substituted heterocycloalkyl; and
R^{f} is selected from the group consisting of hydrogen, unsubstituted alkyl, substituted alkyl, unsubstituted heterocycloalkyl, and substituted heterocycloalkyl.

In one embodiment, X is N. In another embodiment, X is CR^{x}. In one embodiment, R^{x} is hydrogen. In another embodiment, R^{x} is unsubstituted C₁₋₆ alkyl. In yet another embodiment, R^{x} is substituted C₁₋₆ alkyl.

In some embodiments, A is selected from the group consisting of:

unsubstituted morpholinyl;

substituted morpholinyl with one or two substituents selected from the group consisting of oxo, unsubstituted alkyl, and substituted alkyl;

unsubstituted homomorpholinyl;

substituted homomorpholinyl with one or two substituents selected from the group consisting of oxo, unsubstituted alkyl, and substituted alkyl;

unsubstituted thiomorpholinyl;

unsubstituted thiomorpholinyl S-oxide;

unsubstituted thiomorpholinyl sulfone; and

unsubstituted piperidinyl.

In some embodiments, R is

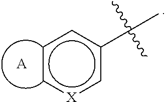

In certain embodiments, R is selected from the group consisting of:

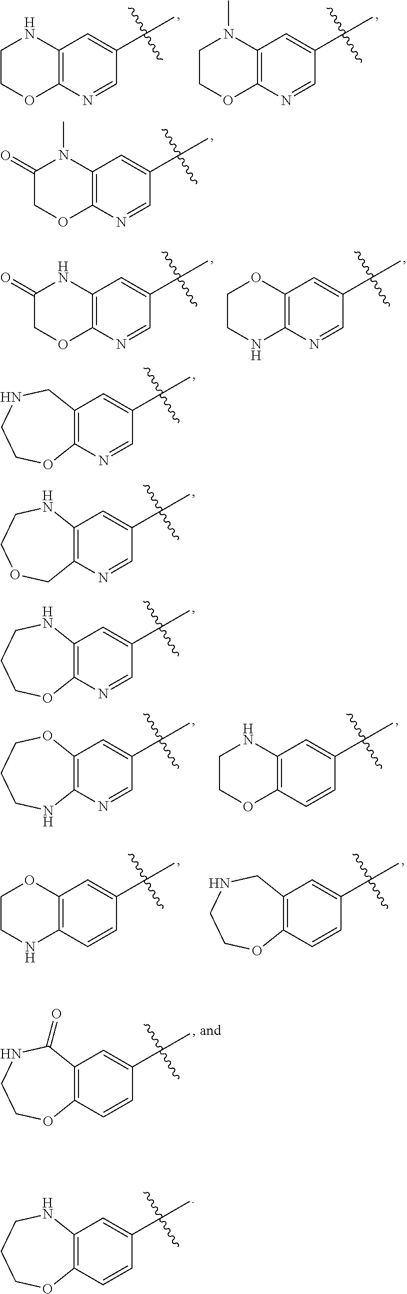

In certain embodiments, R¹ is selected from the group consisting of:

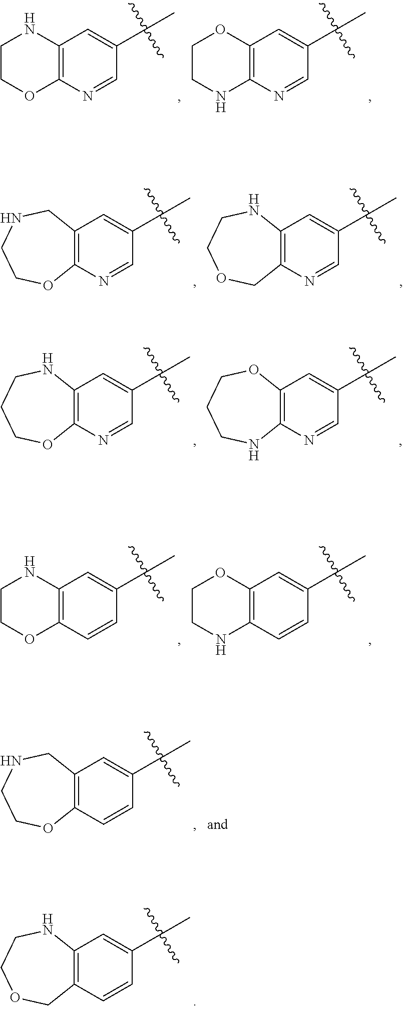

In yet other embodiments, R¹ is selected from the group consisting of:

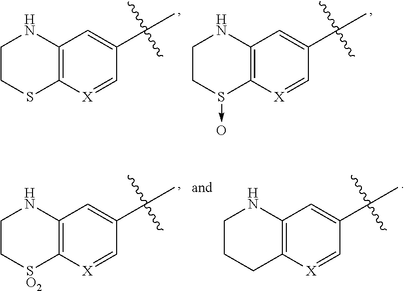

In certain embodiments, R¹ is selected from the group consisting of:

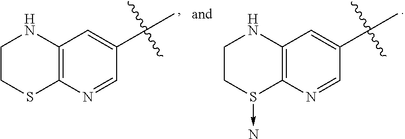 [SMILES: "," ; "and" ; "." ; *c1cnc2c(c1)NCCS2 ; *c1cnc2c(c1)NCCS2=N]

In some embodiments, R¹ is

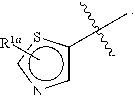

In one embodiment, R¹ is

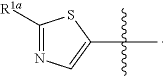

In certain embodiments, R^{1a} is selected from the group consisting of:

hydrogen;

unsubstituted alkyl;

substituted alkyl with one or two substituents selected from the group consisting of hydroxyl and perhaloalkyl;

unsubstituted cycloalkyl;

substituted cycloalkyl with a hydroxyl substituent;

unsubstituted heterocycloalkyl; and

substituted heterocycloalkyl with a hydroxyl substituent.

In certain embodiments, R¹ is selected from the group consisting of:

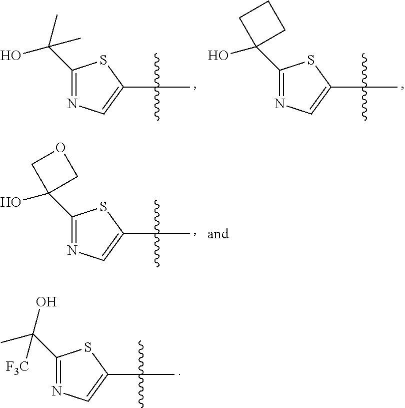

In some embodiments, R^{a} is unsubstituted alkoxy. In certain embodiments, R^{a} is selected from the group consisting of hydrogen, fluoro, methoxy, and ethoxy.

In some embodiments, R^{b} is selected from the group consisting of unsubstituted alkyl, substituted alkyl, unsubstituted cycloalkyl, substituted cycloalkyl, unsubstituted morpholinyl, substituted morpholinyl, unsubstituted piperazinyl, substituted piperazinyl, unsubstituted homomorpholinyl, substituted homomorpholinyl, unsubstituted piperidinyl, substituted piperidinyl, unsubstituted pyrrolidinyl, substituted pyrrolidinyl, unsubstituted azetidinyl, substituted azetidinyl, unsubstituted heterocycloalkenyl, substituted heterocycloalkenyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy.

In other embodiments, R^{b} is selected from the group consisting of unsubstituted morpholinyl, substituted morpholinyl, unsubstituted piperazinyl, and substituted piperazinyl. In certain embodiments, R^{b} is:

unsubstituted morpholinyl; or

substituted morpholinyl with one or two substituents independently selected from the group consisting of unsubstituted alkyl, substituted alkyl, unsubstituted heterocycloalkyl, substituted heterocycloalkyl, substituted amino, and aminocarbonyl.

In other embodiments, R^{b} is:

unsubstituted piperazinyl; or

substituted piperazinyl with one or two substituents independently selected from the group consisting of unsubstituted alkyl, substituted alkyl, unsubstituted heterocycloalkyl, substituted heterocycloalkyl, unsubstituted acyl, and substituted acyl.

In yet other embodiments, R^{b} is selected from the group consisting of methoxy, difluoromethoxy, dimethylamino, unsubstituted morpholinyl, substituted morpholino, substituted piperazinyl, substituted pyrrolidinyl, substituted azetidinyl, unsubstituted homomorpholinyl, substituted piperidinyl, unsubstituted cyclobutanyl, unsubstituted oxetanyl, substituted oxetanyl, unsubstituted dihydropyranyl, unsubstituted tetrahydropyranyl, and unsubstituted imidazolyl.

In some embodiments, R^{a} and R^{b} are taken together with the carbons to which they are attached to form a heterocyclyl ring containing 1-3 heteroatoms selected from the group consisting of N and O.

In some embodiments, R^{c} is hydrogen.

In some embodiments, R^{d} is selected from the group consisting of unsubstituted alkyl, substituted alkyl, unsubstituted cycloalkyl, substituted cycloalkyl, unsubstituted morpholinyl, substituted morpholinyl, unsubstituted piperazinyl, substituted piperazinyl, unsubstituted homomorpholinyl, substituted homomorpholinyl, unsubstituted piperidinyl, substituted piperidinyl, unsubstituted pyrrolidinyl, substituted pyrrolidinyl, unsubstituted azetidinyl, substituted azetidinyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, oxime, and haloalkoxy.

In one embodiment, R^{d} is unsubstituted morpholinyl.

In other embodiments, R^{b} and R^{d} are independently:

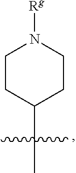

wherein R^{g} is selected from the group consisting of unsubstituted alkyl, substituted alkyl, unsubstituted alkoxy, substituted alkoxy, unsubstituted cycloalkyl, substituted cycloalkyl, unsubstituted heterocycloalkyl, and substituted heterocycloalkyl.

In other embodiments, R^{b} and R^{d} are independently

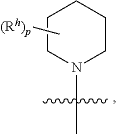

wherein:

p is 0, 1 or 2; and

R^{h} is selected from the group consisting of unsubstituted alkoxy, unsubstituted alkyl, substituted alkyl, hydroxyl, unsubstituted heterocycloalkyl, substituted heterocycloalkyl, halo, oxo, and oxime.

In yet other embodiments, R^{b} and R^{d} are independently

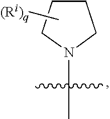

wherein:

q is 0, 1, or 2; and

Rⁱ is selected from the group consisting of unsubstituted amino, unsubstituted heterocycloalkyl, and substituted heterocycloalkyl.

In yet other embodiments, R^{b} and R^{d} are independently

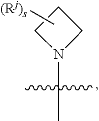

wherein:

s is 0, 1, or 2; and

R^{j} is selected from the group consisting of hydroxyl, unsubstituted alkyl, and substituted alkyl.

In yet other embodiments, R^{b} and R^{d} independently

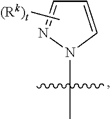

wherein:

t is 0, 1, or 2; and

R^{k} is selected from the group consisting of unsubstituted alkyl or substituted alkyl.

In yet other embodiments, R^{b} and R^{d} are independently

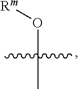

wherein:

R^{m} is selected from the group consisting of hydrogen, unsubstituted alkyl, substituted alkyl, unsubstituted alkoxy, substituted alkoxy, unsubstituted cycloalkyl, and substituted cycloalkyl, unsubstituted heterocycloalkyl, and substituted heterocycloalkyl.

In yet other embodiments, R^{b} and R^{d} independently

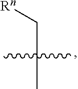

wherein:

Rⁿ is selected from the group consisting of hydrogen, unsubstituted alkyl, substituted alkyl, unsubstituted alkoxy, substituted alkoxy, unsubstituted cycloalkyl, and substituted cycloalkyl, unsubstituted heterocycloalkyl, and substituted heterocycloalkyl.

In yet other embodiments, R^{b} and R^{d} are independently

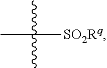

wherein:

R^{q} is unsubstituted alkyl.

In some embodiments, R² is

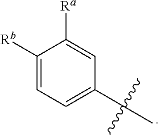

In other embodiments, R² is

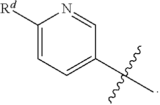

In yet other embodiments, R² is

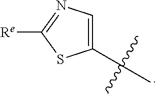

In yet other embodiments, R² is

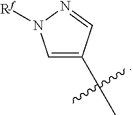

In certain embodiments, R² is selected from the group consisting of:

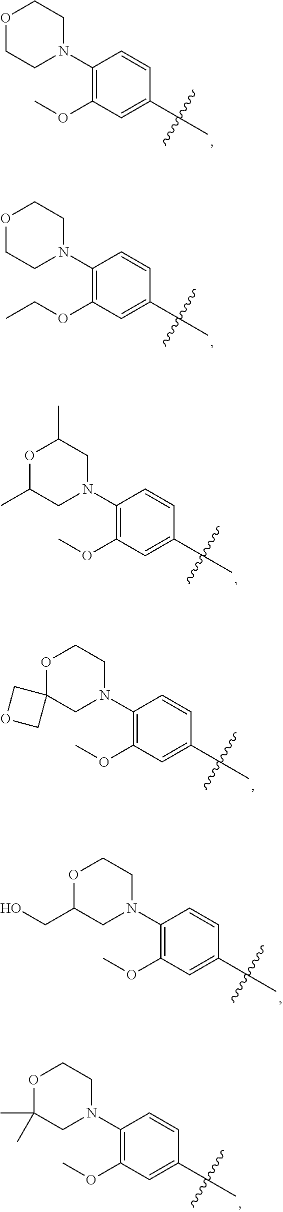 [SMILES: *c1ccc(N2CCOCC2)c(OC)c1 ; *c1ccc(N2CCOCC2)c(OCC)c1 ; *c1ccc(N2CC(C)OC(C)C2)c(OC)c1 ; *c1ccc(N2CCOC3(COC3)C2)c(OC)c1 ; *c1ccc(N2CCOC(CO)C2)c(OC)c1 ; *c1ccc(N2CCOC(C)(C)C2)c(OC)c1]

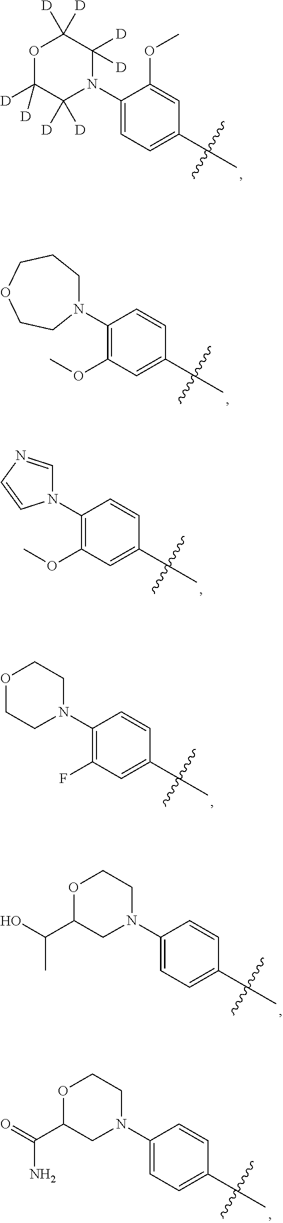 [SMILES: *c1ccc(N2C([2H])([2H])C([2H])([2H])OC([2H])([2H])C2([2H])[2H])c(OC)c1 ; *c1ccc(N2CCCOCC2)c(OC)c1 ; *c1ccc(-n2ccnc2)c(OC)c1 ; *c1ccc(N2CCOCC2)c(F)c1 ; *c1ccc(N2CCOC(C(C)O)C2)cc1 ; *c1ccc(N2CCOC(C(N)=O)C2)cc1]

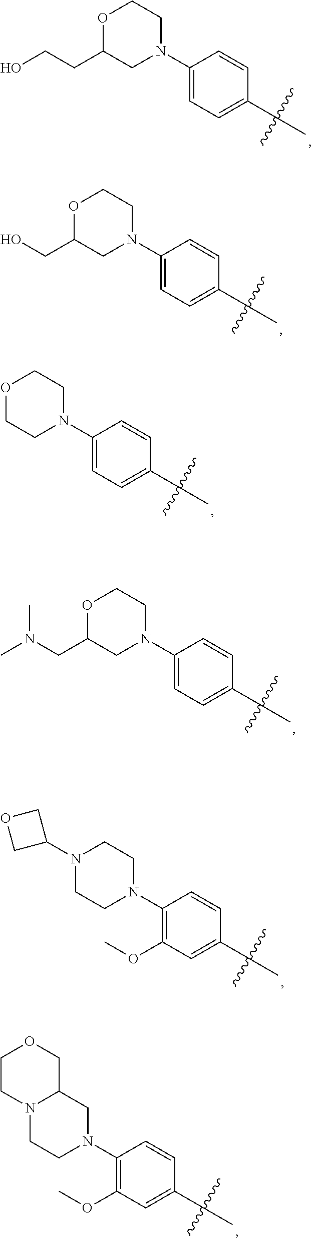 [SMILES: *c1ccc(N2CCOC(CCO)C2)cc1 ; *c1ccc(N2CCOC(CO)C2)cc1 ; *c1ccc(N2CCOCC2)cc1 ; *c1ccc(N2CCOC(CN(C)C)C2)cc1 ; *c1ccc(N2CCN(C3COC3)CC2)c(OC)c1 ; *c1ccc(N2CCN3CCOCC3C2)c(OC)c1]

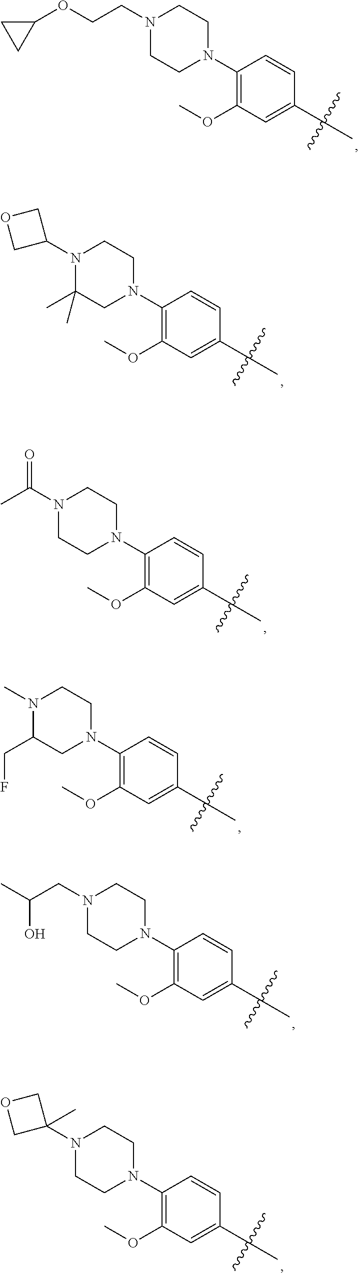 [SMILES: *c1ccc(N2CCN(CCOC3CC3)CC2)c(OC)c1 ; *c1ccc(N2CCN(C3COC3)C(C)(C)C2)c(OC)c1 ; *c1ccc(N2CCN(C(C)=O)CC2)c(OC)c1 ; *c1ccc(N2CCN(C)C(CF)C2)c(OC)c1 ; *c1ccc(N2CCN(CC(C)O)CC2)c(OC)c1 ; *c1ccc(N2CCN(C3(C)COC3)CC2)c(OC)c1]

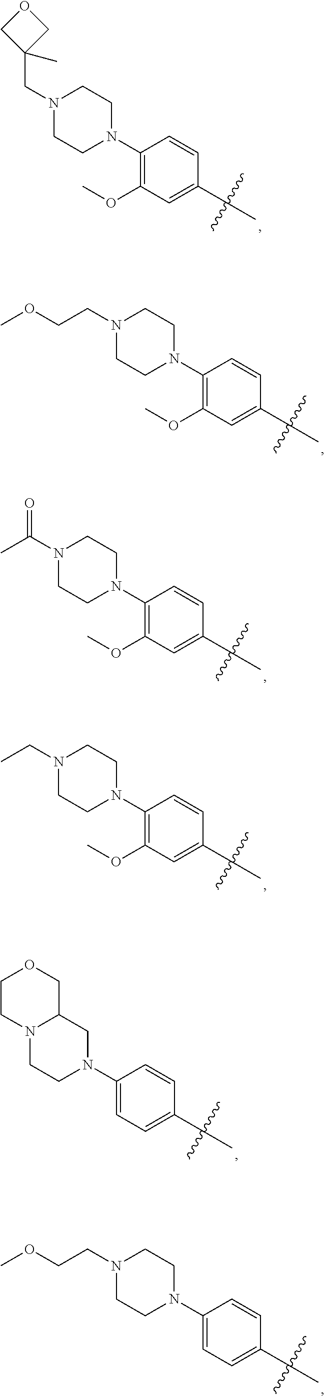 [SMILES: *c1ccc(N2CCN(CC3(C)COC3)CC2)c(OC)c1 ; *c1ccc(N2CCN(CCOC)CC2)c(OC)c1 ; *c1ccc(N2CCN(C(C)=O)CC2)c(OC)c1 ; *c1ccc(N2CCN(CC)CC2)c(OC)c1 ; *c1ccc(N2CCN3CCOCC3C2)cc1 ; *c1ccc(N2CCN(CCOC)CC2)cc1]

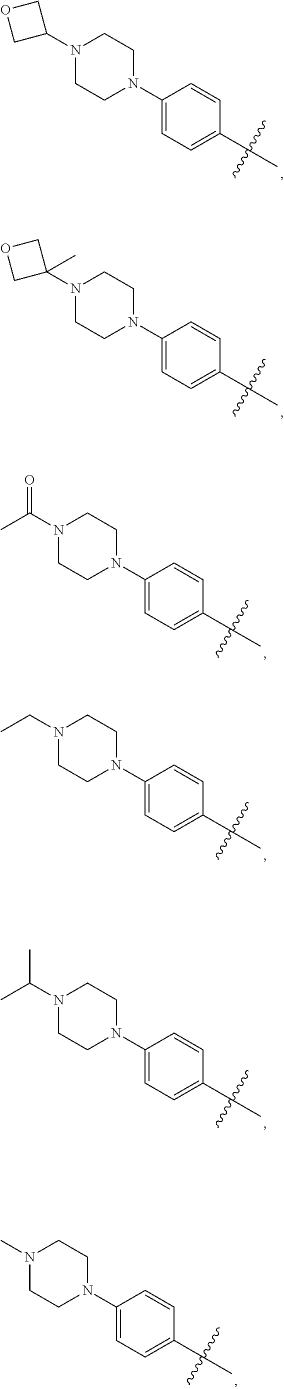 [SMILES: *c1ccc(N2CCN(C3COC3)CC2)cc1 ; *c1ccc(N2CCN(C3(C)COC3)CC2)cc1 ; *c1ccc(N2CCN(C(C)=O)CC2)cc1 ; *c1ccc(N2CCN(CC)CC2)cc1 ; *c1ccc(N2CCN(C(C)C)CC2)cc1 ; *c1ccc(N2CCN(C)CC2)cc1]

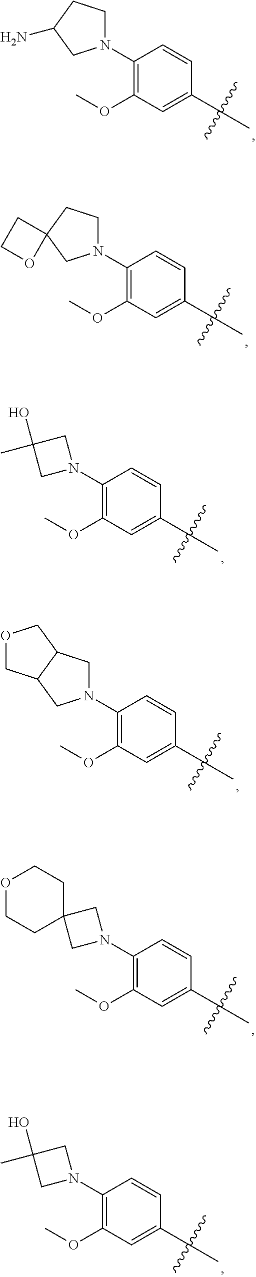 [SMILES: *c1ccc(N2CCC(N)C2)c(OC)c1 ; *c1ccc(N2CCC3(CCO3)C2)c(OC)c1 ; *c1ccc(N2CC(C)(O)C2)c(OC)c1 ; *c1ccc(N2CC3COCC3C2)c(OC)c1 ; *c1ccc(N2CC3(CCOCC3)C2)c(OC)c1 ; *c1ccc(N2CC(C)(O)C2)c(OC)c1]

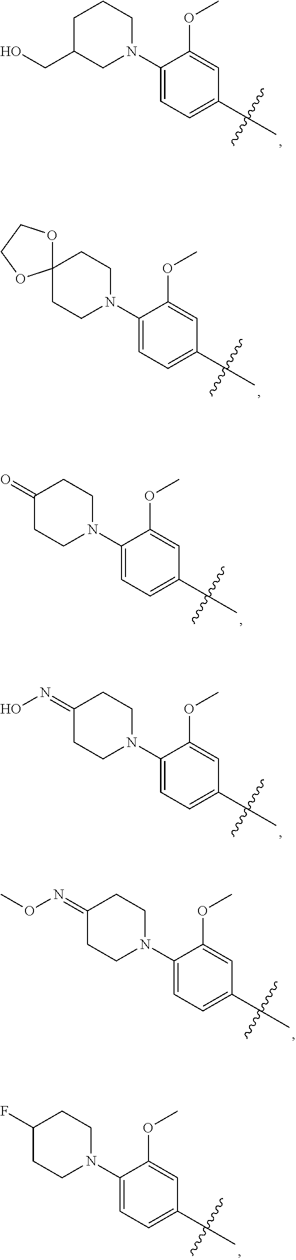 [SMILES: *c1ccc(N2CCCC(CO)C2)c(OC)c1 ; *c1ccc(N2CCC3(CC2)OCCO3)c(OC)c1 ; *c1ccc(N2CCC(=O)CC2)c(OC)c1 ; *c1ccc(N2CCC(=NO)CC2)c(OC)c1 ; *c1ccc(N2CCC(=NOC)CC2)c(OC)c1 ; *c1ccc(N2CCC(F)CC2)c(OC)c1]

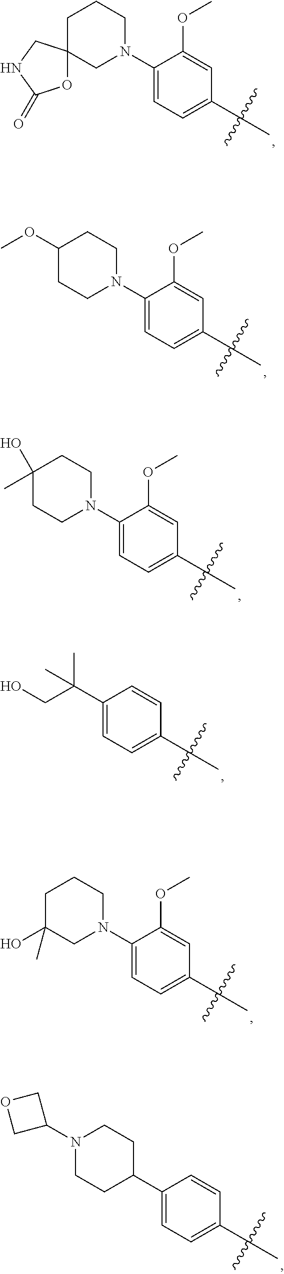 [SMILES: *c1ccc(N2CCCC3(CNC(=O)O3)C2)c(OC)c1 ; *c1ccc(N2CCC(OC)CC2)c(OC)c1 ; *c1ccc(N2CCC(C)(O)CC2)c(OC)c1 ; *c1ccc(C(C)(C)CO)cc1 ; *c1ccc(N2CCCC(C)(O)C2)c(OC)c1 ; *c1ccc(C2CCN(C3COC3)CC2)cc1]

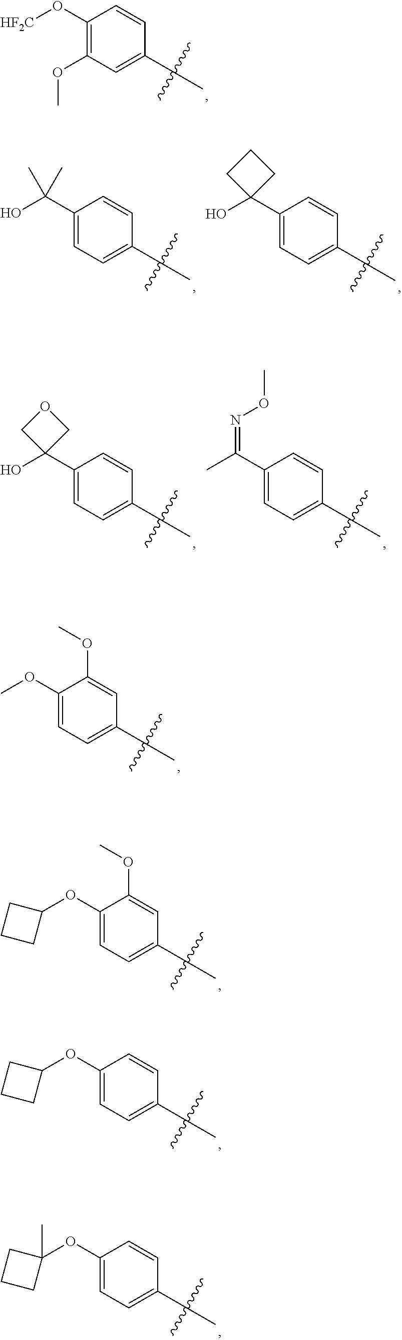

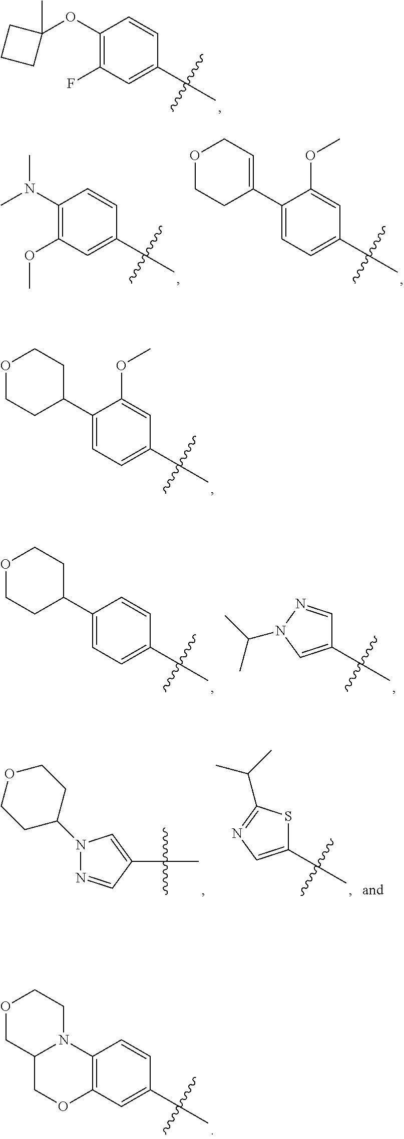

In certain embodiments, R² is selected from the group consisting of:

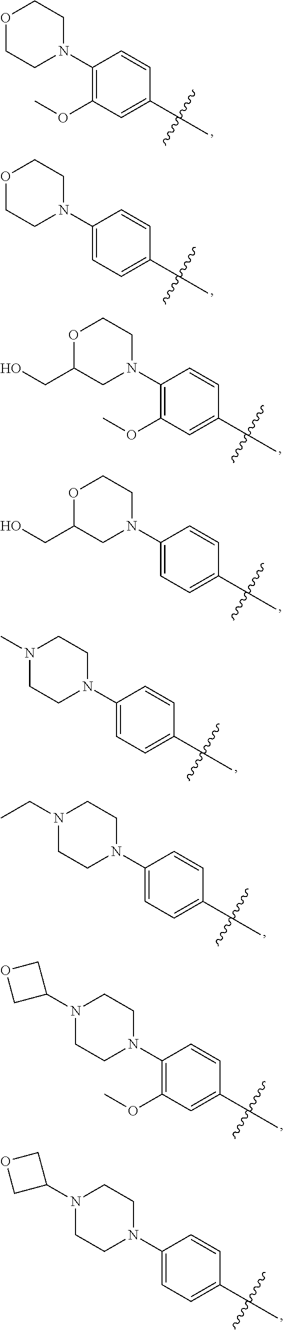

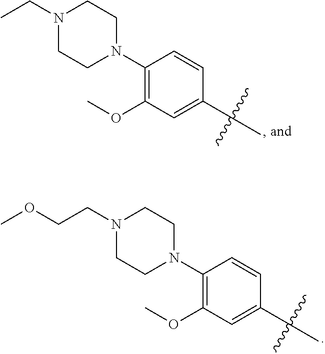 [SMILES: *c1ccc(N2CCN(CC)CC2)c(OC)c1 ; *c1ccc(N2CCN(CCOC)CC2)c(OC)c1]

In one embodiment, R² is selected from the group consisting of

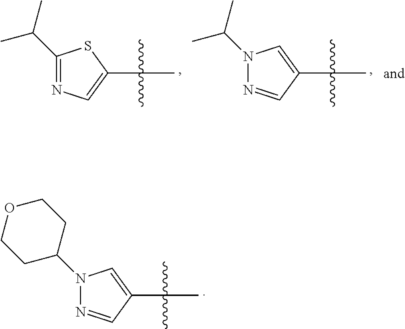

In some embodiments, the compound of formulae I, Ia, Ib, Ic, Id, and/or Ie is other than a compound in Table A below (as applicable). In one variation, provided is a compound of formula I, Ia, Ib, Ic, Id or Ie, or a pharmaceutically acceptable salt thereof, as well as pharmaceutical compositions comprising such compounds, and methods of using such compounds, provided that the compound is other than Compound No. 1x to 89x, or a pharmaceutically acceptable salt thereof.

TABLE A

| NO. | STRUCTURE | NAME |
|---|---|---|
| 1x | 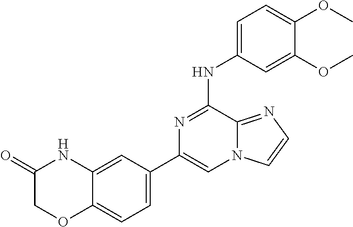 [SMILES: COc1ccc(Nc2nc(-c3ccc4c(c3)NC(=O)CO4)cn3ccnc23)cc1OC] | 6-{8-[(3,4- dimethoxyphenyl)amino]imidazo [1,2-a]pyrazin-6-yl}-3,4-dihydro-2H- 1,4-benzoxazin-3-one |
| 2x | 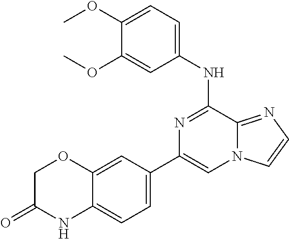 [SMILES: COc1ccc(Nc2nc(-c3ccc4c(c3)OCC(=O)N4)cn3ccnc23)cc1OC] | 7-{8-[(3,4- dimethoxyphenyl)amino]imidazo [1,2-a]pyrazin-6-yl}-3,4-dihydro-2H- 1,4-benzoxazin-3-one |
| 3x | 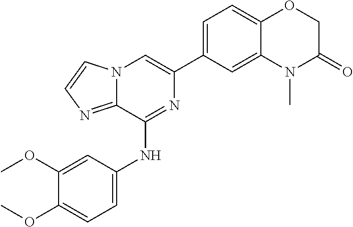 [SMILES: COc1ccc(Nc2nc(-c3ccc4c(c3)N(C)C(=O)CO4)cn3ccnc23)cc1OC] | 6-{8-[(3,4- dimethoxyphenyl)amino]imidazo [1,2-a]pyrazin-6-yl}-4-methyl-3,4- dihydro-2H-1,4-benzoxazin-3-one |
| 4x | 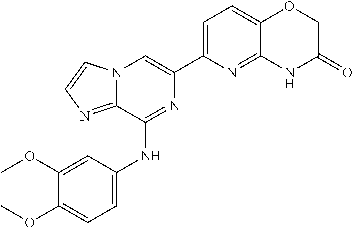 [SMILES: COc1ccc(Nc2nc(-c3ccc4c(n3)NC(=O)CO4)cn3ccnc23)cc1OC] | 6-{8-[(3,4- dimethoxyphenyl)amino]imidazo [1,2-a]pyrazin-6-yl}-2H,3H,4H- pyrido[3,2-b][1,4]oxazin-3-one |
| 5x | 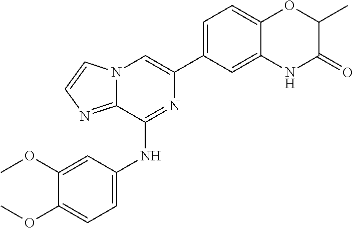 [SMILES: COc1ccc(Nc2nc(-c3ccc4c(c3)NC(=O)C(C)O4)cn3ccnc23)cc1OC] | 6-{8-[(3,4- dimethoxyphenyl)amino]imidazo [1,2-a]pyrazin-6-yl}-2-methyl-3,4- dihydro-2H-1,4-benzoxazin-3-one |
| 6x | 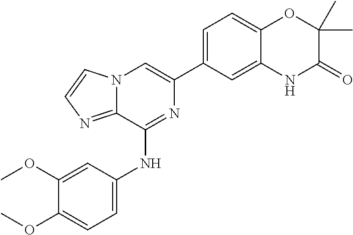 | 6-{8-[(3,4- dimethoxyphenyl)amino]imidazo [1,2-a]pyrazin-6-yl}-2,2-dimethyl-3,4- dihydro-2H-1,4-benzoxazin-3-one |
| 7x | 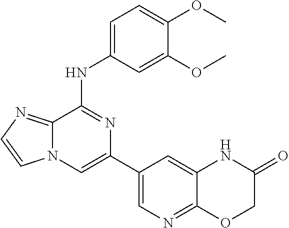 [SMILES: COc1ccc(Nc2nc(-c3cnc4c(c3)NC(=O)CO4)cn3ccnc23)cc1OC] | 7-{8-[(3,4- dimethoxyphenyl)amino]imidazo [1,2-a]pyrazin-6-yl}-1H,2H,3H- pyrido[2,3-b][1,4]oxazin-2-one |
| 8x | 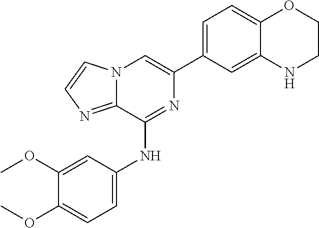 [SMILES: COc1ccc(Nc2nc(-c3ccc4c(c3)NCCO4)cn3ccnc23)cc1OC] | 6-(3,4-dihydro-2H-1,4-benzoxazin- 6-yl)-N-(3,4- dimethoxyphenyl)imidazo[1,2- a]pyrazin-8-amine |
| 9x | 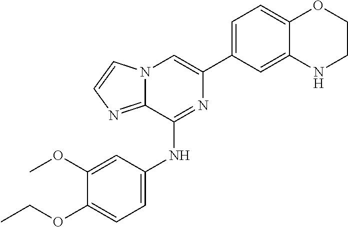 [SMILES: CCOc1ccc(Nc2nc(-c3ccc4c(c3)NCCO4)cn3ccnc23)cc1OC] | 6-(3,4-dihydro-2H-1,4-benzoxazin- 6-yl)-N-(4-ethoxy-3- methoxyphenyl)imidazo[1,2- a]pyrazin-8-amine |
| 10x | 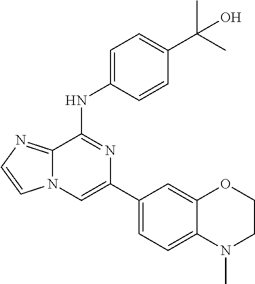 [SMILES: CN1CCOc2cc(-c3cn4ccnc4c(Nc4ccc(C(C)(C)O)cc4)n3)ccc21] | 2-(4-{[6-(4-methyl-3,4-dihydro-2H- 1,4-benzoxazin-7-yl)imidazo[1,2- a]pyrazin-8- yl]amino}phenyl)propan-2-ol |
| 11x | 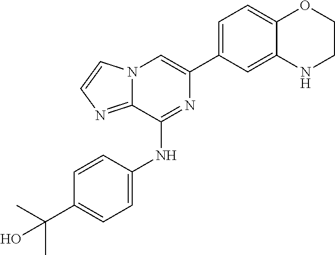 [SMILES: CC(C)(O)c1ccc(Nc2nc(-c3ccc4c(c3)NCCO4)cn3ccnc23)cc1] | 2-(4-{[6-(3,4-dihydro-2H-1,4- benzoxazin-6-yl)imidazo[1,2- a]pyrazin-8- yl]amino}phenyl)propan-2-ol |
| 12x | 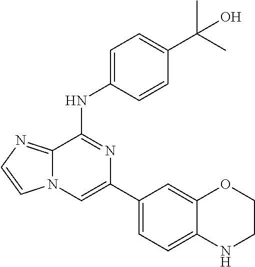 [SMILES: CC(C)(O)c1ccc(Nc2nc(-c3ccc4c(c3)OCCN4)cn3ccnc23)cc1] | 2-(4-{[6-(3,4-dihydro-2H-1,4- benzoxazin-7-yl)imidazo[1,2- a]pyrazin-8- yl]amino}phenyl)propan-2-ol |
| 13x | 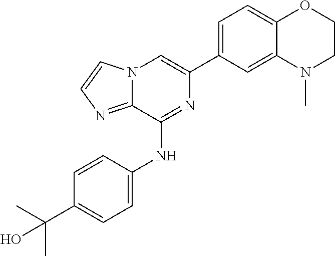 [SMILES: CN1CCOc2ccc(-c3cn4ccnc4c(Nc4ccc(C(C)(C)O)cc4)n3)cc21] | 2-(4-{[6-(4-methyl-3,4-dihydro-2H- 1,4-benzoxazin-6-yl)imidazo[1,2- a]pyrazin-8- yl]amino}phenyl)propan-2-ol |
| 14x | 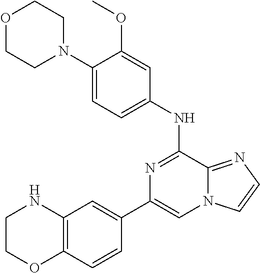 [SMILES: COc1cc(Nc2nc(-c3ccc4c(c3)NCCO4)cn3ccnc23)ccc1N1CCOCC1] | 6-(3,4-dihydro-2H- benzo[b][1,4]oxazin-6-yl)-N-(3- methoxy-4- moipholinophenyl)imidazo[1,2- a]pyrazin-8-amine |
| 15x | 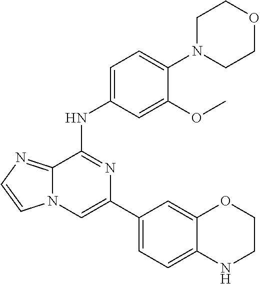 [SMILES: COc1cc(Nc2nc(-c3ccc4c(c3)OCCN4)cn3ccnc23)ccc1N1CCOCC1] | 6-(3,4-dihydro-2H-1,4-benzoxazin- 7-yl)-N-[3-methoxy-4-(morpholin-4- yl)phenyl]imidazo[1,2-a]pyrazin-8- amine |
| 16x | 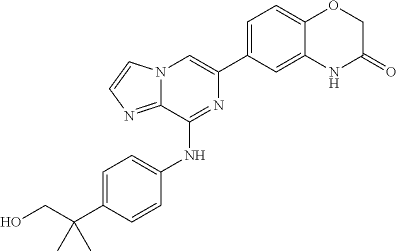 [SMILES: CC(C)(CO)c1ccc(Nc2nc(-c3ccc4c(c3)NC(=O)CO4)cn3ccnc23)cc1] | 6-(8-{[4-(1-hydroxy-2- methylpropan-2- yl)phenyl]amino}imidazo[1,2- a]pyrazin-6-yl)-3,4-dihydro-2H-1,4- benzoxazin-3-one |
| 17x | 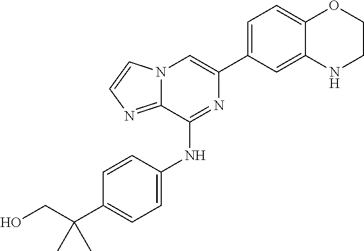 [SMILES: CC(C)(CO)c1ccc(Nc2nc(-c3ccc4c(c3)NCCO4)cn3ccnc23)cc1] | 2-(4-{[6-(3,4-dihydro-2H-1,4- benzoxazin-6-yl)imidazo[1,2- a]pyrazin-8-yl]amino}phenyl)-2- methylpropan-1-ol |
| 18x | 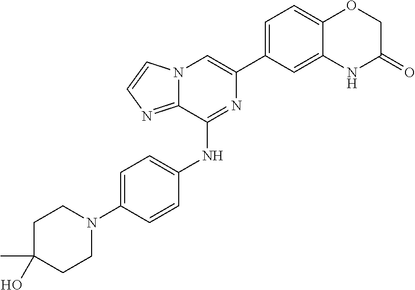 [SMILES: CC1(O)CCN(c2ccc(Nc3nc(-c4ccc5c(c4)NC(=O)CO5)cn4ccnc34)cc2)CC1] | 6-(8-{[4-(4-hydroxy-4- methylpiperidin-1- yl)phenyl]amino}imidazo[1,2- a]pyrazin-6-yl)-3,4-dihydro-2H-1,4- benzoxazin-3-one |
| 19x | 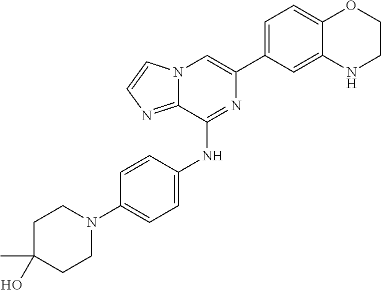 [SMILES: CC1(O)CCN(c2ccc(Nc3nc(-c4ccc5c(c4)NCCO5)cn4ccnc34)cc2)CC1] | 1-(4-{[6-(3,4-dihydro-2H-1,4- benzoxazin-6-yl)imidazo[1,2- a]pyrazin-8-yl]amino}phenyl)-4- methylpiperidin-4-ol |
| 20x | 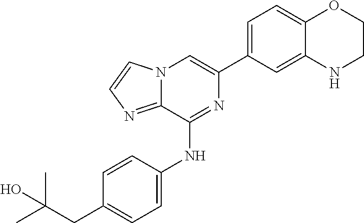 [SMILES: CC(C)(O)Cc1ccc(Nc2nc(-c3ccc4c(c3)NCCO4)cn3ccnc23)cc1] | 1-(4-{[6-(3,4-dihydro-2H-1,4- benzoxazin-6-yl)imidazo[1,2- a]pyrazin-8-yl]amino}phenyl)-2- methylpropan-2-ol |
| 21x | 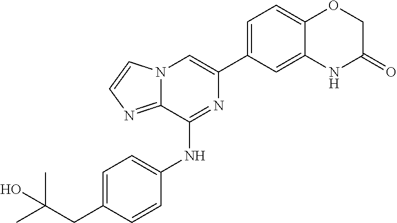 [SMILES: CC(C)(O)Cc1ccc(Nc2nc(-c3ccc4c(c3)NC(=O)CO4)cn3ccnc23)cc1] | 6-(8-{[4-(2-hydroxy-2- methylpropyl)phenyl]amino}imidazo [1,2-a]pyrazin-6-yl)-3,4-dihydro- 2H-1,4-benzoxazin-3-one |
| 22x | 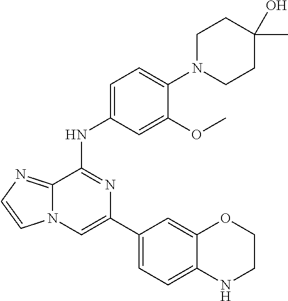 [SMILES: COc1cc(Nc2nc(-c3ccc4c(c3)OCCN4)cn3ccnc23)ccc1N1CCC(C)(O)CC1] | 1-(4-{[6-(3,4-dihydro-2H-1,4- benzoxazin-7-yl)imidazo[1,2- a]pyrazin-8-yl]amino}-2- methoxyphenyl)-4-methylpiperidin- 4-ol |
| 23x | 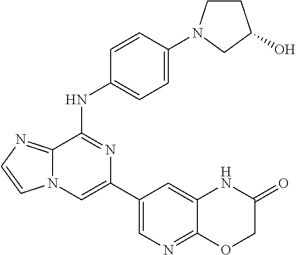 [SMILES: O=C1COc2ncc(-c3cn4ccnc4c(Nc4ccc(N5CC[C@H](O)C5)cc4)n3)cc2N1] | 7-[8-({4-[(3S)-3-hydroxypyrrolidin- 1-yl]phenyl}amino)imidazo[1,2- a]pyrazin-6-yl]-1H,2H,3H- pyrido[2,3-b][1,4]oxazin-2-one |
| 24x | 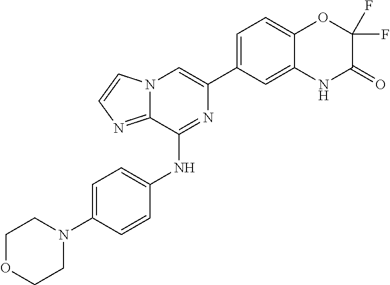 [SMILES: O=C1Nc2cc(-c3cn4ccnc4c(Nc4ccc(N5CCOCC5)cc4)n3)ccc2OC1(F)F] | 2,2-difluoro-6-(8-{[4-(morpholin-4- yl)phenyl]amino}imidazo[1,2- a]pyrazin-6-yl)-3,4-dihydro-2H-1,4- benzoxazin-3-one |
| 25x | 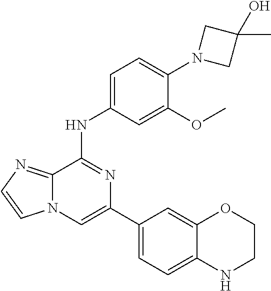 [SMILES: COc1cc(Nc2nc(-c3ccc4c(c3)OCCN4)cn3ccnc23)ccc1N1CC(C)(O)C1] | 1-(4-{[6-(3,4-dihydro-2H-1,4- benzoxazin-7-yl)imidazo[1,2- a]pyrazin-8-yl]amino}-2- methoxyphenyl)-3-methylazetidin- 3-ol |
| 26x | 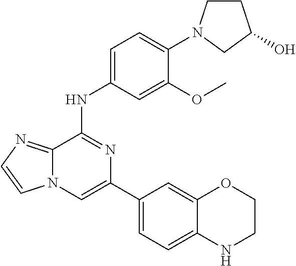 [SMILES: COc1cc(Nc2nc(-c3ccc4c(c3)OCCN4)cn3ccnc23)ccc1N1CC[C@H](O)C1] | (3S)-1-(4-{[6-(3,4-dihydro-2H-1,4- benzoxazin-7-yl)imidazo[1,2- a]pyrazin-8-yl]amino}-2- methoxyphenyl)pyrrolidin-3-ol |
| 27x | 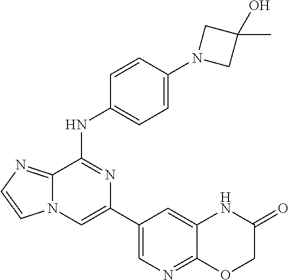 [SMILES: CC1(O)CN(c2ccc(Nc3nc(-c4cnc5c(c4)NC(=O)CO5)cn4ccnc34)cc2)C1] | 7-(8-{[4-(3-hydroxy-3- methylazetidin-1- yl)phenyl]amino}imidazo[1,2- a]pyrazin-6-yl)-1H,2H,3H- pyrido[2,3-b][1,4]oxazin-2-one |
| 28x | 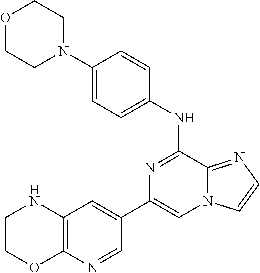 [SMILES: c1cn2cc(-c3cnc4c(c3)NCCO4)nc(Nc3ccc(N4CCOCC4)cc3)c2n1] | 6-(2,3-dihydro-1H-pyrido[2,3- b][1,4]oxazin-7-yl)-N-(4- morpholinophenyl)imidazo[1,2- a]pyrazin-8-amine |
| 29x | 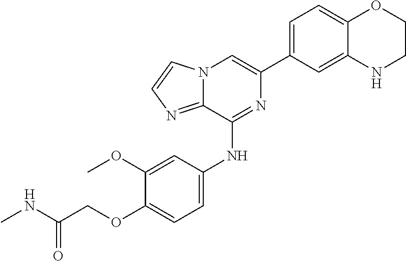 [SMILES: CNC(=O)COc1ccc(Nc2nc(-c3ccc4c(c3)NCCO4)cn3ccnc23)cc1OC] | 2-(4-{[6-(3,4-dihydro-2H-1,4- benzoxazin-6-yl)imidazo[1,2- a]pyrazin-8-yl]amino}-2- methoxyphenoxy)-N- methylacetamide |
| 30x | 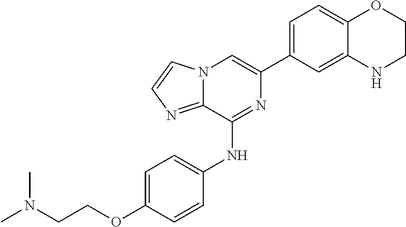 [SMILES: CN(C)CCOc1ccc(Nc2nc(-c3ccc4c(c3)NCCO4)cn3ccnc23)cc1] | 6-(3,4-dihydro-2H-1,4-benzoxazin- 6-yl)-N-{4-[2- (dimethylamino)ethoxy]phenyl} imidazo[1,2-a]pyrazin-8-amine |
| 31x | 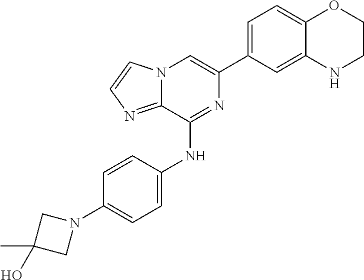 [SMILES: CC1(O)CN(c2ccc(Nc3nc(-c4ccc5c(c4)NCCO5)cn4ccnc34)cc2)C1] | 1-(4-{[6-(3,4-dihydro-2H-1,4- benzoxazin-6-yl)imidazo[1,2- a]pyrazin-8-yl]amino}phenyl)-3- methylazetidin-3-ol |
| 32x | 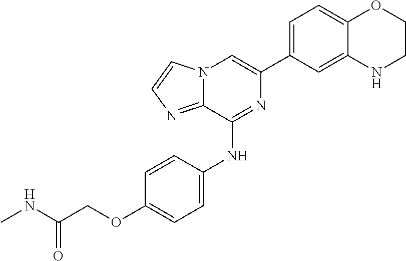 [SMILES: CNC(=O)COc1ccc(Nc2nc(-c3ccc4c(c3)NCCO4)cn3ccnc23)cc1] | 2-(4-{[6-(3,4-dihydro-2H-1,4- benzoxazin-6-yl)imidazo[1,2- a]pyrazin-8-yl]amino}phenoxy)-N- methylacetamide |
| 33x | 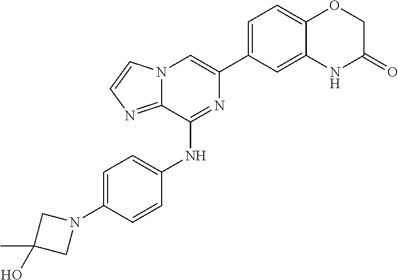 [SMILES: CC1(O)CN(c2ccc(Nc3nc(-c4ccc5c(c4)NC(=O)CO5)cn4ccnc34)cc2)C1] | 6-(8-{[4-(3-hydroxy-3- methylazetidin-1- yl)phenyl]amino}imidazo[1,2- a]pyrazin-6-yl)-3,4-dihydro-2H-1,4- benzoxazin-3-one |
| 34x | 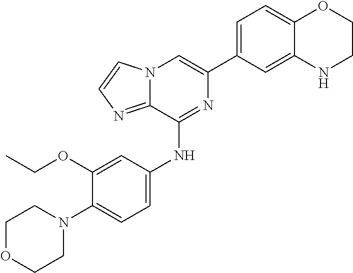 [SMILES: CCOc1cc(Nc2nc(-c3ccc4c(c3)NCCO4)cn3ccnc23)ccc1N1CCOCC1] | 6-(3,4-dihydro-2H-1,4-benzoxazin- 6-yl)-N-[3-ethoxy-4-(morpholin-4- yl)phenyl]imidazo[1,2-a]pyrazin-8- amine |
| 35x | 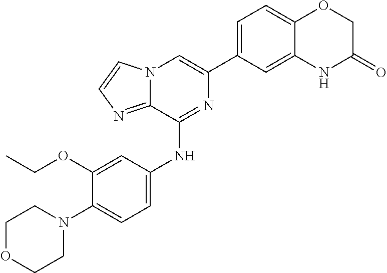 [SMILES: CCOc1cc(Nc2nc(-c3ccc4c(c3)NC(=O)CO4)cn3ccnc23)ccc1N1CCOCC1] | 6-(8-{[3-ethoxy-4-(morpholin-4- yl)phenyl]amino}imidazo[1,2- a]pyrazin-6-yl)-3,4-dihydro-2H-1,4- benzoxazin-3-one |
| 36x | 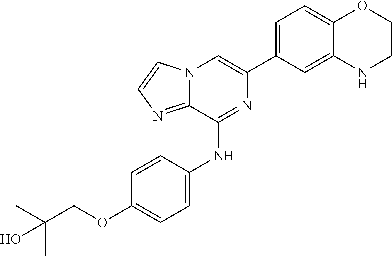 [SMILES: CC(C)(O)COc1ccc(Nc2nc(-c3ccc4c(c3)NCCO4)cn3ccnc23)cc1] | 1-(4-{[6-(3,4-dihydro-2H-1,4- benzoxazin-6-yl)imidazo[1,2- a]pyrazin-8-yl]amino}phenoxy)-2- methylpropan-2-ol |
| 37x | 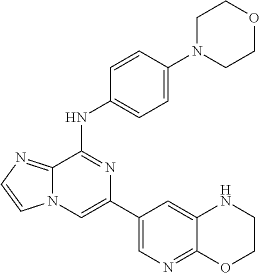 [SMILES: c1cn2cc(-c3cnc4c(c3)NCCO4)nc(Nc3ccc(N4CCOCC4)cc3)c2n1] | N-[4-(morpholin-4-yl)phenyl]-6- {1H,2H,3H-pyrido[2,3- b][1,4]oxazin-7-yl}imidazo[1,2- a]pyrazin-8-amine |
| 38x | 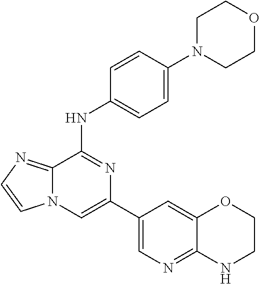 [SMILES: c1cn2cc(-c3cnc4c(c3)OCCN4)nc(Nc3ccc(N4CCOCC4)cc3)c2n1] | N-[4-(morpholin-4-yl)phenyl]-6- {2H,3H,4H-pyrido[3,2- b][1,4]oxazin-7-yl}imidazo[1,2- a]pyrazin-8-amine |
| 39x | 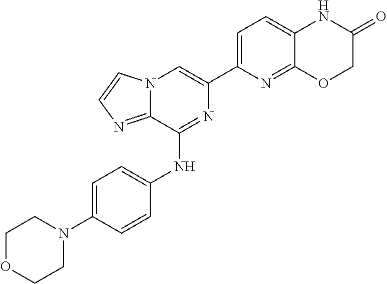 [SMILES: O=C1COc2nc(-c3cn4ccnc4c(Nc4ccc(N5CCOCC5)cc4)n3)ccc2N1] | 6-(8-{[4-(morpholin-4- yl)phenyl]amino}imidazo[1,2- a]pyrazin-6-yl)-1H,2H,3H- pyrido[2,3-b][1,4]oxazin-2-one |
| 40x | 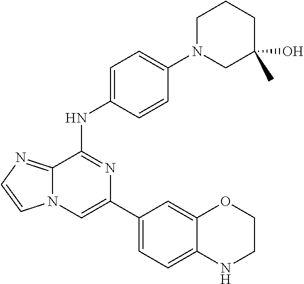 [SMILES: C[C@]1(O)CCCN(c2ccc(Nc3nc(-c4ccc5c(c4)OCCN5)cn4ccnc34)cc2)C1] | (3S)-1-(4-{[6-(3,4-dihydro-2H-1,4- benzoxazin-7-yl)imidazo[1,2- a]pyrazin-8-yl]amino}phenyl)-3- methylpiperidin-3-ol |
| 41x | 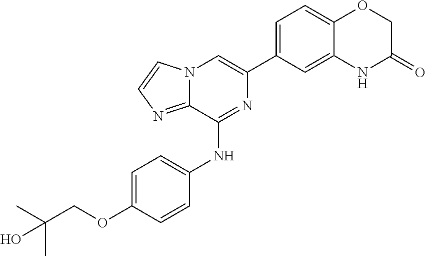 [SMILES: CC(C)(O)COc1ccc(Nc2nc(-c3ccc4c(c3)NC(=O)CO4)cn3ccnc23)cc1] | 6-(8-{[4-(2-hydroxy-2- methylpropoxy)phenyl]amino} imidazo[1,2-a]pyrazin-6-yl)-3,4-dihydro- 2H-1,4-benzoxazin-3-one |
| 42x | 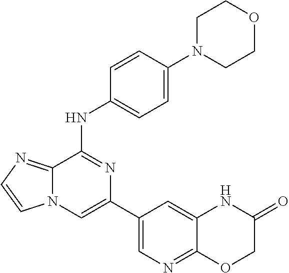 [SMILES: O=C1COc2ncc(-c3cn4ccnc4c(Nc4ccc(N5CCOCC5)cc4)n3)cc2N1] | 7-(8-{[4-(morpholin-4- yl)phenyl]amino}imidazo[1,2- a]pyrazin-6-yl)-1H,2H,3H- pyrido[2,3-b][1,4]oxazin-2-one |
| 43x | 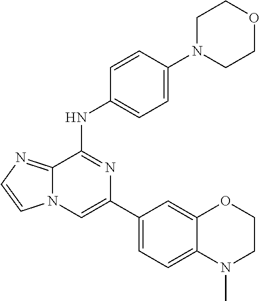 [SMILES: CN1CCOc2cc(-c3cn4ccnc4c(Nc4ccc(N5CCOCC5)cc4)n3)ccc21] | 6-(4-methyl-3,4-dihydro-2H-1,4- benzoxazin-7-yl)-N-[4-(morpholin- 4-yl)phenyl]imidazo[1,2-a]pyrazin- 8-amine |
| 44x | 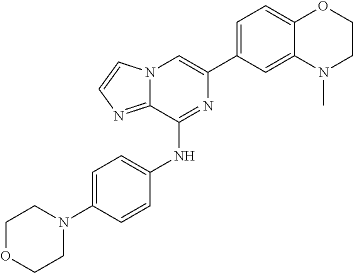 [SMILES: CN1CCOc2ccc(-c3cn4ccnc4c(Nc4ccc(N5CCOCC5)cc4)n3)cc21] | 6-(4-methyl-3,4-dihydro-2H-1,4- benzoxazin-6-yl)-N-[4-(morpholin- 4-yl)phenyl]imidazo[1,2-a]pyrazin- 8-amine |
| 45x | 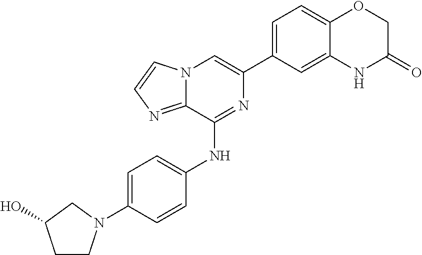 [SMILES: O=C1COc2ccc(-c3cn4ccnc4c(Nc4ccc(N5CC[C@H](O)C5)cc4)n3)cc2N1] | 6-[8-({4-[(3S)-3-hydroxypyrrolidin- 1-yl]phenyl}amino)imidazo[1,2- a]pyrazin-6-yl]-3,4-dihydro-2H-1,4- benzoxazin-3-one |
| 46x | 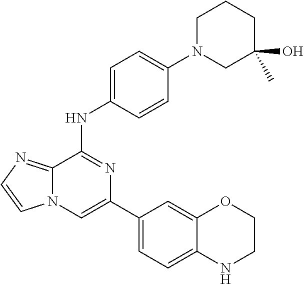 [SMILES: C[C@@]1(O)CCCN(c2ccc(Nc3nc(-c4ccc5c(c4)OCCN5)cn4ccnc34)cc2)C1] | (3R)-1-(4-{[6-(3,4-dihydro-2H-1,4- benzoxazin-7-yl)imidazo[1,2- a]pyrazin-8-yl]amino}phenyl)-3- methylpiperidin-3-ol |
| 47x | 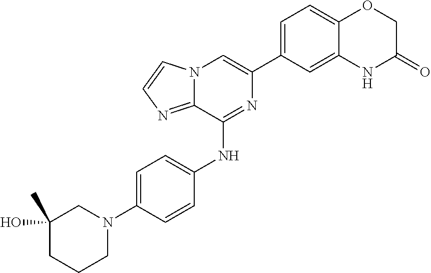 [SMILES: C[C@]1(O)CCCN(c2ccc(Nc3nc(-c4ccc5c(c4)NC(=O)CO5)cn4ccnc34)cc2)C1] | 6-[8-({4-[(3S)-3-hydroxy-3- methylpiperidin-1- yl]phenyl}amino)imidazo[1,2- a]pyrazin-6-yl]-3,4-dihydro-2H-1,4- benzoxazin-3-one |
| 48x | 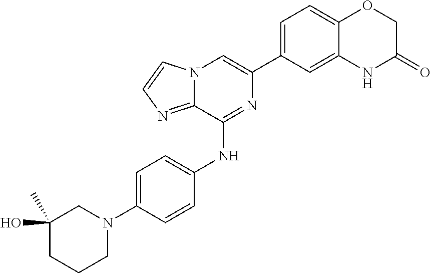 [SMILES: C[C@@]1(O)CCCN(c2ccc(Nc3nc(-c4ccc5c(c4)NC(=O)CO5)cn4ccnc34)cc2)C1] | 6-[8-({4-[(3R)-3-hydroxy-3- methylpiperidin-1- yl]phenyl}amino)imidazo[1,2- a]pyrazin-6-yl]-3,4-dihydro-2H-1,4- benzoxazin-3-one |
| 49x | 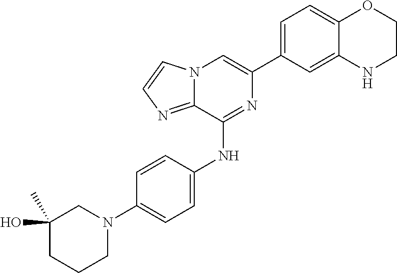 [SMILES: C[C@@]1(O)CCCN(c2ccc(Nc3nc(-c4ccc5c(c4)NCCO5)cn4ccnc34)cc2)C1] | (3R)-1-(4-{[6-(3,4-dihydro-2H-1,4- benzoxazin-6-yl)imidazo[1,2- a]pyrazin-8-yl]amino}phenyl)-3- methylpiperidin-3-ol |
| 50x | 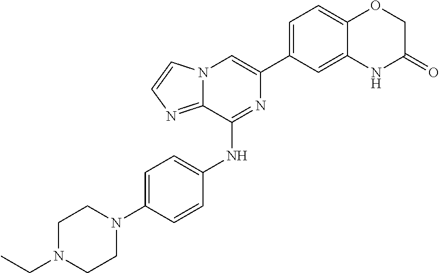 [SMILES: CCN1CCN(c2ccc(Nc3nc(-c4ccc5c(c4)NC(=O)CO5)cn4ccnc34)cc2)CC1] | 6-(8-{[4-(4-ethylpiperazin-1- yl)phenyl]amino}imidazo[1,2- a]pyrazin-6-yl)-3,4-dihydro-2H-1,4- benzoxazin-3-one |
| 51x | 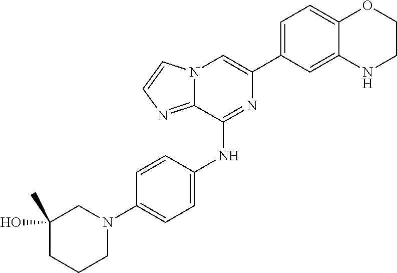 [SMILES: C[C@]1(O)CCCN(c2ccc(Nc3nc(-c4ccc5c(c4)NCCO5)cn4ccnc34)cc2)C1] | (3S)-1-(4-{[6-(3,4-dihydro-2H-1,4- benzoxazin-6-yl)imidazo[1,2- a]pyrazin-8-yl]amino}phenyl)-3- methylpiperidin-3-ol |
| 52x | 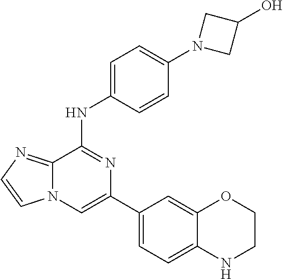 [SMILES: OC1CN(c2ccc(Nc3nc(-c4ccc5c(c4)OCCN5)cn4ccnc34)cc2)C1] | 1-(4-{[6-(3,4-dihydro-2H-1,4- benzoxazin-7-yl)imidazo[1,2- a]pyrazin-8- yl]amino}phenyl)azetidin-3-ol |
| 53x | 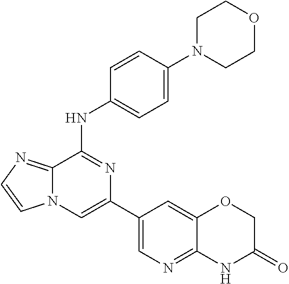 [SMILES: O=C1COc2cc(-c3cn4ccnc4c(Nc4ccc(N5CCOCC5)cc4)n3)cnc2N1] | 7-(8-{[4-(morpholin-4- yl)phenyl]amino}imidazo[1,2- a]pyrazin-6-yl)-2H,3H,4H- pyrido[3,2-b][1,4]oxazin-3-one |
| 54x | 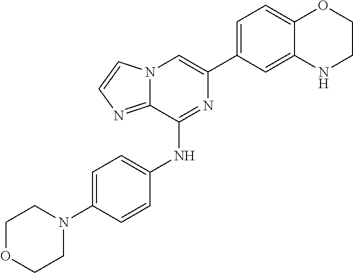 [SMILES: c1cn2cc(-c3ccc4c(c3)NCCO4)nc(Nc3ccc(N4CCOCC4)cc3)c2n1] | 6-(3,4-dihydro-2H-1,4-benzoxazin- 6-yl)-N-[4-(morpholin-4- yl)phenyl]imidazo[1,2-a]pyrazin-8- amine |
| 55x | 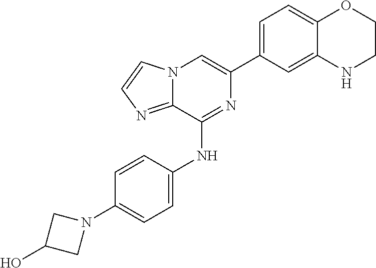 [SMILES: OC1CN(c2ccc(Nc3nc(-c4ccc5c(c4)NCCO5)cn4ccnc34)cc2)C1] | 1-(4-{[6-(3,4-dihydro-2H-1,4- benzoxazin-6-yl)imidazo[1,2- a]pyrazin-8- yl]amino}phenyl)azetidin-3-ol |
| 56x | 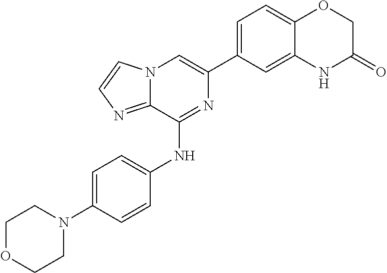 [SMILES: O=C1COc2ccc(-c3cn4ccnc4c(Nc4ccc(N5CCOCC5)cc4)n3)cc2N1] | 6-(8-{[4-(morpholin-4- yl)phenyl]amino}imidazo[1,2- a]pyrazin-6-yl)-3,4-dihydro-2H-1,4- benzoxazin-3-one |
| 57x | 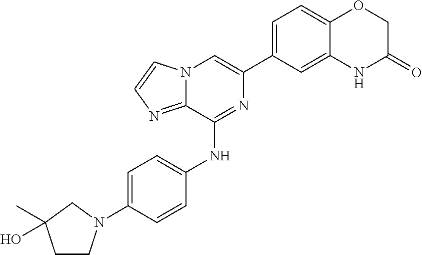 [SMILES: CC1(O)CCN(c2ccc(Nc3nc(-c4ccc5c(c4)NC(=O)CO5)cn4ccnc34)cc2)C1] | 6-(8-{[4-(3-hydroxy-3- methylpyrrolidin-1- yl)phenyl]amino}imidazo[1,2- a]pyrazin-6-yl)-3,4-dihydro-2H-1,4- benzoxazin-3-one |
| 58x | 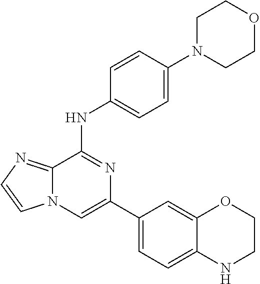 [SMILES: c1cn2cc(-c3ccc4c(c3)OCCN4)nc(Nc3ccc(N4CCOCC4)cc3)c2n1] | 6-(3,4-dihydro-2H-1,4-benzoxazin- 7-yl)-N-[4-(morpholin-4- yl)phenyl]imidazo[1,2-a]pyrazin-8- amine |
| 59x | 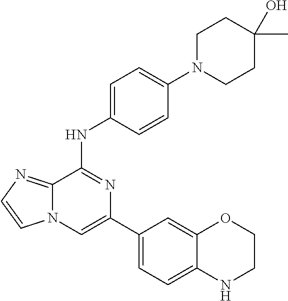 [SMILES: CC1(O)CCN(c2ccc(Nc3nc(-c4ccc5c(c4)OCCN5)cn4ccnc34)cc2)CC1] | 1-(4-{[6-(3,4-dihydro-2H-1,4- benzoxazin-7-yl)imidazo[1,2- a]pyrazin-8-yl]amino}phenyl)-4- methylpiperidin-4-ol |
| 60x | 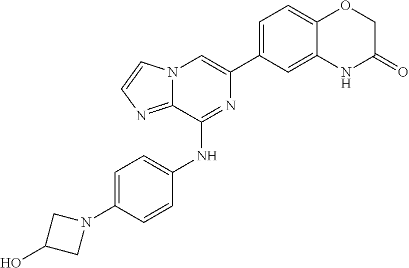 [SMILES: O=C1COc2ccc(-c3cn4ccnc4c(Nc4ccc(N5CC(O)C5)cc4)n3)cc2N1] | 6-(8-{[4-(3-hydroxyazetidin-1- yl)phenyl]amino}imidazo[1,2- a]pyrazin-6-yl)-3,4-dihydro-2H-1,4- benzoxazin-3-one |
| 61x | 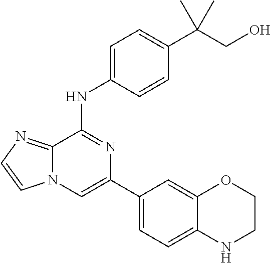 [SMILES: CC(C)(CO)c1ccc(Nc2nc(-c3ccc4c(c3)OCCN4)cn3ccnc23)cc1] | 2-(4-{[6-(3,4-dihydro-2H-1,4- benzoxazin-7-yl)imidazo[1,2- a]pyrazin-8-yl]amino}phenyl)-2- methylpropan-1-ol |
| 62x | 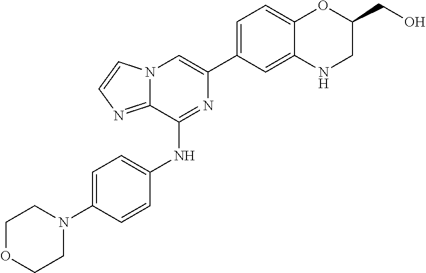 [SMILES: OC[C@H]1CNc2cc(-c3cn4ccnc4c(Nc4ccc(N5CCOCC5)cc4)n3)ccc2O1] | [(2R)-6-(8-{[4-(morpholin-4- yl)phenyl]amino}imidazo[1,2- a]pyrazin-6-yl)-3,4-dihydro-2H-1,4- benzoxazin-2-yl]methanol |
| 63x | 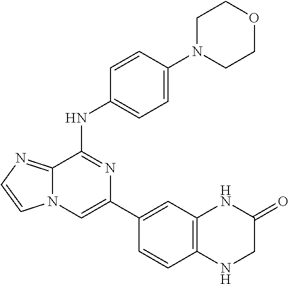 [SMILES: O=C1CNc2ccc(-c3cn4ccnc4c(Nc4ccc(N5CCOCC5)cc4)n3)cc2N1] | 7-(8-{[4-(morpholin-4- yl)phenyl]amino}imidazo[1,2- a]pyrazin-6-yl)-1,2,3,4- tetrahydroquinoxalin-2-one |
| 64x | 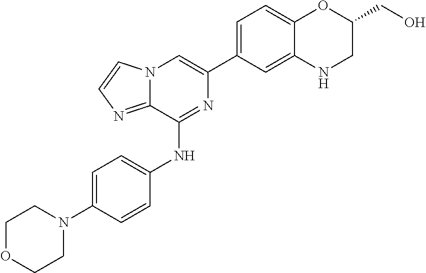 [SMILES: OC[C@@H]1CNc2cc(-c3cn4ccnc4c(Nc4ccc(N5CCOCC5)cc4)n3)ccc2O1] | [(2S)-6-(8-{[4-(morpholin-4- yl)phenyl]amino}imidazo[1,2- a]pyrazin-6-yl)-3,4-dihydro-2H-1,4- benzoxazin-2-yl]methanol |
| 65x | 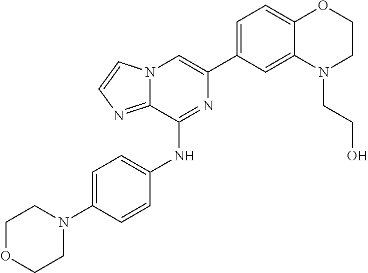 [SMILES: OCCN1CCOc2ccc(-c3cn4ccnc4c(Nc4ccc(N5CCOCC5)cc4)n3)cc21] | 2-[6-(8-{[4-(morpholin-4- yl)phenyl]amino}imidazo[1,2- a]pyrazin-6-yl)-3,4-dihydro-2H-1,4- benzoxazin-4-yl]ethan-1-ol |
| 66x | 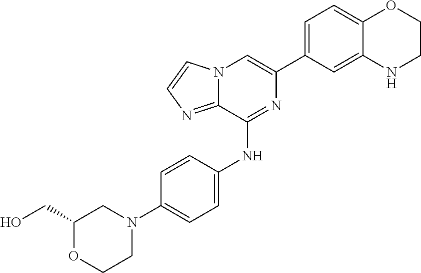 [SMILES: OC[C@@H]1CN(c2ccc(Nc3nc(-c4ccc5c(c4)NCCO5)cn4ccnc34)cc2)CCO1] | [(2S)-4-(4-{[6-(3,4-dihydro-2H-1,4- benzoxazin-6-yl)imidazo[1,2- a]pyrazin-8- yl]amino}phenyl)morpholin-2- yl]methanol |
| 67x | 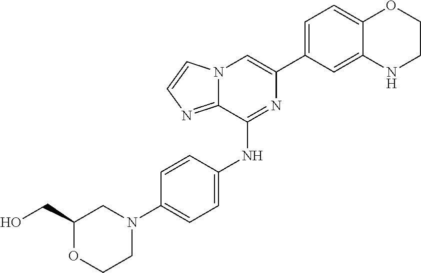 [SMILES: OC[C@H]1CN(c2ccc(Nc3nc(-c4ccc5c(c4)NCCO5)cn4ccnc34)cc2)CCO1] | [(2R)-4-(4-{[6-(3,4-dihydro-2H-1,4- benzoxazin-6-yl)imidazo[1,2- a]pyrazin-8- yl]amino}phenyl)morpholin-2- yl]methanol |
| 68x | 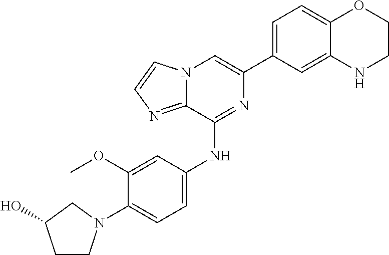 [SMILES: COc1cc(Nc2nc(-c3ccc4c(c3)NCCO4)cn3ccnc23)ccc1N1CC[C@H](O)C1] | (3S)-1-(4-{[6-(3,4-dihydro-2H-1,4- benzoxazin-6-yl)imidazo[1,2- a]pyrazin-8-yl]amino}-2- methoxyphenyl)pyrrolidin-3-ol |
| 69x | 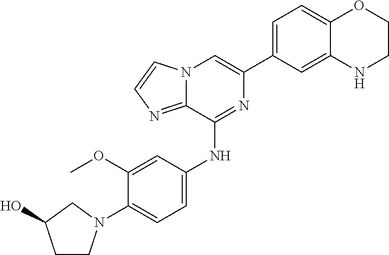 [SMILES: COc1cc(Nc2nc(-c3ccc4c(c3)NCCO4)cn3ccnc23)ccc1N1CC[C@@H](O)C1] | (3R)-1-(4-{[6-(3,4-dihydro-2H-1,4- benzoxazin-6-yl)imidazo[1,2- a]pyrazin-8-yl]amino}-2- methoxyphenyl)pyrrolidin-3-ol |
| 70x | 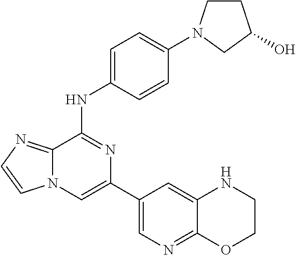 [SMILES: O[C@H]1CCN(c2ccc(Nc3nc(-c4cnc5c(c4)NCCO5)cn4ccnc34)cc2)C1] | (3S)-1-{4-[(6-{1H,2H,3H- pyrido[2,3-b][1,4]oxazin-7- yl}imidazo[1,2-a]pyrazin-8- yl)amino]phenyl}pyrrolidin-3-ol |
| 71x | 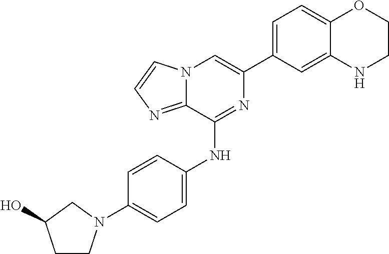 [SMILES: O[C@@H]1CCN(c2ccc(Nc3nc(-c4ccc5c(c4)NCCO5)cn4ccnc34)cc2)C1] | (3R)-1-(4-{[6-(3,4-dihydro-2H-1,4- benzoxazin-6-yl)imidazo[1,2- a]pyrazin-8- yl]amino}phenyl)pyrrolidin-3-ol |
| 72x | 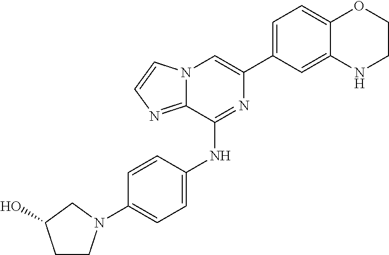 [SMILES: O[C@H]1CCN(c2ccc(Nc3nc(-c4ccc5c(c4)NCCO5)cn4ccnc34)cc2)C1] | (3S)-1-(4-{[6-(3,4-dihydro-2H-1,4- benzoxazin-6-yl)imidazo[1,2- a]pyrazin-8- yl]amino}phenyl)pyrrolidin-3-ol |
| 73x | 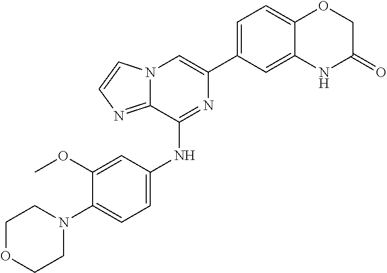 [SMILES: COc1cc(Nc2nc(-c3ccc4c(c3)NC(=O)CO4)cn3ccnc23)ccc1N1CCOCC1] | 6-(8-{[3-methoxy-4-(morpholin-4- yl)phenyl]amino}imidazo[1,2- a]pyrazin-6-yl)-3,4-dihydro-2H-1,4 benzoxazin-3-one |
| 74x | 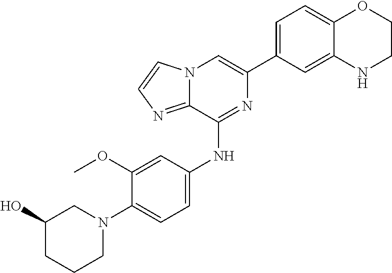 [SMILES: COc1cc(Nc2nc(-c3ccc4c(c3)NCCO4)cn3ccnc23)ccc1N1CCC[C@@H](O)C1] | (3R)-1-(4-{[6-(3,4-dihydro-2H-1,4- benzoxazin-6-yl)imidazo[1,2- a]pyrazin-8-yl]amino}-2- methoxyphenyl)piperidin-3-ol |
| 75x | 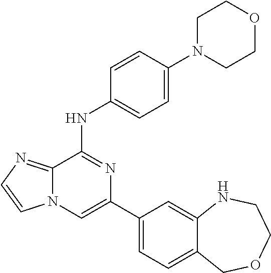 [SMILES: c1cn2cc(-c3ccc4c(c3)NCCOC4)nc(Nc3ccc(N4CCOCC4)cc3)c2n1] | N-[4-(morpholin-4-yl)phenyl]-6- (1,2,3,5-tetrahydro-4,1- benzoxazepin-8-yl)imidazo[1,2- a]pyrazin-8-amine |
| 76x | 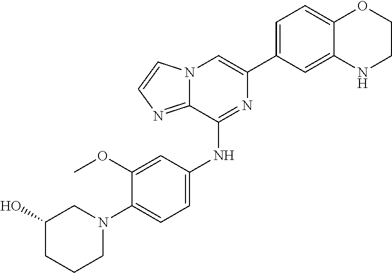 [SMILES: COc1cc(Nc2nc(-c3ccc4c(c3)NCCO4)cn3ccnc23)ccc1N1CCC[C@H](O)C1] | (3S)-1-(4-{[6-(3,4-dihydro-2H-1,4- benzoxazin-6-yl)imidazo[1,2- a]pyrazin-8-yl]amino}-2- methoxyphenyl)piperidin-3-ol |
| 77x | 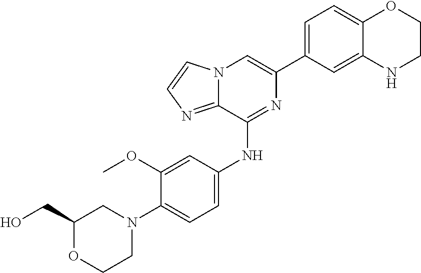 [SMILES: COc1cc(Nc2nc(-c3ccc4c(c3)NCCO4)cn3ccnc23)ccc1N1CCO[C@@H](CO)C1] | [(2R)-4-(4-{[6-(3,4-dihydro-2H-1,4- benzoxazin-6-yl)imidazo[1,2- a]pyrazin-8-yl]amino}-2- methoxyphenyl)morpholin-2- yl]methanol |
| 78x | 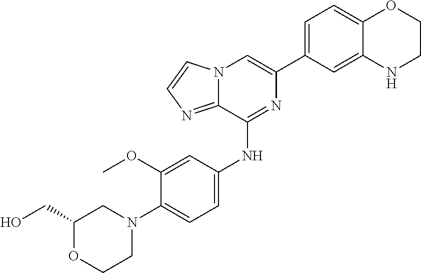 [SMILES: COc1cc(Nc2nc(-c3ccc4c(c3)NCCO4)cn3ccnc23)ccc1N1CCO[C@H](CO)C1] | [(2S)-4-(4-{[6-(3,4-dihydro-2H-1,4- benzoxazin-6-yl)imidazo[1,2- a]pyrazin-8-yl]amino}-2- methoxyphenyl)morpholin-2- yl]methanol |
| 79x | 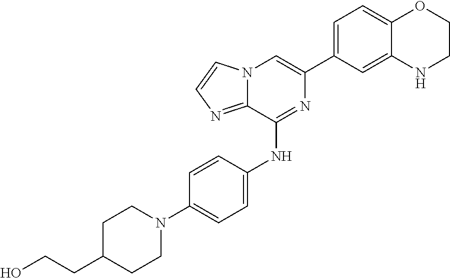 [SMILES: OCCC1CCN(c2ccc(Nc3nc(-c4ccc5c(c4)NCCO5)cn4ccnc34)cc2)CC1] | 2-[1-(4-{[6-(3,4-dihydro-2H-1,4- benzoxazin-6-yl)imidazo[1,2- a]pyrazin-8- yl]amino}phenyl)piperidin-4- yl]ethan-1-ol |
| 80x | 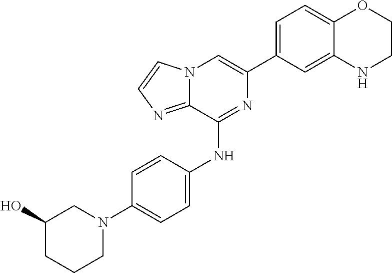 [SMILES: O[C@@H]1CCCN(c2ccc(Nc3nc(-c4ccc5c(c4)NCCO5)cn4ccnc34)cc2)C1] | (3R)-1-(4-{[6-(3,4-dihydro-2H-1,4- benzoxazin-6-yl)imidazo[1,2- a]pyrazin-8- yl]amino}phenyl)piperidin-3-ol |
| 81x | 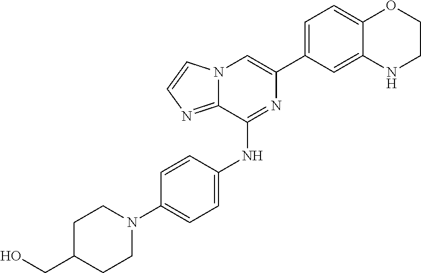 [SMILES: OCC1CCN(c2ccc(Nc3nc(-c4ccc5c(c4)NCCO5)cn4ccnc34)cc2)CC1] | [1-(4-{[6-(3,4-dihydro-2H-1,4- benzoxazin-6-yl)imidazo[1,2- a]pyrazin-8- yl]amino}phenyl)piperidin-4- yl]methanol |
| 82x | 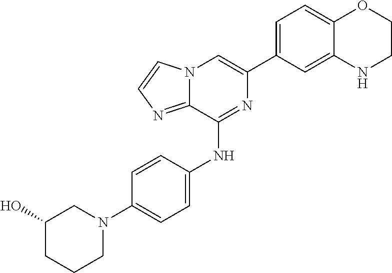 [SMILES: O[C@H]1CCCN(c2ccc(Nc3nc(-c4ccc5c(c4)NCCO5)cn4ccnc34)cc2)C1] | (3S)-1-(4-{[6-(3,4-dihydro-2H-1,4- benzoxazin-6-yl)imidazo[1,2- a]pyrazin-8- yl]amino}phenyl)piperidin-3-ol |
| 83x | 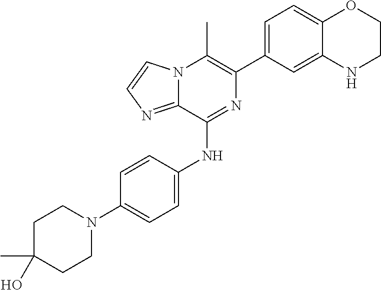 [SMILES: Cc1c(-c2ccc3c(c2)NCCO3)nc(Nc2ccc(N3CCC(C)(O)CC3)cc2)c2nccn12] | 1-(4-{[6-(3,4-dihydro-2H-1,4- benzoxazin-6-yl)-5- methylimidazo[1,2-a]pyrazin-8- yl]amino}phenyl)-4- methylpiperidin-4-ol |
| 84x | 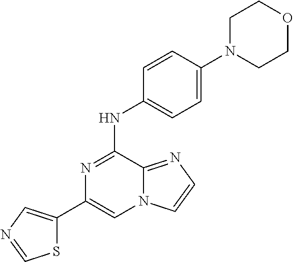 [SMILES: c1cn2cc(-c3cncs3)nc(Nc3ccc(N4CCOCC4)cc3)c2n1] | N-[4-(morpholin-4-yl)phenyl]-6- (1,3-thiazol-5-yl)imidazo[1,2- a]pyrazin-8-amine |
| 85x | 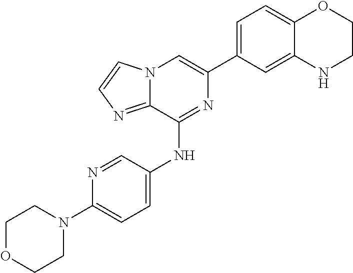 [SMILES: c1cn2cc(-c3ccc4c(c3)NCCO4)nc(Nc3ccc(N4CCOCC4)nc3)c2n1] | N-[6-(3,4-dihydro-2H-1,4-benzoxazin-6- yl)imidazo[1,2-a]pyrazin-8-yl]-6- (morpholin-4-yl)pyridin-3-amine |
| 86x | 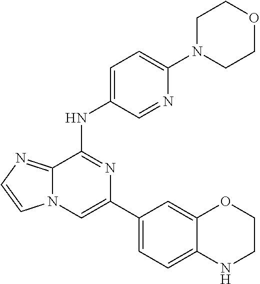 [SMILES: c1cn2cc(-c3ccc4c(c3)OCCN4)nc(Nc3ccc(N4CCOCC4)nc3)c2n1] | N-[6-(3,4-dihydro-2H-1,4-benzoxazin-7- yl)imidazo[1,2-a]pyrazin-8-yl]-6- (morpholin-4-yl)pyridin-3-amine |
| 87x | 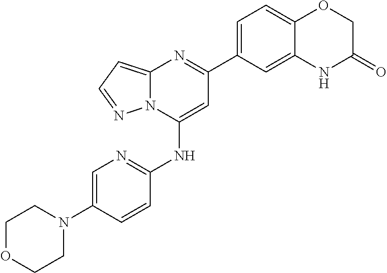 [SMILES: O=C1COc2ccc(-c3cc(Nc4ccc(N5CCOCC5)cn4)n4nccc4n3)cc2N1] | 6-(7-{[5-(morpholin-4-yl)pyridin-2- yl]amino}pyrazolo[1,5-a]pyrimidin-5-yl)- 3,4-dihydro-2H-1,4-benzoxazin-3-one |
| 88x | 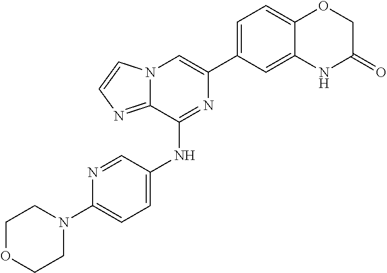 [SMILES: O=C1COc2ccc(-c3cn4ccnc4c(Nc4ccc(N5CCOCC5)nc4)n3)cc2N1] | 6-(8-{[6-(morpholin-4-yl)pyridin-3- yl]amino}imidazo[1,2-a]pyrazin-6-yl)-3,4- dihydro-2H-1,4-benzoxazin-3-one |
| 89x | 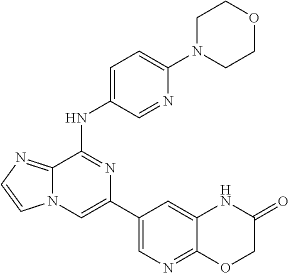 [SMILES: O=C1COc2ncc(-c3cn4ccnc4c(Nc4ccc(N5CCOCC5)nc4)n3)cc2N1] | 7-(8-{[6-(morpholin-4-yl)pyridin-3- yl]amino}imidazo[1,2-a]pyrazin-6-yl)- 1H,2H,3H-pyrido[2,3-b][1,4]oxazin-2-one |
| 90x | 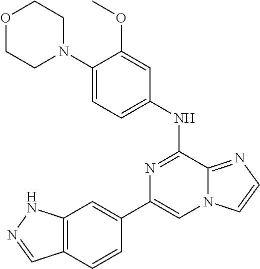 [SMILES: COc1cc(Nc2nc(-c3ccc4cn[nH]c4c3)cn3ccnc23)ccc1N1CCOCC1] | 6-(1H-indazol-6-yl)-N-(3-methoxy-4- morpholinophenyl)imidazo[1,2-a]pyrazin- 8-amine |

In another aspect, provided is a compound having the structure of formula (Ia):

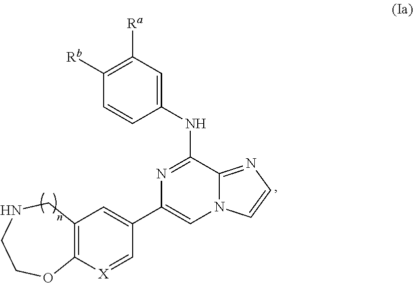

or a pharmaceutically acceptable salt thereof, wherein:

X is N or CH;

n is 0 or 1;

R^{a} is unsubstituted alkoxy; and

R^{b} is selected from the group consisting of unsubstituted morpholinyl, substituted morpholinyl, unsubstituted piperazinyl, and substituted piperazinyl.

In one embodiment, X is N. In another embodiment, X is CH.

In one embodiment, n is 0. In another embodiment, n is 1.

In one embodiment, R^{a} is methoxy. In another embodiment, R^{a} is ethoxy.

In some embodiments, R^{b} is unsubstituted morpholinyl, or substituted morpholinyl with one, two or three substituents independently selected from the group consisting of unsubstituted alkyl and substituted alkyl. In other embodiments, R^{b} is unsubstituted piperazinyl, or substituted piperazinyl with one, two or three substituents independently selected from the group consisting of unsubstituted alkyl, substituted alkyl, unsubstituted heterocycloalkyl, and substituted heterocycloalkyl.

In certain embodiments, R^{b} is selected from the group consisting of:

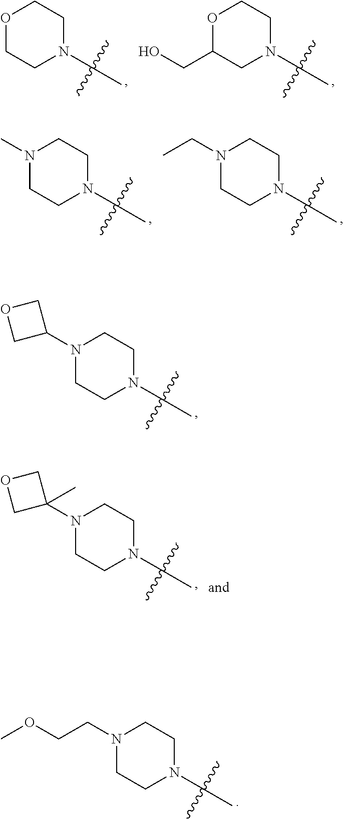

In some embodiments,

X is N or CH;

R^{a} is methoxy;

R^{b} is unsubstituted morpholinyl or substituted morpholinyl; and

n is 0 or 1.

In other embodiments,

X is N;

R^{a} is methoxy;

R^{b} is unsubstituted morpholinyl or substituted morpholinyl; and

n is 0.

In yet other embodiments,

X is CH;

R^{a} is methoxy;

R^{b} is unsubstituted morpholinyl or substituted morpholinyl; and

n is 1.

In yet other embodiments,

X is CH;

R^{a} is methoxy;

R^{b} is unsubstituted piperazinyl or substituted piperazinyl; and

n is 0.

In some embodiments, the compound is not Compound No. 14x, 34x, 77x, 78x or 79x.

In yet another aspect, provided is a compound having the structure of formula (Ib):

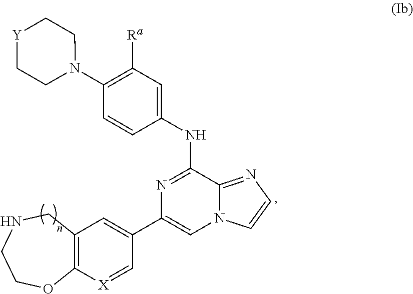

or a pharmaceutically acceptable salt thereof, wherein:

X is N or CH;

n is 0 or 1;

R^{a} is unsubstituted alkoxy;

Y is O or NR^{2a}; and

R^{2a} is selected from the group consisting of unsubstituted alkyl, substituted alkyl, unsubstituted heterocycloalkyl, and substituted heterocycloalkyl.

In one embodiment, X is N. In another embodiment, X is CH.

In one embodiment, n is 0. In another embodiment, n is 1.

In one embodiment, R^{a} is methoxy. In another embodiment, R^{a} is ethoxy.

In some embodiment, Y is O. In other embodiments, Y is NR^{2a}, wherein R^{3a} is unsubstituted heterocycloalkyl. In certain embodiments, R^{2a} is selected from the group consisting of unsubstituted oxetanyl, substituted oxetanyl, unsubstituted tetrahydrofuranyl, substituted tetrahydrofuranyl, unsubstituted tetrahydropyranyl, substituted tetrahydropyranyl, unsubstituted oxepanyl, and substituted oxepanyl.

In some embodiments,

X is CH;

n is 0;

R^{a} is methoxy;

Y is NR^{2a}; and

R^{ea} is substituted heterocycloalkyl.

In other embodiments,

X is N or CH;

n is 0 or 1;

R^{a} is methoxy;

Y is O.

In some embodiment, the compound is not Compound No. 14x, 34x, 77x, 78x or 79x.

In yet another aspect, provided is a compound having the structure of formula (Ic):

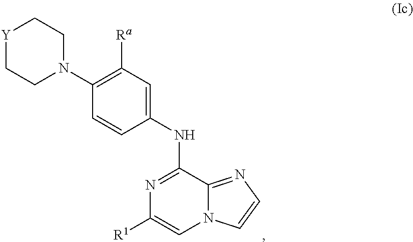

or a pharmaceutically acceptable salt thereof, wherein:

R¹ is

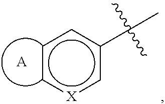

wherein A is selected from the group consisting of unsubstituted morpholinyl, substituted morpholinyl, unsubstituted oxazepanyl, and substituted oxazepanyl;

X is N or CR^{x}, wherein IV is hydrogen or C₁₋₆ alkyl;

R^{a} is unsubstituted alkoxy;

Y is O or NR^{2a}; and

R^{2a} is selected from the group consisting of unsubstituted alkyl, substituted alkyl, unsubstituted heterocycloalkyl, and substituted heterocycloalkyl.

In some embodiments, X is CR^{x}. In one embodiment, R^{x} is hydrogen. In another embodiment, R^{x} is unsubstituted C₁₋₆ alkyl. In yet another embodiment, R^{x} is substituted C₁₋₆ alkyl.

In other embodiments, R is selected from the group consisting of:

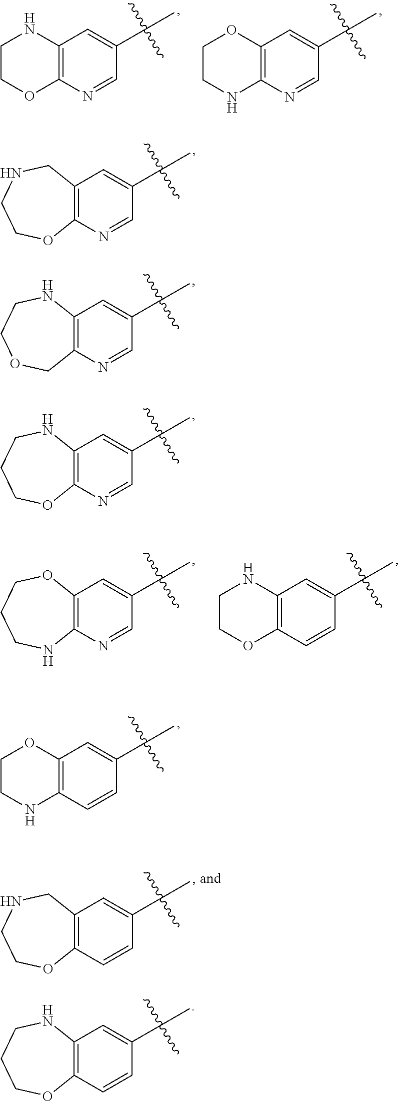

In one embodiment, R^{a} is methoxy. In another embodiment, R^{a} is ethoxy.

In some embodiments, Y is O. In other embodiments, Y is NR^{2a}, wherein R^{2a} is unsubstituted heterocycloalkyl or substituted heterocycloalkyl. In certain embodiments, R^{2a} is selected from the group consisting of unsubstituted oxetanyl, substituted oxetanyl, unsubstituted tetrahydrofuranyl, substituted tetrahydrofuranyl, unsubstituted tetrahydropyranyl, substituted tetrahydropyranyl, unsubstituted oxepanyl, and substituted oxepanyl.

In some embodiments, the compound is not Compound No. 14x, 34x, 77x, 78x or 79x.

In yet another aspect, provided is a compound having the structure of formula (Id):

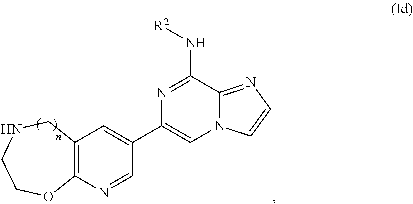

or a pharmaceutically acceptable salt thereof, wherein:

n is 0 or 1;

R² is selected from the group consisting of unsubstituted phenyl, substituted phenyl, unsubstituted pyridinyl, substituted pyridinyl, unsubstituted pyrazolyl, substituted pyrazolyl, unsubstituted thiazolyl, and substituted thiazolyl.

In some embodiments, R² is unsubstituted phenyl.

In other embodiments, R² is substituted phenyl with one or two substituents independently selected from the group consisting of:

unsubstituted alkyl, substituted alkyl, unsubstituted cycloalkyl, substituted cycloalkyl, unsubstituted heterocycloalkyl, substituted heterocycloalkyl, unsubstituted aryl, substituted aryl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, and oxime;

or R^{a} and R^{b} are taken together with the carbons to which they are attached to form a heterocyclyl ring or heteroaryl ring containing 1-3 heteroatoms selected from the group consisting of N and O.

In yet other embodiments, R² is

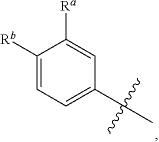

wherein:

R^{a} is hydrogen, halo or unsubstituted alkoxy; and

R^{b} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted aryl, substituted aryl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, and oxime;

or R^{a} and R^{b} are taken together with the carbons to which they are attached to form a heterocyclyl ring or heteroaryl ring containing 1-3 heteroatoms selected from the group consisting of N and O.

In some embodiments, R² is unsubstituted pyridinyl. In other embodiments, R² is substituted pyridinyl with one or two substituents independently selected from the group consisting of unsubstituted morpholinyl and substituted morpholinyl.

In yet other embodiments, R² is

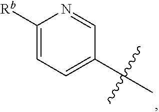

wherein:

R^{b} is selected from the group consisting of unsubstituted alkyl, substituted alkyl, unsubstituted cycloalkyl, substituted cycloalkyl, unsubstituted heterocycloalkyl, substituted heterocycloalkyl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, and oxime;

In some embodiments, R² is unsubstituted pyrazolyl. In other embodiments, R² is substituted pyrazolyl with one or two substituents independently selected from the group consisting of unsubstituted alkyl and substituted alkyl.

In some embodiments, R² is unsubstituted thiazolyl. In other embodiments, R² is substituted thiazolyl with one or two substituents independently selected from the group consisting of unsubstituted alkyl and substituted alkyl.

In some embodiments, the compound is not Compound No. 28x or 37x.

In yet another aspect, provided is a compound having the structure of formula (Ie):

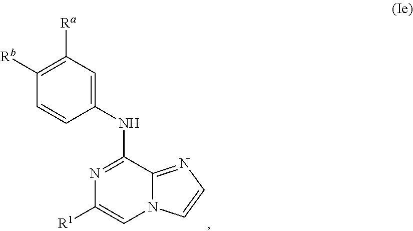

or a pharmaceutically acceptable salt thereof, wherein:

R¹ is substituted thiazolyl;

R^{a} is hydrogen, halo or unsubstituted alkoxy; and

R^{b} is selected from the group consisting of unsubstituted alkyl, substituted alkyl, unsubstituted alkoxy, substituted alkoxy, unsubstituted sulfonyl, substituted sulfonyl, unsubstituted morpholinyl, substituted morpholinyl, unsubstituted homomorpholinyl, substituted homomorpholinyl, unsubstituted piperazinyl, substituted piperazinyl, unsubstituted piperidinyl, substituted piperidinyl, unsubstituted pyrrolidinyl, substituted pyrrolidinyl, unsubstituted azetidinyl, and substituted azetidinyl.

In some embodiments, R^{a} is methoxy.

In some embodiments, R^{b} is unsubstituted morpholinyl.

In certain embodiments, R¹ is substituted thiazolyl with one or two substituents selected from the group consisting of unsubstituted alkyl, substituted alkyl, unsubstituted cycloalkyl, substituted cycloalkyl, unsubstituted heterocycloalkyl, and substituted heterocycloalkyl.

In one embodiment, R¹ is selected from the group consisting of:

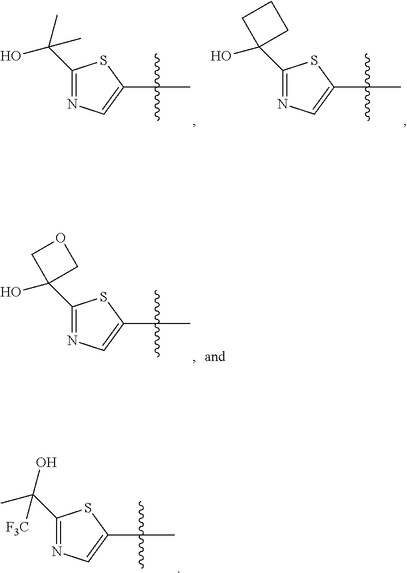

Representative compounds of formulae I, Ia, Ib, Ic, Id, and/or Ie are shown in Table 1 below. The values of IC₅₀ were determined as described herein; for example, in Example B2.

TABLE 1
Representative Compounds

| | | | | IC₅₀ |
|---|---|---|---|---|
| NO. | STRUCTURE | NAME | EX. | (uM) |
| 1 | 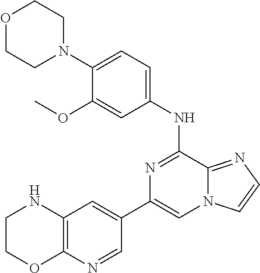 [SMILES: COc1cc(Nc2nc(-c3cnc4c(c3)NCCO4)cn3ccnc23)ccc1N1CCOCC1] | 6-(2,3-dihydro-1H-pyrido[2,3- b][1,4]oxazin-7-yl)-N-(3- methoxy-4- morpholinophenyl)imidazo[1,2- a]pyrazin-8-amine | 5 | 0.003 |
| 2 | 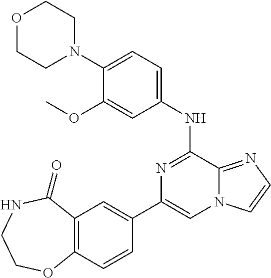 [SMILES: COc1cc(Nc2nc(-c3ccc4c(c3)C(=O)NCCO4)cn3ccnc23)ccc1N1CCOCC1] | 7-(8-((3-methoxy-4- morpholinophenyl)amino)imidazo [1,2-a]pyrazin-6-yl)-3,4- dihydrobenzo[f][1,4]oxazepin- 5(2H)-one | 7 | 0.006 |
| 3 | 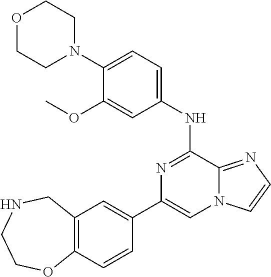 [SMILES: COc1cc(Nc2nc(-c3ccc4c(c3)CNCCO4)cn3ccnc23)ccc1N1CCOCC1] | N-(3-methoxy-4- morpholinopheny])-6-(2,3,4,5- tetrahydrobenzo[f][1,4]oxazepin- 7-yl)imidazo[1,2-a]pyrazin-8- amine | 8 | 0.007 |
| 4 | 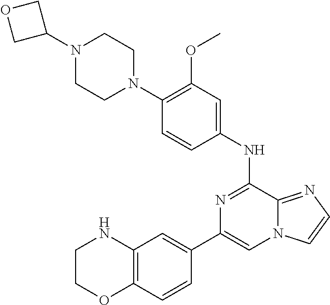 [SMILES: COc1cc(Nc2nc(-c3ccc4c(c3)NCCO4)cn3ccnc23)ccc1N1CCN(C2COC2)CC1] | 6-(3,4-dihydro-2H- benzo[b][1,4]oxazin-6-yl)-N-(3- methoxy-4-(4-(oxetan-3- yl)piperazin-1- yl)phenyl)imidazo[1,2-a]pyrazin- 8-amine | 12 | 0.004 |
| 5A | 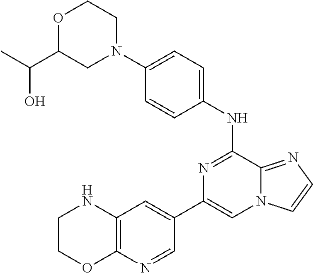 [SMILES: CC(O)C1CN(c2ccc(Nc3nc(-c4cnc5c(c4)NCCO5)cn4ccnc34)cc2)CCO1] | l-(4-(4-((6-(2,3-dihydro-1H- pyrido[2,3-b][1,4]oxazin-7- yl)imidazo[1,2-a]pyrazin-8- yl)amino)phenyl)morpholin-2- yl)ethanol | — | — |
| 5 | 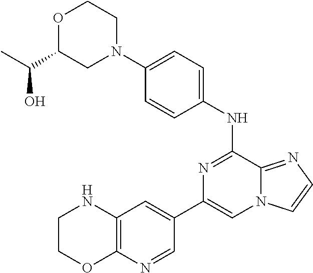 [SMILES: C[C@H](O)[C@H]1CN(c2ccc(Nc3nc(-c4cnc5c(c4)NCCO5)cn4ccnc34)cc2)CCO1] | (S)-1-((R)-4-(4-((6-(2,3-dihydro- 1H-pyrido[2,3-b][1,4]oxazin-7- yl)imidazo[1,2-a]pyrazin-8- yl)amino)phenyl)morpholin-2- yl)ethanol | 13 | 0.006 |
| 6 | 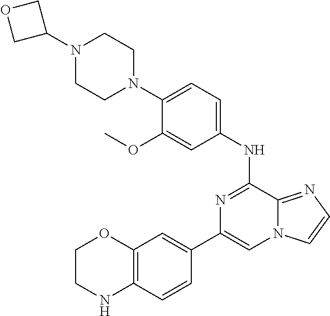 [SMILES: COc1cc(Nc2nc(-c3ccc4c(c3)OCCN4)cn3ccnc23)ccc1N1CCN(C2COC2)CC1] | 6-(3,4-dihydro-2H- benzo[b][1,4]oxazin-7-yl)-N-(3- methoxy-4-(4-(oxetan-3- yl)piperazin-1- yl)phenyl)imidazo[1,2-a]pyrazin- 8-amine | 14 | 0.007 |
| 7 | 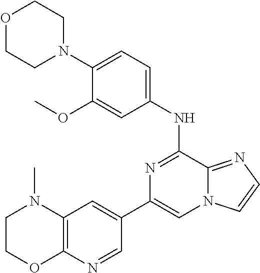 [SMILES: COc1cc(Nc2nc(-c3cnc4c(c3)N(C)CCO4)cn3ccnc23)ccc1N1CCOCC1] | N-(3-methoxy-4- morpholinophenyl)-6-(1-methyl- 2,3-dihydro-1H-pyrido[2,3- b][1,4]oxazin-7-yl)imidazo[1,2- a]pyrazin-8-amine | 15 | 0.006 |
| 8 | 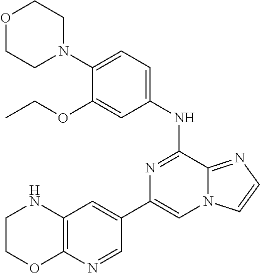 [SMILES: CCOc1cc(Nc2nc(-c3cnc4c(c3)NCCO4)cn3ccnc23)ccc1N1CCOCC1] | 6-(2,3-dihydro-1H-pyrido[2,3- b][1,4]oxazin-7-yl)-N-(3-ethoxy- 4- morpholinophenyl)imidazo[1,2- a]pyrazin-8-amine | 16 | 0.007 |
| 9A | 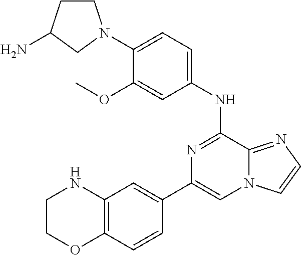 [SMILES: COc1cc(Nc2nc(-c3ccc4c(c3)NCCO4)cn3ccnc23)ccc1N1CCC(N)C1] | N-(4-(3-aminopyirolidin-1-yl)-3- methoxyphenyl)-6-(3,4-dihydro- 2H-benzo[b][1,4]oxazin-6- yl)imidazo[1,2-a]pyrazin-8- amine | — | — |
| 9 | 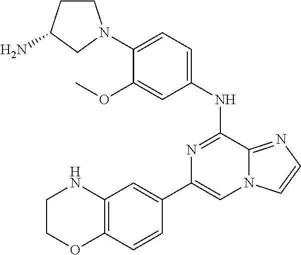 [SMILES: COc1cc(Nc2nc(-c3ccc4c(c3)NCCO4)cn3ccnc23)ccc1N1CC[C@@H](N)C1] | (R)-N-(4-(3-aminopyrrolidin-1- yl)-3-methoxyphenyl)-6-(3,4- dihydro-2H- benzo[b][1,4]oxazin-6- yl)imidazo[1,2-a]pyrazin-8- amine | 17 | 0.006 |
| 12 | 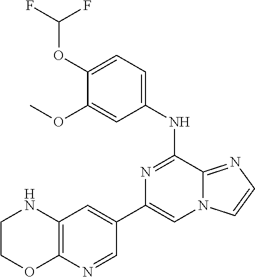 [SMILES: COc1cc(Nc2nc(-c3cnc4c(c3)NCCO4)cn3ccnc23)ccc1OC(F)F] | N-(4-(difluoromethoxy)-3- methoxyphenyl)-6-(2,3-dihydro- 1H-pyrido[2,3-b][1,4]oxazin-7- yl)imidazo[1,2-a]pyrazin-8- amine | 18 | 0.025 |
| 13 | 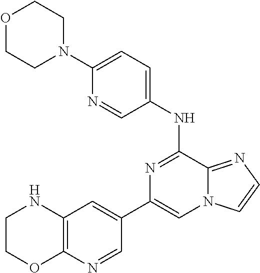 [SMILES: c1cn2cc(-c3cnc4c(c3)NCCO4)nc(Nc3ccc(N4CCOCC4)nc3)c2n1] | 6-(2,3-dihydro-1H-pyrido[2,3- b][1,4]oxazin-7-yl)-N-(6- morpholinopyridin-3- yl)imidazo[1,2-a]pyrazin-8- amine | 19 | 0.02 |
| 15 | 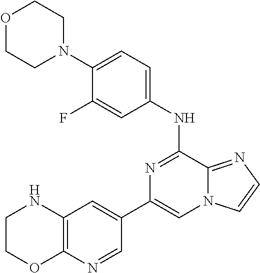 [SMILES: Fc1cc(Nc2nc(-c3cnc4c(c3)NCCO4)cn3ccnc23)ccc1N1CCOCC1] | 6-(2,3-dihydro-1H-pyrido[2,3- b][1,4]oxazin-7-yl)-N-(3-fluoro- 4- morpholinophenyl)imidazo[1,2- a]pyrazin-8-amine | 20 | 0.004 |
| 16A | 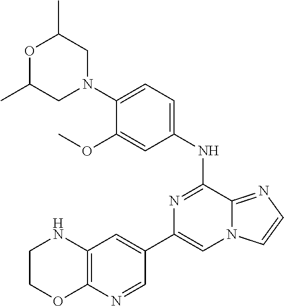 [SMILES: COc1cc(Nc2nc(-c3cnc4c(c3)NCCO4)cn3ccnc23)ccc1N1CC(C)OC(C)C1] | 6-(2,3-dihydro-1H-pyrido[2,3- b][1,4]oxazin-7-yl)-N-(4-(2,6- dimethylmorpholino)-3- methoxyphenyl)imidazo[1,2- a]pyrazin-8-amine | — | — |
| 16 | 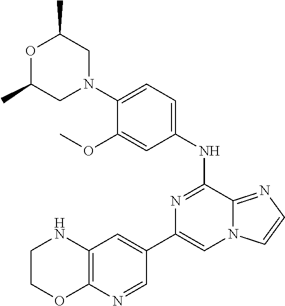 [SMILES: COc1cc(Nc2nc(-c3cnc4c(c3)NCCO4)cn3ccnc23)ccc1N1C[C@@H](C)O[C@@H](C)C1] | 6-(2,3-dihydro-1H-pyrido[2,3- b][1,4]oxazin-7-yl)-N-(4- ((2S,6R)-2,6- dimethylmorpholino)-3- methoxyphenyl)imidazo[1,2- a]pyrazin-8-amine | 21 | 0.006 |
| 17 | 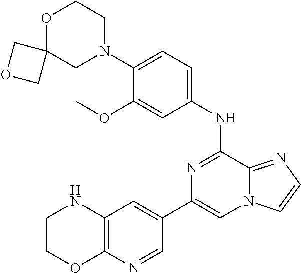 [SMILES: COc1cc(Nc2nc(-c3cnc4c(c3)NCCO4)cn3ccnc23)ccc1N1CCOC2(COC2)C1] | 6-(2,3-dihydro-1H-pyrido[2,3- b][1,4]oxazin-7-yl)-N-(3- methoxy-4-(2,5-dioxa-8- azaspiro[3.5]nonan-8- yl)phenyl)imidazo[1,2-a]pyrazin- 8-amine | 22 | 0.014 |
| 18A | 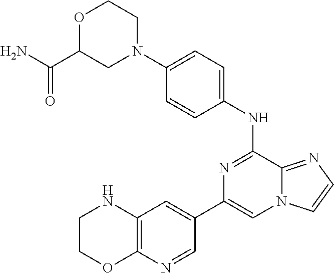 [SMILES: NC(=O)C1CN(c2ccc(Nc3nc(-c4cnc5c(c4)NCCO5)cn4ccnc34)cc2)CCO1] | 4-(4-((6-(2,3-dihydro-1H- pyrido[2,3-b][1,4]oxazin-7- yl)imidazo[1,2-a]pyrazin-8- yl)amino)phenyl)morpholine-2- carboxamide | — | — |
| 18 | 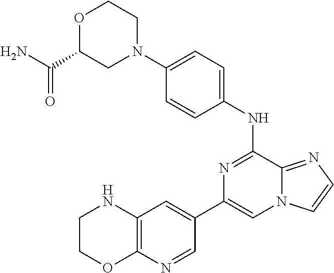 [SMILES: NC(=O)[C@H]1CN(c2ccc(Nc3nc(-c4cnc5c(c4)NCCO5)cn4ccnc34)cc2)CCO1] | (R)-4-(4-((6-(2,3-dihydro-1H- pyrido[2,3-b][1,4]oxazin-7- yl)imidazo[1,2-a]pyrazin-8- yl)amino)phenyl)morpholine-2- carboxamide | 23 | 0.006 |
| 19 | 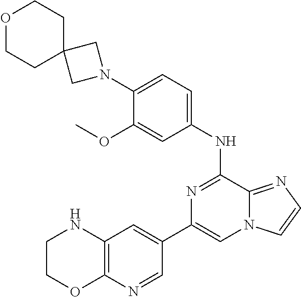 [SMILES: COc1cc(Nc2nc(-c3cnc4c(c3)NCCO4)cn3ccnc23)ccc1N1CC2(CCOCC2)C1] | 6-(2,3-dihydro-1H-pyrido[2,3- b][1,4]oxazin-7-yl)-N-(3- methoxy-4-(7-oxa-2- azaspiro[3.5]nonan-2- yl)phenyl)imidazo[1,2-a]pyrazin- 8-amine | 24 | 0.033 |
| 20 | 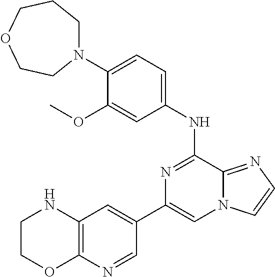 [SMILES: COc1cc(Nc2nc(-c3cnc4c(c3)NCCO4)cn3ccnc23)ccc1N1CCCOCC1] | 6-(2,3-dihydro-1H-pyrido[2,3- b][1,4]oxazin-7-yl)-N-(3- methoxy-4-(1,4-oxazepan-4- yl)phenyl)imidazo[1,2-a]pyrazin- 8-amine | 25 | 0.006 |
| 21A | 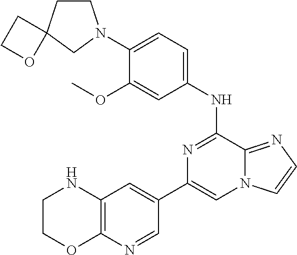 [SMILES: COc1cc(Nc2nc(-c3cnc4c(c3)NCCO4)cn3ccnc23)ccc1N1CCC2(CCO2)C1] | 6-(2,3-dihydro-1H-pyrido[2,3- b][1,4]oxazin-7-yl)-N-(3- methoxy-4-(1-oxa-6- azaspiro[3.4]octan-6- yl)phenyl)imidazo[1,2-a]pyrazin- 8-amine | — | — |
| 21 | 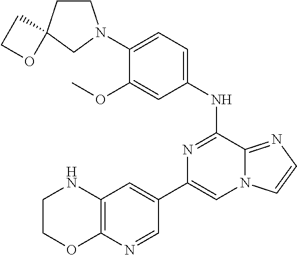 [SMILES: COc1cc(Nc2nc(-c3cnc4c(c3)NCCO4)cn3ccnc23)ccc1N1CC[C@@]2(CCO2)C1] | (R)-6-(2,3-dihydro-1H- pyrido[2,3-b][1,4]oxazin-7-yl)- N-(3-methoxy-4-(1-oxa-6- azaspiro[3.4]octan-6- yl)phenyl)imidazo[1,2-a]pyrazin- 8-amine | 26 | 0.010 |
| 22A | 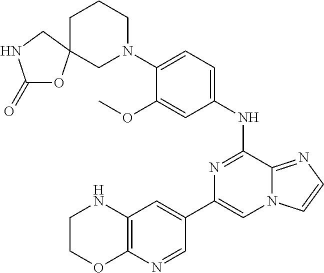 [SMILES: COc1cc(Nc2nc(-c3cnc4c(c3)NCCO4)cn3ccnc23)ccc1N1CCCC2(CNC(=O)O2)C1] | 7-(4-((6-(2,3-dihydro-1H- pyrido[2,3-b][1,4]oxazin-7- yl)imidazo[1,2-a]pyrazin-8- yl)amino)-2-methoxyphenyl)-1- oxa-3,7-diazaspiro[4.5]decan-2- one | — | — |
| 22 | 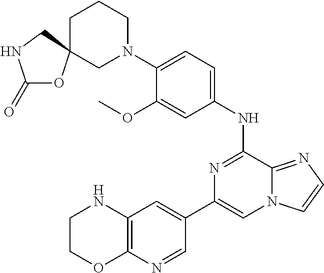 [SMILES: COc1cc(Nc2nc(-c3cnc4c(c3)NCCO4)cn3ccnc23)ccc1N1CCC[C@]2(CNC(=O)O2)C1] | (S)-7-(4-((6-(2,3-dihydro-1H- pyrido[2,3-b][1,4]oxazin-7- yl)imidazo[1,2-a]pyrazin-8- yl)amino)-2-methoxyphenyl)-1- oxa-3,7-diazaspiro[4.5]decan-2- one | 27 | 0.007 |
| 23 | 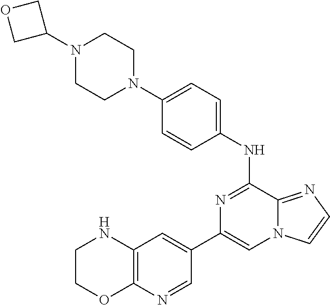 [SMILES: c1cn2cc(-c3cnc4c(c3)NCCO4)nc(Nc3ccc(N4CCN(C5COC5)CC4)cc3)c2n1] | 6-(2,3-dihydro-1H-pyrido[2,3- b][1,4]oxazin-7-yl)-N-(4-(4- (oxetan-3-yl)piperazin-1- yl)phenyl)imidazo[1,2-a]pyrazin- 8-amine | 31 | 0.007 |
| 24 | 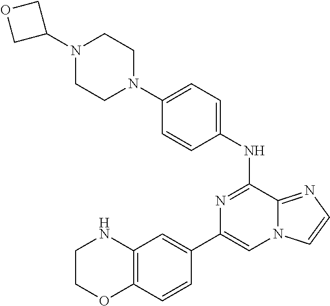 [SMILES: c1cn2cc(-c3ccc4c(c3)NCCO4)nc(Nc3ccc(N4CCN(C5COC5)CC4)cc3)c2n1] | 6-(3,4-dihydro-2H- benzo[b][1,4]oxazin-6-yl)-N-(4- (4-(oxetan-3-yl)piperazin-1- yl)phenyl)imidazo[1,2-a]pyrazin- 8-amine | 32 | 0.011 |
| 25 | 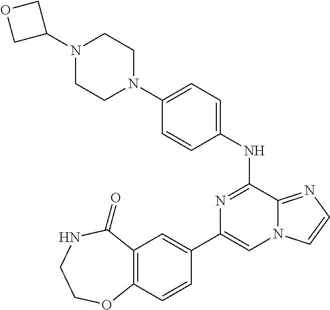 [SMILES: O=C1NCCOc2ccc(-c3cn4ccnc4c(Nc4ccc(N5CCN(C6COC6)CC5)cc4)n3)cc21] | 7-(8-((4-(4-(oxetan-3- yl)piperazin-1- yl)phenyl)amino)imidazo[1,2- a]pyrazin-6-yl)-3,4- dihydrobenzo[f][1,4]oxazepin- 5(2H)-one | 33 | 0.012 |
| 26 | 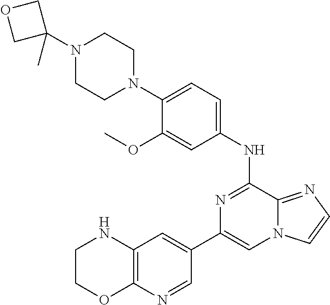 [SMILES: COc1cc(Nc2nc(-c3cnc4c(c3)NCCO4)cn3ccnc23)ccc1N1CCN(C2(C)COC2)CC1] | 6-(2,3-dihydro-1H-pyrido[2,3- b][1,4]oxazin-7-yl)-N-(3- methoxy-4-(4-(3-methyloxetan- 3-yl)piperazin-1- yl)phenyl)imidazo[1,2-a]pyrazin- 8-amine | 37 | 0.005 |
| 27 | 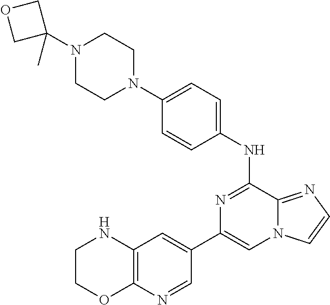 [SMILES: CC1(N2CCN(c3ccc(Nc4nc(-c5cnc6c(c5)NCCO6)cn5ccnc45)cc3)CC2)COC1] | 6-(2,3-dihydro-1H-pyrido[2,3- b][1,4]oxazin-7-yl)-N-(4-(4-(3- methyloxetan-3-yl)piperazin-1- yl)phenyl)imidazo[1,2-a]pyrazin- 8-amine | 38 | 0.007 |
| 28 | 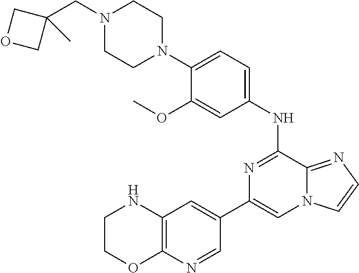 [SMILES: COc1cc(Nc2nc(-c3cnc4c(c3)NCCO4)cn3ccnc23)ccc1N1CCN(CC2(C)COC2)CC1] | 6-(2,3-dihydro-1H-pyrido[2,3- b][1,4]oxazin-7-yl)-N-(3- methoxy-4-(4-((3-methyloxetan- 3-yl)methyl)piperazin-1- yl)phenyl)imidazo[1,2-a]pyrazin- 8-amine | 41 | 0.004 |
| 29 | 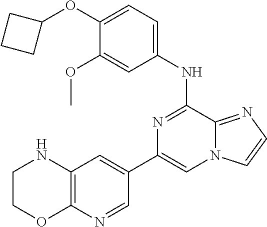 [SMILES: COc1cc(Nc2nc(-c3cnc4c(c3)NCCO4)cn3ccnc23)ccc1OC1CCC1] | N-(4-cyclobutoxy-3- methoxyphenyl)-6-(2,3-dihydro- 1H-pyrido[2,3-b][1,4]oxazin-7- yl)imidazo[1,2-a]pyrazin-8- amine | 45 | 0.007 |
| 30 | 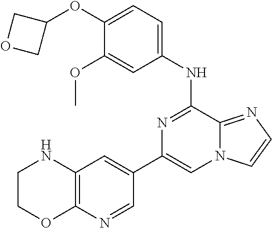 [SMILES: COc1cc(Nc2nc(-c3cnc4c(c3)NCCO4)cn3ccnc23)ccc1OC1COC1] | 6-(2,3-dihydro-1H-pyrido[2,3- b][1,4]oxazin-7-yl)-N-(3- methoxy-4-(oxetan-3- yloxy)phenyl)imidazo[1,2- a]pyrazin-8-amine | 46 | 0.002 |
| 31 | 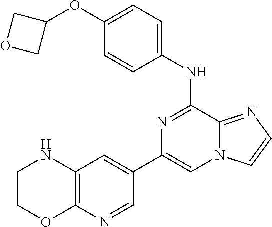 [SMILES: c1cn2cc(-c3cnc4c(c3)NCCO4)nc(Nc3ccc(OC4COC4)cc3)c2n1] | 6-(2,3-dihydro-1H-pyrido[2,3- b][1,4]oxazin-7-yl)-N-(4- (oxetan-3- yloxy)phenyl)imidazo[1,2- a]pyrazin-8-amine | 47 | 0.007 |
| 32 | 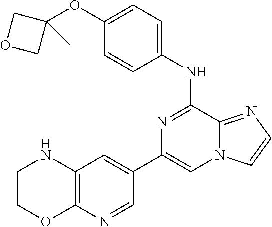 [SMILES: CC1(Oc2ccc(Nc3nc(-c4cnc5c(c4)NCCO5)cn4ccnc34)cc2)COC1] | 6-(2,3-dihydro-1H-pyrido[2,3- b][1,4]oxazin-7-yl)-N-(4-((3- methyloxetan-3- yl)oxy)phenyl)imidazo[1,2- a]pyrazin-8-amine | 48 | 0.006 |
| 33 | 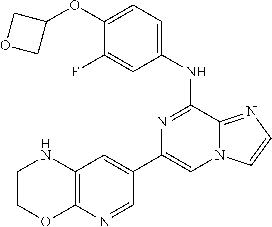 [SMILES: Fc1cc(Nc2nc(-c3cnc4c(c3)NCCO4)cn3ccnc23)ccc1OC1COC1] | 6-(2,3-dihydro-1H-pyrido[2,3- b][1,4]oxazin-7-yl)-N-(3-fluoro- 4-(oxetan-3- yloxy)phenyl)imidazo[1,2- a]pyrazin-8-amine | 49 | 0.010 |
| 34 | 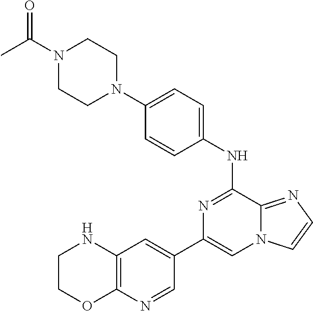 [SMILES: CC(=O)N1CCN(c2ccc(Nc3nc(-c4cnc5c(c4)NCCO5)cn4ccnc34)cc2)CC1] | 1-(4-(4-((6-(2,3-dihydro-1H- pyrido[2,3-b][1,4]oxazin-7- yl)imidazo[1,2-a]pyrazin-8- yl)amino)pheny])piperazin-1- yl)ethanone | 50 | 0.004 |
| 35 | 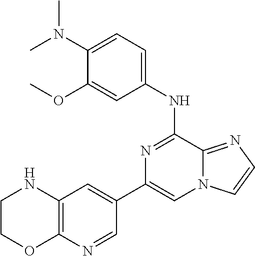 [SMILES: COc1cc(Nc2nc(-c3cnc4c(c3)NCCO4)cn3ccnc23)ccc1N(C)C] | N4-(6-(2,3-dihydro-1H- pyrido[2,3-b][1,4]oxazin-7- yl)imidazo[1,2-a]pyrazin-8-yl)- 2-methoxy-N1,N1- dimethylbenzene-1,4-diamine | 51 | 0.002 |
| 36 | 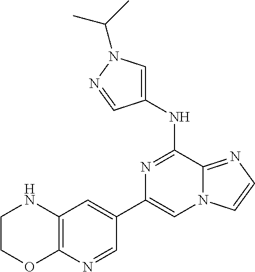 [SMILES: CC(C)n1cc(Nc2nc(-c3cnc4c(c3)NCCO4)cn3ccnc23)cn1] | 6-(2,3-dihydro-1H-pyrido[2,3- b][1,4]oxazin-7-yl)-N-(1- isopropyl-1H-pyrazol-4- yl)imidazo[1,2-a]pyrazin-8- amine | 53 | 0.008 |
| 37 | 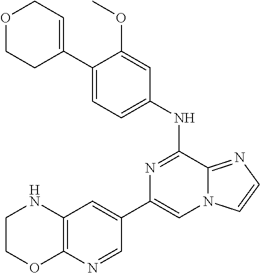 [SMILES: COc1cc(Nc2nc(-c3cnc4c(c3)NCCO4)cn3ccnc23)ccc1C1=CCOCC1] | 6-(2,3-dihydro-1H-pyrido[2,3- b][1,4]oxazin-7-yl)-N-(4-(3,6- dihydro-2H-pyran-4-yl)-3- methoxyphenyl)imidazo[1,2- a]pyrazin-8-amine | 56 | 0.013 |
| 38 | 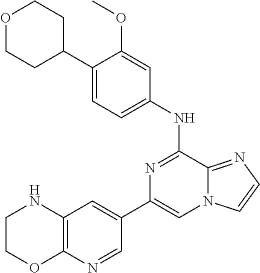 [SMILES: COc1cc(Nc2nc(-c3cnc4c(c3)NCCO4)cn3ccnc23)ccc1C1CCOCC1] | 6-(2,3-dihydro-1H-pyrido[2,3- b][1,4]oxazin-7-yl)-N-(3- methoxy-4-(tetrahydro-2H- pyran-4-yl)phenyl)imidazo[1,2- a]pyrazin-8-amine | 57 | 0.014 |
| 39 | 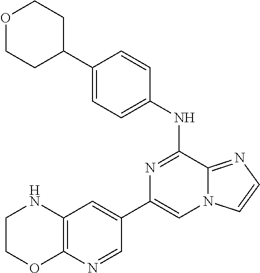 [SMILES: c1cn2cc(-c3cnc4c(c3)NCCO4)nc(Nc3ccc(C4CCOCC4)cc3)c2n1] | 6-(2,3-dihydro-1H-pyrido[2,3- b][1,4]oxazin-7-yl)-N-(4- (tetrahydro-2H-pyran-4- yl)phenyl)imidazo[1,2-a]pyrazin- 8-amine | 59 | 0.007 |
| 40 | 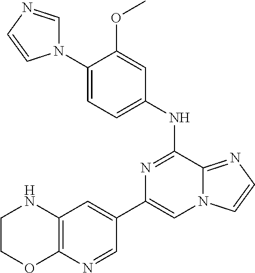 [SMILES: COc1cc(Nc2nc(-c3cnc4c(c3)NCCO4)cn3ccnc23)ccc1-n1ccnc1] | N-(4-(1H-imidazol-1-yl)-3- methoxyphenyl)-6-(2,3-dihydro- 1H-pyrido[2,3-b][1,4]oxazin-7- yl)imidazo[1,2-a]pyrazin-8- amine | 63 | 0.021 |
| 43A | 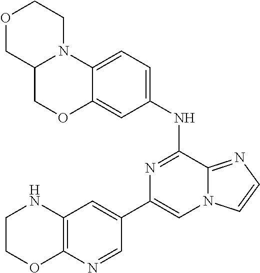 [SMILES: c1cn2cc(-c3cnc4c(c3)NCCO4)nc(Nc3ccc4c(c3)OCC3COCCN43)c2n1] | N-(6-(2,3-dihydro-1H- pyrido[2,3-b][1,4]oxazin-7- yl)imidazo[1,2-a]pyrazin-8-yl)- 2,4,4a,5-tetrahydro-1H- benzo[b][1,4]oxazino[4,3- d][1,4]oxazin-8-amine | — | — |
| 43 | 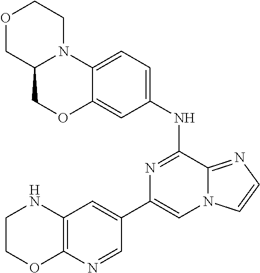 [SMILES: c1cn2cc(-c3cnc4c(c3)NCCO4)nc(Nc3ccc4c(c3)OC[C@H]3COCCN43)c2n1] | (R)-N-(6-(2,3-dihydro-1H- pyrido[2,3-b][1,4]oxazin-7- yl)imidazo[1,2-a]pyrazin-8-yl)- 2,4,4a,5-tetrahydro-1H- benzo[b][1,4]oxazino[4,3- d][1,4]oxazin-8-amine | 64 | 0.009 |
| 44 | 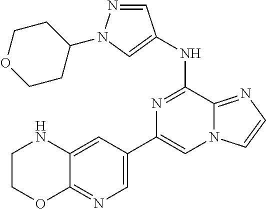 [SMILES: c1cn2cc(-c3cnc4c(c3)NCCO4)nc(Nc3cnn(C4CCOCC4)c3)c2n1] | 6-(2,3-dihydro-1H-pyrido[2,3- b][1,4]oxazin-7-yl)-N-(1- (tetrahydro-2H-pyran-4-yl)-1H- pyrazol-4-yl)imidazo[1,2- a]pyrazin-8-amine | 65 | 0.005 |
| 45 | 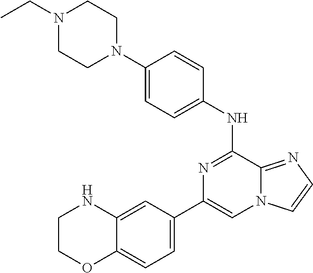 [SMILES: CCN1CCN(c2ccc(Nc3nc(-c4ccc5c(c4)NCCO5)cn4ccnc34)cc2)CC1] | 6-(3,4-dihydro-2H- benzo[b][1,4]oxazin-6-yl)-N-(4- (4-ethylpiperazin-1- yl)phenyl)imidazo[1,2-a]pyrazin- 8-amine | 66 | 0.037 |
| 47 | 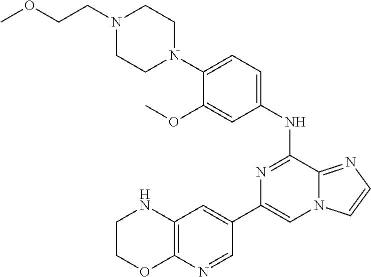 [SMILES: COCCN1CCN(c2ccc(Nc3nc(-c4cnc5c(c4)NCCO5)cn4ccnc34)cc2OC)CC1] | 6-(2,3-dihydro-1H-pyrido[2,3- b][1,4]oxazin-7-yl)-N-(3- methoxy-4-(4-(2- methoxyethyl)piperazin-1- yl)phenyl)imidazo[1,2-a]pyrazin- 8-amine | 67 | 0.003 |
| 48 | 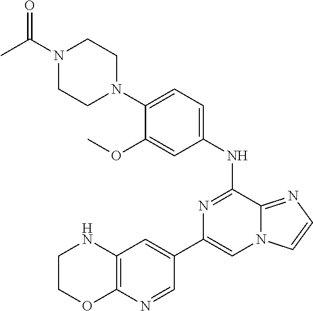 [SMILES: COc1cc(Nc2nc(-c3cnc4c(c3)NCCO4)cn3ccnc23)ccc1N1CCN(C(C)=O)CC1] | l-(4-(4-((6-(2,3-dihydro-1H- pyrido[2,3-b][1,4]oxazin-7- yl)imidazo[1,2-a]pyrazin-8- yl)amino)-2- methoxyphenyl)piperazin-1- yl)ethanone | 68 | 0.003 |
| 49 | 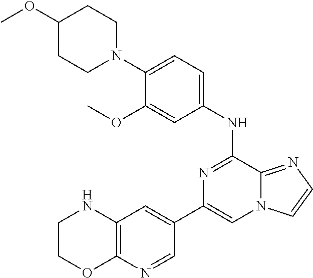 [SMILES: COc1cc(Nc2nc(-c3cnc4c(c3)NCCO4)cn3ccnc23)ccc1N1CCC(OC)CC1] | 6-(2,3-dihydro-1H-pyrido[2,3- b][1,4]oxazin-7-yl)-N-(3- methoxy-4-(4-methoxypiperidin- 1-yl)phenyl)imidazo[1,2- a]pyrazin-8-amine | 69 | 0.005 |
| 50 | 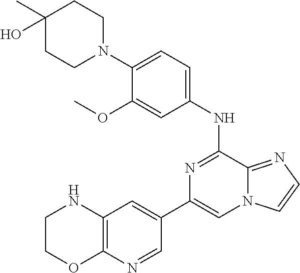 [SMILES: COc1cc(Nc2nc(-c3cnc4c(c3)NCCO4)cn3ccnc23)ccc1N1CCC(C)(O)CC1] | 1-(4-((6-(2,3-dihydro-1H- pyrido[2,3-b][1,4]oxazin-7- yl)imidazo[1,2-a]pyrazin-8- yl)amino)-2-methoxyphenyl)-4- methylpiperidin-4-ol | 70 | 0.007 |
| 51 | 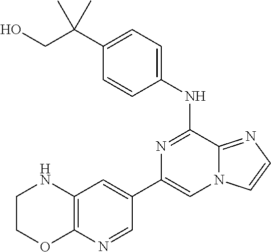 [SMILES: CC(C)(CO)c1ccc(Nc2nc(-c3cnc4c(c3)NCCO4)cn3ccnc23)cc1] | 2-(4-((6-(2,3-dihydro-1H- pyrido[2,3-b][1,4]oxazin-7- yl)imidazo[1,2-a]pyrazin-8- yl)amino)phenyl)-2- methylpropan-1-ol | 71 | 0.004 |
| 52A | 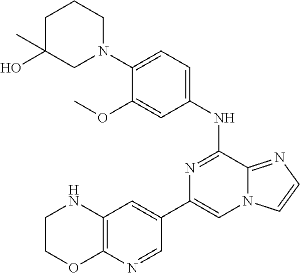 [SMILES: COc1cc(Nc2nc(-c3cnc4c(c3)NCCO4)cn3ccnc23)ccc1N1CCCC(C)(O)C1] | 1-(4-((6-(2,3-dihydro-1H- pyrido[2,3-b][1,4]oxazin-7- yl)imidazo[1,2-a]pyrazin-8- yl)amino)-2-methoxyphenyl)-3- methylpiperidin-3-ol | — | — |
| 52 | 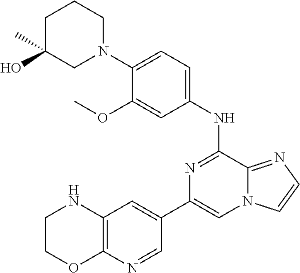 [SMILES: COc1cc(Nc2nc(-c3cnc4c(c3)NCCO4)cn3ccnc23)ccc1N1CCC[C@](C)(O)C1] | (S)-1-(4-((6-(2,3-dihydro-1H- pyrido[2,3-b][1,4]oxazin-7- yl)imidazo[1,2-a]pyrazin-8- yl)amino)-2-methoxyphenyl)-3- methylpiperidin-3-ol | 72 | 0.007 |
| 53 | 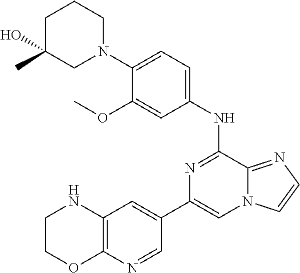 [SMILES: COc1cc(Nc2nc(-c3cnc4c(c3)NCCO4)cn3ccnc23)ccc1N1CCC[C@@](C)(O)C1] | (R)-1-(4-((6-(2,3-dihydro-1H- pyrido[2,3-b][1,4]oxazin-7- yl)imidazo[1,2-a]pyrazin-8- yl)amino)-2-methoxyphenyl)-3- methylpiperidin-3-ol | 73 | 0.010 |
| 54 | 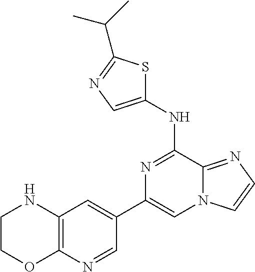 [SMILES: CC(C)c1ncc(Nc2nc(-c3cnc4c(c3)NCCO4)cn3ccnc23)s1] | N-(6-(2,3-dihydro-1H- pyrido[2,3-b][1,4]oxazin-7- yl)imidazo[1,2-a]pyrazin-8-yl)- 2-isopropylthiazol-5-amine | 74 | 0.004 |
| 55 | 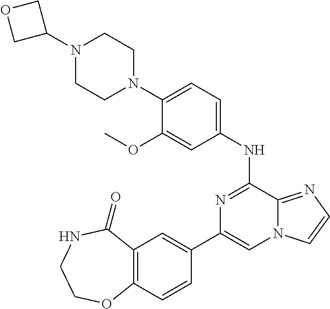 [SMILES: COc1cc(Nc2nc(-c3ccc4c(c3)C(=O)NCCO4)cn3ccnc23)ccc1N1CCN(C2COC2)CC1] | 7-(8-((3-methoxy-4-(4-(oxetan- 3-yl)piperazin-1- yl)phenyl)amino)imidazo[1,2- a]pyrazin-6-yl)-3,4- dihydrobenzo[f][1,4]oxazepin- 5(2H)-one | 75 | 0.008 |
| 56A | 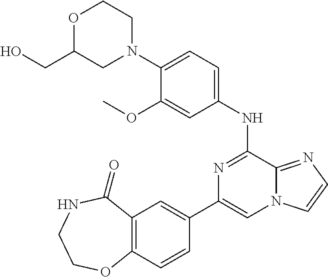 [SMILES: COc1cc(Nc2nc(-c3ccc4c(c3)C(=O)NCCO4)cn3ccnc23)ccc1N1CCOC(CO)C1] | 7-(8-((4-(2- (hydroxymethyl)morpholino)-3- methoxyphenyl)amino)imidazo [1,2-a]pyrazin-6-yl)-3,4- dihydrobenzo[f][1,4]oxazepin- 5(2H)-one | — | — |
| 56 | 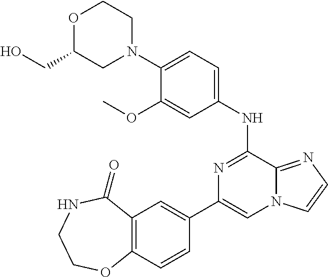 [SMILES: COc1cc(Nc2nc(-c3ccc4c(c3)C(=O)NCCO4)cn3ccnc23)ccc1N1CCO[C@@H](CO)C1] | (R)-7-(8-((4-(2- (hydroxymethyl)morpholinyl)-3- methoxyphenyl)amino)imidazo [1,2-a]pyrazin-6-yl)-3,4- dihydrobenzo[f][1,4]oxazepin- 5(2H)-one | 76 | 0.037 |
| 57 | 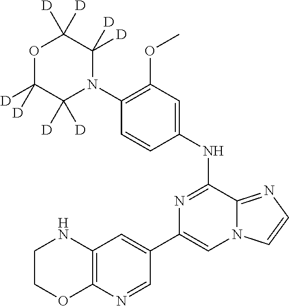 [SMILES: [2H]C1([2H])OC([2H])([2H])C([2H])([2H])N(c2ccc(Nc3nc(-c4cnc5c(c4)NCCO5)cn4ccnc34)cc2OC)C1([2H])[2H]] | 6-(2,3-dihydro-1H-pyrido[2,3- b][1,4]oxazin-7-yl)-N-(3- methoxy-4- octadeuteratedmorpholinophenyl) imidazo[1,2-a]pyrazin-8-amine | 77 | 0.004 |
| 58 | 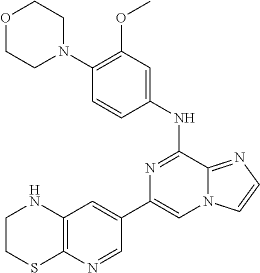 [SMILES: COc1cc(Nc2nc(-c3cnc4c(c3)NCCS4)cn3ccnc23)ccc1N1CCOCC1] | 6-(2,3-dihydro-1H-pyrido[2,3- b][1,4]thiazin-7-yl)-N-(3- methoxy-4- morpholinophenyl)imidazo[1,2- a]pyrazin-8-amine | 79 | 0.002 |
| 59 | 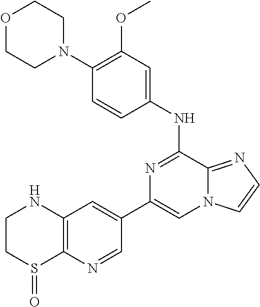 [SMILES: COc1cc(Nc2nc(-c3cnc4c(c3)NCCS4=O)cn3ccnc23)ccc1N1CCOCC1] | 7-(8-((3-methoxy-4- morpholinophenyl)amino)imidazo [1,2-a]pyrazin-6-yl)-2,3- dihydro-1H-pyrido[2,3- b][1,4]thiazine 4-oxide | 80 | 0.010 |
| 60 | 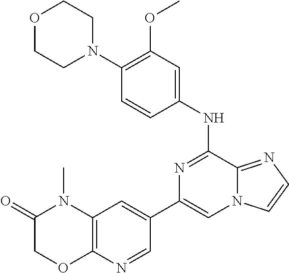 [SMILES: COc1cc(Nc2nc(-c3cnc4c(c3)N(C)C(=O)CO4)cn3ccnc23)ccc1N1CCOCC1] | 7-(8-((3-methoxy-4- morpholinophenyl)amino)imidazo [1,2-a]pyrazin-6-yl)-1-methyl- 1H-pyrido[2,3-b][1,4]oxazin- 2(3H)-one | 83 | 0.039 |
| 61A | 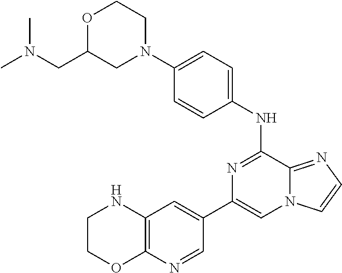 [SMILES: CN(C)CC1CN(c2ccc(Nc3nc(-c4cnc5c(c4)NCCO5)cn4ccnc34)cc2)CCO1] | 6-(2,3-dihydro-1H-pyrido[2,3- b][1,4]oxazin-7-yl)-N-(4-(2- ((dimethylamino)methyl) morpholino)phenyl)imidazo[1,2- a]pyrazin-8-amine | — | — |
| 61 | 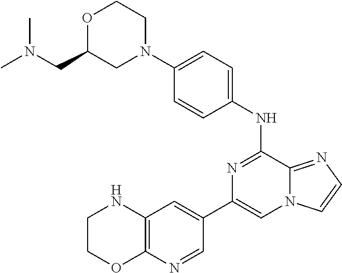 [SMILES: CN(C)C[C@@H]1CN(c2ccc(Nc3nc(-c4cnc5c(c4)NCCO5)cn4ccnc34)cc2)CCO1] | (R)-6-(2,3-dihydro-1H- pyrido[2,3-b][1,4]oxazin-7-yl)- N-(4-(2- ((dimethylamino)methyl) morpholino)phenyl)imidazo[1,2- a]pyrazin-8-amine | 86 | 0.009 |
| 62 | 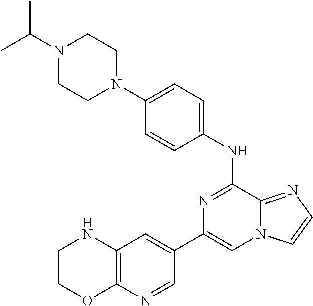 [SMILES: CC(C)N1CCN(c2ccc(Nc3nc(-c4cnc5c(c4)NCCO5)cn4ccnc34)cc2)CC1] | 6-(2,3-dihydro-1H-pyrido[2,3- b][1,4]oxazin-7-yl)-N-(4-(4- isopropylpiperazin-1- yl)phenyl)imidazo[1,2-a]pyrazin- 8-amine | 87 | 0.008 |
| 63 | 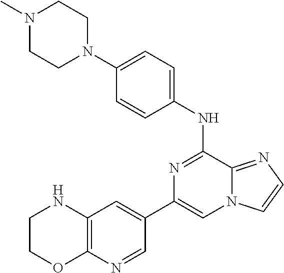 [SMILES: CN1CCN(c2ccc(Nc3nc(-c4cnc5c(c4)NCCO5)cn4ccnc34)cc2)CC1] | 6-(2,3-dihydro-1H-pyrido[2,3- b][1,4]oxazin-7-yl)-N-(4-(4- methylpiperazin-1- yl)phenyl)imidazo[1,2-a]pyrazin- 8-amine | 88 | 0.009 |
| 64 | 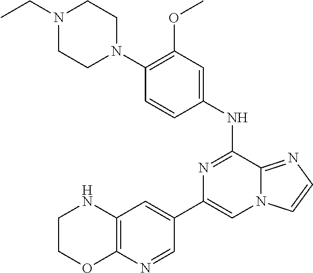 [SMILES: CCN1CCN(c2ccc(Nc3nc(-c4cnc5c(c4)NCCO5)cn4ccnc34)cc2OC)CC1] | 6-(2,3-dihydro-1H-pyrido[2,3- b][1,4]oxazin-7-yl)-N-(4-(4- ethylpiperazin-1-yl)-3- methoxyphenyl)imidazo[1,2- a]pyrazin-8-amine | 89 | 0.008 |
| 65 | 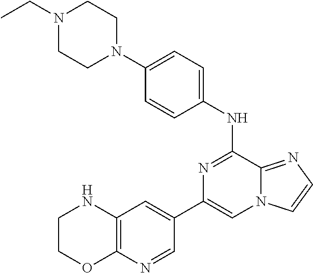 [SMILES: CCN1CCN(c2ccc(Nc3nc(-c4cnc5c(c4)NCCO5)cn4ccnc34)cc2)CC1] | 6-(2,3-dihydro-1H-pyrido[2,3- b][1,4]oxazin-7-yl)-N-(4-(4- ethylpiperazin-1- yl)phenyl)imidazo[1,2-a]pyrazin- 8-amine | 90 | 0.014 |
| 66 | 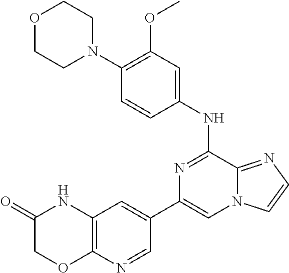 [SMILES: COc1cc(Nc2nc(-c3cnc4c(c3)NC(=O)CO4)cn3ccnc23)ccc1N1CCOCC1] | 7-(8-((3-methoxy-4- morpholinophenyl)amino)imidazo [1,2-a]pyrazin-6-yl)-1H- pyrido[2,3-b][1,4]oxazin-2(3H)- one | 91 | 0.003 |
| 67 | 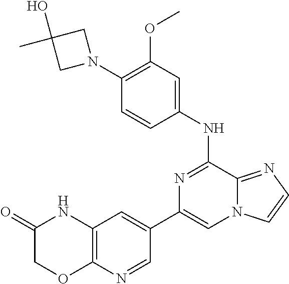 [SMILES: COc1cc(Nc2nc(-c3cnc4c(c3)NC(=O)CO4)cn3ccnc23)ccc1N1CC(C)(O)C1] | 7-(8-((4-(3-hydroxy-3- methylazetidin-1-yl)-3- methoxyphenyl)amino)imidazo [1,2-a]pyrazin-6-yl)-1H- pyrido[2,3-b][1,4]oxazin-2(3H)- one | 92 | 0.014 |
| 68 | 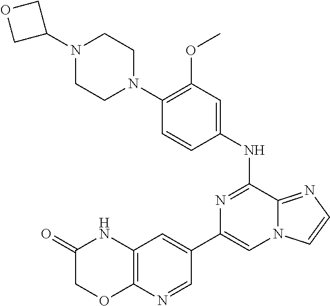 [SMILES: COc1cc(Nc2nc(-c3cnc4c(c3)NC(=O)CO4)cn3ccnc23)ccc1N1CCN(C2COC2)CC1] | 7-(8-((3-methoxy-4-(4-(oxetan- 3-yl)piperazin-1- yl)phenyl)amino)imidazo[1,2- a]pyrazin-6-yl)-1H-pyrido[2,3- b][1,4]oxazin-2(3H)-one | 93 | 0.006 |
| 69A | 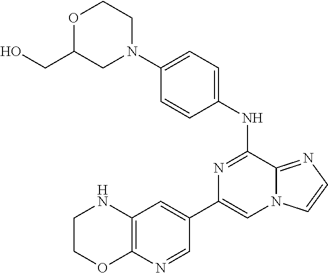 [SMILES: OCC1CN(c2ccc(Nc3nc(-c4cnc5c(c4)NCCO5)cn4ccnc34)cc2)CCO1] | (4-(4-((6-(2,3-dihydro-1H- pyrido[2,3-b][1,4]oxazin-7- yl)imidazo[1,2-a]pyrazin-8- yl)amino)phenyl)morpholin-2- yl)methanol | — | — |
| 69 | 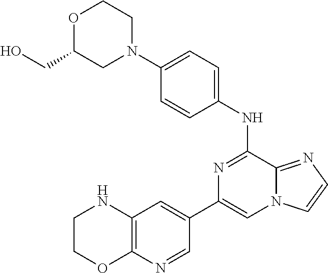 [SMILES: OC[C@H]1CN(c2ccc(Nc3nc(-c4cnc5c(c4)NCCO5)cn4ccnc34)cc2)CCO1] | (R)-(4-(4-((6-(2,3-dihydro-1H- pyrido[2,3-b][1,4]oxazin-7- yl)imidazo[1,2-a]pyrazin-8- yl)amino)phenyl)morpholin-2- yl)methanol | 94 | 0.008 |
| 70 | 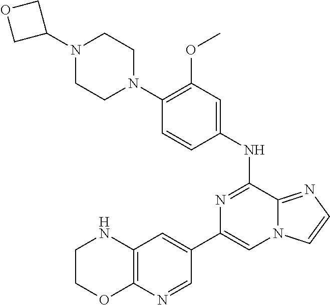 [SMILES: COc1cc(Nc2nc(-c3cnc4c(c3)NCCO4)cn3ccnc23)ccc1N1CCN(C2COC2)CC1] | 6-(2,3-dihydro-1H-pyrido[2,3- b][1,4]oxazin-7-yl)-N-(3- methoxy-4-(4-(oxetan-3- yl)piperazin-1- yl)phenyl)imidazo[1,2-a]pyrazin- 8-amine | 95 | 0.011 |
| 71A | 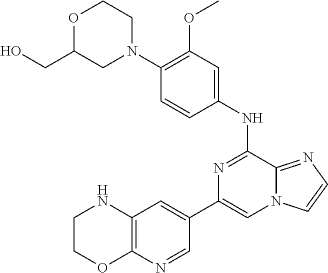 [SMILES: COc1cc(Nc2nc(-c3cnc4c(c3)NCCO4)cn3ccnc23)ccc1N1CCOC(CO)C1] | (4-(4-((6-(2,3-dihydro-1H- pyrido[2,3-b][1,4]oxazin-7- yl)imidazo[1,2-a]pyrazin-8- yl)amino)-2- methoxyphenyl)morpholin-2- yl)methanol | — | — |
| 71 | 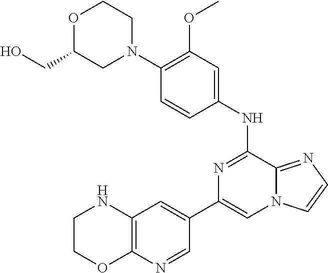 [SMILES: COc1cc(Nc2nc(-c3cnc4c(c3)NCCO4)cn3ccnc23)ccc1N1CCO[C@@H](CO)C1] | (R)-(4-(4-((6-(2,3-dihydro-1H- pyrido[2,3-b][1,4]oxazin-7- yl)imidazo[1,2-a]pyrazin-8- yl)amino)-2- methoxyphenyl)morpholin-2- yl)methanol | 96 | 0.015 |
| 72A | 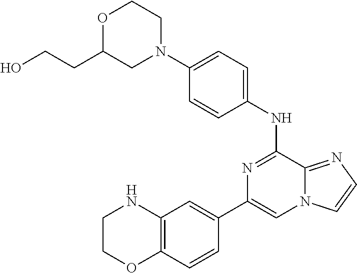 [SMILES: OCCC1CN(c2ccc(Nc3nc(-c4ccc5c(c4)NCCO5)cn4ccnc34)cc2)CCO1] | 2-(4-(4-((6-(3,4-dihydro-2H- benzo[b][1,4]oxazin-6- yl)imidazo[1,2-a]pyrazin-8- yl)amino)phenyl)morpholin-2- yl)ethanol | — | — |
| 72 | 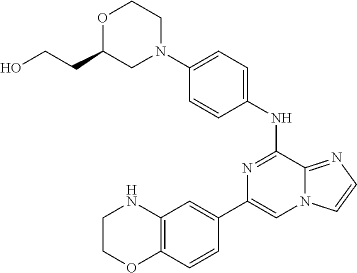 [SMILES: OCC[C@@H]1CN(c2ccc(Nc3nc(-c4ccc5c(c4)NCCO5)cn4ccnc34)cc2)CCO1] | (R)-2-(4-(4-((6-(3,4-dihydro-2H- benzo[b][1,4]oxazin-6- yl)imidazo[1,2-a]pyrazin-8- yl)amino)phenyl)morpholin-2- yl)ethanol | 100 | 0.012 |
| 76A | 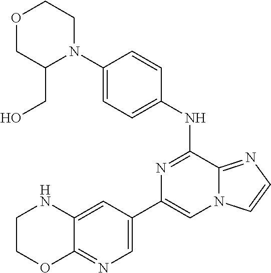 [SMILES: OCC1COCCN1c1ccc(Nc2nc(-c3cnc4c(c3)NCCO4)cn3ccnc23)cc1] | (4-(4-((6-(2,3-dihydro-1H- pyrido[2,3-b][1,4]oxazin-7- yl)imidazo[1,2-a]pyrazin-8- yl)amino)phenyl)morpholin-3- yl)methanol | — | — |
| 76 | 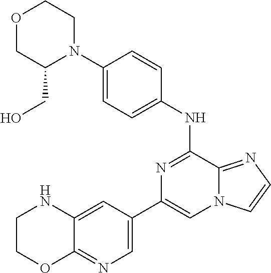 [SMILES: OC[C@@H]1COCCN1c1ccc(Nc2nc(-c3cnc4c(c3)NCCO4)cn3ccnc23)cc1] | (R)-(4-(4-((6-(2,3-dihydro-1H- pyrido[2,3-b][1,4]oxazin-7- yl)imidazo[1,2-a]pyrazin-8- yl)amino)phenyl)morpholin-3- yl)methanol | 109 | 0.008 |
| 77 | 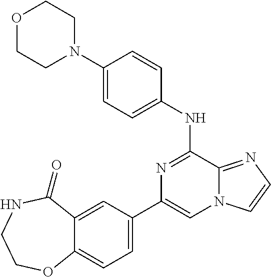 [SMILES: O=C1NCCOc2ccc(-c3cn4ccnc4c(Nc4ccc(N5CCOCC5)cc4)n3)cc21] | 7-(8-((4- morpholinophenyl)amino)imidazo [1,2-a]pyrazin-6-yl)-3,4- dihydrobenzo[f][1,4]oxazepin- 5(2H)-one | 110 | 0.019 |
| 78 | 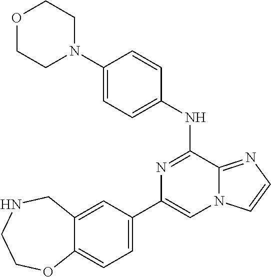 [SMILES: c1cn2cc(-c3ccc4c(c3)CNCCO4)nc(Nc3ccc(N4CCOCC4)cc3)c2n1] | N-(4-morpholinophenyl)-6- (2,3,4,5- tetrahydrobenzo[f][1,4]oxazepin- 7-yl)imidazo[1,2-a]pyrazin-8- amine | 111 | 0.018 |
| 80A | 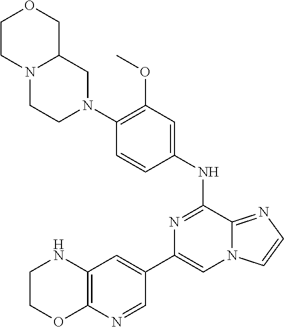 [SMILES: COc1cc(Nc2nc(-c3cnc4c(c3)NCCO4)cn3ccnc23)ccc1N1CCN2CCOCC2C1] | 6-(2,3-dihydro-1H-pyrido[2,3- b][1,4]oxazin-7-yl)-N-(4- (hexahydropyrazino[2,1- c][1,4]oxazin-8(1H)-yl)-3- methoxyphenyl)imidazo[1,2- a]pyrazin-8-amine | — | — |
| 80 | 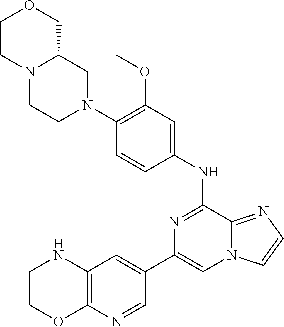 [SMILES: COc1cc(Nc2nc(-c3cnc4c(c3)NCCO4)cn3ccnc23)ccc1N1CCN2CCOC[C@@H]2C1] | (S)-6-(2,3-dihydro-1H- pyrido[2,3-b][1,4]oxazin-7-yl)- N-(4-(hexahydropyrazino[2,1- c][1,4]oxazin-8(1H)-yl)-3- methoxyphenyl)imidazo[1,2- a]pyrazin-8-amine | 115 | 0.010 |
| 81 | 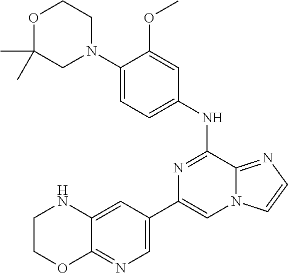 [SMILES: COc1cc(Nc2nc(-c3cnc4c(c3)NCCO4)cn3ccnc23)ccc1N1CCOC(C)(C)C1] | 6-(2,3-dihydro-1H-pyrido[2,3- b][1,4]oxazin-7-yl)-N-(4-(2,2- dimethylmorpholino)-3- methoxyphenyl)imidazo[1,2- a]pyrazin-8-amine | 119 | 0.014 |
| 82A | 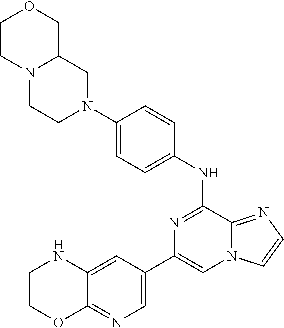 [SMILES: c1cn2cc(-c3cnc4c(c3)NCCO4)nc(Nc3ccc(N4CCN5CCOCC5C4)cc3)c2n1] | 6-(2,3-dihydro-1H-pyrido[2,3- b][1,4]oxazin-7-yl)-N-(4- (hexahydropyrazino[2,1- c][1,4]oxazin-8(1H)- yl)phenyl)imidazo[1,2-a]pyrazin- 8-amine | — | — |
| 82 | 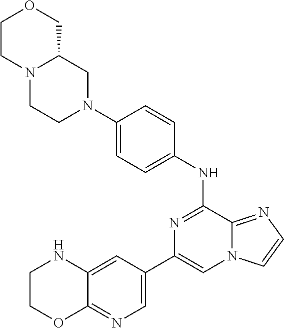 [SMILES: c1cn2cc(-c3cnc4c(c3)NCCO4)nc(Nc3ccc(N4CCN5CCOC[C@@H]5C4)cc3)c2n1] | (S)-6-(2,3-dihydro-1H- pyrido[2,3-b][1,4]oxazin-7-yl)- N-(4-(hexahydropyrazino[2,1- c][1,4]oxazin-8(1H)- yl)phenyl)imidazo[1,2-a]pyrazin- 8-amine | 123 | 0.003 |
| 83 | 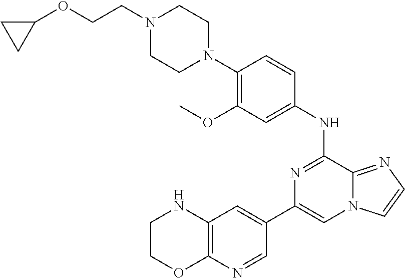 [SMILES: COc1cc(Nc2nc(-c3cnc4c(c3)NCCO4)cn3ccnc23)ccc1N1CCN(CCOC2CC2)CC1] | N-(4-(4-(2- cyclopropoxyethyl)piperazin-1- yl)-3-methoxyphenyl)-6-(2,3- dihydro-1H-pyrido[2,3- b][1,4]oxazin-7-yl)imidazo[1,2- a]pyrazin-8-amine | 127 | 0.006 |
| 84 | 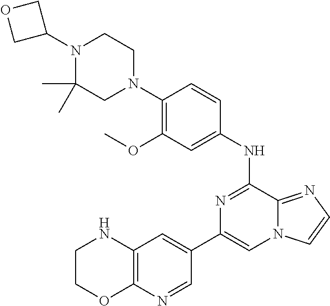 [SMILES: COc1cc(Nc2nc(-c3cnc4c(c3)NCCO4)cn3ccnc23)ccc1N1CCN(C2COC2)C(C)(C)C1] | 6-(2,3-dihydro-1H-pyrido[2,3- b][1,4]oxazin-7-yl)-N-(4-(3,3- dimethyl-4-(oxetan-3- yl)piperazin-1-yl)-3- methoxyphenyl)imidazo[1,2- a]pyrazin-8-amine | 133 | 0.010 |
| 85A | 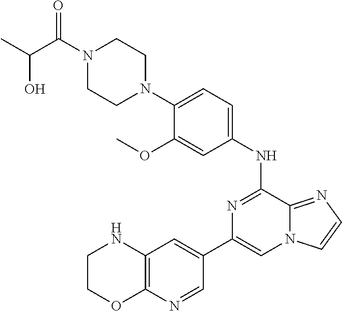 [SMILES: COc1cc(Nc2nc(-c3cnc4c(c3)NCCO4)cn3ccnc23)ccc1N1CCN(C(=O)C(C)O)CC1] | 1-(4-(4-((6-(2,3-dihydro-1H- pyrido[2,3-b][1,4]oxazin-7- yl)imidazo[1,2-a]pyrazin-8- yl)amino)-2- methoxyphenyl)piperazin-1-yl)- 2-hydroxypropan-1-one | — | — |
| 85 | 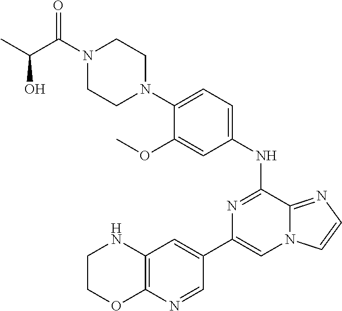 [SMILES: COc1cc(Nc2nc(-c3cnc4c(c3)NCCO4)cn3ccnc23)ccc1N1CCN(C(=O)[C@H](C)O)CC1] | (S)-1-(4-(4-((6-(2,3-dihydro-1H- pyrido[2,3-b][1,4]oxazin-7- yl)imidazo[1,2-a]pyrazin-8- yl)amino)-2- methoxyphenyl)piperazin-1-yl)- 2-hydroxypropan-1-one | 137 | 0.003 |
| 86 | 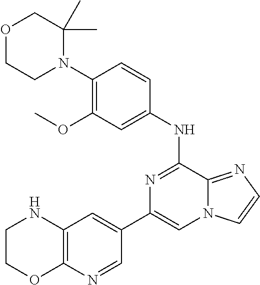 [SMILES: COc1cc(Nc2nc(-c3cnc4c(c3)NCCO4)cn3ccnc23)ccc1N1CCOCC1(C)C] | 6-(2,3-dihydro-1H-pyrido[2,3- b][1,4]oxazin-7-yl)-N-(4-(3,3- dimethylmorpholino)-3- methoxyphenyl)imidazo[1,2- a]pyrazin-8-amine | 142 | 0.013 |
| 87A | 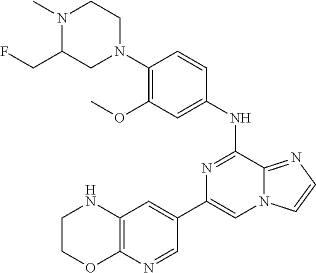 [SMILES: COc1cc(Nc2nc(-c3cnc4c(c3)NCCO4)cn3ccnc23)ccc1N1CCN(C)C(CF)C1] | 6-(2,3-dihydro-1H-pyrido[2,3- b][1,4]oxazin-7-yl)-N-(4-(3- (fluoromethyl)-4- methylpiperazin-1-yl)-3- methoxyphenyl)imidazo[1,2- a]pyrazin-8-amine | — | — |
| 87 | 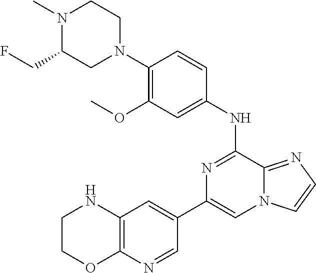 [SMILES: COc1cc(Nc2nc(-c3cnc4c(c3)NCCO4)cn3ccnc23)ccc1N1CCN(C)[C@@H](CF)C1] | (R)-6-(2,3-dihydro-1H- pyrido[2,3-b][1,4]oxazin-7-yl)- N-(4-(3-(fluoromethyl)-4- methylpiperazin-1-yl)-3- methoxyphenyl)imidazo[1,2- a]pyrazin-8-amine | 151 | 0.008 |
| 88A | 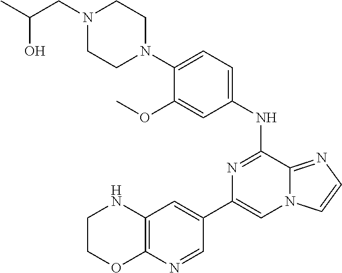 [SMILES: COc1cc(Nc2nc(-c3cnc4c(c3)NCCO4)cn3ccnc23)ccc1N1CCN(CC(C)O)CC1] | 1-(4-(4-((6-(2,3-dihydro-1H- pyrido[2,3-b][1,4]oxazin-7- yl)imidazo[1,2-a]pyrazin-8- yl)amino)-2- methoxyphenyl)piperazin-1- yl)propan-2-ol | — | — |
| 88 | 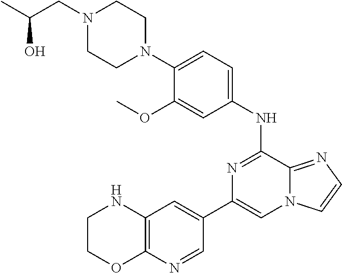 [SMILES: COc1cc(Nc2nc(-c3cnc4c(c3)NCCO4)cn3ccnc23)ccc1N1CCN(C[C@H](C)O)CC1] | (S)-1-(4-(4-((6-(2,3-dihydro-1H- pyrido[2,3-b][1,4]oxazin-7- yl)imidazo[1,2-a]pyrazin-8- yl)amino)-2- methoxyphenyl)piperazin-1- yl)propan-2-ol | 156 | 0.005 |
| 89 | 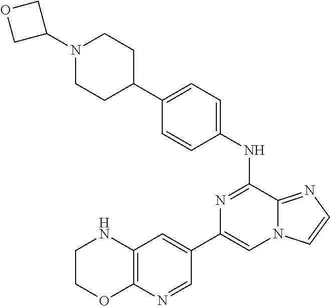 [SMILES: c1cn2cc(-c3cnc4c(c3)NCCO4)nc(Nc3ccc(C4CCN(C5COC5)CC4)cc3)c2n1] | 6-(2,3-dihydro-1H-pyrido[2,3- b][1,4]oxazin-7-yl)-N-(4-(1- (oxetan-3-yl)piperidin-4- yl)phenyl)imidazo[1,2-a]pyrazin- 8-amine | 162 | 0.007 |
| 90 | 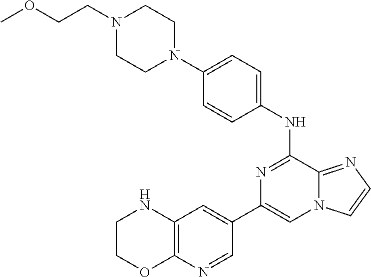 [SMILES: COCCN1CCN(c2ccc(Nc3nc(-c4cnc5c(c4)NCCO5)cn4ccnc34)cc2)CC1] | 6-(2,3-dihydro-1H-pyrido[2,3- b][1,4]oxazin-7-yl)-N-(4-(4-(2- methoxyethyl)piperazin-1- yl)phenyl)imidazo[1,2-a]pyrazin- 8-amine | 166 | 0.011 |
| 91 | 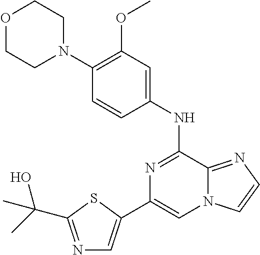 [SMILES: COc1cc(Nc2nc(-c3cnc(C(C)(C)O)s3)cn3ccnc23)ccc1N1CCOCC1] | 2-(5-(8-((3-methoxy-4- morpholinophenyl)amino)imidazo [1,2-a]pyrazin-6-yl)thiazol-2- yl)propan-2-ol | 168 | 0.003 |
| 92 | 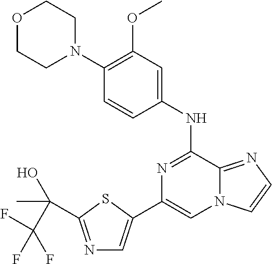 [SMILES: COc1cc(Nc2nc(-c3cnc(C(C)(O)C(F)(F)F)s3)cn3ccnc23)ccc1N1CCOCC1] | 1,1,1-trifluoro-2-(5-(8-((3- methoxy-4- morpholinophenyl)amino)imidazo [1,2-a]pyrazin-6-yl)thiazol-2- yl)propan-2-ol | 170 | 0.003 |
| 93 | 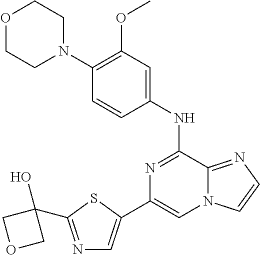 [SMILES: COc1cc(Nc2nc(-c3cnc(C4(O)COC4)s3)cn3ccnc23)ccc1N1CCOCC1] | 3-(5-(8-((3-methoxy-4- morpholinophenyl)amino)imidazo [1,2-a]pyrazin-6-yl)thiazol-2- yl)oxetan-3-ol | 172 | 0.009 |
| 94 | 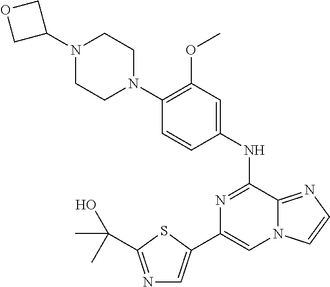 [SMILES: COc1cc(Nc2nc(-c3cnc(C(C)(C)O)s3)cn3ccnc23)ccc1N1CCN(C2COC2)CC1] | 2-(5-(8-((3-methoxy-4-(4- (oxetan-3-yl)piperazin-1- yl)phenyl)amino)imidazo[1,2- a]pyrazin-6-yl)thiazol-2- yl)propan-2-ol | 173 | 0.010 |
| 95 | 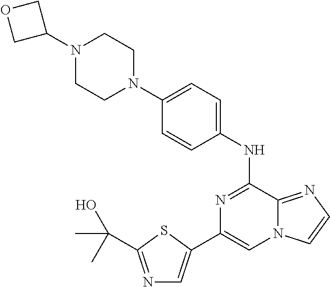 [SMILES: CC(C)(O)c1ncc(-c2cn3ccnc3c(Nc3ccc(N4CCN(C5COC5)CC4)cc3)n2)s1] | 2-(5-(8-((4-(4-(oxetan-3- yl)piperazin-1- yl)phenyl)amino)imidazo[1,2- a]pyrazin-6-yl)thiazol-2- yl)propan-2-ol | 174 | 0.014 |
| 96 | 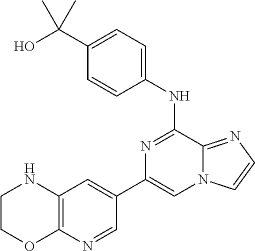 [SMILES: CC(C)(O)c1ccc(Nc2nc(-c3cnc4c(c3)NCCO4)cn3ccnc23)cc1] | 2-(4-((6-(2,3-dihydro-1H- pyrido[2,3-b][1,4]oxazin-7- yl)imidazo[1,2-a]pyrazin-8- yl)amino)phenyl)propan-2-ol | 175 | 0.004 |
| 98 | 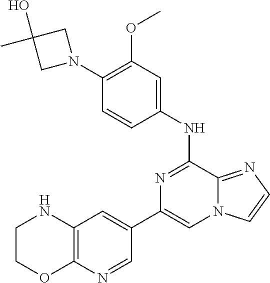 [SMILES: COc1cc(Nc2nc(-c3cnc4c(c3)NCCO4)cn3ccnc23)ccc1N1CC(C)(O)C1] | 1-(4-((6-(2,3-dihydro-1H- pyrido[2,3-b][1,4]oxazin-7- yl)imidazo[1,2-a]pyrazin-8- yl)amino)-2-methoxyphenyl)-3- methylazetidin-3-ol | 176 | 0.033 |
| 99 | 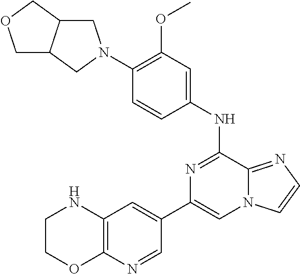 [SMILES: COc1cc(Nc2nc(-c3cnc4c(c3)NCCO4)cn3ccnc23)ccc1N1CC2COCC2C1] | 6-(2,3-dihydro-1H-pyrido[2,3- b][1,4]oxazin-7-yl)-N-(3- methoxy-4-(tetrahydro-1H- furo[3,4-c]pyrrol-5(3H)- yl)phenyl)imidazo[1,2-a]pyrazin- 8-amine | 177 | 0.016 |
| 100A | 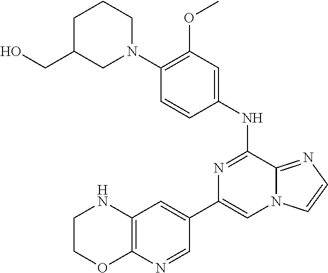 [SMILES: COc1cc(Nc2nc(-c3cnc4c(c3)NCCO4)cn3ccnc23)ccc1N1CCCC(CO)C1] | (1-(4-((6-(2,3-dihydro-1H- pyrido[2,3-b][1,4]oxazin-7- yl)imidazo[1,2-a]pyrazin-8- yl)amino)-2- methoxyphenyl)piperidin-3- yl)methanol | — | — |
| 100 | 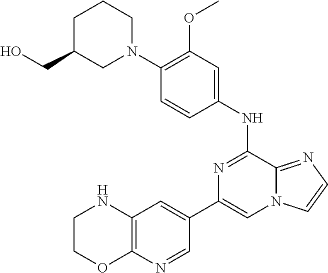 [SMILES: COc1cc(Nc2nc(-c3cnc4c(c3)NCCO4)cn3ccnc23)ccc1N1CCC[C@H](CO)C1] | (S)-(1-(4-((6-(2,3-dihydro-1H- pyrido[2,3-b][1,4]oxazin-7- yl)imidazo[1,2-a]pyrazin-8- yl)amino)-2- methoxyphenyl)piperidin-3- yl)methanol | 178 | 0.008 |
| 101 | 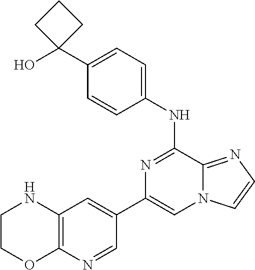 [SMILES: OC1(c2ccc(Nc3nc(-c4cnc5c(c4)NCCO5)cn4ccnc34)cc2)CCC1] | 1-(4-((6-(2,3-dihydro-1H- pyrido[2,3-b][1,4]oxazin-7- yl)imidazo[1,2-a]pyrazin-8- yl)amino)phenyl)cyclobutanol | 181 | 0.007 |
| 102 | 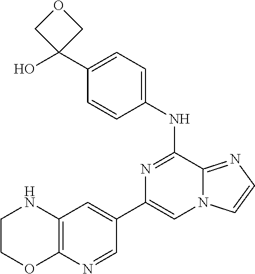 [SMILES: OC1(c2ccc(Nc3nc(-c4cnc5c(c4)NCCO5)cn4ccnc34)cc2)COC1] | 3-(4-((6-(2,3-dihydro-1H- pyrido[2,3-b][1,4]oxazin-7- yl)imidazo[1,2-a]pyrazin-8- yl)amino)phenyl)oxetan-3-ol | 182 | 0.008 |
| 103 | 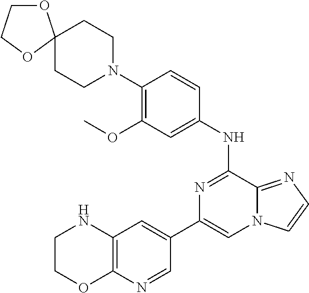 [SMILES: COc1cc(Nc2nc(-c3cnc4c(c3)NCCO4)cn3ccnc23)ccc1N1CCC2(CC1)OCCO2] | 6-(2,3-dihydro-1H-pyrido[2,3- b][1,4]oxazin-7-yl)-N-(3- methoxy-4-(1,4-dioxa-8- azaspiro[4.5]decan-8- yl)phenyl)imidazo[1,2-a]pyrazin- 8-amine | 183 | 0.003 |
| 104 | 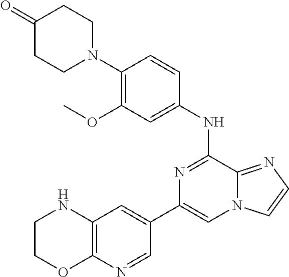 [SMILES: COc1cc(Nc2nc(-c3cnc4c(c3)NCCO4)cn3ccnc23)ccc1N1CCC(=O)CC1] | 1-(4-((6-(2,3-dihydro-1H- pyrido[2,3-b][1,4]oxazin-7- yl)imidazo[1,2-a]pyrazin-8- yl)amino)-2- methoxyphenyl)piperidin-4-one | 184 | 0.004 |
| 105 | 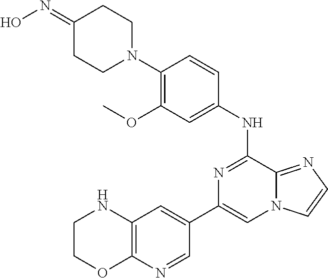 [SMILES: COc1cc(Nc2nc(-c3cnc4c(c3)NCCO4)cn3ccnc23)ccc1N1CCC(=NO)CC1] | 1-(4-((6-(2,3-dihydro-1H- pyrido[2,3-b][1,4]oxazin-7- yl)imidazo[1,2-a]pyrazin-8- yl)amino)-2- methoxyphenyl)piperidin-4-one oxime | 185 | 0.005 |
| 106 | 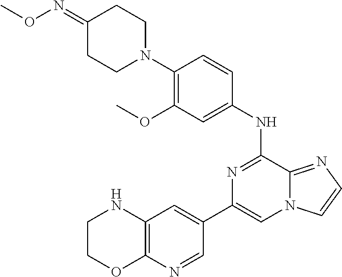 [SMILES: CON=C1CCN(c2ccc(Nc3nc(-c4cnc5c(c4)NCCO5)cn4ccnc34)cc2OC)CC1] | 1-(4-((6-(2,3-dihydro-1H- pyrido[2,3-b][1,4]oxazin-7- yl)imidazo[1,2-a]pyrazin-8- yl)amino)-2- methoxyphenyl)piperidin-4-one O-methyl oxime | 186 | 0.006 |
| 107 | 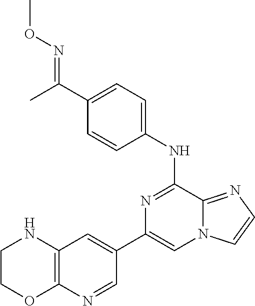 [SMILES: CO/N=C(\C)c1ccc(Nc2nc(-c3cnc4c(c3)NCCO4)cn3ccnc23)cc1] | (E)-1-(4-((6-(2,3-dihydro-1H- pyrido[2,3-b][1,4]oxazin-7- yl)imidazo[1,2-a]pyrazin-8- yl)amino)phenyl)ethanone O- methyl oxime | 188 | 0.017 |
| 108 | 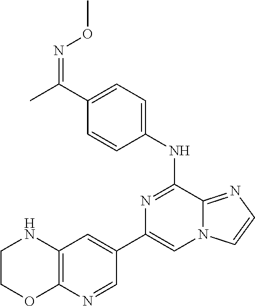 [SMILES: CO/N=C(/C)c1ccc(Nc2nc(-c3cnc4c(c3)NCCO4)cn3ccnc23)cc1] | (Z)-1-(4-((6-(2,3-dihydro-1H- pyrido[2,3-b][1,4]oxazin-7- yl)imidazo[1,2-a]pyrazin-8- yl)amino)phenyl)ethanone O- methyl oxime | 189 | 0.007 |
| 109 | 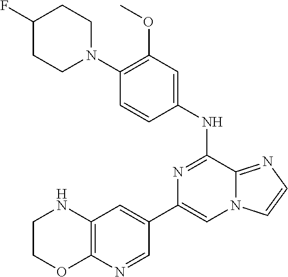 [SMILES: COc1cc(Nc2nc(-c3cnc4c(c3)NCCO4)cn3ccnc23)ccc1N1CCC(F)CC1] | 6-(2,3-dihydro-1H-pyrido[2,3- b][1,4]oxazin-7-yl)-N-(4-(4- fluoropiperidin-1-yl)-3- methoxyphenyl)imidazo[1,2- a]pyrazin-8-amine | 192 | 0.007 |
| 111 | 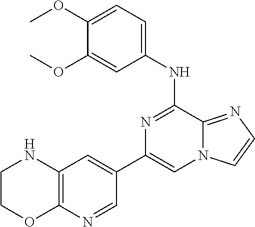 [SMILES: COc1ccc(Nc2nc(-c3cnc4c(c3)NCCO4)cn3ccnc23)cc1OC] | 6-(2,3-dihydro-1H-pyrido[2,3- b][1,4]oxazin-7-yl)-N-(3,4- dimethoxyphenyl)imidazo[1,2- a]pyrazin-8-amine | 193 | 0.007 |

Provided are also compounds of Formula I, Ia, Ib, Ic, Id or Ie in which from 1 to n hydrogens attached to a carbon atom may be replaced by a deuterium atom or D, in which n is the number of hydrogens in the molecule. As known in the art, the deuterium atom is a non-radioactive isotope of the hydrogen atom. Such compounds exhibit may increase resistance to metabolism, and thus may be useful for increasing the half-life of the compounds of Formula I, Ia, Ib, Ic, Id or Ie when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogens have been replaced by deuterium.

In some embodiments, compounds described herein may include pharmaceutically acceptable salts, pharmaceutically acceptable esters, tautomeric forms, polymorphs, and produgs of such compounds.

In certain embodiments, compounds described herein include their optical isomers, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high pressure liquid chromatography (HPLC) column. In addition, such compounds include Z- and E-forms (or cis- and trans-forms) of compounds with carbon-carbon double bonds. In certain embodiments, where compounds described herein exist in various tautomeric forms, the compounds of formula I, Ia, Ib, Ic, Id or Ie include all tautomeric forms of the compound. Such compounds also include crystal forms including polymorphs and clathrates.

Compositions provided herein that include a compound of Formula I, Ia, Ib, Ic, Id or le may include racemic mixtures or mixtures containing an enantiomeric excess of one enantiomer or single diastereomers or diastereomeric mixtures. All such isomeric forms of these compounds are expressly included herein the same as if each and every isomeric form were specifically and individually listed.

In certain embodiments, compounds described herein may also include crystalline and amorphous forms of those compounds. For example, compounds described herein may include polymorphs, pseudopolymorphs, solvates, hydrates, unsolvated polymorphs (including anhydrates), conformational polymorphs, and amorphous forms of the compounds, as well as mixtures thereof. “Crystalline form” or “polymorph” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to. Compounds described herein also include pharmaceutically acceptable forms of the recited compounds, including chelates, non-covalent complexes, prodrugs, and mixtures thereof.

Compounds described herein may be characterized using methods that are commonly known in the art, including biochemical assays with PTK biotinylated peptide, ramos cell pBLNK(Y96) assays, B-cell or T-cell proliferation assays, inhibition assays for CD63, CD69 or CD86, degranulation assays in bone-marrow derived mouse mast cell (BMMC) degranulation, and passive cutaneous anaphylaxis (PCA) assays.

“Pharmaceutically acceptable salts” include, but are not limited to salts with inorganic acids, such as hydrochlorate, phosphate, diphosphate, hydrobromate, sulfate, sulfinate, nitrate; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4. Similarly, pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

As noted above, prodrugs also fall within the scope of compounds described herein. In some embodiments, the “prodrugs” described herein include any compound that becomes a compound of Formula I, Ia, Ib, Ic, Id or Ie when administered to a patient, e.g., upon metabolic processing of the prodrug. Examples of prodrugs include derivatives of functional groups, such as a carboxylic acid group, in the compounds described herein. Exemplary prodrugs of a carboxylic acid group include, but are not limited to, carboxylic acid esters such as alkyl esters, hydroxyalkyl esters, arylalkyl esters, and aryloxyalkyl esters.

A “solvate” is formed by the interaction of a solvent and a compound. In some embodiments, the term “compound” includes solvates of compounds. Similarly, in other embodiments, “salts” include solvates of salts. Suitable solvates are pharmaceutically acceptable solvates, such as hydrates, including monohydrates and hemi-hydrates.

A “chelate” is formed by the coordination of a compound to a metal ion at two (or more) points. In some embodiments, the term “compound” includes chelates of compounds. Similarly, in other embodiments, “salts” include chelates of salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). In certain embodiments, such non-covalent complexes may be included in the term “compound”.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry (G. C. Pimentel and A. L. McClellan, The Hydrogen Bond, Freeman, San Francisco, 1960; R. Taylor and O. Kennard, “Hydrogen Bond Geometry in Organic Crystals”, Accounts of Chemical Research, 17, pp. 320-326 (1984)).

As used herein the terms “group”, “radical” or “fragment” are synonymous and are intended to indicate functional groups or fragments of molecules attachable to a bond or other fragments of molecules.

The term “active agent” is used to indicate a compound of formula I, Ia, Ib, Ic, Id or Ie, or a pharmaceutically acceptable salt, ester, prodrug, or solvate thereof which has biological activity. In some embodiments, an “active agent” is a compound having pharmaceutical utility. For example an active agent may be an anti-cancer therapeutic.

The term “therapeutically effective amount” of a compound of formula I, Ia, Ib, Ic, Id or Ie, or a pharmaceutically acceptable salt, ester, prodrug, or solvate thereof, means an amount effective, when administered to a human or non-human patient, to provide a therapeutic benefit such as amelioration of symptoms, slowing of disease progression, or prevention of disease, e.g., a therapeutically effective amount may be an amount sufficient to decrease the symptoms of a disease responsive to inhibition of Syk activity. In some embodiments, a therapeutically effective amount is an amount sufficient to reduce cancer symptoms, the symptoms of an allergic disorder, the symptoms of an autoimmune and/or inflammatory disease, or the symptoms of an acute inflammatory reaction. In some embodiments a therapeutically effective amount is an amount sufficient to decrease the number of detectable cancerous cells in an organism, detectably slow, or stop the growth of a cancerous tumor. In some embodiments, a therapeutically effective amount is an amount sufficient to shrink a cancerous tumor. In some circumstances a patient suffering from cancer may not present symptoms of being affected. In some embodiments, a therapeutically effective amount of a compound of formula I, Ia, Ib, Ic, Id or Ie, or a pharmaceutically acceptable salt, ester, prodrug, or solvate thereof, is an amount sufficient to prevent a significant increase or significantly reduce the detectable level of cancerous cells or cancer markers in the patient's blood, serum, or tissues. In methods described herein for treating allergic disorders and/or autoimmune and/or inflammatory diseases and/or acute inflammatory reactions, a therapeutically effective amount may also be an amount sufficient, when administered to a patient, to detectably slow progression of the disease, or prevent the patient to whom the compound of formula I, Ia, Ib, Ic, Id or Ie, or a pharmaceutically acceptable salt, ester, prodrug, or solvate thereof, given from presenting symptoms of the allergic disorders and/or autoimmune and/or inflammatory disease, and/or acute inflammatory response. In some methods described herein for treating allergic disorders and/or autoimmune and/or inflammatory diseases and/or acute inflammatory reactions, a therapeutically effective amount may also be an amount sufficient to produce a detectable decrease in the amount of a marker protein or cell type in the patient's blood or serum. For example, in some embodiments a therapeutically effective amount is an amount of a compound of formula I, Ia, Ib, Ic, Id or Ie, or a pharmaceutically acceptable salt, ester, prodrug, or solvate thereof sufficient to significantly decrease the activity of B-cells. In another example, in some embodiments a therapeutically effective amount is an amount of a compound of formula I, Ia, Ib, Ic, Id or Ie, or a pharmaceutically acceptable salt, ester, prodrug, or solvate thereof sufficient to significantly decrease the number of B-cells. In another example, in some embodiments a therapeutically effective amount is an amount of a compound of formula I, Ia, Ib, Ic, Id or Ie, or a pharmaceutically acceptable salt, ester, prodrug, or solvate thereof to decrease the level of antiacetylcholine receptor antibody in a patient's blood with the disease myasthenia gravis.

The term “inhibition” indicates a significant decrease in the baseline activity of a biological activity or process. “Inhibition of Syk activity” refers to a decrease in Syk activity as a direct or indirect response to the presence of a compound of formula I, Ia, Ib, Ic, Id or Ie, or a pharmaceutically acceptable salt, ester, prodrug, or solvate thereof, relative to the activity of Syk in the absence of the compound of formula I, Ia, Ib, Ic, Id or Ie, or a pharmaceutically acceptable salt, ester, prodrug, or solvate thereof. The decrease in activity may be due to the direct interaction of the compound with Syk, or due to the interaction of the compounds described herein with one or more other factors that in turn affect Syk activity. For example, the presence of the compounds of formula I, Ia, Ib, Ic, Id or Ie, or a pharmaceutically acceptable salt, ester, prodrug, or solvate thereof, may decrease Syk activity by directly binding to the Syk, by causing (directly or indirectly) another factor to decrease Syk activity, or by (directly or indirectly) decreasing the amount of Syk present in the cell or organism.

Inhibition of Syk activity also refers to observable inhibition of Syk activity in a standard biochemical assay for Syk activity, such as the ATP hydrolysis assay described below. In some embodiments, the compound of formula I, Ia, Ib, Ic, Id or Ie has an IC₅₀ value less than or equal to 1 micromolar. In some embodiments, the compound of formula I, Ia, Ib, Ic, Id or Ie has an IC₅₀ value less than or equal to less than 100 nanomolar. In some embodiments, the compound of formula I, Ia, Ib, Ic, Id or Ie has an IC₅₀ value less than or equal to 10 nanomolar.

“Inhibition of B-cell activity” refers to a decrease in B-cell activity as a direct or indirect response to the presence of a compound of formula I, Ia, Ib, Ic, Id or Ie, or a pharmaceutically acceptable salt, ester, prodrug, or solvate thereof, relative to the activity of B-cells in the absence of the compound. The decrease in activity may be due to the direct interaction of the compound with Syk or with one or more other factors that in turn affect B-cell activity.

Inhibition of B-cell activity also refers to observable inhibition of CD86 expression in a standard assay such as the assay described below. In some embodiments, the compound of formula I, Ia, Ib, Ic, Id or Ie has an IC₅₀ value less than or equal to 10 micromolar. In some embodiments, the compound of formula I, Ia, Ib, Ic, Id or Ie has an IC₅₀ value less than or equal to less than 1 micromolar. In some embodiments, the compound of formula I, Ia, Ib, Ic, Id or Ie has an IC₅₀ value less than or equal to 500 nanomolar.

“B-cell activity” also includes activation, redistribution, reorganization, or capping of one or more various B-cell membrane receptors, or membrane-bound immunoglobulins, e.g, IgM, IgG, and IgD. Most B-cells also have membrane receptors for Fe portion of IgG in the form of either antigen-antibody complexes or aggregated IgG. B-cells also carry membrane receptors for the activated components of complement, e.g., C3b, C3d, C4, and Clq. These various membrane receptors and membrane-bound immunoglobulins have membrane mobility and can undergo redistribution and capping that can initiate signal transduction.

B-cell activity also includes the synthesis or production of antibodies or immunoglobulins. Immunoglobulins are synthesized by the B-cell series and have common structural features and structural units. Five immunoglobulin classes, i.e., IgG, IgA, IgM, IgD, and IgE, are recognized on the basis of structural differences of their heavy chains including the amino acid sequence and length of the polypeptide chain. Antibodies to a given antigen may be detected in all or several classes of immunoglobulins or may be restricted to a single class or subclass of immunoglobulin. Autoantibodies or autoimmune antibodies may likewise belong to one or several classes of immunoglobulins. For example, rheumatoid factors (antibodies to IgG) are most often recognized as an IgM immunoglobulin, but can also consist of IgG or IgA.

In addition, B-cell activity also is intended to include a series of events leading to B-cell clonal expansion (proliferation) from precursor B lymphocytes and differentiation into antibody-synthesizing plasma cells which takes place in conjunction with antigen-binding and with cytokine signals from other cells.

“Inhibition of B-cell proliferation” refers to inhibition of proliferation of abnormal B-cells, such as cancerous B-cells, e.g., lymphoma B-cells and/or inhibition of normal, non-diseased B-cells. The term “inhibition of B-cell proliferation” indicates any significant decrease in the number of B-cells, either in vitro or in vivo. Thus an inhibition of B-cell proliferation in vitro would be any significant decrease in the number of B-cells in an in vitro sample contacted with a compound of formula I, Ia, Ib, Ic, Id or Ie, as compared to a matched sample not contacted with the compound of formula I, Ia, Ib, Ic, Id or Ie. Inhibition of B-cell proliferation also refers to observable inhibition of B-cell proliferation in a standard thymidine incorporation assay for B-cell proliferation, such as the assay described herein. In some embodiments, the compound of formula I, Ia, Ib, Ic, Id or Ie an IC₅₀ value less than or equal to 10 micromolar. In some embodiments, the compound of formula I, Ia, Ib, Ic, Id or Ie has an IC₅₀ value less than or equal to less than 1 micromolar. In some embodiments, the compound of formula I, Ia, Ib, Ic, Id or Ie an IC₅₀ value less than or equal to 100 nanomolar.

An “allergy” or “allergic disorder” refers to acquired hypersensitivity to a substance (allergen). Allergic conditions include eczema, allergic rhinitis or coryza, hay fever, bronchial asthma, urticaria (hives) and food allergies, and other atopic conditions.

“Asthma” refers to a disorder of the respiratory system characterized by inflammation, narrowing of the airways and increased reactivity of the airways to inhaled agents. Asthma is frequently, although not exclusively associated with atopic or allergic symptoms.

By “significant” is meant any detectable change that is statistically significant in a standard parametric test of statistical significance such as Student's Ttest, where p <0.05.

A “disease responsive to inhibition of Syk activity” is a disease in which inhibiting Syk kinase provides a therapeutic benefit such as an amelioration of symptoms, decrease in disease progression, prevention or delay of disease onset, or inhibition of aberrant activity of certain cell-types (monocytes, B-cells, and mast cells).

“Treatment” or “treating” means any treatment of a disease in a patient, including:

a) inhibiting the disease;

b) slowing or arresting the development of clinical symptoms; and/or

c) relieving the disease, that is, causing the regression of clinical symptoms.

“Prevention” or “preventing” means any treatment of a disease that causes the clinical symptoms of the disease not to develop.

“Patient” refers to an animal, such as a mammal, that has been or will be the object of treatment, observation or experiment. The methods described herein may be useful in both human therapy and veterinary applications. In some embodiments, the patient is a mammal; in some embodiments the patient is human; and in some embodiments the patient is chosen from cats and dogs.

The compounds of formula I, Ia, Ib, Ic, Id or Ie, or a pharmaceutically acceptable salt, ester, prodrug, or solvate thereof, may be used to inhibit PI3K activity therapeutically or prophylactically. Also, the compounds of formula I, Ia, Ib, Ic, Id or Ie, or a pharmaceutically acceptable salt, ester, prodrug, or solvate thereof, may be used in combination with other therapeutic agents. The therapeutic agents may be in the forms of compounds, antibodies, polypeptides, or polynucleotides. Also, the therapeutic agents may be those that inhibit or modulate the activities of Bruton's tyrosine kinase, spleen tyrosin kinase, apoptosis signal-regulating kinase, Janus kinase, lysyl oxidase, lysyl oxidase-like proteins, or matrix metallopeptidase.

Kits that include a compound of formula I, Ia, Ib, Ic, Id or Ie, or a pharmaceutically acceptable salt, ester, prodrug, or solvate thereof, and suitable packaging are provided. In one embodiment, a kit further includes instructions for use. In one aspect, a kit includes a compound of formula I, Ia, Ib, Ic, Id or Ie, or a pharmaceutically acceptable salt, ester, prodrug, or solvate thereof, and instructions for use of the compounds in the treatment of the diseases or conditions described herein.

Articles of manufacture that include a compound of formula I, Ia, Ib, Ic, Id or Ie, or a pharmaceutically acceptable salt, ester, prodrug, or solvate thereof, in a suitable container are provided. The container may be a vial, jar, ampoule, preloaded syringe, and intravenous bag.

Methods for obtaining the novel compounds described herein will be apparent to those of ordinary skill in the art, suitable procedures being described, for example, in the reaction schemes and examples below, and in the references cited herein.

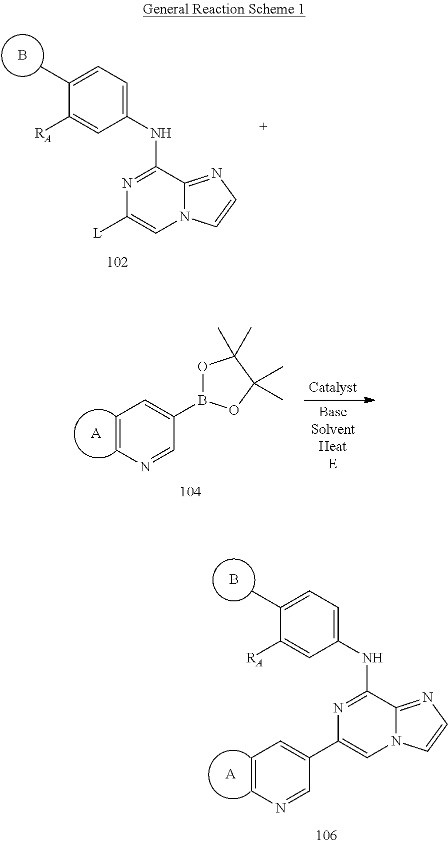

In certain examples of the formulae described herein, compounds of type 106 can be prepared by reacting appropriately compound 102 with compound 104, in the presence of a catalyst, base and solvent, at elevated temperatures. Suitable catalysts will be apparent to those skilled in the art, including for example palladium catalysts (e.g., PdCl₂dppf, Pd(PPh₃)₄. A selection of bases effective for this reaction will be apparent to those skilled in the art, such as for example, sodium carbonate (Na₂CO₃) or potassium carbonate (K₂CO₃). A selection of solvents effective for this reaction will also be apparent to those skilled in the art, such as for example, organic solvents (e.g., toluene, isopropanol, dimethoxyethane) and water. The reaction is generally performed at elevated temperatures (e.g., between 50° C. and 200° C.), depending on the specific materials, catalysts, bases and solvents used. It should be understood that modifications to the specific materials are intended. For example, for Compound 102, L is a leaving group such as a halo group (e.g., F, Cl, Br); R_{A} can be hydrogen, halo or alkoxy; and moeity B can a heterocyclyl group (e.g., optionally substituted morpholinyl, optionally substituted homomorpholinyl, optionally substituted piperazinyl, optionally substituted piperidinyl, optionally substituted pyrrolidinyl, optionally substituted azetidinyl). For Compound 104, ring A can be a heterocyclyl group (e.g., optionally substituted morpholinyl, optionally substituted oxazepanyl, optionally substituted thiomorpholinyl, optionally substituted thiomorpholinyl S-oxide, optionally substituted thiomorpholinyl sulfone, and optionally substituted piperidinyl).

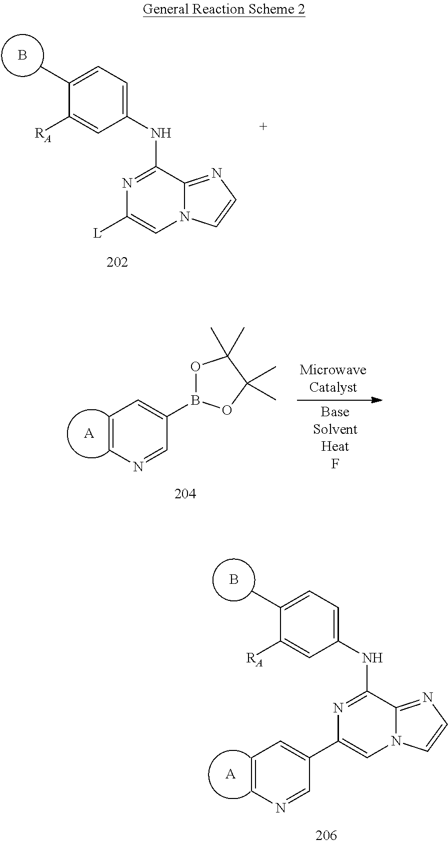

In other examples of the formulae described herein, compounds of type 206 can be prepared by reacting appropriately compound 202 with compound 204, in the presence of a catalyst, base and solvent, at elevated temperatures in the microwave. Suitable catalysts will be apparent to those skilled in the art, including for example palladium catalysts (e.g., PdCl₂dppf, Pd(PPh₃)₄. A selection of bases effective for this reaction will be apparent to those skilled in the art, such as for example, sodium carbonate (Na₂CO₃) or potassium carbonate (K₂CO₃). A selection of solvents effective for this reaction will also be apparent to those skilled in the art, such as for example, organic solvents (e.g., toluene, isopropanol, dimethoxymethane) and water. The reaction is generally performed at elevated temperatures (e.g., between 50° C. and 200° C.), depending on the specific materials, catalysts, bases and solvents used. It should be understood that modifications to the specific materials are intended. For example, for Compound 202, L is a leaving group such as a halo group (e.g., F, Cl, Br); R_{A} can be hydrogen, halo or alkoxy; and moeity B can a heterocyclyl group (e.g., optionally substituted morpholinyl, optionally substituted homomorpholinyl, optionally substituted piperazinyl, optionally substituted piperidinyl, optionally substituted pyrrolidinyl, optionally substituted azetidinyl). For Compound 204, ring A can be a heterocyclyl group (e.g., optionally substituted morpholinyl, optionally substituted oxazepanyl, optionally substituted thiomorpholinyl, optionally substituted thiomorpholinyl S-oxide, optionally substituted thiomorpholinyl sulfone, and optionally substituted piperidinyl).

Provided is also a method of treating a patient, for example, a mammal, such as a human, having a disease responsive to inhibition of Syk activity, comprising administrating to the patient having such a disease, an effective amount of the compound of formula I, Ia, Ib, Ic, Id or Ie, or a pharmaceutically acceptable salt, ester, prodrug, or solvate thereof.

In some embodiments, the compounds of formula I, Ia, Ib, Ic, Id or Ie, or a pharmaceutically acceptable salt, ester, prodrug, or solvate thereof may also inhibit other kinases, such that disease, disease symptoms, and conditions associated with these kinases is also treated. In other embodiments, a compound having a deuterium atom may have a reduced rate of metabolism and be suitable for certain therapeutic treatments.

Methods of treatment also include inhibiting Syk activity and/or inhibiting B-cell activity, by inhibiting ATP binding or hydrolysis by Syk or by some other mechanism, in vivo, in a patient suffering from a disease responsive to inhibition of Syk activity, by administering an effective concentration of a compound of formula I, Ia, Ib, Ic, Id or Ie, or a pharmaceutically acceptable salt, ester, prodrug, or solvate thereof. An example of an effective concentration would be that concentration sufficient to inhibit Syk activity in vitro. An effective concentration may be ascertained experimentally, for example by assaying blood concentration of the compound of formula I, Ia, Ib, Ic, Id or Ie, or a pharmaceutically acceptable salt, ester, prodrug, or solvate thereof, or theoretically, by calculating bioavailability.

In some embodiments, the condition responsive to inhibition of Syk activity and/or B-cell activity is cancer, an allergic disorder and/or an autoimmune and/or inflammatory disease, and/or an acute inflammatory reaction.

Also provided is a method of treating a patient having cancer, an allergic disorder and/or an autoimmune and/or inflammatory disease, and/or an acute inflammatory reaction, by administering an effective amount of the compound of formula I, Ia, Ib, Ic, Id or Ie, or a pharmaceutically acceptable salt, ester, prodrug, or solvate thereof.

In some embodiments, the conditions and diseases that can be affected using the compounds of formula I, Ia, Ib, Ic, Id or Ie described herein, include, but are not limited to: allergic disorders, including but not limited to eczema, allergic rhinitis or coryza, hay fever, bronchial asthma, urticaria (hives) and food allergies, and other atopic conditions; autoimmune and/or inflammatory diseases, including but not limited to psoriasis, ulcerative colitis, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic obstructive pulmonary disease (COPD), adult respiratory distress syndrome (ARDS), chronic Idiopathic thrombocytopenic purpura (ITP), Addison's disease, Parkinson's disease, Alzheimer's disease, diabetes, septic shock, and myasthenia gravis; acute inflammatory reactions, including but not limited to skin sunburn, inflammatory pelvic disease, inflammatory bowel disease, urethritis, uvitis, sinusitis, pneumonitis, encephalitis, meningitis, myocarditis, nephritis, osteomyelitis, myositis, hepatitis gastritis, enteritis, dermatitis, gingivitis, appendicitis, pancreatitis, and cholocystitis; polycystic kidney disease, and cancer, including but not limited to, B-cell lymphoma, lymphoma (including Hodgkin's and non-Hodgkins lymphoma), hairy cell leukemia, multiple myeloma, chronic and acute myelogenous leukemia, chronic and acute lymphocytic leukemia, and ovarian cancer.

Syk is a known inhibitor of apoptosis in lymphoma B-cells. Defective apoptosis contributes to the pathogenesis and drug resistance of human leukemias and lymphomas. Thus, further provided is a method of promoting or inducing apoptosis in cells expressing Syk comprising contacting the cell with a compound of formula I, Ia, Ib, Ic, Id or Ie, or a pharmaceutically acceptable salt, ester, prodrug, or solvate thereof.

Also provided are methods of treatment in which a compound of formula I, Ia, Ib, Id or Ie, or a pharmaceutically acceptable salt, ester, prodrug, or solvate thereof is the only active agent given to a patient and also includes methods of treatment in which the compound of formula I, Ia, Ib, Ic, Id or Ie, or a pharmaceutically acceptable salt, ester, prodrug, or solvate thereof is given to a patient in combination with one or more additional active agents.

In some embodiments, a method of treating cancer, an allergic disorder and/or an autoimmune and/or inflammatory disease, and/or an acute inflammatory reaction comprises administering to a patient in need thereof an effective amount of the compound of formula I, Ia, Ib, Ic, Id or Ie, or a pharmaceutically acceptable salt, ester, prodrug, or solvate thereof, together with a second active agent, which can be useful for treating a cancer, an allergic disorder and/or an autoimmune and/or inflammatory disease, and/or an acute inflammatory reaction. For example the second agent may be an anti-inflammatory agent. Treatment with the second active agent may be prior to, concomitant with, or following treatment with the compound of formula I, Ia, Ib, Ic, Id or Ie, or a pharmaceutically acceptable salt, ester, prodrug, or solvate thereof. In some embodiments, the compound of formula I, Ia, Ib, Ic, Id or Ie, or a pharmaceutically acceptable salt, ester, prodrug, or solvate thereof is combined with another active agent in a single dosage form. Suitable antitumor therapeutics that may be used in combination with the compound of formula I, Ia, Ib, Ic, Id or Ie, or a pharmaceutically acceptable salt, ester, prodrug, or solvate thereof include, but are not limited to, chemotherapeutic agents, for example mitomycin C, carboplatin, taxol, cisplatin, paclitaxel, etoposide, doxorubicin, or a combination comprising at least one of the foregoing chemotherapeutic agents. Radiotherapeutic antitumor agents may also be used, alone or in combination with chemotherapeutic agents.

The compounds of formula I, Ia, Ib, Ic, Id or Ie described herein can be useful as chemosensitizing agents, and, thus, can be useful in combination with other chemotherapeutic drugs, in particular, drugs that induce apoptosis. Additionally, agents that are targeted molecular therapeutics in complementary and related pathways can be useful.

A method for increasing sensitivity of cancer cells to chemotherapy, comprising administering to a patient undergoing chemotherapy a chemotherapeutic agent together with the compound of formula I, Ia, Ib, Ic, Id or Ie, or a pharmaceutically acceptable salt, ester, prodrug, or solvate thereof in an amount sufficient to increase the sensitivity of cancer cells to the chemotherapeutic agent is also provided herein.

Examples of other chemotherapeutic drugs that can be used in combination with the compounds of formula I, Ia, Ib, Ic, Id or Ie, or a pharmaceutically acceptable salt, ester, prodrug, or solvate thereof include topoisomerase I inhibitors (e.g., camptothesin or topotecan), topoisomerase II inhibitors (e.g., daunomycin and etoposide), alkylating agents (e.g., cyclophosphamide, melphalan and BCNU), tubulin directed agents (e.g., taxol and vinblastine), and biological agents (e.g., antibodies such as anti CD20 antibody, I DEC 8, immunotoxins, and cytokines).

In some embodiments, the compound of formula I, Ia, Ib, Ic, Id or Ie, or a pharmaceutically acceptable salt, ester, prodrug, or solvate thereof are used in combination with Rituxan® (Rituximab) or other agents that work by selectively depleting CD20+ B-cells. Additional targeted molecular therapeutics would be chemical entities that inhibit related pathways include MEK inhibitors, PI3K inhibitors and PIM inhibitors.

Included herein are methods of treatment in which the compound of formula I, Ia, Ib, Ic, Id or Ie, or a pharmaceutically acceptable salt, ester, prodrug, or solvate thereof is administered in combination with an anti-inflammatory agent. Anti-inflammatory agents include but are not limited to NSAIDs, non-specific and COX-2 specific cyclooxygenase enzyme inhibitors, gold compounds, corticosteroids, methotrexate, tumor necrosis factor receptor (TNF) receptors antagonists, immunosuppressants and methotrexate.

Examples of NSAIDs include, but are not limited to ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors (i.e., a compound that inhibits COX-2 with an IC₅₀ that is at least 50-fold lower than the IC₅₀ for COX-1) such as celecoxib, valdecoxib, lumiracoxib, etoricoxib and/or rofecoxib.

In a further embodiment, the anti-inflammatory agent is a salicylate. Salicylates include but are not limited to acetylsalicylic acid or aspirin, sodium salicylate, and choline and magnesium salicylates.

The anti-inflammatory agent may also be a corticosteroid. For example, the corticosteroid may be chosen from cortisone, dexamethasone, methylprednisolone, prednisolone, prednisolone sodium phosphate, and prednisone.

In some embodiments, the anti-inflammatory therapeutic agent is a gold compound such as gold sodium thiomalate or auranofin.

In some embodiments, the anti-inflammatory agent is a metabolic inhibitor such as a dihydrofolate reductase inhibitor, such as methotrexate or a dihydroorotate dehydrogenase inhibitor, such as leflunomide.

In some embodiments, combinations in which at least one anti-inflammatory compound is an anti-CS monoclonal antibody (such as eculizumab or pexelizumab), a TNF antagonist, such as entanercept, or infliximab, which is an antiTNF alpha monoclonal antibody are used.

In some embodiments, combinations in which at least one active agent is an immunosuppressant compound such as methotrexate, leflunomide, cyclosporine, tacrolimus, azathioprine, or mycophenolate mofetil are used.

Oral administration is another route for administration of compounds in accordance with the invention. Administration may be via, for example, capsule or enteric coated tablets. In making the pharmaceutical compositions that include at least one compound of formula I. Ia, Ib, Ic, Id or Ie, or a pharmaceutically acceptable salt, ester, prodrug, or solvate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound of formula I, Ia, Ib, Ic, Id or Ie, or a pharmaceutically acceptable salt, ester, prodrug, or solvate thereof, can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

In certain embodiments, dosage levels may be from 0.1 mg to 140 mg per kilogram of body weight per day. Such dosage levels may, in certain instances, be useful in the treatment of the above-indicated conditions. In other embodiments, dosage levels may be from 0.5 mg to 7 g per patient per day. The amount of active ingredient that may be combined with the vehicle to produce a single dosage form will vary depending upon the host treated and the particular mode of administration. Dosage unit forms will generally contain from 1 mg to 500 mg of an active ingredient

Frequency of dosage may also vary depending on the compound used and the particular disease treated. In some embodiments, for example, for the treatment of an allergic disorder and/or autoimmune and/or inflammatory disease, a dosage regimen of 4 times daily or less is used. In some embodiments, a dosage regimen of 1 or 2 times daily is used. It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease in the patient undergoing therapy.

For preparing solid compositions such as tablets, the principal active ingredient may be mixed with a pharmaceutical excipient to form a solid preformulation composition containing a homogeneous mixture of a compound of compound of formula I, Ia, Ib, Ic, Id or Ie, or a pharmaceutically acceptable salt, ester, prodrug, or solvate thereof, When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

A labeled form of a compound of formula I, Ia, Ib, Ic, Id or Ie, or a pharmaceutically acceptable salt, ester, prodrug, or solvate thereof, can be used as a diagnostic for identifying and/or obtaining compounds that have the function of modulating an activity of a kinase as described herein. The compounds of formula I, Ia, Ib, Ic, Id or Ie, or a pharmaceutically acceptable salt, ester, prodrug, or solvate thereof, may additionally be used for validating, optimizing, and standardizing bioassays.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

### EXAMPLES

The following Examples are merely illustrative and are not meant to limit any aspects of the present disclosure in any way.

### Example 1

### Preparation of 4-(2-methoxy-4-nitrophenyl)morpholine

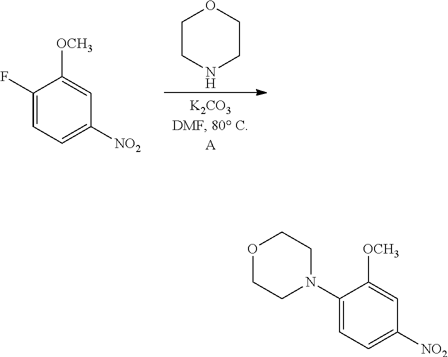 [SMILES: COc1cc([N+](=O)[O-])ccc1F ; C1COCCN1 ; COc1cc([N+](=O)[O-])ccc1N1CCOCC1]

A mixture of 1-fluoro-2-methoxy-4-nitrobenzene (100 g, 585 mmol), morpholine (61 g, 702 mmol) and potassium carbonate (120 g, 877 mmol) in dimethylformamide (1 L) was stirred at 80° C. for 4 hours. The reaction was cooled to room temperature, diluted with ethyl acetate (2 L), filtered. The filter cake was washed with ethyl acetate (1 L). The filtrate was washed with water (2×1 L), then 5% aqueous lithium chloride (2 L), followed by brine (2 L). The organic phase was dried over sodium sulfate, filtered and the filtrate was concentrated under reduced pressure to afford 4-(2-methoxy-4-nitrophenyl)morpholine as a yellow-orange solid.

### Example 2

### Preparation of 4-(2-methoxy-4-aminophenyl)morpholine

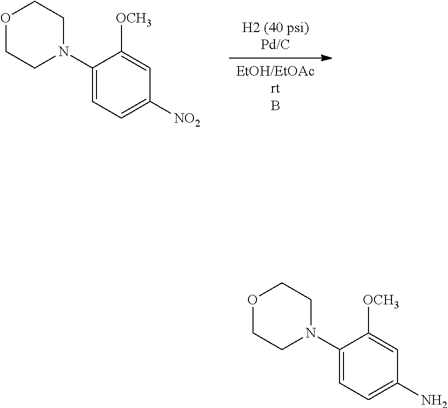 [SMILES: COc1cc([N+](=O)[O-])ccc1N1CCOCC1 ; COc1cc(N)ccc1N1CCOCC1]

To a suspension of 4-(2-methoxy-4-nitrophenyl)morpholine (97.8 g, 420 mmol) in ethanol (2 L) was added palladium on carbon (Pd/C) (11 g). The mixture was stirred at room temperature for 5 hours under a 50 psi atmosphere of hydrogen gas. The reaction mixture was purged, filtered, and washed with ethanol. The filtrate was concentrated in vacuo to give the title compound as a brown solid.

### Example 3

### Preparation of (6-bromo-imidazo[1,2-a]pyrazin-8-yl)-(3-methoxy-4-morpholin-4-ylphenyl)-amine

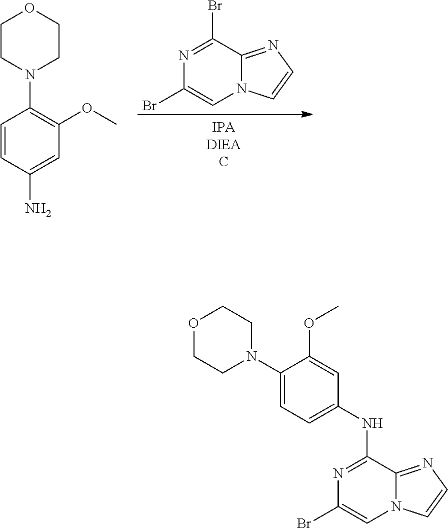

A mixture of 4-(2-methoxy-4-aminophenyl)morpholine (6.5 g, 31.3 mmol), 6,8 dibromoimidazo[1,2-a]pyrazine (8.66 g, 31.3) and N,N-diisopropylethylamine (10.9 mL, 62.6 mmol) in isopropanol was heated at reflux for 30 hours. After being cooled down to room temperature, the solid was filtered and washed with isopropanol (2×), dried to give the title compound.

### Example 4

### Preparation of 7-(4,4,5,5-tetramethyl-1,3,2-dioxaborolan-2-yl)-2,3-dihydro-1H-pyrido[2,3-b][1,4]oxazine

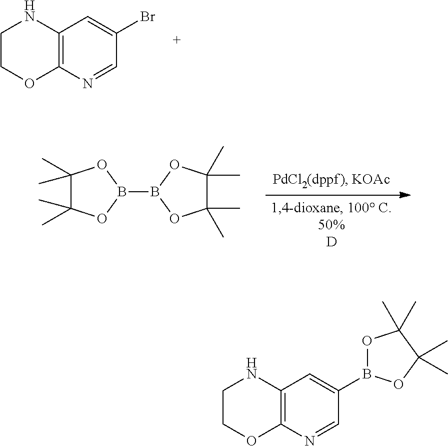

In a dry 1 L 3-neck flask under nitrogen was placed 7-bromo-2,3-dihydro-1H-pyrido[2,3-b][1,4]oxazine (20 g, 93 mmol), pinacole-diboron (28 g, 110 mmol), potassium acetate (30 g, 305 mmol), and 1,1′-bis(diphenylphosphino)ferrocene]dichloropalladium(II) (PdCl₂dppf) (6 g, 7.3 mmol). 600 mL 1,4-dioxane was added and the reaction heated with stirring to 100° C. for about 2 hours whereupon LCMS indicated complete consumption of the bromide. The reaction mixture was cooled and filtered through celite washing through with ethyl acetate, filtered and concentrated to give a dark solid. A large 4″ diameter Buchner funnel with glass frit was filled with 2″ of silica gel and slurried with ethyl acetate and allowed to settle. The crude material was dissolved in ethyl acetate and run though the silica bed with vacuum assist, eluting with about 4 L of ethyl acetate and leaving behind a black residue atop the silica. The elutant was then concentrated to give a yellow solid which was triturated with minimal diethyl ether and the solids collected by filtration and washed with ether then dried to give the desired product as a pure white solid. The mother liquor was reconcentrated and retriturated to result in the desired product.

### Example 5a

### Preparation of 6-(2,3-dihydro-1H-pyrido[2,3-b][1,4]oxazin-7-yl)-N-(3-methoxy-4-morpholinophenyl)imidazo[1,2-a]pyrazin-8-amine

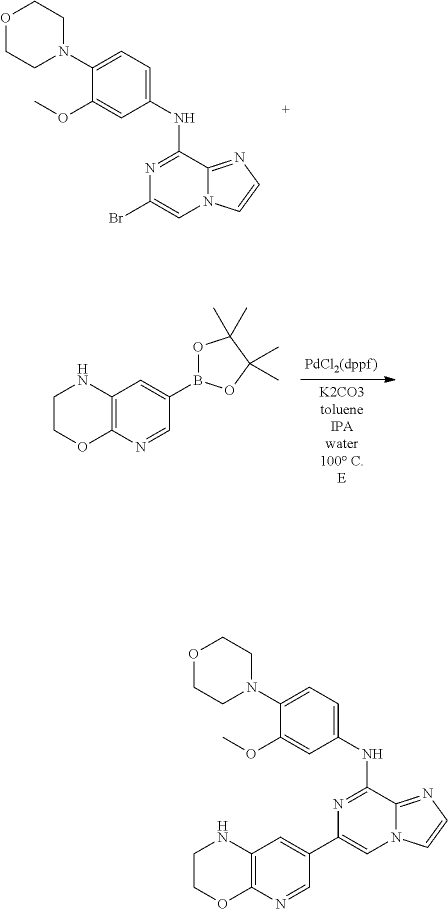 [SMILES: COc1cc(Nc2nc(Br)cn3ccnc23)ccc1N1CCOCC1 ; CC1(C)OB(c2cnc3c(c2)NCCO3)OC1(C)C ; COc1cc(Nc2nc(-c3cnc4c(c3)NCCO4)cn3ccnc23)ccc1N1CCOCC1]

In a dry 1 L 3-neck flask under nitrogen was placed 50 mL toluene, 25 mL isopropanol and 25 mL water and nitrogen was bubbled through for 15 minutes at 60° C. The temperature was raised to 100° C. and then 6-bromo-N-(3-methoxy-4-morpholinophenyl)imidazo[1,2-a]pyrazin-8-amine (4.04 g, 10 mmol, 1.0 equivs.), 7-(4,4,5,5-tetramethyl-1,3,2-dioxaborolan-2-yl)-2,3-dihydro-1H-pyrido[2,3-b][1,4]oxazine (1.50 equivs, 15 mmol, 3.93 g) and PdCl₂dppf (5 mol %, 0.5 mmol, 408 mg) were all added and the reaction was allowed to proceed for 3 hours. At that point the reaction was deemed complete by LCMS analysis and the solution was concentrated at 50° C. The sample was stripped from 100 mL of 1:1 methylene chloride/ethyl acetate at which point the sample crashed out of solution. The remaining aqueous solution was decanted off and the solid was redissolved in 10% methanol/methylene chloride and passed through a plug of silicon dioxide (SiO₂). The filtrate was collected, concentrated and redissolved in methylene chloride and washed 2× saturated sodium bicarbonate and 1× brine followed by drying over sodium sulfate. The drying solution was then passed through a 2″ plug of Celite washing with methylene chloride until no UV activity seen. The filtrate was then concentrated down to where the sample started to precipitate. Ethyl acetate was then added and the sample was sonicated for 5 minutes, filtered, washed with hexanes and vacuum dried. This yielded an off white solid. All filtrates were then combined and placed through a second celite plug and concentrated. This sample was triturated with 1:1 ethyl acetate/hexanes and refiltered and dried to yield an off-white solid.

M.P. 233-234° C.; [M+H]=460.6; ¹H NMR (300 MHz, DMSO) (δ): 9.50 (s, 1H), 8.43 (s, 1H), 8.01 (d, 1H), 7.97 (d, 1H), 7.92 (d, 1H), 7.71 (dd, 1H), 7.62 (d, 1H), 7.47 (d, 1H) 6.91 (d, 1H), 6.22 (m, 1H), 4.31 (bt, 2H), 3.82 (s, 3H), 3.73 (bt, 4H), 3.32 (bt, 2H), 2.95 (bt, 4H).

### Example 5b

### Preparation of 6-(2,3-dihydro-1H-pyrido[2,3-b][1,4]oxazin-7-yl)-N-(3-methoxy-4-morpholinophenyl)imidazo[1,2-a]pyrazin-8-amine

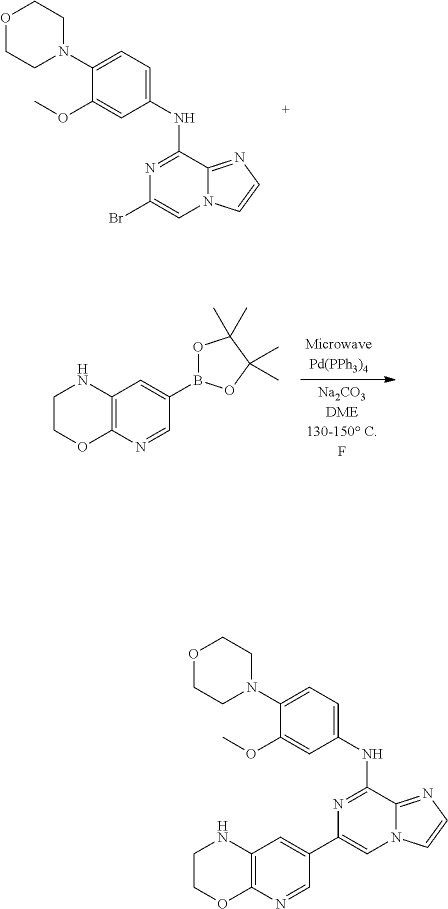 [SMILES: COc1cc(Nc2nc(Br)cn3ccnc23)ccc1N1CCOCC1 ; CC1(C)OB(c2cnc3c(c2)NCCO3)OC1(C)C ; COc1cc(Nc2nc(-c3cnc4c(c3)NCCO4)cn3ccnc23)ccc1N1CCOCC1]

In a 10-20 mL Biotage microwave vial was placed 6-bromo-N-(3-methoxy-4-morpholinophenyl)imidazo[1,2-a]pyrazin-8-amine (565 mg, 1.4 mmol, 1.0 equivs.), 7-(4,4,5,5-tetramethyl-1,3,2-dioxaborolan-2-yl)-2,3-dihydro-1H-pyrido[2,3-h][1,4]oxazine (1.2 equivs, 1.68 mmol, 440 mg) and added dimethoxyethane 9 mL followed by 1.5 mL 2N sodium bicarbonate and palladium tetrakis (80 mg, 5 mol %). The vial was heated to 150° C. in the microwave for 20 min after which time LCMS indicated complete consumption of the bromide and a large peak corresponding to the desired product. The reaction was diluted with ethyl acetate and water, separated, and extracted 2× with ethyl acetate, and then washed with brine 2×, dried over sodium sulfate, filtered and concentrated under vacuum. The residue was dissolved in methylene chloride and loaded onto an ISCO 40 g gold silica column and eluted with a gradient from 10-80% of methylene chloride and a premixed solution of solvent B (60% methylene chloride, 30% diethyl ether, 10% methanol). The fractions were combined and concentrated to dryness then triturated with ether and the solids collected by filtration to give the desired product.

### Example 6

### Preparation of 7-(4,4,5,5-tetramethyl-1,3,2-dioxaborolan-2-yl)-3,4-dihydrobenzo[f][1,4]oxazepin-5(2H)-one

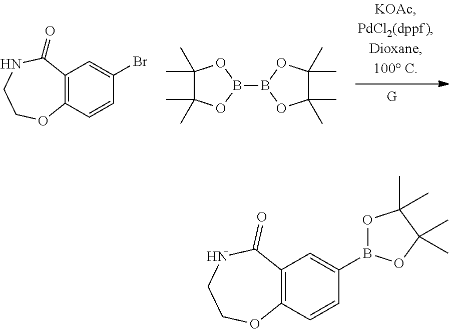

This compound was synthesized according to procedure D in Example 4 above.

### Example 7

### Preparation of 7-(8-((3-methoxy-4-morpholinophenyl)amino)imidazo[1,2-a]pyrazin-6-yl)-3,4-dihydrobenzo[f][1,4]oxazepin-5(2H)-one

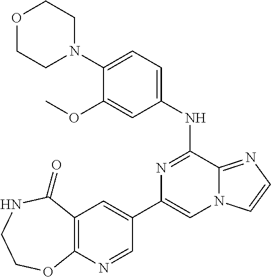 [SMILES: COc1cc(Nc2nc(-c3cnc4c(c3)C(=O)NCCO4)cn3ccnc23)ccc1N1CCOCC1]

This compound was synthesized according to procedure E in Example 5a above.

¹H NMR (300 MHz, DMSO-d6): δ 9.51 (s, 1H), 8.58 (s, 1H), 8.43-8.39 (m, 2H), 8.04-8.01 (m, 2H), 9.97 (d, 1H), 7.62 (d, 1H), 7.48 (d, 1H), 7.11 (d, 1H), 6.85 (d, 1H), 4.33 (t, 2H), 3.81 (s, 3H), 3.72 (t, 4H), 3.3.35 (dd, 2H), 2.93 (t, 4H) LCMS [M+H]: 487.4.

### Example 8

### Preparation of N-(3-methoxy-4-morpholinophenyl)-6-(2,3,4,5-tetrahydrobenzo[f][1,4]oxazepin-7-yl)imidazo[1,2-a]pyrazin-8-amine

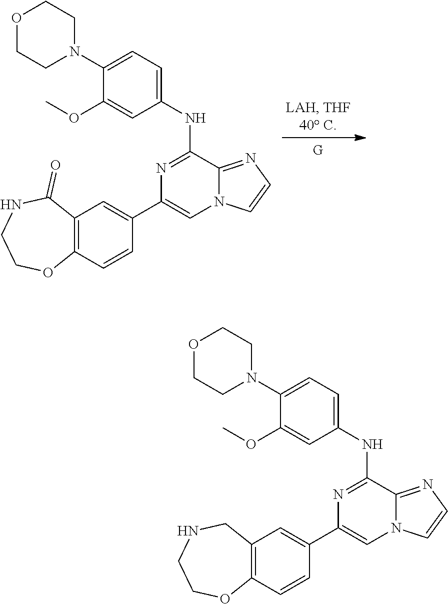 [SMILES: COc1cc(Nc2nc(-c3ccc4c(c3)C(=O)NCCO4)cn3ccnc23)ccc1N1CCOCC1 ; COc1cc(Nc2nc(-c3ccc4c(c3)CNCCO4)cn3ccnc23)ccc1N1CCOCC1]

In a dry 500 mL 3-neck flask equipped with a condenser, addition funnel and magnetic stirbar under nitrogen was added (6-bromo-imidazo[1,2-a]pyrazin-8-yl)-(3-methoxy-4-morpholin-4-ylphenyl)-amine (5.3 g, 10.9 mmol) followed by anhydrous tetrahydrofuran (120 mL). The flask was cooled in an ice/water bath and 1 M Lithium Aluminum hydride (65 mL, 65 mmol) was added dropwise with vigorous stirring over about 30 minutes. The reaction was allowed to stir an additional 15 min after the addition was complete then allowed to warm to room temperature for about 20 min then heated to 40° C. overnight about 16 hours at which time LCMS showed <3% SM remaining, a large product peak, and a smaller but significant impurity peak (less polar than the SM and product). The reaction mixture was cooled in and ice/water bath and quenched via the Fieser 1,2,3 method: Slowly added 2.5 mL of water dropwise, followed by 5 mL 15% NaOH and stirred for 5 min followed by an additional 7.5 mL water, then stirred for 1 hr at room temp. The slurry was then filtered through a pad of celite washing through with ethyl acetate then dried over sodium sulfate, filtered and concentrated to dryness via rotovap. The residue was dissolved in methanol/methylene chloride, and adsorbed onto silica gel about 30 g and placed in an ISCO solid load cartridge. A column was then run using an ISCO 80 g gold silica column eluting with methylene chloride (solvent A) and premixed 10% methanol/methylene chloride (solvent B). Column gradient: 10% B to 60% B over 5CV then held for 5 CV then ramped to 100% B over 10CV. The major impurity began eluting at 60% B and partially co-eluted with the desired product. Clean fractions were combined and concentrated to dryness via rotovap. NMR indicated significant methylene chloride so the product was dissolved in absolute ethanol (about 20-30 mL) and spun on the rotovap at 50° C. for about 40 min then reduced to dryness. This process was then repeated with diethyl ether at 40° C. and the resulting solid dried under vacuum overnight. NMR and LCMS both show desired, N-(3-methoxy-4-morpholinophenyl)-6-(2,3,4,5-tetrahydrobenzo[f][1,4]oxazepin-7-yl)imidazo[1,2-a]pyrazin-8-amine as a white solid.

¹H NMR (300 MHz, DMSO-d6): δ 9.48 (s, 1H), 8.50 (s, 1H), 8.07 (d, 1H), 7.94 (s, 1H), 7.81-7.76 (m, 2H), 7.61 (s, 1H), 7.55 (d, 1H), 7.04 (d, 1H), 6.87 (d, 1H), 3.96 (t, 2H), 3.85 (s, 2H), 3.83 (s, 3H), 3.71 (t, 4H), 3.04 (t, 2H), 2.93 (t, 4H). LCMS [M+H]: 473.5.

### Example 9

### Preparation of 1-(2-Methoxy-4-nitrophenyl)-4-(oxetan-3-yl)piperazine

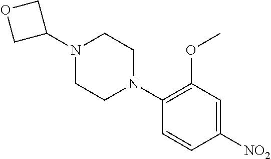 [SMILES: COc1cc([N+](=O)[O-])ccc1N1CCN(C2COC2)CC1]

To a round-bottomed flask equipped with a stirring bar and a nitrogen gas tee, 1-(oxetan-3-yl)piperazine (8.00 g, 56.26 mmol), 1-fluoro-2-methoxy-4-nitrobenzene (9.63 g, 56.26 mmol), potassium carbonate (K₂CO₃) (38.88 g, 281.29 mmol), and N-methyl-2-pyrrolidone (NMP) (100 mL) were added. The resulting mixture was heated at 100° C. overnight. Water (500 mL) was added and the resulting mixture was extracted with ethyl acetate (200 mL×3), the combined organic phases were washed with H₂O (50 mL×1), brine (50 mL×1), and dried over sodium sulfate. The organic phase was filtered and removed solvent in vacuo, and passed a silica gel column (methanol:methylene chloride=5:95), yellow solids were obtained as the desired product.

### Example 10

### Preparation of 3-Methoxy-4-(4-(oxetan-3-yl)piperazin-1-yl)aniline

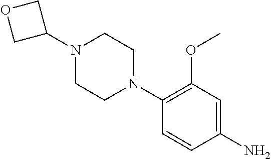 [SMILES: COc1cc(N)ccc1N1CCN(C2COC2)CC1]

To a hydrogenation bottle, 1-(2-methoxy-4-nitrophenyl)-4-(oxetan-3-yl)piperazine (16 g, 54.55 mmol), methanol:CH₂Cl₂ (9:1, 120 mL) were added. The suspension was bubbled nitrogen gas for 5 minutes, following the addition of palladium on carbon (Pd/C) (10%, 2.32 g, 2.18 mmol), the mixture was hydrogenated in a Parr shaking-type hydrogenator (50 psi) for 2 hrs. The resulting suspension was filtered through celite, the celite was washed with methanol (50 mL×2), and the combined filtrate was removed solvent to afford brown solids as the desired product.

### Example 11

### Preparation of 6-Bromo-N-(3-methoxy-4-(4-(oxetan-3-yl)piperazin-1-yl)phenyl)imidazo[1,2-a]pyrazin-8-amine

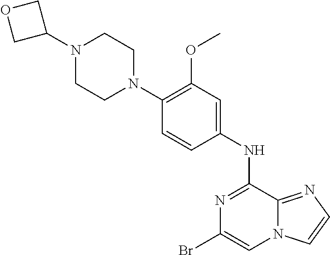 [SMILES: COc1cc(Nc2nc(Br)cn3ccnc23)ccc1N1CCN(C2COC2)CC1]

To a 300 mL seal tube equipped with a stirring bar, 3-methoxy-4-(4-(oxetan-3-yl)piperazin-1-yl)aniline (10.00 g, 37.97 mmol), 6,8-dibromoimidazo[1,2-a]pyrazine (10.52 g, 37.97 mmol), isopropanol (152 mL), and diisopropylethylamine (9.82 g, 75.95 mmol) were added, the seal tube was sealed and heated at 85° C. overnight. Saturated aqueous sodium bicarbonate (150 mL) was added and the resulting mixture was extracted with methanol:methylene chloride (1:3, 150 mL×3), the combined organic phases were washed with brine (30 mL×1), dried over sodium sulfate, and removed solvent. The resulting residue was passed a silica gel column (methanol:methylene chloride=5:95) and yellow solids were obtained as the desired product.

### Example 12

### Preparation of 6-(3,4-Dihydro-2H-benzo[b][1,4]oxazin-6-yl)-N-(3-methoxy-4-(4-(oxetan-3-yl)piperazin-1-yl)phenyl)imidazo[1,2-a]pyrazin-8-amine

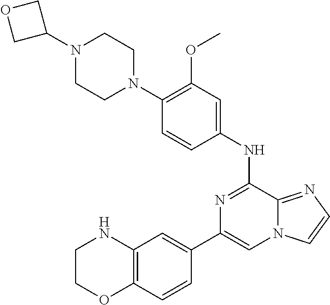 [SMILES: COc1cc(Nc2nc(-c3ccc4c(c3)NCCO4)cn3ccnc23)ccc1N1CCN(C2COC2)CC1]

This compound was synthesized according to procedure E in Example 5a above.

MP 199-201° C. ¹H NMR (300 MHz, d₆-DMSO): δ 9.41 (s, 1H), 8.33 (s, 1H), 7.99 (d, 1H), 7.96 (d, 1H), 7.68 (dd, 1H), 7.60 (d, 1H), 7.24 (d, 1H), 7.12 (dd, 1H), 6.91 (d, 1H), 6.73 (d, 1H), 5.88 (s, 1H), 4.56 (t, 2H), 4.47 (t, 2H), 4.16 (t, 2H), 3.82 (s, 3H), 3.47 (p, 1H), 2.98 (broad s, 4H), 2.41 (broad s, 4H); MS (ESI+) m/z 514.6 (M+H).

### Example 13

### Preparation of (S)-1-((R)-4-(4-((6-(2,3-dihydro-1H-pyrido[2,3-b][1,4]oxazin-7-yl)imidazo[1,2-a]pyrazin-8-yl)amino)phenyl)morpholin-2-yl)ethanol

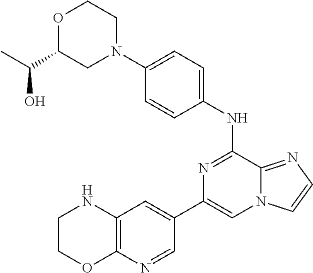 [SMILES: C[C@H](O)[C@H]1CN(c2ccc(Nc3nc(-c4cnc5c(c4)NCCO5)cn4ccnc34)cc2)CCO1]

This compound was synthesized according to procedure E in Example 5a above.

MS (ESI+) m/z 475.5 (M+H); ¹H NMR [300 MHz, d₆-DMSO, (δ, ppm)]: 9.44 (s, 1H), 8.39 (s, 1H), 7.96 (m, 4H), 7.59 (s, 1H), 7.43 (d, 1H), 6.97 (d, 1H), 6.25 (bs, 1H), 4.61 (d, 1H), 4.29 (bt, 2H), 3.96 (dd, 2H), 3.65 (m, 2H), 3.44 (m, 3H), 3.27 (m, 2H), 2.62 (m, 1H), 1.10 (d, 3H).

### Example 14

### Preparation of 6-(3,4-dihydro-2H-benzo[b][1,4]oxazin-7-yl)-N-(3-methoxy-4-(4-(oxetan-3-yl)piperazin-1-yl)phenyl)imidazo[1,2-a]pyrazin-8-amine

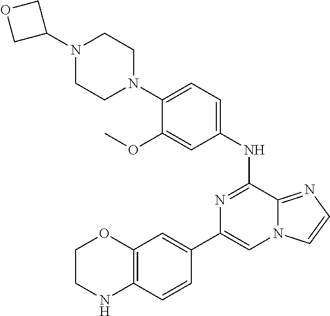 [SMILES: COc1cc(Nc2nc(-c3ccc4c(c3)OCCN4)cn3ccnc23)ccc1N1CCN(C2COC2)CC1]

This analog was synthesized according to procedure E in Example 5a above.

MS (ESI+) m/z 514.5 (M+H); NMR (δ, ppm): 9.37 (s, 1H), 8.34 (s, 1H), 8.07 (d, 1H), 7.88 (d, 1H), 7.56 (d, 1H), 7.52 (dd, 1H), 7.34 (m, 2H), 6.86 (d, 1H), 6.60 (d, 1H), 5.98 (bs, 1H), 4.54 (t, 2H), 4.45 (t, 2H), 4.14 (bt, 2H), 3.83 (s, 3H), 3.45 (p, 1H), 3.29 (m, 2H), 3.27 (m, 2H), 2.96 (m, 41H), 2.39 (m, 4H).

### Example 15

### Preparation of N-(3-methoxy-4-morpholinophenyl)-6-(1-methyl-2,3-dihydro-1H-pyrido[2,3-b][1,4]oxazin-7-yl)imidazo[1,2-a]pyrazin-8-amine

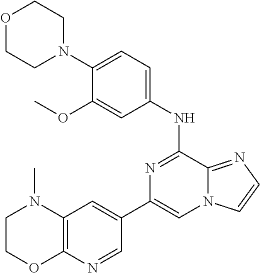 [SMILES: COc1cc(Nc2nc(-c3cnc4c(c3)N(C)CCO4)cn3ccnc23)ccc1N1CCOCC1]

This compound was synthesized according to procedure E in Example 5a above.

MS (ESI+) m/z 474.2 (M+H); NMR (δ, ppm): 9.52 (s, 1H), 8.56 (s, 1H), 8.11 (d, 1H), 7.95 (d, 1H), 7.86 (d, 1H), 7.71 (dd, 1H), 7.63 (d, 1H), 7.50 (d, 1H), 6.88 (d, 1H), 6.60 (d, 1H), 4.40 (bt, 2H), 3.81 (s, 3H), 3.74 (bt, 4H), 3.32 (s, 3H), 3.29 (m, 2H), 2.95 (m, 4H).

### Example 16

### Preparation of 6-(2,3-dihydro-1H-pyrido[2,3-b][1,4]oxazin-7-yl)-N-(3-ethoxy-4-morpholinophenyl)imidazo[1,2-a]pyrazin-8-amine

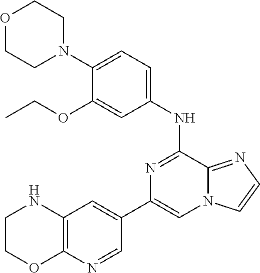 [SMILES: CCOc1cc(Nc2nc(-c3cnc4c(c3)NCCO4)cn3ccnc23)ccc1N1CCOCC1]

This compound was synthesized according to procedure F in Example 5b above. Starting material 6-bromo-N-(3-ethoxy-4-morpholinophenyl)imidazo[1,2-a]pyrazin-8-amine was synthesized using procedure A utilizing 3-ethoxy-4-fluoronitrobenzene.

MS (ESI+) m/z 474.5 (M+H); NMR (δ, ppm): 9.47 (s, 1H), 8.40 (s, 1H), 7.97 (d, 1H), 7.95 (d, 1H), 7.91 (d, 1H), 7.68 (dd, 1H), 7.60 (d, 1H), 7.44 (d, 1H), 6.87 (d, 1H), 6.20 (bs, 1H), 4.29 (bt, 2H), 4.05 (q, 2H), 3.71 (bt, 4H), 3.31 (m, 2H), 2.94 (bt, 4H), 1.35 (t, 3H).

### Example 17

### Preparation of (R)—N-(4-(3-aminopyrrolidin-1-yl)-3-methoxyphenyl)-6-(3,4-dihydro-2H-benzo[b][1,4]oxazin-6-yl)imidazo[1,2-a]pyrazin-8-amine

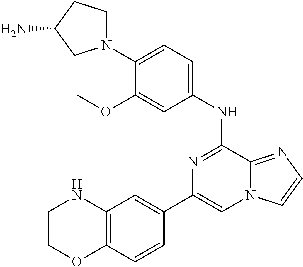 [SMILES: COc1cc(Nc2nc(-c3ccc4c(c3)NCCO4)cn3ccnc23)ccc1N1CC[C@@H](N)C1]

This compound was synthesized using procedure E in Example 5a above, followed by Boc removal procedure H.

MS (ESI+) m/z 458.6 (M+H); NMR (δ, ppm): 9.26 (s, 1H), 8.27 (s, 1H), 7.93 (bs, 1H), 7.89 (bs, 1H), 7.60 (bs, 1H), 7.57 (bs, 1H), 7.21 (s, 1H), 7.10 (dd, 1H), 6.71 (d, 1H), 6.65 (d, 1H), 5.85 (bs, 1H), 4.14 (bt, 2H), 3.78 (s, 3H), 3.29 (m, 2H), 2.88 (m, 2H), 2.03 (m, 1H), 1.56 (m, 1H).

### Example 18

### Preparation of N-(4-(difluoromethoxy)-3-methoxyphenyl)-6-(2,3-dihydro-1H-pyrido[2,3-b][1,4]oxazin-7-yl)imidazo[1,2-a]pyrazin-8-amine

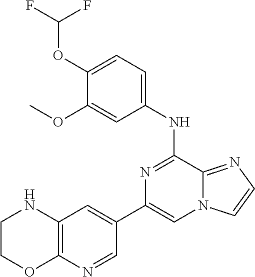 [SMILES: COc1cc(Nc2nc(-c3cnc4c(c3)NCCO4)cn3ccnc23)ccc1OC(F)F]

This compound was synthesized using procedure F in Example 5b above.

¹H NMR (300 MHz, DMSO) (δ): 9.75 (s, 1H), 8.47 (s, 1H), 8.15 (d, 2H), 7.98 (t, 2H), 7.74 (dd, 1H), 7.62 (d, 1H), 7.45 (d, 1H), 7.18 (t, 1H), 6.23 (s, 1H), 4.38 (t, 2H), 3.84 (s, 3H), 3.31 (d, 2H). MS (M+H) for C₂₁H₁₈N₆O₃: 441.50 (MH⁺).

### Example 19

### Preparation of 6-(2,3-dihydro-1H-pyrido[2,3-b][1,4]oxazin-7-yl)-N-(6-morpholinylpyridin-3-yl)imidazo[1,2-a]pyrazin-8-amine

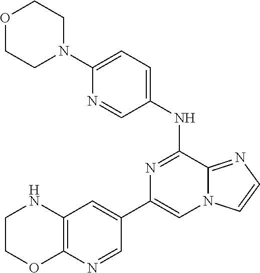 [SMILES: c1cn2cc(-c3cnc4c(c3)NCCO4)nc(Nc3ccc(N4CCOCC4)nc3)c2n1]

This compound was synthesized according to procedure F in Example 5b above. Starting material 6-bromo-N-(6-morpholinylpyridin-3-yl)imidazo[1,2-a]pyrazin-8-amine was synthesized using procedure A utilizing 2-chloro-5-nitropyridine.

¹H NMR (300 MHz, DMSO) (δ): ¹H NMR (300 MHz, DMSO) ( ): 9.61 (s, 1H), 8.812 (d, 1H), 8.38 (s, 1H), 8.26 (dd, 1H), 7.95 (d, 1H), 7.93 (d, 1H), 7.60 (d, 1H), 7.36 (d, 1H), 6.89 (d, 1H), 6.21 (bs, 1H), 4.28 (t, 2H), 3.71 (t, 4H), 3.39 (t, 4H), 3.29 (m, 2H). MS (M+H) for CHNO: 431.34 (MH⁺).

### Example 20

### Preparation of 6-(2,3-dihydro-1H-pyrido[2,3-b][1,4]oxazin-7-yl)-N-(3-fluoro-4-morpholinophenyl)imidazo[1,2-a]pyrazin-8-amine

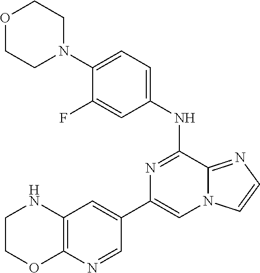 [SMILES: Fc1cc(Nc2nc(-c3cnc4c(c3)NCCO4)cn3ccnc23)ccc1N1CCOCC1]

This compound was synthesized according to procedure F in Example 5b above. Starting material 6-bromo-N-(3-fluoro-4-morpholinophenyl)imidazo[1,2-a]pyrazin-8-amine was synthesized using procedure A utilizing 3,4-difluoronitrobenzene.

¹H NMR (300 MHz, DMSO) (δ): 9.76 (s, 1H), 8.44 (s, 1H), 7.96 (t, 3H), 7.94 (d, 1H), 7.62 (d, 1H), 7.40 (s, 1H), 7.04 (t, 1H), 6.23 (s, 1H), 4.29 (t, 2H), 3.73 (t, 1H), 3.29 (t, 2H), 2.97 (t, 4H). MS (M+H) for C₂₃H₂₂N₇O₂: 488.39 (MH⁺).

### Example 21

### Preparation of 6-(2,3-dihydro-1H-pyrido[2,3-b][1,4]oxazin-7-yl)-N-(4-((2S,6R)-2,6-dimethylmorpholinyl)-3-methoxyphenyl)imidazo[1,2-a]pyrazin-8-amine

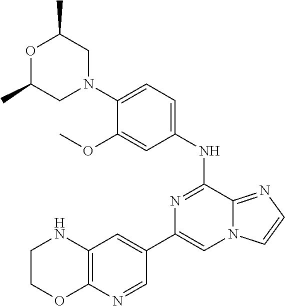 [SMILES: COc1cc(Nc2nc(-c3cnc4c(c3)NCCO4)cn3ccnc23)ccc1N1C[C@@H](C)O[C@@H](C)C1]

This compound was synthesized according to procedure F in Example 5b.

¹H NMR (300 MHz, DMSO) (δ): 9.45 (s, 1H), 8.41 (s, 1H), 7.98 (d, 2H), 7.86 (d, 1H), 7.75 (dd, 1H), 7.60 (s, 1H), 7.44 (d, 1H), 6.90 (d, 1H), 6.19 (s, 1H), 4.25 (t, 1H), 3.80 (s, 4H), 3.75 (q, 2H), 3.23 (dd, 2H), 2.22 (t, 2H), 1.15 (s, 6H). MS (M+H) for C₂₆H₂₉N₇O₃: 488.46 (MH⁺).

### Example 22

### Preparation of 6-(2,3-dihydro-1H-pyrido[2,3-b][1,4]oxazin-7-yl)-N-(3-methoxy-4-(2,5-dioxa-8-azaspiro[3.5]nonan-8-yl)phenyl)imidazo[1,2-a]pyrazin-8-amine

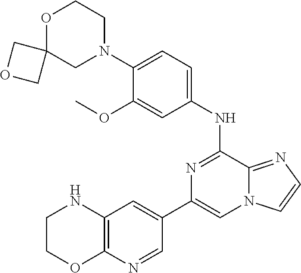 [SMILES: COc1cc(Nc2nc(-c3cnc4c(c3)NCCO4)cn3ccnc23)ccc1N1CCOC2(COC2)C1]

This compound was synthesized according to procedure F in Example 5b above.

¹H NMR (300 MHz, DMSO) (δ): 9.41 (s, 1H), 8.39 (s, 1H), 7.98 (d, 1H), 7.86 (d, 1H), 7.59 (dd, 1H), 7.58 (d, 1H), 7.44 (d, 1H), 6.69 (d, 1H), 6.19 (s, 1H), 4.29 (t, 1H), 3.87 (q, 4H), 3.78 (s, 3H), 3.29 (t, 2H), 2.01 (t, 2H). MS (M+H) for C₂₆H₂₇N₇O₄: 502.41 (MH⁺).

### Example 23

### Preparation of (R)-4-(4-((6-(2,3-dihydro-1H-pyrido[2,3-b][1,4]oxazin-7-yl)imidazo[1,2-a]pyrazin-8-yl)amino)phenyl)morpholine-2-carboxamide

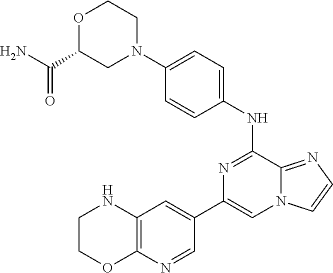 [SMILES: NC(=O)[C@H]1CN(c2ccc(Nc3nc(-c4cnc5c(c4)NCCO5)cn4ccnc34)cc2)CCO1]

This compound was synthesized according to procedure F in Example 5b above.

¹H NMR (300 MHz, DMSO) (δ): 9.47 (s, 1H), 8.41 (s, 1H), 7.96 (dd, 2H), 7.93 (d, 2H), 7.59 (s, 1H), 7.45 (s, 1H), 7.30 (d, 1H), 6.97 (d, 1H), 6.28 (s, 1H), 4.28 (t, 2H), 4.00 (dt, 2H), 3.70 (m, 2H), 3.43 (d, 1H), 3.31 (t, 2H), 2.72 (m, 1H), 2.60 (t, 1H). MS (M+H) for C₂₄H₂₄N₈O₃: 473.46

### Example 24

### Preparation of 6-(2,3-dihydro-1H-pyrido[2,3-b][1,4]oxazin-7-yl)-N-(3-methoxy-4-(7-oxa-2-azaspiro[3.5]nonan-2-yl)phenyl)imidazo[1,2-a]pyrazin-8-amine

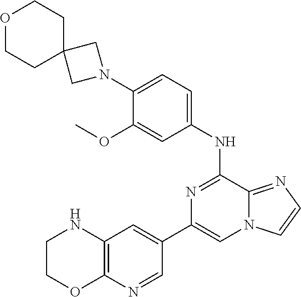 [SMILES: COc1cc(Nc2nc(-c3cnc4c(c3)NCCO4)cn3ccnc23)ccc1N1CC2(CCOCC2)C1]

This compound was synthesized according to procedure F in Example 5b above.

¹H NMR (300 MHz, DMSO) (δ): 9.34 (s, 1H), 8.37 (s, 1H), 7.98 (d, 2H), 7.92 (d, 1H), 7.79 (d, 1H), 7.79 (d, 1H), 7.58 (d, 1H), 7.55 (d, 1H), 7.43 (d, 1H), 6.38 (d, 1H), 6.18 (s, 2H), 4.28 (t, 2H), 3.74 (s, 3H), 3.59 (s, 4H), 3.53 (t, 4H), 1.71 (t, 4H). MS (M+H) for C₂₇H₂₉N₇O₃: 500.43 (MH⁺).

### Example 25

### Preparation of 6-(2,3-dihydro-1H-pyrido[2,3-b][1,4]oxazin-7-yl)-N-(3-methoxy-4-(1,4-oxazepan-4-yl)phenyl)imidazo[1,2-a]pyrazin-8-amine

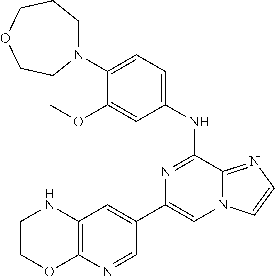 [SMILES: COc1cc(Nc2nc(-c3cnc4c(c3)NCCO4)cn3ccnc23)ccc1N1CCCOCC1]

This compound was synthesized according to procedure F in Example 5b above.

¹H NMR (300 MHz, DMSO) (δ): 9.45 (s, 1H), 8.41 (s, 1H), 7.98 (d, 2H), 7.96 (d, 1H), 7.80 (dd, 1H), 7.65 (d, 1H), 7.60 (d, 1H), 7.45 (d, 1H), 6.90 (d, 1H), 6.20 (s, 1H), 4.30 (t, 2H), 3.80 (s, 3H), 3.75 (q, 4H), 2.22 (q, 4H), 1.95 (q, 2H). MS (M+H) for C₂₅H₂₇N₇O₃: 474.40 (MH⁺).

### Example 26

### Preparation of (R)-6-(2,3-dihydro-1H-pyrido[2,3-b][1,4]oxazin-7-yl)-N-(3-methoxy-4-(1-oxa-6-azaspiro[3.4]octan-6-yl)phenyl)imidazo[1,2-a]pyrazin-8-amine

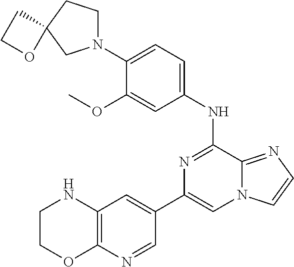 [SMILES: COc1cc(Nc2nc(-c3cnc4c(c3)NCCO4)cn3ccnc23)ccc1N1CC[C@@]2(CCO2)C1]

This compound was synthesized according to procedure F in Example 5b above.

¹H NMR (300 MHz, DMSO) (δ): 9.38 (s, 1H), 8.38 (s, 1H), 7.98 (d, 1H), 7.93 (d, 1H), 7.84 (s, 1H), 7.59 (d, 1H), 7.58 (d, 1H), 7.44 (d, 1H), 6.66 (d, 1H), 6.19 (s, 1H), 4.35 (t, 4H), 3.78 (s, 1H), 3.43 (s, 2H), 3.3 (t, 2H), 3.15 (m, 2H), 2.66 (m, 2H), 2.20 (m, 2H). MS (M+H) for C₂₆H₂₇N₇O₃: 486.40 (MH⁺).

### Example 27

### Preparation of (S)-7-(4-((6-(2,3-dihydro-1H-pyrido[2,3-b][1,4]oxazin-7-yl)imidazo[1,2-a]pyrazin-8-yl)amino)-2-methoxyphenyl)-1-oxa-3,7-diazaspiro[4.5]decan-2-one

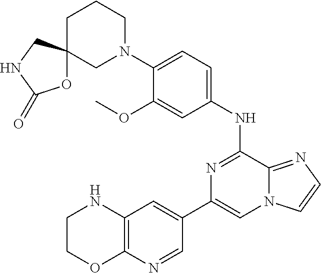 [SMILES: COc1cc(Nc2nc(-c3cnc4c(c3)NCCO4)cn3ccnc23)ccc1N1CCC[C@]2(CNC(=O)O2)C1]

This compound was synthesized according to procedure F in Example 5b above.

¹H NMR (300 MHz, DMSO) (δ): 9.51 (s, 1H), 8.44 (s, 1H), 7.99 (d, 1H), 7.97 (d, 1H), 7.95 (d, 1H), 7.67 (dd, 1H), 7.62 (d, 1H), 7.55 (s, 1H), 7.47 (d, 1H), 6.93 (d, 1H), 4.31 (t, 2H), 3.82 (s, 3H), 3.72 (d, 1H), 3.12 (d, 2H), 1.8 (m, 2H), 1.6 (m, 1H). MS (M+H) for C₂₇H₂₈N₈O₄: 529.78 (MH⁺).

### Example 28

### Preparation of 1-(4-nitrophenyl)-4-(oxetan-3-yl)piperazine

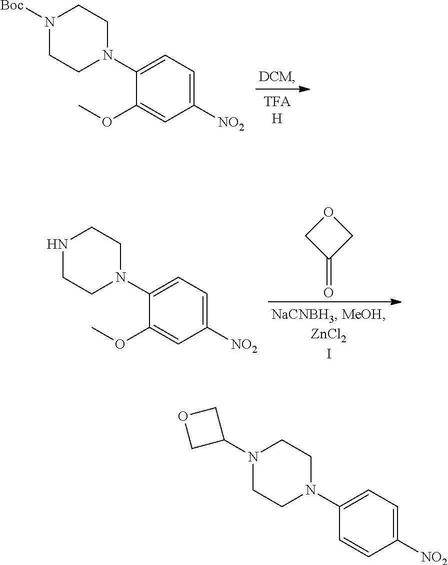 [SMILES: COc1cc([N+](=O)[O-])ccc1N1CCN(C(=O)OC(C)(C)C)CC1 ; COc1cc([N+](=O)[O-])ccc1N1CCNCC1 ; O=C1COC1 ; O=[N+]([O-])c1ccc(N2CCN(C3COC3)CC2)cc1]

In a 500 mL round bottom flask, 1-(oxetan-3-yl)piperazine (3.02 g, 21.26 mmoles), potassium carbonate (5.87 g, 42.52 mmoles), 1-fluoro-4-nitrobenzene (3.00 g, 21.26 mmoles) was combined in acetonitrile (33 mL) and stirred under nitrogen overnight at 100° C. The mixture was diluted with water (100 mL) and extracted with methylene chloride (100 mL×3), dried over anhydrous sodium carbonate, filtered and the filtrate was concentrated. The residue was dissolved in minimal methylene chloride using a sonicator and crashed out with hexane. The precipitate was filtered, washed with hexane and dried to afford the title compound as an orange solid.

### Example 29

### Preparation of 4-(4-(oxetan-3-yl)piperazin-1-yl)aniline

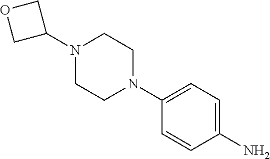 [SMILES: Nc1ccc(N2CCN(C3COC3)CC2)cc1]

In a hydrogenation vessel, 1-(4-nitrophenyl)-4-(oxetan-3-yl)piperazine (4.70 g, 17.85 mmoles) was dissolved as much as possible in methanol (26 mL) and methylene chloride (5 mL). Pd/C (10%) (2.85 g, 2.68 mmoles) was added and the reaction was stored under nitrogen. The reaction was shaken on the Parr hydrogenator at 45 PSI. After 15 minutes, the reaction was fully recharged to 45 PSI and shaken for an additional hour. The material was filtered over celite, washed with 25% methanol/methylene chloride and concentrated to provide the title compound as a light brown solid.

### Example 30

### Preparation of 6-bromo-N-(4-(4-(oxetan-3-yl)piperazin-1-yl)phenyl)imidazo[1,2-a]pyrazin-8-amine

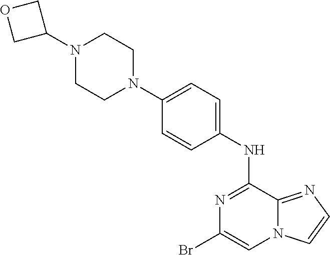 [SMILES: Brc1cn2ccnc2c(Nc2ccc(N3CCN(C4COC4)CC3)cc2)n1]

To 4-(4-(oxetan-3-yl)piperazin-1-yl)aniline (2.00 g, 8.57 mmoles), hunig's base (3.29 mL) and 6,8-dibromoimidazo[1,2-a]pyrazine (2.37 g, 8.57 mmoles) was added in dimethylformamide (43 mL). The reaction was stirred at 85° C. in a pressure tube for overnight. The material was quenched with saturated sodium bicarbonate, extracted with methylene chloride (120 mL×3), the organic layers were combined and washed with water (120 mL×3), dried over anhydrous sodium carbonate and concentrated. The crude material was purified using a 120 g Isco column and eluted off using a stepwise gradient of 0-60% (10% methanol/methylene chloride). The desired fractions were combined and concentrated to provide the title compound as a light yellow solid.

### Example 31

### Preparation of 6-(2,3-dihydro-1H-pyrido[2,3-b][1,4]oxazin-7-yl)-N-(4-(4-(oxetan-3-yl)piperazin-1-yl)phenyl)imidazo[1,2-a]pyrazin-8-amine

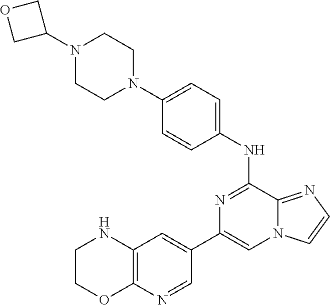 [SMILES: c1cn2cc(-c3cnc4c(c3)NCCO4)nc(Nc3ccc(N4CCN(C5COC5)CC4)cc3)c2n1]

This compound was synthesized according to procedure F in Example 5b above.

¹H NMR (300 MHz, DMSO) (δ): 9.45 (s, 1H), 8.41 (s, 1H), 7.96 (dt, 4H), 7.61 (d, 1H), 7.45 (d, 1H), 6.98 (d, 2H), 6.25 (s, 1H), 4.52 (t, 2H), 4.46 (t, 2H), 3.45 (q, 1H), 3.31 (t, 2H), 3.14 (t, 4H), 2.18 (t, 4H). MS (M+H) for C₂₆H₂₈N₈O₂: 485.52 (MH⁺),

### Example 32

### Preparation of 6-(3,4-dihydro-2H-benzo[b][1,4]oxazin-6-yl)-N-(4-(4-(oxetan-3-yl)piperazin-1-yl)phenyl)imidazo[1,2-a]pyrazin-8-amine

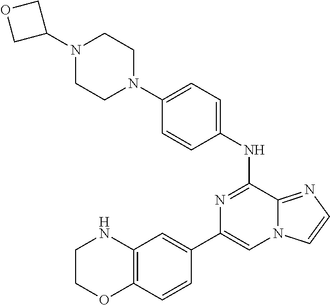 [SMILES: c1cn2cc(-c3ccc4c(c3)NCCO4)nc(Nc3ccc(N4CCN(C5COC5)CC4)cc3)c2n1]

This compound was synthesized according to procedure F in Example 5b above.

¹H NMR (300 MHz, DMSO) (δ): 9.35 (s, 1H), 8.31 (s, 1H), 8.01 (d, 2H), 7.94 (d, 1H), 7.81 (d, 1H), 7.23 (d, 2H), 7.07 (dd, 1H), 6.98 (d, 2H), 5.92 (s, 1H), 4.57 (t, 2H), 4.48 (t, 3H), 4.16 (t, 2H), 3.45 (q, 1H), 3.31 (t, 2H), 3.14 (t, 4H), 2.42 (t, 4H). MS (M+H) for C₂₇H₂₉N₇O₂: 484.45 (MH⁺).

### Example 33

### Preparation of 7-(8-((4-(4-(oxetan-3-yl)piperazin-1-yl)phenyl)amino)imidazo[1,2-a]pyrazin-6-yl)-3,4-dihydrobenzo[f][1,4]oxazepin-5(2H)-one

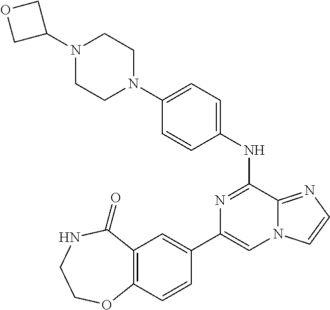 [SMILES: O=C1NCCOc2ccc(-c3cn4ccnc4c(Nc4ccc(N5CCN(C6COC6)CC5)cc4)n3)cc21]

This compound was synthesized according to procedure F in Example 5b above.

¹H NMR (300 MHz, DMSO) (δ): 9.45 (s, 1H), 8.56 (s, 1H), 8.45 (d, 1H), 8.40 (t, 1H), 8.1 (dd, 1H), 7.96 (m, 3H), 7.6 (d, 1H), 7.21 (d, 1H), 6.94 (d, 1H), 4.51 (t, 2H), 4.49 (t, 2H), 4.32 (dd, 2H), 3.45 (p, 1H), 3.38 (dd, 1H), 3.15 (bt, 4H), 2.40 (bt, 4H). MS (M+H) for C₂₈H₂₉N₇O₃: 512.56 (MH⁺).

### Example 34

### Preparation of 3-(phenylsulfonylmethylene)oxetane

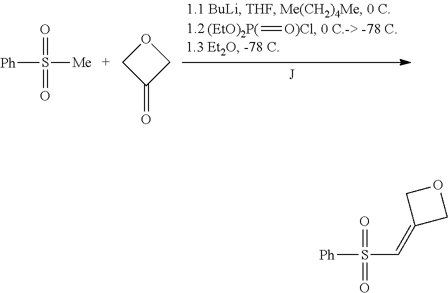

In a dry 100 mL flask, methylsulfonylbenzene (1.00 g, 6.41 mmoles) in a solution of dry tetrahydrofuran was 2.5 M n-buLi added at 0° C. over 10 mins then stirred for 30 mins. Chlorodiethylphosphonate (1.1 mL) was added dropwise and continued to stir for 30 mins before cooling to −78 C. Oxetan-3-one (0.65 g, 9.04 mmoles) in dry diethylether (1.0 mL) was added and stirred for 1.5 h. The reaction was filtered through a silica plug and to get pure product.

### Example 35

### Preparation of 1-benzyl-4-(3-methyloxetan-3-yl)piperazine

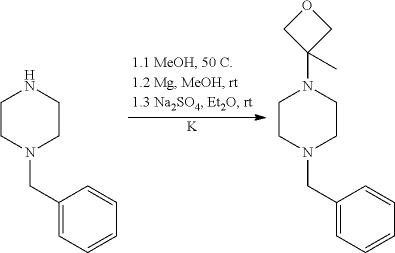

A solution of N-benzyl piperazine (0.19 g, 1.09 mmoles) and 3-(phenylsulfonylmethylene)oxetane (0.21 g, 1.00 mmoles) in methanol (5.0 mL) was stirred for 20 h at 50 C. Mg turnings (0.13 g, 4.99 mmoles) were added and the mixture stirred in ultrasound bath to start reaction (slight bubbling) and stirred ovn. Additional Mg turnings (0.13 g, 4.99 mmoles) was added and stirred for an additional 16 hours. diethylether (15 mL) was added, followed by sodium sulfate hydrate (Na₂SO₄*10H₂O) and stirred for 20 minutes, filtered, dried over sodium sulfate, filtered and concentrated. The crude material was purified using a 12 g silica column and eluted with a gradient from 0-40% ethyl acetate-hexane. The desired fractions were combined and concentrated to dryness under reduced pressure to provide the title compound.

### Example 36

### Preparation of 1-(3-methyloxetan-3-yl)piperazine

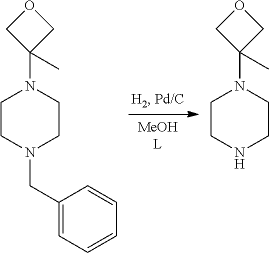 [SMILES: CC1(N2CCN(Cc3ccccc3)CC2)COC1 ; CC1(N2CCNCC2)COC1]

In a hydrogenation vessel, 1-(benzyl)-4-(3-methyloxetan-3-yl)piperazine (0.18 g, 0.72 mmoles) was dissolved in ethanol (2.4 mL), Pd/C (10%) (0.11 g, 0.12 mmoles) was added and the reaction was stored under nitrogen. The reaction was shaken on the Parr hydrogenator at 45 PSI for 1 h. The reaction was filtered over celite, washed with 25% methanol/methylene chloride and concentrated to dryness under reduced pressure to provide the title compound.

¹H NMR (300 MHz, DMSO) (δ): 9.42 (s, 1H), 8.39 (s, 1H), 7.96 (dd, 4H), 7.98 (d, 1H), 7.43 (d, 1H), 6.96 (d, 2H), 6.24 (s, 1H), 4.44 (d, 2H), 4.28 (t, 1H), 4.25 (d, 2H), 3.31 (d, 2H), 3.25 (t, 4H), 2.47 (t, 4H), 1.29 (s, 3H). MS (M+H) for C₂₇H₃₀N₈O₂: 499.59 (MH⁺).

### Example 37

### Preparation of 6-(2,3-dihydro-1H-pyrido[2,3-b][1,4]oxazin-7-yl)-N-(3-methoxy-4-(4-(3-methyloxetan-3-yl)piperazin-1-yl)phenyl)imidazo[1,2-a]pyrazin-8-amine

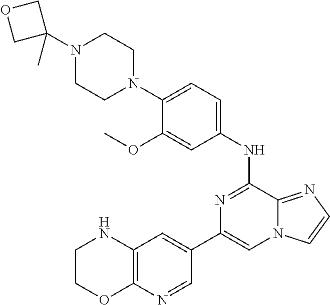 [SMILES: COc1cc(Nc2nc(-c3cnc4c(c3)NCCO4)cn3ccnc23)ccc1N1CCN(C2(C)COC2)CC1]

This compound was synthesized according to procedure F in Example 5b above.

¹H NMR (300 MHz, DMSO) (δ): 9.47 (s, 1H), 8.41 (s, 1H), 7.95 (t, 3H), 7.65 (d, 3H), 7.40 (s, 1H), 6.95 (d, 1H), 6.20 (s, 1H), 4.29 (t, 6H), 3.79 (t, 3H), 3.31 (t, 2H), 1.31 (s, 3H). MS (M+H) for C₂₈H₃₂N₈O₃: 529.42 (MH⁺).

### Example 38

### Preparation of 6-(2,3-dihydro-1H-pyrido[2,3-b][1,4]oxazin-7-yl)-N-(4-(4-(3-methyloxetan-3-yl)piperazin-1-yl)phenyl)imidazo[1,2-a]pyrazin-8-amine

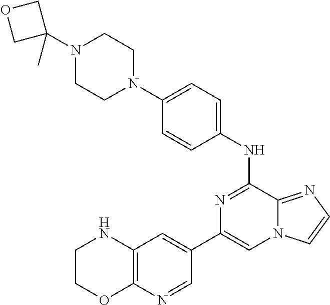 [SMILES: CC1(N2CCN(c3ccc(Nc4nc(-c5cnc6c(c5)NCCO6)cn5ccnc45)cc3)CC2)COC1]

This compound was synthesized according to procedure F in Example 5b above.

¹H NMR (300 MHz, DMSO) (δ): 9.42 (s, 1H), 8.39 (s, 1H), 7.98-8.92 (m, 4H), 7.59 (d, 1H), 7.43 (d, 1H), 4.44 (d, 2H), 4.29 (t, 2H), 4.14 (d, 2H), 3.12 (t, 4H), 2.44 (t, 4H), 1.29 (s, 3H). MS (M+H) for C₂₈H₃₂N₈O₃: 499.59 (MH⁺).

### Example 39

### Preparation of 3-methyloxetane-3-carbaldehyde

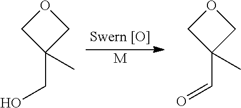

To oxalyl chloride (6.8 mL, 10.18 g, 80.21 mmoles) in methylene chloride (67.4 mL), was added DMSO (11.7 mL) at −78 C and stirred for 15 mins. (3-methyloxetan-3-yl)methanol (2.5 mL, 2.56 g, 80.21 mmoles) in methylene chloride (53.9 mL) was cannulated in and stirred at −78° C. for 1.5 h. N,N-Diisopropylethylamine (43.7 mL, 16.20 g, 125.33 mmoles) was added and stirred for 30 mins, warmed to 0° C. for 10 mins and then rt. The reaction was diluted with methylene chloride (100 mL) and extracted with ammonium chloride (3×200 mL), dried over anhydrous MgSO4, filtered and concentrate to dryness under pressure. The crude material was purified on a 40 g silica column, eluted off with 4:1 (hexanes:ethyl acetate), the desired fractions were concentrated to dryness under reduced pressure to provide the title compound.

### Example 40

### Preparation of 1-(2-methoxy-4-nitrophenyl)-4-(3-methyloxetan-3-yl)methyl)piperazine

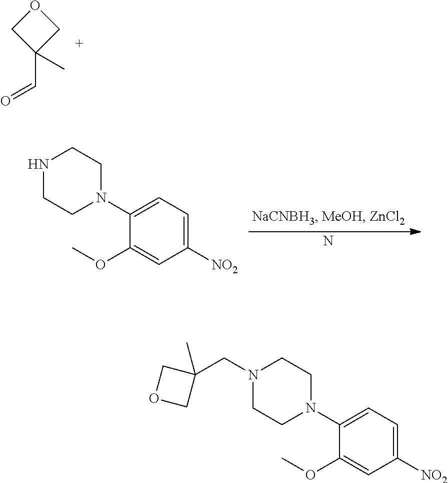

In a flask was placed 1-(2-methoxy-4-nitrophenyl)piperazine (100 mg, 0.42 mmol) and dissolved in methanol (3 mL) followed by addition of sodium cyanoborohydride (175 mg, 1.26 mmol), 3-methyloxetane-3-carbaldehyde (127 mg, 1.26 mmol), then zinc chloride (86 mg, 0.63 mmol) and heated to 48° C. for 1 hr. Let cool, diluted with methylene chloride (12 mL) and water, separated, dried over anh. sodium sulfate, filtered and concentrated. Adsorbed onto silica gel and purified via column chromatography: ISCO 12 g column, silica, 0% to 20% to 30% stepwise solvent B (9:1 premixed methylene chloride:methanol) solvent A methylene chloride., combined fractions to give the named product.

### Example 41

### Preparation of 6-(2,3-dihydro-1H-pyrido[2,3-b][1,4]oxazin-7-yl)-N-(3-methoxy-4-(4-((3-methyloxetan-3-yl)methyl)piperazin-1-yl)phenyl)imidazo[1,2-a]pyrazin-8-amine

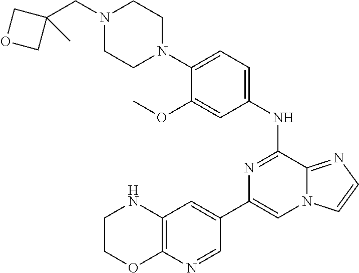 [SMILES: COc1cc(Nc2nc(-c3cnc4c(c3)NCCO4)cn3ccnc23)ccc1N1CCN(CC2(C)COC2)CC1]

This compound was synthesized according to procedure F in Example 5b above.

¹H NMR (300 MHz, DMSO): (δ): 9.43 (s, 1H), 8.40 (s, 1H), 7.99 (d, 1H), 7.95 (s, 1H), 7.90 (d, 1H), 7.65 (dd, 1H), 7.60 (s, 1H), 7.45 (d, 1H), 6.87 (d, 1H), 6.2 (s, 1H), 4.40 (d, 2H), 4.25 (t, 2H), 4.2 (d, 2H), 3.8 (3, 1H), 3.3 (t, 2H), 2.9 (s, 4H), 2.46 (2, 4H), 2.40 (q, 4H). MS (M+H) for C₂₉H₃₄N₈O₃: 543.54 (MH⁺).

### Example 42

### Preparation of 1-cyclobutoxy-2-methoxy-4-nitrobenzene

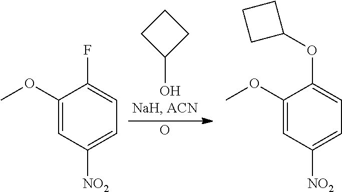

In a 50 mL round bottom flask, cyclobutanol (0.42 g, 5.84 mmoles), sodium hydride

(60%) (0.47 g, 11.69 mmoles), 1-fluoro-2-methoxy-4-nitrobenzene (1.00 g, 5.84 mmoles) was combined in acetonitrile (9 mL) and stirred under nitrogen overnight at 35° C. The mixture was quenched with water (30 mL) and extracted with ethyl acetate (30 mL×3), dried over anhydrous sodium carbonate, filtered and the filtrate was concentrated. The residue was purified on a 40 g silica column and eluted off using a gradient of 0-40% ethyl acetate/hexane. The desired fractions were concentrated to dryness under reduced pressure to provide title compound.

### Example 43

### Preparation of 4-cyclobutoxy-3-methoxyaniline

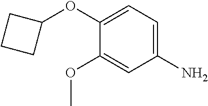

In a hydrogenation vessel, 1-cyclobutoxy-2-methoxy-4-nitrobenzene (0.52 g, 2.33 mmoles) was dissolved in methanol (8 mL), Pd/C (10%) (0.37 g, 0.35 mmoles) was added and the reaction was stored under nitrogen. The reaction was shaken on the Parr hydrogenator at 45 PSI for 1 hour. The material was filtered over celite, washed with 25% methanol/methylene chloride and concentrated to provide the title compound.

### Example 44

### Preparation of 6-bromo-N-(4-cyclobutoxy-3-methoxyphenyl)imidazo[1,2-a]pyrazin-8-amine

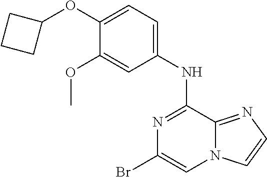 [SMILES: COc1cc(Nc2nc(Br)cn3ccnc23)ccc1OC1CCC1]

To 4-cyclobutoxy-3-methoxyaniline (0.43 g, 2.23 mmoles), hunig's base (0.85 mL) and 6,8-dibromoimidazo[1,2-a]pyrazine (0.28 g, 1.01 mmoles) was added in isopropanol (11 mL). The reaction was stirred at 85° C. in a pressure tube for overnight. The material was quenched with saturated sodium bicarbonate, extracted with methylene chloride (30 mL×3), the organic layers were combined and washed with water (30 mL×3), dried over anhydrous sodium carbonate and concentrated. The crude material was purified using a 24 g Isco column and eluted with a gradient from 10-80% of methylene chloride and a premixed solution of solvent B (90% methylene chloride and 10% methanol). The desired fractions were combined and concentrated to provide the title compound.

### Example 45

### Preparation of N-(4-cyclobutoxy-3-methoxyphenyl)-6-(2,3-dihydro-1H-pyrido[2,3-b][1,4]oxazin-7-yl)imidazo[1,2-a]pyrazin-8-amine

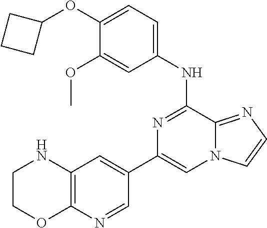 [SMILES: COc1cc(Nc2nc(-c3cnc4c(c3)NCCO4)cn3ccnc23)ccc1OC1CCC1]

This compound was synthesized according to procedure F in Example 5b above.

¹H NMR (300 MHz, DMSO): (δ): 9.46 (s, 1H), 8.40 (s, 1H), 7.98 (d, 1H), 7.93 (dd, 2H), 7.60 (dt, 2H), 7.43 (d, 1H), 6.79 (d, 2H), 6.19 (s, 1H), 4.60 (p, 1H), 4.28 (t, 2H), 3.77 (s, 3H), 3.31 (t, 2H), 2.40 (p, 2H), 2.25 (p, 2H), 1.65 (dq, 2H). MS (M+H) for C₂₄H₂₄N₆O₃: 445.45 (MH⁺).

### Example 46

### Preparation of 6-(2,3-dihydro-1H-pyrido[2,3-b][1,4]oxazin-7-yl)-N-(3-methoxy-4-(oxetan-3-yloxy)phenyl)imidazo[1,2-a]pyrazin-8-amine

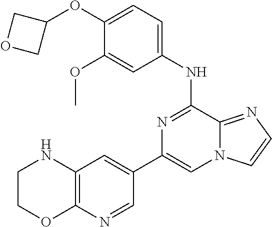 [SMILES: COc1cc(Nc2nc(-c3cnc4c(c3)NCCO4)cn3ccnc23)ccc1OC1COC1]

This compound was synthesized according to procedure F in Example 5b above.

¹H NMR (300 MHz, DMSO): (δ): 9.53 (s, 1H), 8.41 (s, 1H), 7.99 (dd, 2H), 7.95 (d, 1H), 7.60 (dd, 2H), 7.43 (d, 1H), 6.65 (d, 2H), 6.18 (s, 1H), 5.16 (s, 1H), 4.8 (t, 2H), 4.70 (q, 2H), 4.28 (t, 2H), 3.80 (s, 3H), 3.31 (t, 2H). MS (M+H) for C₂₃H₂₂N₆O₄: 447.35 (MH⁺).

### Example 47

### Preparation of 6-(2,3-dihydro-1H-pyrido[2,3-b][1,4]oxazin-7-yl)-N-(4-(oxetan-3-yloxy)phenyl)imidazo[1,2-a]pyrazin-8-amine

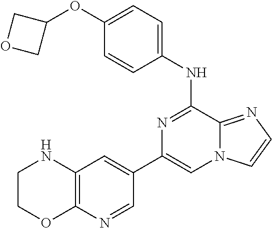 [SMILES: c1cn2cc(-c3cnc4c(c3)NCCO4)nc(Nc3ccc(OC4COC4)cc3)c2n1]

This compound was synthesized according to procedure F in Example 5b above.

¹H NMR (300 MHz, DMSO): (δ): 9.58 (s, 1H), 8.42 (s, 1H), 8.05 (dd, 2H), 7.95 (dd, 2H), 7.12 (d, 1H), 7.42 (d, 1H), 6.80 (d, 2H), 5.25 (p, 1H), 4.92 (t, 2H), 4.53 (t, 2H), 4.30 (t, 2H), 3.31 (d, 2H). MS (M+H) for C₂₂H₂₀N₆O₃: 417.59 (MH⁺).

### Example 48

### Preparation of 6-(2,3-dihydro-1H-pyrido[2,3-b][1,4]oxazin-7-yl)-N-(4-((3-methyloxetan-3-yl)oxy)phenyl)imidazo[1,2-a]pyrazin-8-amine

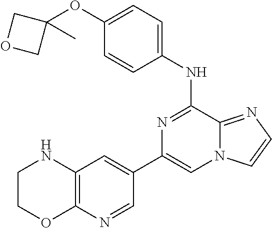 [SMILES: CC1(Oc2ccc(Nc3nc(-c4cnc5c(c4)NCCO5)cn4ccnc34)cc2)COC1]

This compound was synthesized according to procedure F in Example 5b above.

¹H NMR (300 MHz, DMSO): (δ): 9.65 (s, 1H), 8.50 (s, 1H), 8.20 (dt, 2H), 8.03 (t, 2H), 7.69 (d, 1H), 7.50 (d, 1H), 7.6 (d, 1H), 6.81 (dd, 2H), 6.31 (s, 1H), 4.83 (d, 2H), 4.65 (d, 2H), 4.37 (t, 2H), 1.75 (s, 3H). MS (M+H) for C₂₃H₂₂N₆O₃: 431.49 (MH⁺).

### Example 49

### Preparation of 6-(2,3-dihydro-1H-pyrido[2,3-b][1,4]oxazin-7-yl)-N-(3-fluoro-4-(oxetan-3-yloxy)phenyl)imidazo[1,2-a]pyrazin-8-amine

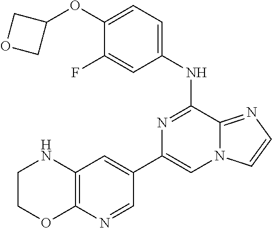 [SMILES: Fc1cc(Nc2nc(-c3cnc4c(c3)NCCO4)cn3ccnc23)ccc1OC1COC1]

This compound was synthesized according to procedure F in Example 5b above. Starting material 6-(2,3-dihydro-1H-pyrido[2,3-b][1,4]oxazin-7-yl)-N-(3-fluoro-4-(oxetan-3-yloxy)phenyl)imidazo[1,2-a]pyrazin-8-amine was synthesized using procedure 0 utilizing 3,4-difluoronitrobenzene.

¹H NMR (300 MHz, DMSO): (δ): 9.79 (s, 1H), 8.43 (s, 1H), 8.2 (dd, 1H), 7.97 (dd, 2H), 7.90 (dt, 1H), 7.62 (s, 1H), 7.38 (d, 1H), 6.87 (t, 1H), 6.22 (s, 1H), 5.28 (p, 1H), 4.92 (t, 2H), 4.48 (t, 2H), 4.29 (t, 2H), 3.31 (d, 2H). MS (M+H) for C₂₂H₁₉N₆O₃: 435.17 (MH⁺).

### Example 50

### Preparation of 1-(4-(4-((6-(2,3-dihydro-1H-pyrido[2,3-b][1,4]oxazin-7-yl)imidazo[1,2-a]pyrazin-8-yl)amino)phenyl)piperazin-1-yl)ethanone

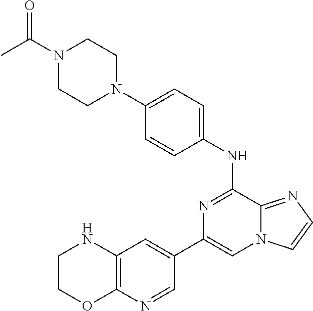 [SMILES: CC(=O)N1CCN(c2ccc(Nc3nc(-c4cnc5c(c4)NCCO5)cn4ccnc34)cc2)CC1]

This compound was synthesized according to procedure F in Example 5b above.

¹H NMR (300 MHz, DMSO): (δ): 9.46 (s, 1H), 8.40 (s, 1H), 7.94 (t, 4H), 7.58 (s, 1H), 7.43 (s, 1H), 7.00 (d, 2H), 6.23 (s, 1H), 4.38 (t, 2H), 3.57 (d, 4H), 3.10 (dt, 4H), 2.03 (s, 3H). MS (M+H) for C₂₅H₂₆N₈O₂: 471.54 (MH⁺).

### Example 51

### Preparation of N4-(6-(2,3-dihydro-1H-pyrido[2,3-b][1,4]oxazin-7-yl)imidazo[1,2-a]pyrazin-8-yl)-2-methoxy-N1,N1-dimethylbenzene-1,4-diamine

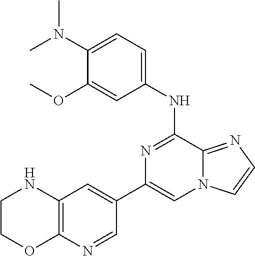 [SMILES: COc1cc(Nc2nc(-c3cnc4c(c3)NCCO4)cn3ccnc23)ccc1N(C)C]

This compound was synthesized according to procedure F in Example 5b above.

¹H NMR (300 MHz, DMSO): (δ): 9.43 (s, 1H), 8.40 (s, 1H), 7.96 (dd, 2H), 7.86 (d, 1H), 7.70 (dd, 1H), 7.52 (s, 1H), 7.45 (d, 1H), 6.87 (d, 1H), 6.20 (s, 1H), 4.23 (t, 2H), 3.80 (s, 3H), 3.31 (t, 2H), 2.66 (s, 1H). MS (M+H) for C₂₂H₂₃N₇O₂: 418.38 (MH⁺).

### Example 52

### Preparation of 6-bromo-N-(1-isopropyl-1H-pyrazol-4-yl)imidazo[1,2-a]pyrazin-8-amine

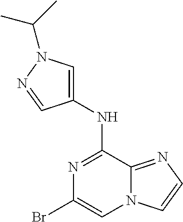 [SMILES: CC(C)n1cc(Nc2nc(Br)cn3ccnc23)cn1]

This compound was prepared by direct analogy to procedure C in Example 3 above, using instead 1-isopropyl-1H-pyrazol-4-amine as a starting material to afford 6-bromo-N-(1-isopropyl-1H-pyrazol-4-yl)imidazo[1,2-a]pyrazin-8-amine as a red solid.

### Example 53

### Preparation of 6-(2,3-dihydro-1H-pyrido[2,3-b][1,4]oxazin-7-yl)-N-(1-isopropyl-1H-pyrazol-4-yl)imidazo[1,2-a]pyrazin-8-amine

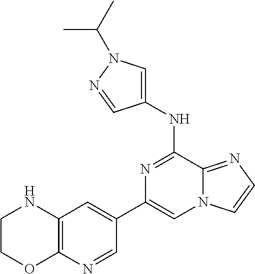 [SMILES: CC(C)n1cc(Nc2nc(-c3cnc4c(c3)NCCO4)cn3ccnc23)cn1]

This compound was synthesized according to procedure F in Example 5b above.

¹H NMR (300 MHz, d₆-DMSO): δ 9.914 (s, 1H), 8.33 (s, 1H), 8.19 (s, 1H), 7.99 (d, 1H), 7.91 (m, 2H), 7.57 (s, 1H), 7.47 (d, 1H), 4.34-4.54 (m, 1H), 4.27-4.32 (m 2H), 3.28-3.4 (m, 6 h), 1.42 (d, 6H); MS (ESI+) m/z 377.51 (M+H).

### Example 54

### Preparation of tert 4-(3,6-dihydro-2H-pyran-4-yl)-3-methoxyaniline

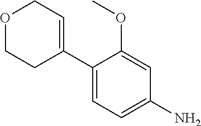

A 50 mL sealed tube with a magnetic stirrer was charged with 4-bromo-3-methoxyaniline (1.2 g, 5.9 mmol), 2-(3,6-dihydro-2H-pyran-4-yl)-4,4,5,5-tetramethyl-1,3,2-dioxaborolane (1.92 g, 6.2 mmol), 20 mL 1N sodium bicarbonate, and 50 mL dioxane 50 mL, and tetrakis (triphenylphosphine) palladium(0) (0.29 g, 0.25 mmol). The reaction mixture was heated at 100° C. for 5 hours. After this time, the mixture was cooled to room temperature, partitioned between ethyl acetate (50 mL) and water (30 mL). The organic phase was separated, and the aqueous layer was extracted with ethyl acetate (30 mL×3). The combined organic phases were washed with brine, dried over sodium sulfate and concentrated under reduced pressure. The residue was purified by flash column chromatography (silica, 0-100% Ethyl Acetate) to give 4-(3,6-dihydro-2H-pyran-4-yl)-3-methoxyaniline as a white solid.

### Example 55

### Preparation of 6-bromo-N-(4-(3,6-dihydro-2H-pyran-4-yl)-3-methoxyphenyl)imidazo[1,2-a]pyrazin-8-amine

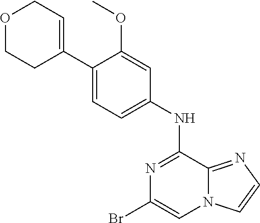 [SMILES: COc1cc(Nc2nc(Br)cn3ccnc23)ccc1C1=CCOCC1]

This compound was prepared by direct analogy to procedure C in Example 3 above, using instead 4-(3,6-dihydro-2H-pyran-4-yl)-3-methoxyaniline as a starting material to afford 6-bromo-N-(1-isopropyl-1H-pyrazol-4-yl)imidazo[1,2-a]pyrazin-8-amine as a white solid.

### Example 56

### Preparation of 6-(2,3-dihydro-1H-pyrido[2,3-b][1,4]oxazin-7-yl)-N-(4-(3,6-dihydro-2H-pyran-4-yl)-3-methoxyphenyl)imidazo[1,2-a]pyrazin-8-amine

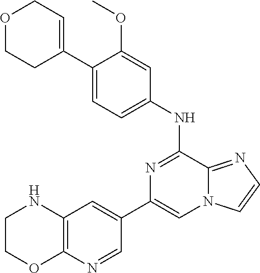 [SMILES: COc1cc(Nc2nc(-c3cnc4c(c3)NCCO4)cn3ccnc23)ccc1C1=CCOCC1]

This compound was synthesized according to procedure F in Example 5b above. MS (ESI+) m/z 457.53 (M+H).

### Example 57

### Preparation of 6-(2,3-dihydro-1H-pyrido[2,3-b][1,4]oxazin-7-yl)-N-(3-methoxy-4-(tetrahydro-2H-pyran-4-yl)phenyl)imidazo[1,2-a]pyrazin-8-amine

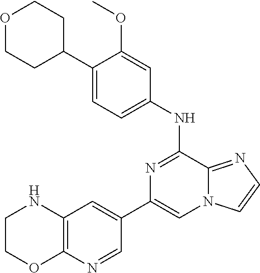 [SMILES: COc1cc(Nc2nc(-c3cnc4c(c3)NCCO4)cn3ccnc23)ccc1C1CCOCC1]

A Parr reactor bottle was charged with 10% palladium on carbon (300 mg, 20% weight), 6-(2,3-dihydro-1H-pyrido[2,3-b][1,4]oxazin-7-yl)-N-(4-(3,6-dihydro-2H-pyran-4-yl)-3-methoxyphenyl)imidazo[1,2-a]pyrazin-8-amine (300 mg, 0.66 mmol), and ethyl acetate (20 mL). The bottle was attached to a Parr hydrogenator, evacuated, charged with hydrogen gas to a pressure of 50 psi and shaken for 3 hours. The reaction mixture was then filtered through a pad of Celite 521 and the solids were washed with ethanol (2×25 mL), and the combined filtrate was concentrated on a rotary evaporator to afford 6-(2,3-dihydro-1H-pyrido[2,3-b][1,4]oxazin-7-yl)-N-(3-methoxy-4-(tetrahydro-2H-pyran-4-yl)phenyl)imidazo[1,2-a]pyrazin-8-amine as a white solid.

¹H NMR (300 MHz, d₆-DMSO): δ 9.55 (s, 1H), 8.43 (s, 1H), 7.95-8.11 (m, 3H), 7.60-7.69 (m, 3H), 7.46 (d, 1H), 7.14 (d, 2H), 6.20 (s, 1H), 4.27-4.31 (m, 2H), 3.80-3.96 (m, 5H), 3.20-3.44 (m, 8H), 2.99-3.18 (m, 1H), 2.48 (d, 1H) 1.60-1.65 (m, 1H); MS (ESI+) m/z 459.47 (M+H).

### Example 58

### Preparation of 6-bromo-N-(4-(tetrahydro-2H-pyran-4-yl)phenyl)imidazo[1,2-a]pyrazin-8-amine

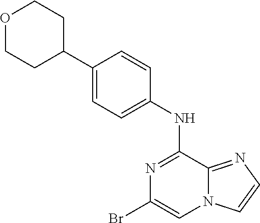 [SMILES: Brc1cn2ccnc2c(Nc2ccc(C3CCOCC3)cc2)n1]

This compound was prepared by direct analogy to procedure C in Example 3 above, using instead 4-(tetrahydro-2H-pyran-4-yl)aniline as a starting material to afford 6-bromo-N-(1-isopropyl-1H-pyrazol-4-yl)imidazo[1,2-a]pyrazin-8-amine as a white solid.

### Example 59

### Preparation of 6-(2,3-dihydro-1H-pyrido[2,3-b][1,4]oxazin-7-yl)-N-(4-(tetrahydro-2H-pyran-4-yl)phenyl)imidazo[1,2-a]pyrazin-8-amine

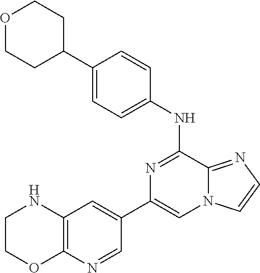 [SMILES: c1cn2cc(-c3cnc4c(c3)NCCO4)nc(Nc3ccc(C4CCOCC4)cc3)c2n1]

This compound was synthesized according to procedure F in Example 5b above.

¹H NMR (300 MHz, d₆-DMSO): δ 9.57 (s, 1H), 8.41 (s, 1H), 7.95-8.11 (m, 4H), 7.61 (d, 1H), 7.44 (d, 1H), 7.22 (d, 2H), 6.25 (s, 1H), 4.27-4.31 (m, 3H), 3.90-3.96 (m, 6H), 2.48-2.775 (m, 1H), 1.62-1.71 (m, 4H) 1.052 (s, 1H); MS (ESI+) m/z 429.47 (M+H).

### Example 60

### Preparation of 1-(2-methoxy-4-nitrophenyl)-1H-imidazole

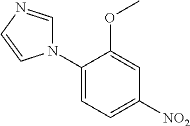 [SMILES: COc1cc([N+](=O)[O-])ccc1-n1ccnc1]

This compound was prepared by direct analogy to procedure A in Example 1 above, using instead 1H-imidazole to afford 1-(2-methoxy-4-nitrophenyl)-1H-imidazole.

### Example 61

### Preparation of 4-(1H-imidazol-1-yl)-3-methoxyaniline

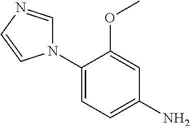

This compound was prepared by direct analogy to procedure B in Example 2 above, using instead 1-(2-methoxy-4-nitrophenyl)-1H-imidazole to afford 4-(1H-imidazol-1-yl)-3-methoxyaniline.

### Example 62

### Preparation of N-(4-(1H-imidazol-1-yl)-3-methoxyphenyl)-6-bromoimidazo[1,2-a]pyrazin-8-amine

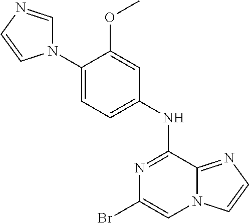 [SMILES: COc1cc(Nc2nc(Br)cn3ccnc23)ccc1-n1ccnc1]

This compound was prepared by direct analogy to procedure C in Example 3 above, using instead 4-(1H-imidazol-1-yl)-3-methoxyaniline to afford N-(4-(1H-imidazol-1-yl)-3-methoxyphenyl)-6-bromoimidazo[1,2-a]pyrazin-8-amine.

### Example 63

### Preparation of N-(4-(1H-imidazol-1-yl)-3-methoxyphenyl)-6-(2,3-dihydro-1H-pyrido[2,3-b][1,4]oxazin-7-yl)imidazo[1,2-a]pyrazin-8-amine

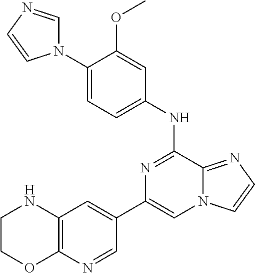 [SMILES: COc1cc(Nc2nc(-c3cnc4c(c3)NCCO4)cn3ccnc23)ccc1-n1ccnc1]

This compound was synthesized according to procedure F in Example 5b above.

¹H NMR (300 MHz, d₆-DMSO): δ 9.91 (s, 1H), 8.51 (s, 1H), 8.27 (d, 1H), 8.00 (s, 2H) 7.81-7.85 (m, 2H), 7.66 (s, 1H), 7.46 (d, 1H), 7.33-7.40 (m, 2H), 6.205 (s, 1H) 4.25-4.32 (m, 2H), 3.84 (s, 1H), 2.4-2.49 (m, 2H); MS (ESI+) m/z 441.43 (M+H).

### Example 64

### Preparation of N-(6-(2,3-dihydro-1H-pyrido[2,3-b][1,4]oxazin-7-yl)imidazo[1,2-a]pyrazin-8-yl)-2,4,4a,5-tetrahydro-1H-benzo[b][1,4]oxazino[4,3-d][1,4]oxazin-8-amine

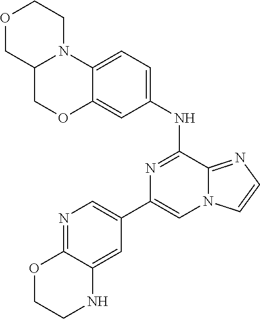 [SMILES: c1cn2cc(-c3cnc4c(c3)NCCO4)nc(Nc3ccc4c(c3)OCC3COCCN43)c2n1]

This compound was synthesized according to procedure F in Example 5b above. MS (ESI+) m/z 458.42 (M+H).

### Example 65

### Preparation of 6-(2,3-dihydro-1H-pyrido[2,3-b][1,4]oxazin-7-yl)-N-(1-(tetrahydro-2H-pyran-4-yl)-1H-pyrazol-4-yl)imidazo[1,2-a]pyrazin-8-amine

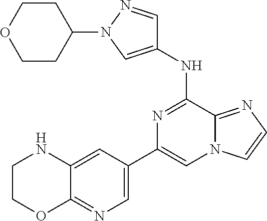 [SMILES: c1cn2cc(-c3cnc4c(c3)NCCO4)nc(Nc3cnn(C4CCOCC4)c3)c2n1]

This compound was synthesized according to procedure F in Example 5b above. MS (ESI+) m/z 419.29 (M+H).

### Example 66

### Preparation of 6-(3,4-dihydro-2H-benzo[b][1,4]oxazin-6-yl)-N-(4-(4-ethylpiperazin-1-yl)phenyl)imidazo[1,2-a]pyrazin-8-amine

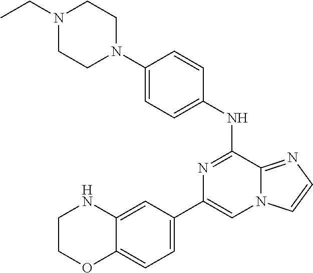 [SMILES: CCN1CCN(c2ccc(Nc3nc(-c4ccc5c(c4)NCCO5)cn4ccnc34)cc2)CC1]

This compound was synthesized according to procedure F in Example 5b above. MS (ESI+) m/z 456.52 (M+H).

### Example 67

### Preparation of 6-(2,3-dihydro-1H-pyrido[2,3-b][1,4]oxazin-7-yl)-N-(3-methoxy-4-(4-(2-methoxyethyl)piperazin-1-yl)phenyl)imidazo[1,2-a]pyrazin-8-amine

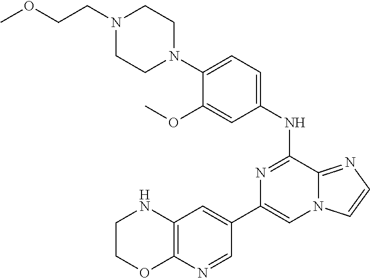 [SMILES: COCCN1CCN(c2ccc(Nc3nc(-c4cnc5c(c4)NCCO5)cn4ccnc34)cc2OC)CC1]

This compound was synthesized according to procedure F in Example 5b above.

¹H NMR (300 MHz, DMSO-d6): δ 9.46 (s, 1H), 8.41 (s, 1H), 7.99 (d, 1H), 7.95 (s, 1H), 7.89 (d, 1H), 7.65 (d, 1H), 7.60 (s, 1H), 7.45 (s, 1H), 6.87 (d, 1H), 6.20 (bs, 1H), 4.29 (t, 2 h), 3.79 (s, 1H), 3.45 (t, 2H), 3.30 (m, 2H), 3.23 (s, 3H), 2.93 (m, 4H), 2.53 (m, 4H). LCMS [M+H]: 517.5.

### Example 68

### Preparation of 1-(4-(4-((6-(2,3-dihydro-1H-pyrido[2,3-b][1,4]oxazin-7-yl)imidazo[1,2-a]pyrazin-8-yl)amino)-2-methoxyphenyl)piperazin-1-yl)ethanone

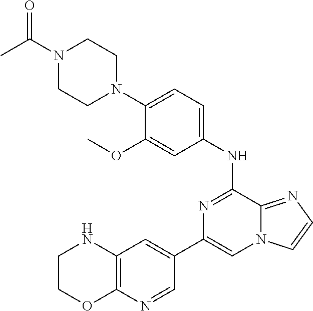 [SMILES: COc1cc(Nc2nc(-c3cnc4c(c3)NCCO4)cn3ccnc23)ccc1N1CCN(C(C)=O)CC1]

This compound was synthesized according to procedure F in Example 5b above.

¹H NMR (300 MHz, DMSO-d6): δ 9.50 (s, 1H), 8.41 (s, 1H) 7.99 (d, 1H), 7.95 (s, 1H), 7.93 (d, 1H), 7.67 (d, 1H), 7.61 (s, 1H) 7.44 (s, 1H), 6.89 (d, 1H), 6.20 (bs, 1H), 4.29 (t, 2H), 3.81 (s, 3H), 3.54 (m, 4H), 3.30 (m, 2H), 2.87-2.93 (m, 4H), 2.20 (s, 3H). LCMS [M+H]: 501.6.

### Example 69

### Preparation of 6-(2,3-dihydro-1H-pyrido[2,3-b][1,4]oxazin-7-yl)-N-(3-methoxy-4-(4-methoxypiperidin-1-yl)phenyl)imidazo[1,2-a]pyrazin-8-amine

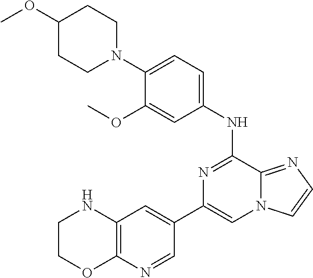 [SMILES: COc1cc(Nc2nc(-c3cnc4c(c3)NCCO4)cn3ccnc23)ccc1N1CCC(OC)CC1]

This compound was synthesized according to procedure F in Example 5b above.

¹H NMR (300 MHz, DMSO-d6): δ 9.45 (s, 1H), 8.41 (s, 1H), 7.98 (d, 1H), 7.94 (s, 1H), 7.88 (s, 1H), 7.65 (d, 1H), 7.60 (s, 1H), 7.45 (d, 1H), 6.89 (d, 1H), 6.20 (bs, 1H), 4.28 (t, 2H), 3.79 (s, 3H), 3.30 (m, 2H), 3.25 (s, 3H), 3.18 (m, 2H), 2.65 (t, 2H), 1.93 (m, 2H), 1.57 (m, 2H). LCMS [M+H]: 488.3.

### Example 70

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)-4-methylpiperidin-4-ol

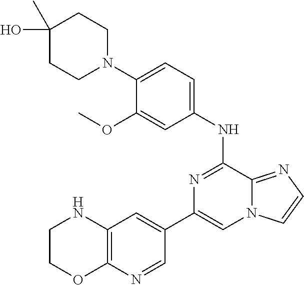 [SMILES: COc1cc(Nc2nc(-c3cnc4c(c3)NCCO4)cn3ccnc23)ccc1N1CCC(C)(O)CC1]

This compound was synthesized according to procedure F in Example 5b above.

¹H NMR (300 MHz, DMSO-d6): δ 9.43 (s, 1H), 8.41 (s, 1H), 7.99 (d, 1H), 7.94 (s, 1H), 7.86 (d, 1H), 7.64 (d, 1H), 7.60 (s, 1H), 7.45 (d, 1H), 6.91 (d, 1H), 6.20 (bs, 1H), 4.29 (t, 2H), 4.17 (s, 1H), 3.79 (s, 3H), 3.30 (m, 2H), 2.92 (t, 4H), 1.58 (m, 4H), 1.15 (s, 3H). LCMS [M+H]: 488.5.

### Example 71

### Preparation of 2-(4-((6-(2,3-dihydro-1H-pyrido[2,3-b][1,4]oxazin-7-yl)imidazo[1,2-a]pyrazin-8-yl)amino)phenyl)-2-methylpropan-1-ol

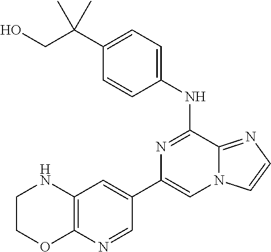 [SMILES: CC(C)(CO)c1ccc(Nc2nc(-c3cnc4c(c3)NCCO4)cn3ccnc23)cc1]

This compound was synthesized according to procedure F in Example 5b above.

¹H NMR (300 MHz, DMSO-d6): δ 9.53 (s, 1H), 8.44 (s, 1H), 8.02 (d, 2H), 7.96 (d, 1H), 7.95 (s, 1H), 7.61 (s, 1H), 7.45 (d, 1H), 7.34 (d, 2H), 6.27 (bs, 1H), 4.62 (t, 1H), 4.29 (t, 2H), 3.41 (d, 2H), 3.32 (m, 2H), 1.23 (s, 6H). LCMS [M+H]: 417.5.

### Example 72

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)-3-methylpiperidin-3-ol

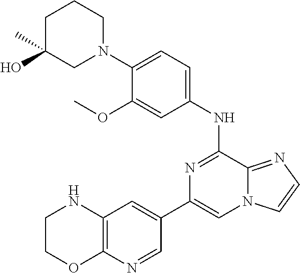 [SMILES: COc1cc(Nc2nc(-c3cnc4c(c3)NCCO4)cn3ccnc23)ccc1N1CCC[C@](C)(O)C1]

This compound was synthesized according to procedure F in Example 5b above.

¹H NMR (300 MHz, DMSO-d6): δ 9.45 (s, 1H), 8.41 (s, 1H), 7.98 (d, 1H), 7.95 (s, 1H), 7.87 (d, 1H), 7.65 (d, 1H), 7.60 (s, 1H), 7.45 (d, 1H), 6.87 (d, 1H), 6.21 (bs, 1H), 4.30 (t, 2H), 4.23 (s, 1H), 3.80 (s, 3H), 3.30 (m, 2H), 2.86 (m, 2H), 2.75 (m, 1H), 1.75 (m, 1H), 1.50 (m, 3H), 1.22 (s, 3H). LCMS [M+H]: 488.5.

### Example 73

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)-3-methylpiperidin-3-ol

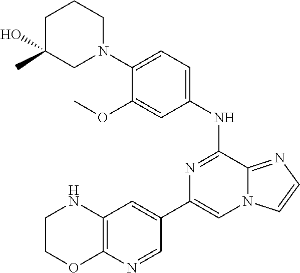 [SMILES: COc1cc(Nc2nc(-c3cnc4c(c3)NCCO4)cn3ccnc23)ccc1N1CCC[C@@](C)(O)C1]

This compound was synthesized according to procedure F in Example 5b above.

¹H NMR (300 MHz, DMSO-d6): δ 9.45 (s, 1H), 8.41 (s, 1H), 7.98 (d, 1H), 7.95 (s, 1H), 7.87 (d, 1H), 7.65 (d, 1H), 7.60 (s, 1H), 7.45 (d, 1H), 6.87 (d, 1H), 6.21 (bs, 1H), 4.30 (t, 2H), 4.23 (s, 1H), 3.80 (s, 3H), 3.30 (m, 2H), 2.86 (m, 2H), 2.75 (m, 1H), 1.75 (m, 1H), 1.50 (m, 3H), 1.22 (s, 3H). LCMS [M+H]: 488.5.

### Example 74

### Preparation of N-(6-(2,3-dihydro-1H-pyrido[2,3-b][1,4]oxazin-7-yl)imidazo[1,2-a]pyrazin-8-yl)-2-isopropylthiazol-5-amine

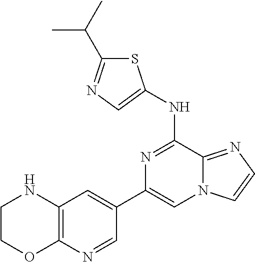 [SMILES: CC(C)c1ncc(Nc2nc(-c3cnc4c(c3)NCCO4)cn3ccnc23)s1]

This compound was synthesized according to procedure F in Example 5b above.

¹H NMR (300 MHz, DMSO-d6): δ 11.2 (s, 1H), 8.47 (s, 1H), 8.09 (d, 1H), 7.99 (d, 1H), 7.68 (s, 1H), 7.65 (d, 1H), 7.53 (d, 1H), 4.6 (t, 2H), 3.65 (m (2H), 3.45 (dddd, 1H), 1.34 (d, 6H). LCMS [M+H]: 394.4.

### Example 75

### Preparation of 7-(8-((3-methoxy-4-(4-(oxetan-3-yl)piperazin-1-yl)phenyl)amino)imidazo[1,2-a]pyrazin-6-yl)-3,4-dihydrobenzo[f][1,4]oxazepin-5(2H)-one

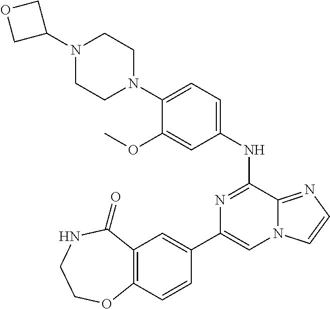 [SMILES: COc1cc(Nc2nc(-c3ccc4c(c3)C(=O)NCCO4)cn3ccnc23)ccc1N1CCN(C2COC2)CC1]

This compound was synthesized according to procedure F in Example 5b above.

¹H NMR (300 MHz, DMSO-d6): δ 9.50 (s, 1H), 8.58 (s, 1H), 8.45-8.38 (m, 2H), 8.06 (d, 1H), 8.25 (d, 1H), 7.97 (s, 1H), 7.62 (s, 1H), 7.54 (d, 1H), 7.11 (d, 1H), 7.86 (d, 1H), 4.55 (t, 2H), 4.45 (t, 2H), 4.33 (t, 2H), 3.80 (s, 3H), 3.45 (p, 1H), 3.35 (dd, 2H), 3.00-2.92 (m, 4H), 2.44-2.37 (m, 4H), LCMS [M+H]: 542.5.

### Example 76

### Preparation of (R)-7-(8-((4-(2-(hydroxymethyl)morpholinyl)-3-methoxyphenyl)amino)imidazo[1,2-a]pyrazin-6-yl)-3,4-dihydrobenzo[f][1,4]oxazepin-5(2H)-one

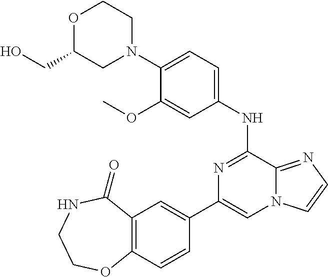 [SMILES: COc1cc(Nc2nc(-c3ccc4c(c3)C(=O)NCCO4)cn3ccnc23)ccc1N1CCO[C@@H](CO)C1]

This compound was synthesized according to procedure F in Example 5b above.

¹H NMR (300 MHz, DMSO-d6): δ 9.54 (s, 1H), 8.59 (s, 1H), 8.49-8.42 (m, 2H), 8.07 (d, 1H), 8.02 (d, 1H), 7.97 (s, 1H), 7.62 (s, 1H), 7.60 (d, 1H), 7.12 (d, 1H), 6.70 (d, 1H), 4.72 (t, 1H), 4.32 (t, 2H), 3.88-3.82 (m, 1H), 3.70-3.54 (m, 2H), 3.50-3.41 (m, 1H), 3.40-3.34 (m, 3H), 3.32-3.25 (m, 1H), 3.21-3.14 (m, 1H), 2.62 (t, 1H), 2.38 (t, 1H) LCMS [M+H]: 517.4.

### Example 77

### Preparation of 6-(2,3-dihydro-1H-pyrido[2,3-b][1,4]oxazin-7-yl)-N-(3-methoxy-4-octadeuteratedmorpholinophenyl)imidazo[1,2-a]pyrazin-8-amine

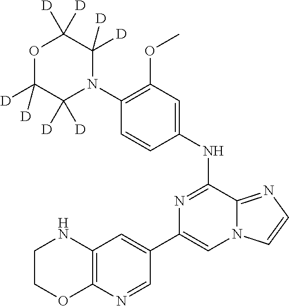 [SMILES: [2H]C1([2H])OC([2H])([2H])C([2H])([2H])N(c2ccc(Nc3nc(-c4cnc5c(c4)NCCO5)cn4ccnc34)cc2OC)C1([2H])[2H]]

This compound was synthesized according to procedure F in Example 5b above.

¹H NMR (300 MHz, DMSO-d6): δ 9.50 (s, 1H), 8.43 (s, 1H), 8.01 (d, 1H), 7.97 (d, 1H), 7.92 (d, 1H), 7.70 (dd, 1H), 7.62 (d, 1H), 7.47 (d, 1H), 6.90 (d, 1H), 6.22 (broad s, 1H), 4.30 (t, 2H), 3.82 (s, 3H), ppm; MS (ESI+) m/z 468.5 (M+H).

### Example 78

### Preparation of 7-(4,4,5,5-tetramethyl-1,3,2-dioxaborolan-2-yl)-2,3-dihydro-1H-pyrido[2,3-b][1,4]thiazine

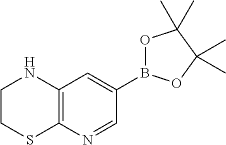 [SMILES: CC1(C)OB(c2cnc3c(c2)NCCS3)OC1(C)C]

This compound was synthesized according to procedure D in Example 4 above.

### Example 79

### Preparation of 6-(2,3-dihydro-1H-pyrido[2,3-b][1,4]thiazin-7-yl)-N-(3-methoxy-4-morpholinophenyl)imidazo[1,2-a]pyrazin-8-amine

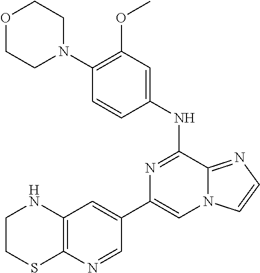 [SMILES: COc1cc(Nc2nc(-c3cnc4c(c3)NCCS4)cn3ccnc23)ccc1N1CCOCC1]

This compound was synthesized according to procedure F in Example 5b above.

¹H NMR (300 MHz, DMSO-d6): δ 9.53 (s, 1H), 8.48 (s, 1H), 8.25 (d, 1H), 7.98 (d, 1H), 7.90 (d, 1H), 7.74 (dd, J=2.1 Hz, 1H), 7.64 (d, 1H), 7.38 (d, 1H), 6.92 (d, 1H), 6.39 (broad s, 1H), 3.88 (s, 3H), 3.73 (broad m, 4H), 3.48-3.52 (m, 2H), 3.14-3.18 (m, 2H), 2.95 (broad m, 4H) ppm; m/z 476.40 (M+H).

### Example 80

### Preparation of 7-(8-((3-methoxy-4-morpholinophenyl)amino)imidazo[1,2-a]pyrazin-6-yl)-2,3-dihydro-1H-pyrido[2,3-b][1,4]thiazine 4-oxide

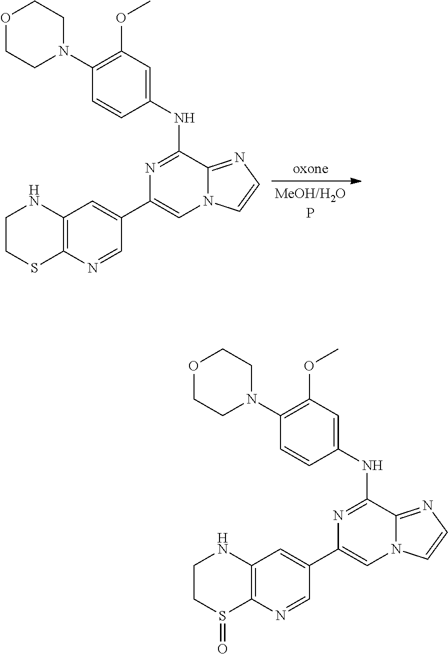 [SMILES: COc1cc(Nc2nc(-c3cnc4c(c3)NCCS4)cn3ccnc23)ccc1N1CCOCC1 ; COc1cc(Nc2nc(-c3cnc4c(c3)NCCS4=O)cn3ccnc23)ccc1N1CCOCC1]

To a round-bottomed flask equipped with a stirring bar, 6-(2,3-dihydro-1H-pyrido[2,3-b][1,4]thiazin-7-yl)-N-(3-methoxy-4-morpholinophenyl)imidazo[1,2-a]pyrazin-8-amine (300 mg, 0.63 mmol), oxone (465.5 mg, 0.76 mmol), methanol (10 mL), water (10 mL), were added. The resulting mixture was stirred at room temperature for 2 hrs. Removed all the solvent in vacuo and the resulting residue was added to methylene chloride (200 mL), the solution was washed with water (30 mL×3), brine (30 mL×1), dried over magnesium sulfate, filtered, and removed solvent in vacuo. Silica gel column (methanol:methylene chloride=10:90) gave the desired product as off-white solids.

¹H NMR (300 MHz, DMSO-d₆): δ 9.62 (s, 1H), 8.69 (s, 1H), 8.45 (d, 1H), 8.02 (d, 1H), 7.84 (d, 1H), 7.78 (d, 1H), 7.77 (dd, 1H), 7.67 (d, 1H), 7.39 (broad s, 1H), 6.94 (d, 1H), 3.81 (s, 3H), 3.72-3.75 (m, 4H), 3.55-3.59 (m, 2H), 3.15-3.20 (m, 1H), 2.94-2.97 (m, 4H), 2.78-2.86 (m, 1H), ppm; MS (ESI+) m/z 492.5 (M+H).

### Example 81

### Preparation of 7-bromo-1-methyl-1H-pyrido[2,3-b][1,4]oxazin-2(3H)-one

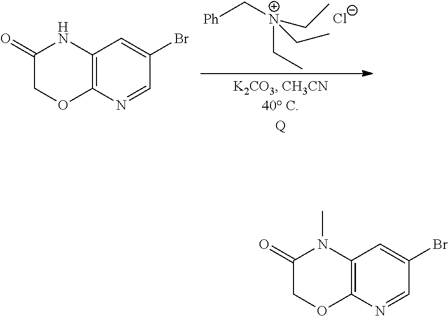

To a round-bottomed flask equipped with a stirring bar, 7-bromo-1H-pyrido[2,3-b][1,4]oxazin-2(3H)-one (5.24 g, 22.9 mmol), acetonitrile (261 mL), benzyltriethylammonium chloride (1.90 g, 11.45 mmol), potassium carbonate (7.91 g, 57.26 mmol), were added. Following the addition of methyl iodide (3.90 g, 27.48 mmol), the mixture was stirred at 60° C. for 7 hrs. Removed all the solvent in vacuo and the resulting residue was added methylene chloride (300 mL), the organic phase was washed with water (30 mL×1), brine (30 mL×1), dried over sodium sulfate, filtered, and removed solvent in vacuo. Silica gel column (methanol:methylene chloride=7.5:92.5) gave the desired product as white solids.

### Example 82

### Preparation of 1-methyl-7-(4,4,5,5-tetramethyl-1,3,2-dioxaborolan-2-yl)-1H-pyrido[2,3-b][1,4]oxazin-2(3H)-one

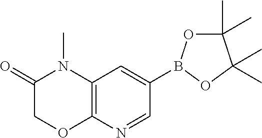 [SMILES: CN1C(=O)COc2ncc(B3OC(C)(C)C(C)(C)O3)cc21]

This compound was synthesized according to procedure D in Example 4 above.

### Example 83

### Preparation of 7-(8-((3-methoxy-4-morpholinophenyl)amino)imidazo[1,2-a]pyrazin-6-yl)-1-methyl-1H-pyrido[2,3-b][1,4]oxazin-2(3H)-one

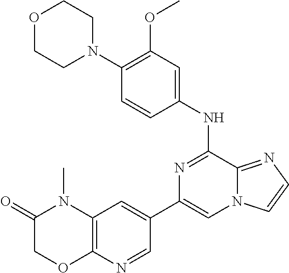 [SMILES: COc1cc(Nc2nc(-c3cnc4c(c3)N(C)C(=O)CO4)cn3ccnc23)ccc1N1CCOCC1]

This compound was synthesized according to procedure F in Example 5b above.

¹H NMR (300 MHz, DMSO-d₆): δ 9.61 (s, 1H), 8.70 (s, 1H), 8.51 (d, 1H), 8.00 (d, 1H), 7.99 (d, 1H), 7.82 (d, 1H), 7.71 (dd, 1H), 7.67 (d, 1H), 6.80 (d, 1H), 4.91 (s, 2H), 3.81 (s, 3H), 3.71-3.75 (m, 4H), 3.38 (s, 3H), 2.94-2.98 (m, 4H) ppm; MS (ESI+) m/z 488.1 (M+H).

### Example 84

### Preparation of (S)-(4-(4-(6-bromoimidazo[1,2-a]pyrazin-8-ylamino)phenyl)morpholin-2-yl)methyl methanesulfonate

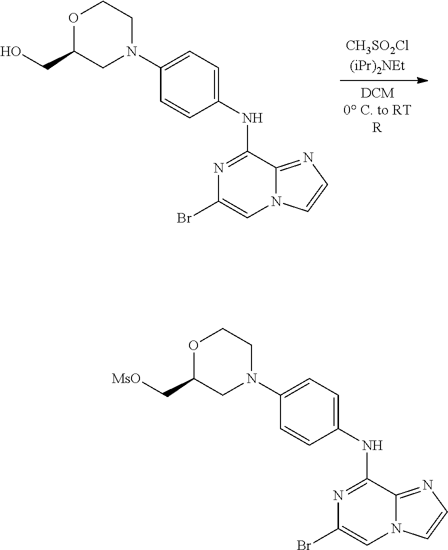 [SMILES: OC[C@@H]1CN(c2ccc(Nc3nc(Br)cn4ccnc34)cc2)CCO1 ; CS(=O)(=O)OC[C@@H]1CN(c2ccc(Nc3nc(Br)cn4ccnc34)cc2)CCO1]

To a microwave tube equipped with a stirring bar, nitrogen gas tee, (S)-(4-(4-(6-bromoimidazo[1,2-a]pyrazin-8-ylamino)phenyl)morpholin-2-yl)methanol (553 mg, 1.37 mmol), methylene chloride (50 mL) were added and cooled to 0° C. in a ice/water bath. 30 minutes later, diisopropylethylamine (884 mg, 6.84 mmol) and methanesulfonyl chloride (251 mg, 2.19 mmol) were added in sequence. 30 minutes later, methylene chloride (150 mL) was added and the resulting mixture was washed with water (20 mL×2), dried over sodium sulfate, filtered, and removed solvent in vacuo. This crude material was directly used in the next step.

### Example 85

### Preparation of (R)-6-bromo-N-(4-(2-((dimethylamino)methyl)morpholinyl)phenyl)imidazo[1,2-a]pyrazin-8-amine

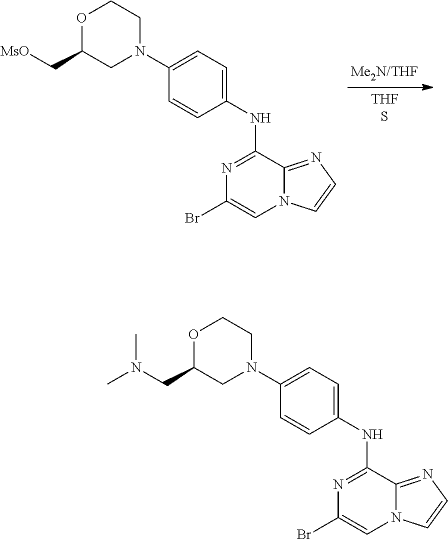 [SMILES: CS(=O)(=O)OC[C@@H]1CN(c2ccc(Nc3nc(Br)cn4ccnc34)cc2)CCO1 ; CN(C)C[C@@H]1CN(c2ccc(Nc3nc(Br)cn4ccnc34)cc2)CCO1]

The mesylate residue was transferred to a seal tube, Me₂NH in tetrahydrofuran (THF) (2 M, 12 mL, 24 mmol) was added. The seal tube was sealed and heated at 110° C. overnight. All volatiles was removed in vacuo and methylene chloride (200 mL) was added to the residue, the organic phase was washed with water (30 mL×2), brine (30 mL×1), dried over sodium sulfate, filtered, and removed solvent in vacuo. Silica gel column chromatography (methanol:methylene chloride=7.5:92.5) gave the desired product as a yellow solids.

### Example 86

### Preparation of (R)-6-(2,3-dihydro-1H-pyrido[2,3-b][1,4]oxazin-7-yl)-N-(4-(2-((dimethylamino)methyl)morpholinyl)phenyl)imidazo[1,2-a]pyrazin-8-amine

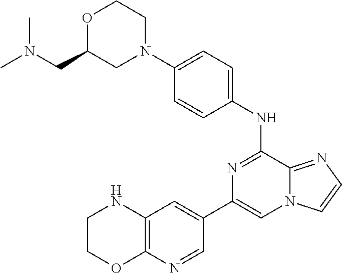 [SMILES: CN(C)C[C@@H]1CN(c2ccc(Nc3nc(-c4cnc5c(c4)NCCO5)cn4ccnc34)cc2)CCO1]

This compound was synthesized according to procedure F in Example 5b above.

¹H NMR (300 MHz, DMSO-d₆): δ 9.46 (s, 1H), 8.40 (s, 1H), 8.00 (d, 2H), 7.98 (s, 1H), 7.94 (s, 1H), 7.60 (s, 1H), 7.45 (m, 1H), 6.98 (d, 2H), 6.26 (broad s, 1H), 4.30 (m, 2H), 3.90-3.96 (m, 1H), 3.44-3.72 (m, 5H), 3.16-3.26 (m, 1H), 2.62-2.70 (m, 1H), 2.34-2.38 (m, 3 H), 2.20 (s, 6H) ppm; MS (ESI+) m/z 487.5 (M+H).

### Example 87

### Preparation of 6-(2,3-dihydro-1H-pyrido[2,3-b][1,4]oxazin-7-yl)-N-(4-(4-isopropylpiperazin-1-yl)phenyl)imidazo[1,2-a]pyrazin-8-amine

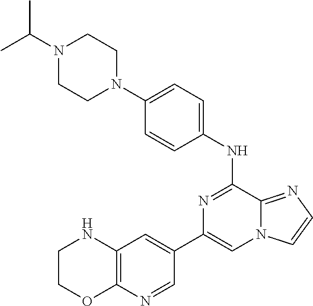 [SMILES: CC(C)N1CCN(c2ccc(Nc3nc(-c4cnc5c(c4)NCCO5)cn4ccnc34)cc2)CC1]

This compound was synthesized according to procedure F in Example 5b above.

¹H NMR (300 MHz, DMSO-d₆): δ 9.41 (s, 1H), 8.39 (s, 1H), 7.96 (s, 1H), 7.93 (d, 2H), 7.92 (s, 1H), 7.59 (d, 1H), 7.42 (d, 1H), 6.94 (d, 2H), 6.23 (broad s, 1H), 4.30 (m, 2H), 3.09 (m, 4H), 2.65 (quintet, 1H), 2.58 (m, 4H), 1.00 (d, 6H) ppm; MS (ESI+) m/z 471.5 (M+H).

### Example 88

### Preparation of 6-(2,3-dihydro-1H-pyrido[2,3-b][1,4]oxazin-7-yl)-N-(4-(4-methylpiperazin-1-yl)phenyl)imidazo[1,2-a]pyrazin-8-amine

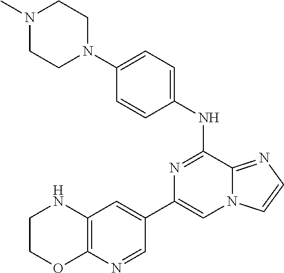 [SMILES: CN1CCN(c2ccc(Nc3nc(-c4cnc5c(c4)NCCO5)cn4ccnc34)cc2)CC1]

This compound was synthesized according to procedure F in Example 5b above.

¹H NMR (300 MHz, DMSO-d₆): δ 9.44 (s, 1H), 8.41 (s, 1H), 7.98 (s, 1H), 7.95 (d, 2H), 7.94 (s, 1H), 7.60 (d, 1H), 7.45 (d, 1H), 6.97 (d, 2H), 6.25 (broad s, 1H), 5.75 (s, 1H), 4.30 (m, 2H), 3.11 (m, 4H), 2.47 (m, 4H), 2.23 (s, 3H) ppm; MS (ESI+) m/z 443.5 (M+H).

### Example 89

### Preparation of 6-(2,3-dihydro-1H-pyrido[2,3-b][1,4]oxazin-7-yl)-N-(4-(4-ethylpiperazin-1-yl)-3-methoxyphenyl)imidazo[1,2-a]pyrazin-8-amine

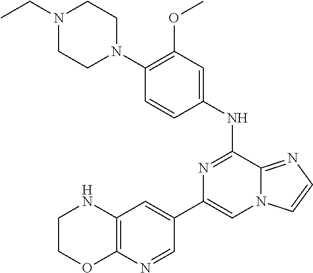 [SMILES: CCN1CCN(c2ccc(Nc3nc(-c4cnc5c(c4)NCCO5)cn4ccnc34)cc2OC)CC1]

This compound was synthesized according to procedure F in Example 5b above.

¹H NMR (300 MHz, DMSO-d₆): δ 9.46 (s, 1H), 8.40 (s, 1H), 7.99 (d, 1H), 7.95 (d, 1H), 7.88 (d, 1H), 7.67 (dd, 1H), 7.60 (d, 1H), 7.44 (d, 1H), 6.88 (d, 1H), 6.20 (broad s, 1H), 4.30 (m, 2H), 3.80 (s, 3H), 2.94 (broad m, 4H), 2.35 (q, 2H), 1.01 (t, 3H), ppm; MS (ESI+) m/z 487.5 (M+H).

### Example 90

### Preparation of 6-(2,3-dihydro-1H-pyrido[2,3-b][1,4]oxazin-7-yl)-N-(4-(4-ethylpiperazin-1-yl)phenyl)imidazo[1,2-a]pyrazin-8-amine

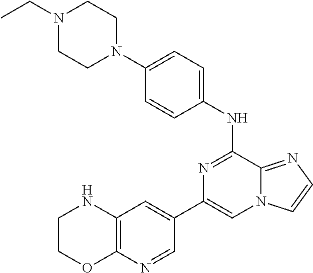 [SMILES: CCN1CCN(c2ccc(Nc3nc(-c4cnc5c(c4)NCCO5)cn4ccnc34)cc2)CC1]

This compound was synthesized according to procedure F in Example 5b above.

¹H NMR (300 MHz, DMSO-d₆): δ 9.43 (s, 1H), 8.40 (s, 1H), 7.98 (s, 1H), 7.97 (d, 2H), 7.95 (s, 1H), 7.60 (d, 1H), 7.45 (d, 1H), 6.98 (d, 2H), 6.25 (broad s, 1H), 4.30 (m, 2H), 3.12 (m, 4H), 2.38 (q, 2H), 1.03 (t, 3H) ppm; MS (ESI+) m/z 457.5 (M+H).

### Example 91

### Preparation of 7-(8-(3-methoxy-4-morpholinophenylamino)imidazo[1,2-a]pyrazin-6-yl)-1H-pyrido[2,3-b][1,4]oxazin-2(3H)-one

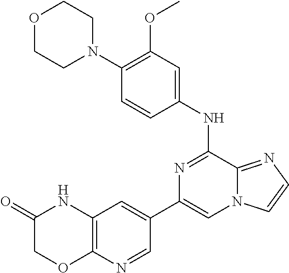 [SMILES: COc1cc(Nc2nc(-c3cnc4c(c3)NC(=O)CO4)cn3ccnc23)ccc1N1CCOCC1]

This compound was synthesized according to procedure F in Example 5b above.

¹H NMR (300 MHz, DMSO-d₆): δ 11.13 (s, 1H), 9.60 (s, 1H), 8.55 (s, 1H), 8.40 (d, 1H), 8.00 (d, 1H), 7.75-7.83 (m, 3H), 7.65 (d, 1H), 6.92 (d, 1H), 4.84 (s, 2H), 3.80 (s, 3H), 3.74 (m, 4H), 2.96 (m, 4H) ppm; MS (ESI+) m/z 474.5 (M+H).

### Example 92

### Preparation of 7-(8-((4-(3-hydroxy-3-methylazetidin-1-yl)-3-methoxyphenyl)amino)imidazo[1,2-a]pyrazin-6-yl)-1H-pyrido[2,3-b][1,4]oxazin-2(3H)-one

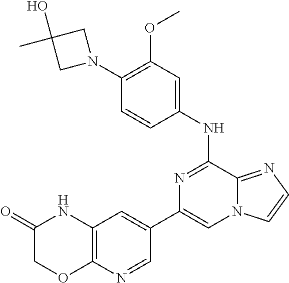 [SMILES: COc1cc(Nc2nc(-c3cnc4c(c3)NC(=O)CO4)cn3ccnc23)ccc1N1CC(C)(O)C1]

This compound was synthesized according to procedure F in Example 5b above.

¹H NMR (300 MHz, DMSO-d₆): δ 11.1 (s, 1H), 9.45 (s, 1H), 8.50 (s, 1H), 8.39 (s, 1 H), 7.98 (s, 1H), 7.63-7.79 (m, 4H), 6.45 (d, 1H), 5.33 (s, 1H), 4.83 (s, 2H), 3.76 (m, 2H), 3.74 (s, 3H), 3.57 (m, 2H), 1.46 (s, 3H), 1.07 (s, 1H) ppm; MS (ESI+) m/z 474.4 (M+H).

### Example 93

### Preparation of 7-(8-((3-methoxy-4-(4-(oxetan-3-yl)piperazin-1-yl)phenyl)amino)imidazo[1,2-a]pyrazin-6-yl)-1H-pyrido[2,3-b][1,4]oxazin-2(3H)-one

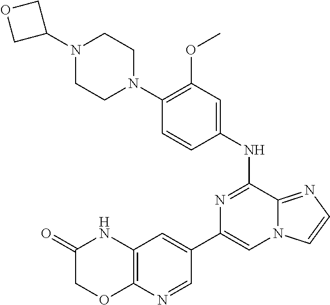 [SMILES: COc1cc(Nc2nc(-c3cnc4c(c3)NC(=O)CO4)cn3ccnc23)ccc1N1CCN(C2COC2)CC1]

This compound was synthesized according to procedure F in Example 5b above.

¹H NMR (300 MHz, DMSO-d₆): δ 11.12 (s, 1H), 9.60 (s, 1H), 8.54 (s, 1H), 8.40 (d, 1H), 8.00 (s, 1H), 7.71 (m, 2H), 7.74 (dm, 1H), 7.65 (s, 1H), 6.93 (d, 1H), 5.76 (s, 1H), 4.74 (s, 2H), 4.57 (t, 2H), 4.48 (t2H), 3.79 (s, 3H), 3.0 (broad s, 4H), 2.41 (broad s, 4. H) ppm; MS (ESI+) m/z 529.5 (M+H).

### Example 94

### Preparation of (R)-(4-(4-((6-(2,3-dihydro-1H-pyrido[2,3-b][1,4]oxazin-7-yl)imidazo[1,2-a]pyrazin-8-yl)amino)phenyl)morpholin-2-yl)methanol

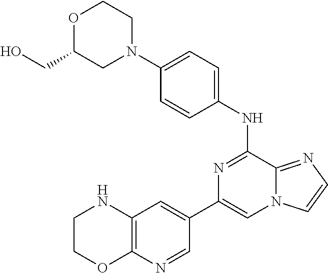 [SMILES: OC[C@H]1CN(c2ccc(Nc3nc(-c4cnc5c(c4)NCCO5)cn4ccnc34)cc2)CCO1]

This compound was synthesized according to procedure F in Example 5b above.

¹H NMR (300 MHz, DMSO-d₆): δ 9.47 (s, 1H), ppm; MS (ESI+) m/z (M+H). 8.41 (s, 1H), 7.95-8.02 (m, 4H), 7.61 (m, 1H), 7.46 (d, 1H), 6.98 (d, 1H), 6.27 (broad s, 1H), 4.77 (t, 1H), 4.31 (m, 2H), 3.93-3.97 (m, 1H), 3.42-3.69 (m, 7H), 2.62-2.70 (m, 1H), 2.41 (m, 1H) ppm; MS (ESI+) m/z 460.5 (M+H).

### Example 95

### Preparation of 6-(2,3-dihydro-1H-pyrido[2,3-b][1,4]oxazin-7-yl)-N-(3-methoxy-4-(4-(oxetan-3-yl)piperazin-1-yl)phenyl)imidazo[1,2-a]pyrazin-8-amine

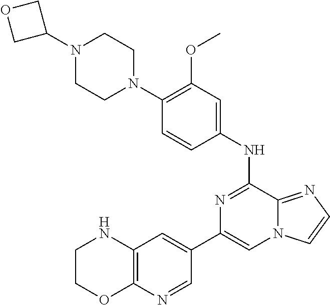 [SMILES: COc1cc(Nc2nc(-c3cnc4c(c3)NCCO4)cn3ccnc23)ccc1N1CCN(C2COC2)CC1]

This compound was synthesized according to procedure F in Example 5b above.

¹H NMR (300 MHz, DMSO-d₆): δ 9.47 (s, 1H), 8.41 (s, 1H), 7.99 (d, 1H), 7.95 (d, 1H), 7.90 (d, 1H), 7.66 (dd, 1H), 7.60 (d, 1H), 7.45 (d, 1H), 6.89 (d, 1H), 6.20 (broad s, 1H), 4.54 (t, 2H), 4.45 (t, 2H), 4.29 (m, 2H), 3.79 (s, 3H), 3.45 (quintet, 1H), 2.96 (m, 4H), 2.39 (m, 4H) ppm; MS (ESI+) m/z 515.5 (M+H).

### Example 96

### Preparation of (R)-(4-(4-((6-(2,3-dihydro-1H-pyrido[2,3-b][1,4]oxazin-7-yl)imidazo[1,2-a]pyrazin-8-yl)amino)-2-methoxyphenyl)morpholin-2-yl)methanol

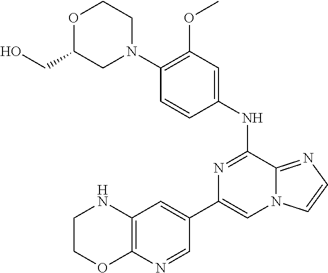 [SMILES: COc1cc(Nc2nc(-c3cnc4c(c3)NCCO4)cn3ccnc23)ccc1N1CCO[C@@H](CO)C1]

This compound was synthesized according to procedure F in Example 5b above.

¹H NMR (300 MHz, DMSO-d₆): δ 9.52 (s, 1H), 8.43 (s, 1H), 8.01 (d, 1H), 7.97 (d, 1H), 7.91 (d, 1H), 7.72 (dd, 1H), 7.63 (d, J=0.6 Hz, 1H), 6.90 (d, 1H), 6.24 (broad s, 1H), 4.72 (t, 1H), 4.30 (m, 2H), 3.88 (m, 1H), 3.57-3.71 (m, 3H), 3.35-3.50 (m, 3H), 3.20 (m, 1H), 2.60-2.67 (m, 1H), 2.37-2.45 (m, 1H) ppm; MS (ESI+) m/z 490.6 (M+H).

### Example 97

### Preparation of 2-(4-(4-nitrophenyl)morpholin-2-yl)ethanol

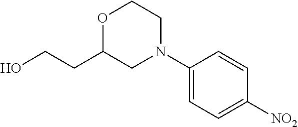 [SMILES: O=[N+]([O-])c1ccc(N2CCOC(CCO)C2)cc1]

A mixture of 1-fluoro-4-nitrobenzene (2.05 g, 14.5 mmol), 2-(morpholin-2-yl)ethanol (2.00 g, 15.2 mmol) and anhydrous N,N-diisopropylethylamine (3.78 g, 29.1 mmol) in acetonitrile (100 mL) was stirred at reflux for 16 hours. After this time, the reaction was cooled to room temperature, filtered through diatomaceous earth and the filtrate concentrated under reduced pressure. The resulting residue was diluted in methylene chloride (100 mL), washed with water (2×75 mL), then brine (75 mL) and dried over sodium sulfate. The drying agent was removed by filtration and the filtrate concentrated under reduced pressure. The resulting residue was purified by chromatography (silica, 1:1 hexanes/ethyl acetate) to afford 2-(4-(4-nitrophenyl)morpholin-2-yl)ethanol as a yellow solid: ¹H NMR (400 MHz, DMSO-d₆): δ 8.07 (d, 2H), 7.05 (d, 2H), 4.49 (t, 1H), 3.97-3.89 (m, 2H), 3.83 (d, 1H), 3.64-3.51 (m, 4H), 2.98-2.91 (m, 1H), 2.68 (t, 1H), 1.66-1.62 (m, 2H).

### Example 98a Preparation of 2-(4-(4-aminophenyl)morpholin-2-yl)ethanol

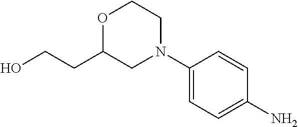 [SMILES: Nc1ccc(N2CCOC(CCO)C2)cc1]

A 500-mL Parr hydrogenation bottle was purged with nitrogen and charged with 2-(4-(4-nitrophenyl)morpholin-2-yl)ethanol (3.00 g, 11.9 mmol), ethanol (150 mL) and 10% palladium on carbon (50% wet, 600 mg dry weight). The bottle was evacuated, charged with hydrogen gas to a pressure of 40 psi and shaken for 2 h at room temperature on a Parr hydrogenation apparatus. After this time, the hydrogen gas was evacuated and nitrogen charged into the bottle. The catalyst was removed by filtration through a pad of diatomaceous earth and the filter cake washed with methanol (50 mL). The filtrate was concentrated under reduced pressure to afford 2-(4-(4-aminophenyl)morpholin-2-yl)ethanol as a brown oil which was used in the next step: ¹H NMR (400 MHz, DMSO-d₆): δ 6.68 (d, 2H), 6.49 (d, 2H), 4.55 (s, 2H), 4.42 (t, 1H), 3.85 (d, 1H), 3.65-3.48 (m, 4H), 3.25 (d, 1H), 3.16 (d, 1H), 2.54-2.50 (m, 1H, merged with DMSO peak), 2.23 (t, 1H), 1.63-1.56 (m, 2H).

### Example 98b

### Resolution of racemic 2-(4-(4-aminophenyl)morpholin-2-yl)ethanol

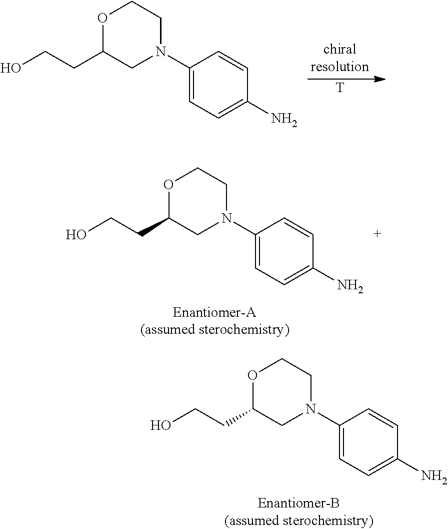

Resolution of racemic 2-(4-(4-aminophenyl)morpholin-2-yl)ethanol (2.21 g, 9.95 mmol) was achieved using a Chiralcel OJ column (2:3 heptane/ethanol) detecting at 240 nm. The separated enantiomers were concentrated under reduced pressure to afford 2-(4-(4-aminophenyl)morpholin-2-yl)ethanol, 1^{st} Eluting Isomer, Enantiomer-A (1.05 g, 48%) and 2-(4-(4-aminophenyl)morpholin-2-yl)ethanol, 2^{nd} Eluting Isomer, Enantiomer-B (810 mg, 37%).

### Example 99

### Preparation of 2-(4-(4-(6-bromoimidazo[1,2-a]pyrazin-8-ylamino)phenyl)morpholin-2-yl)ethanol, Enantiomer-A

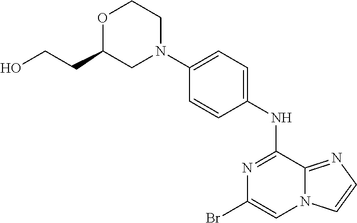 [SMILES: OCC[C@@H]1CN(c2ccc(Nc3nc(Br)cn4ccnc34)cc2)CCO1]

This compound was synthesized according procedure C in Example 3 above.

¹H NMR (400 MHz, DMSO-d₆): δ 9.77 (s, 1H), 8.18 (s, 1H), 7.91 (s, 1H), 7.79 (d, 2H), 7.60 (s, 1H), 6.95 (d, 2H), 4.47 (t, 1H), 3.92 (dd, 1H), 3.68-3.48 (m, 5H), 3.44 (d, 1H), 2.67-2.61 (m, 1H), 2.36 (t, 1H), 1.66-1.62 (m, 2H).

### Example 100

### Preparation of (R)-2-(4-(4-((6-(3,4-dihydro-2H-benzo[b][1,4]oxazin-6-yl)imidazo[1,2-a]pyrazin-8-yl)amino)phenyl)morpholin-2-yl)ethanol, Enantiomer-A

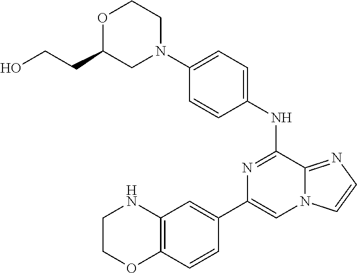 [SMILES: OCC[C@@H]1CN(c2ccc(Nc3nc(-c4ccc5c(c4)NCCO5)cn4ccnc34)cc2)CCO1]

This compound was synthesized according to procedure F in Example 5b above.

¹H NMR (400 MHz, DMSO-d₆): δ 13.18 (s, 1H), 9.50 (s, 1H), 8.66 (s, 1H), 8.19 (s, 1H), 8.09 (s, 1H), 8.04 (d, 2H), 7.98 (d, 1H), 7.84 (d, 1H), 7.72 (dd, 1H), 7.64 (d, 1H), 7.01 (d, 2H), 4.48 (bs, 1H), 3.94 (dd, 1H), 3.72-3.49 (m, 5H), 3.48 (d, 1H), 2.71-2.64 (m, 1H), 2.40 (t, 1H), 1.65-1.61 (m, 2H); ESI MS m/z 456.4 [M+H]⁺; HPLC, 4.96 min, >99% (AUC); optical rotation [α]²⁵_{D} −2.1° (c 0.70, DMSO).

### Example 101

### Preparation of methyl 2-(4-nitrophenyl)acetate

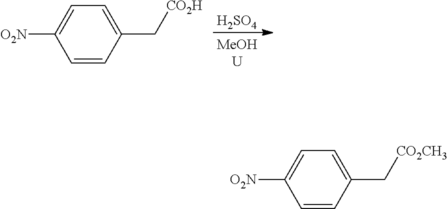

A solution of 2-(4-nitrophenyl)acetic acid (10.0 g, 55.2 mmol) in methanol (50 mL) was treated with 18 M sulfuric acid (5 mL) and the mixture stirred at reflux for 2 hours. After this time, the reaction was slowly cooled to 5° C., let stand for 30 minutes and the resulting suspension filtered. The filter cake was washed with methanol and dried to a constant weight under vacuum to afford methyl 2-(4-nitrophenyl)acetate as an off-white solid: ¹H NMR (300 MHz, DMSO-d₆): δ 8.19 (d, 2H), 7.57 (d, 2H), 3.90 (s, 2H), 3.64 (s, 3H).

### Example 102

### Preparation of methyl 2-methyl-2-(4-nitrophenyl)propanoate

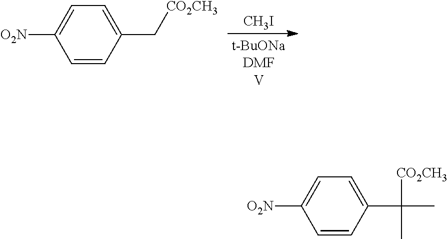 [SMILES: COC(=O)Cc1ccc([N+](=O)[O-])cc1 ; "CH3I" ; COC(=O)C(C)(C)c1ccc([N+](=O)[O-])cc1]

A solution of dimethylformamide (100 mL) at 0° C. was treated with sodium tert-butoxide (3.75 g, 39.0 mmol) in one portion and the suspension was stirred for 5 minutes. Methyl 2-(4-nitrophenyl)acetate (7.50 g, 38.4 mmol) was then added in one portion. This was followed by the addition of methyl iodide (8.76 g, 61.7 mmol) over 1 hour, maintaining the temperature below 10° C. After the addition was complete, the mixture was stirred at 0-5° C. for 15 minutes. A second addition of sodium tert-butoxide (3.75 g, 39.0 mmol) and methyl iodide (8.76 g, 61.7 mmol) was made, as described above, and the mixture was stirred at 0-5° C. for a further 20 minutes. A third addition of sodium tert-butoxide (375 mg, 3.90 mmol) and methyl iodide (880 mg, 6.17 mmol) was made, as described above, and the mixture was stirred at 0-5° C. for a further 30 minutes. After this time, a mixture of water (100 mL) and acetic acid (0.83 mL) was slowly added and the resulting solution extracted with ethyl acetate (2×100 mL). The combined organic layers were washed with 0.5 M hydrochloric acid (2×50 mL), then brine (50 mL) and dried over sodium sulfate. The drying agent was removed by filtration and the filtrate concentrated under reduced pressure to afford methyl 2-methyl-2-(4-nitrophenyl)propanoate as an off-white solid: ¹H NMR (300 MHz, DMSO-d₆): δ 8.19 (d, 2H), 7.61 (d, 2H), 3.62 (s, 3H), 1.56 (s, 6H).

### Example 103

### Preparation of 2-methyl-2-(4-nitrophenyl)propan-1-ol

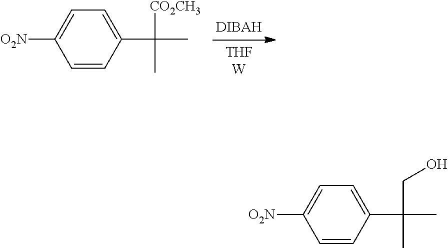 [SMILES: COC(=O)C(C)(C)c1ccc([N+](=O)[O-])cc1 ; CC(C)(CO)c1ccc([N+](=O)[O-])cc1]

A solution of methyl 2-methyl-2-(4-nitrophenyl)propanoate (1.00 g, 4.50 mmol) in anhydrous tetrahydrofuran (20 mL) was cooled to −5° C., under a nitrogen atmosphere, in an ice/brine cooling bath. 1 M Diisobutylaluminum hydride in tetrahydrofuran (9.9 mL) was then added dropwise and the reaction stirred at 0° C. for 2 hours. After this time, the reaction was carefully treated with 10% hydrochloric acid (20 mL) maintaining the temperature below 25° C. Note: Extreme caution was used with the addition of 10% hydrochloric acid due to an exotherm and gas evolution. The initial addition was performed dropwise and the temperature was allowed to equilibrate between drops. After the addition was complete, the layers were separated and the aqueous phase was extracted with methylene chloride (3×50 mL) and the combined organic layers were dried over sodium sulfate. The drying agent was removed by filtration and the filtrate was concentrated under reduced pressure to afford 2-methyl-2-(4-nitrophenyl)propan-1-ol as a colorless solid: ¹H NMR (300 MHz, DMSO-d₆): δ 8.15 (d, 2H), 7.66 (d, 2H), 4.80 (t, 1H), 3.47 (d, 2H), 1.27 (s, 6H).

### Example 104

### Preparation of 2-(4-aminophenyl)-2-methylpropan-1-ol

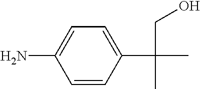

This compound was synthesized according to procedure B in Example 2 above.

¹H NMR (300 MHz, DMSO-d₆): δ 7.00 (d, 2H), 6.49 (d, 2H), 4.77 (bs, 2H), 4.48 (t, 1H), 3.29 (d, 2H), 1.14 (s, 6H).

### Example 105

### Preparation of 2-(4-(6-bromoimidazo[1,2-a]pyrazin-8-ylamino)phenyl)-2-methylpropan-1-ol

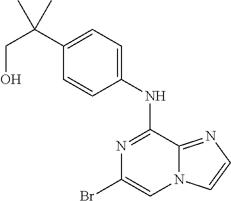 [SMILES: CC(C)(CO)c1ccc(Nc2nc(Br)cn3ccnc23)cc1]

This compound was synthesized according procedure C in Example 3 above.

¹H NMR (300 MHz, DMSO-d₆): δ 9.88 (s, 1H), 8.24 (s, 1H), 7.94 (s, 1H), 7.83 (d, 2H), 7.63 (s, 1H), 7.34 (d, 2H), 4.65 (s, 1H), 3.41 (s, 2H), 1.23 (s, 6H).

### Example 106

### Preparation of (R)-(4-(4-nitrophenyl)morpholin-3-yl)methanol

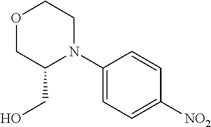 [SMILES: O=[N+]([O-])c1ccc(N2CCOC[C@H]2CO)cc1]

A mixture of 1-fluoro-4-nitrobenzene (0.82 g, 5.8 mmol), (R)-morpholin-3-ylmethanol (1.5 g, 17.4 mmol), and potassium carbonate (2.4 g, 17.4 mmol) in dimethylamine (15 mL) was stirred at 95° C. for 40 hours. The reaction was cooled to room temperature, and diluted with ethyl acetate (10 mL) and water (5 mL). The organic layers were extracted with ethyl acetate (5 mL×3). The combined organic layers were washed with water (2×5 mL), brine (1×5 mL), dried using sodium sulfate, and concentrated under reduced pressure. The resulting residue was purified by flash chromatography (hexane:ethyl acetate, 65:45) to afford (R)-(4-(4-nitrophenyl)morpholin-3-yl)methanol as a yellow oil.

### Example 107

### Preparation of (R)-(4-(4-aminophenyl)morpholin-3-yl)methanol

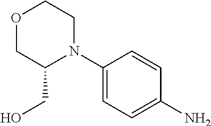 [SMILES: Nc1ccc(N2CCOC[C@H]2CO)cc1]

This compound was prepared by the procedure analogous to that of procedure B in Example 2 above, using (R)-(4-(4-nitrophenyl)morpholin-3-yl)methanol (150 mg, 0.63 mmol) as a starting material to give (R)-(4-(4-aminophenyl)morpholin-3-yl)methanol as a yellow solid.

### Example 108

### Preparation of (R)-(4-(4-(6-bromoimidazo[1,2-a]pyrazin-8-ylamino)phenyl)morpholin-3-yl)methanol

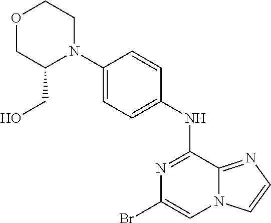 [SMILES: OC[C@@H]1COCCN1c1ccc(Nc2nc(Br)cn3ccnc23)cc1]

A 40-mL sealed tube equipped with a magnetic stirring bar was charged with 6,8-dibromoimidazo[1,2-a]pyrazine (170 mg, 0.63 mmol), (R)-(4-(4-aminophenyl)morpholin-3-yl)methanol (130 mg, 0.63 mmol), and camphor-10-sulphonic acid (150 mg, 0.63 mmol) in i-PrOH (10 mL). After the reaction mixture was stirred at 85° C. for 16 hours, it was cooled to room temperature, and diluted with ethyl acetate (5 mL) and water (5 mL). The organic layers were extracted with ethyl acetate (5 mL×3). The combined organic layers were washed with water (2×5 mL), brine (1×5 mL), dried with sodium sulfate, and concentrated under reduced pressure. The resulting residue was purified by flash chromatography (dichloromethane:methanol, 95:5) to afford (R)-(4-(4-(6-bromoimidazo[1,2-a]pyrazin-8-ylamino)phenyl)morpholin-3-yl)methanol as a white solid.

### Example 109

### Preparation of (R)-(4-(4-((6-(2,3-dihydro-1H-pyrido[2,3-b][1,4]oxazin-7-yl)imidazo[1,2-a]pyrazin-8-yl)amino)phenyl)morpholin-3-yl)methanol

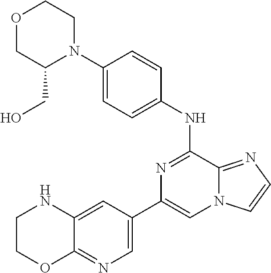 [SMILES: OC[C@@H]1COCCN1c1ccc(Nc2nc(-c3cnc4c(c3)NCCO4)cn3ccnc23)cc1]

This compound was synthesized according to procedure F in Example 5b above.

¹H NMR (δ, ppm): 9.43 (s, 1H), 8.40 (s, 1H), 7.99 (m, 4H), 7.60 (d, 1H), 7.44 (d, 1H), 6.93 (d, 2H), 6.22 (s, 1H), 4.69 (m, 1H), 4.31 (m, 2H), 4.11 (m, 1H), 3.90 (m, 1H), 3.61 (m, 4H), 3.21 (m, 4H), 3.00 (m, 1H); MS (ESI+) m/z 460.5 (M+H).

### Example 110

### Preparation of 7-(8-((4-morpholinophenyl)amino)imidazo[1,2-a]pyrazin-6-yl)-3,4-dihydrobenzo[f][1,4]oxazepin-5(2H)-one

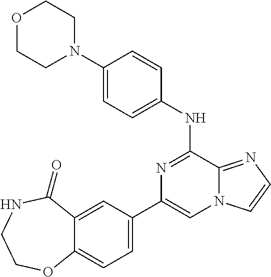 [SMILES: O=C1NCCOc2ccc(-c3cn4ccnc4c(Nc4ccc(N5CCOCC5)cc4)n3)cc21]

This compound was synthesized according to procedure F in Example 5b above

¹H NMR (δ, ppm): 9.49 (s, 1H), 8.58 (s, 1H), 8.45 (m, 2H), 8.10 (m, 4H), 7.62 (s, 1H), 7.12 (d, 1H), 6.95 (d, 2H), 4.34 (m, 2H), 3.38 (m, 4H), 3.34 (m, 2H), 3.08 (m, 4H); MS (ESI+) m/z 457.4 (M+H).

### Example 111

### Preparation of N-(4-morpholinophenyl)-6-(2,3,4,5-tetrahydrobenzo[f][1,4]oxazepin-7-yl)imidazo[1,2-a]pyrazin-8-amine

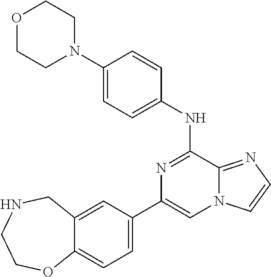 [SMILES: c1cn2cc(-c3ccc4c(c3)CNCCO4)nc(Nc3ccc(N4CCOCC4)cc3)c2n1]

This compound was synthesized according to procedure G in Example 7 above.

MS (ESI+) m/z 443.5 (M+H); NMR (δ, ppm): 9.45 (s, 1H), 8.50 (s, 1H), 7.96 (m, 2H), 7.80 (d, 1H), 7.76 (m, 2H), 7.60 (d, 1H), 7.04 (d, 1H), 7.00 (m, 2H), 3.99 (m, 2H), 3.92 (m, 2H), 3.75 (m, 4H), 3.17 (m, 6H).

### Example 112

### Preparation of (S)-8-(2-methoxy-4-nitrophenyl)octahydropyrazino[2,1-c][1,4]oxazine

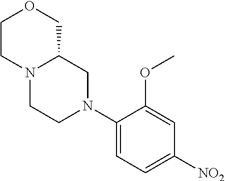 [SMILES: COc1cc([N+](=O)[O-])ccc1N1CCN2CCOC[C@@H]2C1]

This compound was prepared utilizing procedure A in Example 1 above, using (5)-octahydropyrazino[2,1-c][1,4]oxazine dihydrochloride (500 mg, 2.3 mmol) as a starting material to give (S)-8-(2-methoxy-4-nitrophenyl)octahydropyrazino[2,1-c][1,4]oxazine as a yellow solid.

### Example 113

### Preparation of (S)-4-(hexahydropyrazino[2,1-c][1,4]oxazin-8(1H)-yl)-3-methoxyaniline

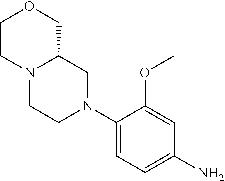 [SMILES: COc1cc(N)ccc1N1CCN2CCOC[C@@H]2C1]

This compound was prepared utilizing procedure B in Example 2 above, using instead (S)-8-(2-methoxy-4-nitrophenyl)octahydropyrazino[2,1-c][1,4]oxazine as a starting material to give (S)-4-(hexahydropyrazino[2,1-c][1,4]oxazin-8(1H)-yl)-3-methoxyaniline as a yellow oil.

### Example 114

### Preparation of (S)-6-bromo-N-(4-(hexahydropyrazino[2,1-c][1,4]oxazin-8(1H)-yl)-3-methoxyphenyl)imidazo[1,2-a]pyrazin-8-amine

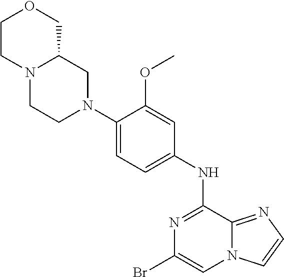 [SMILES: COc1cc(Nc2nc(Br)cn3ccnc23)ccc1N1CCN2CCOC[C@@H]2C1]

This compound was prepared using procedure C in Example 3 above, using instead
(S)-4-(hexahydropyrazino[2,1-c][1,4]oxazin-8(1H)-yl)-3-methoxyaniline (526 g, 2.0 mmol) to afford (S)-6-bromo-N-(4-(hexahydropyrazino[2,1-c][1,4]oxazin-8 (1H)-yl)-3-methoxyphenyl)imidazo[1,2-a]pyrazin-8-amine as a white solid.

### Example 115

### Preparation of (S)-6-(2,3-dihydro-1H-pyrido[2,3-b][1,4]oxazin-7-yl)-N-(4-(hexahydropyrazino[2,1-c][1,4]oxazin-8(1H)-yl)-3-methoxyphenyl)imidazo[1,2-a]pyrazin-8-amine

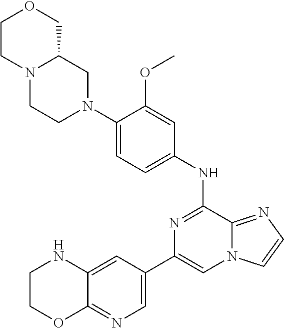 [SMILES: COc1cc(Nc2nc(-c3cnc4c(c3)NCCO4)cn3ccnc23)ccc1N1CCN2CCOC[C@@H]2C1]

This compound was synthesized according to procedure F in Example 5b above.

MS (ESI+) m/z 515.5 (M+H); NMR (δ, ppm): 9.49 (s, 1H), 8.43 (s, 1H), 8.00 (d, 1H), 7.97 (d, 1H), 7.89 (d, 1H), 7.70 (dd, 1H), 7.62 (d, 1H), 7.46 (d, 1H), 6.88 (d, 1H), 6.23 (s, 1H), 4.29 (m, 2H), 3.81 (s, 3H), 3.78 (m, 1H), 3.65 (m, 1H), 3.54 (m, 1H), 3.31 (m, 2H), 3.12 (m, 2H), 2.71 (m, 3H), 2.49 (m, 1H), 2.29 (m, 4H).

### Example 116

### Preparation of 4-(2-methoxy-4-nitrophenyl)-2,2-dimethylmorpholine

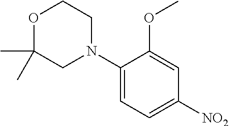 [SMILES: COc1cc([N+](=O)[O-])ccc1N1CCOC(C)(C)C1]

This compound was prepared utilizing procedure A in Example 1 above, using instead 2,2-dimethylmorpholine (500 mg, 4.3 mmol) as a starting material to give 4-(2-methoxy-4-nitrophenyl)-2,2-dimethylmorpholine as a yellow solid.

### Example 117

### Preparation of 4-(2,2-dimethylmorpholinyl)-3-methoxyaniline

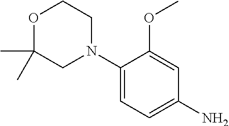 [SMILES: COc1cc(N)ccc1N1CCOC(C)(C)C1]

This compound was prepared utilizing procedure B in Example 2 above, using instead 4-(2-methoxy-4-nitrophenyl)-2,2-dimethylmorpholine (960 mg, 3.7 mmol) as a starting material to give 4-(2,2-dimethylmorpholinyl)-3-methoxyaniline as a yellow solid.

### Example 118

### Preparation of 6-bromo-N-(4-(2,2-dimethylmorpholinyl)-3-methoxyphenyl)imidazo[1,2-a]pyrazin-8-amine

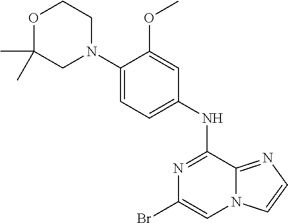 [SMILES: COc1cc(Nc2nc(Br)cn3ccnc23)ccc1N1CCOC(C)(C)C1]

This compound was prepared utilizing procedure C in Example C above, using instead 4-(2,2-dimethylmorpholinyl)-3-methoxyaniline (810 mg, 3.4 mmol) as a starting material to give 6-bromo-N-(4-(2,2-dimethylmorpholinyl)-3-methoxyphenyl)imidazo[1,2-a]pyrazin-8-amine as a white solid.

### Example 119

### Preparation of 6-(2,3-dihydro-1H-pyrido[2,3-b][1,4]oxazin-7-yl)-N-(4-(2,2-dimethylmorpholinyl)-3-methoxyphenyl)imidazo[1,2-a]pyrazin-8-amine

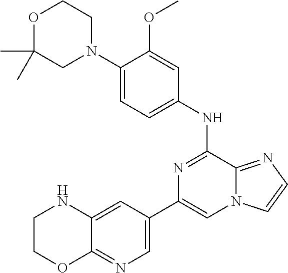 [SMILES: COc1cc(Nc2nc(-c3cnc4c(c3)NCCO4)cn3ccnc23)ccc1N1CCOC(C)(C)C1]

This compound was synthesized according to procedure F in Example 5b above.

MS (ESI+) m/z 488.4 (M+H); NMR (δ, ppm): 9.49 (s, 1H), 8.43 (s, 1H), 8.00 (d, 1H), 7.96 (d, 1H), 7.91 (d, 1H), 7.70 (dd, 1H), 7.68 (d, 1H), 7.46 (d, 1H), 6.88 (d, 1H), 6.23 (s, 1H), 4.30 (m, 2H), 3.81 (s, 3H), 3.74 (m, 2H), 3.31 (m, 2H), 2.89 (m, 2H), 2.73 (s, 2H), 6.19 (s, 6H).

### Example 120

### Preparation of (S)-8-(4-nitrophenyl)octahydropyrazino[2,1-c][1,4]oxazine

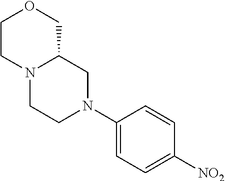 [SMILES: O=[N+]([O-])c1ccc(N2CCN3CCOC[C@@H]3C2)cc1]

This compound was prepared utilizing procedure A in Example 2 above, using instead (S)-octahydropyrazino[2,1-c][1,4]oxazine dihydrochloride (500 mg, 2.3 mmol) to give (S)-8-(4-nitrophenyl)octahydropyrazino[2,1-c][1,4]oxazine as a yellow solid.

### Example 121

### Preparation of (S)-4-(hexahydropyrazino[2,1-c][1,4]oxazin-8(1H)-yl)aniline

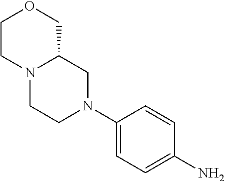 [SMILES: Nc1ccc(N2CCN3CCOC[C@@H]3C2)cc1]

This compound was prepared utilizing procedure B in Example 2 above, using instead (S)-8-(4-nitrophenyl)octahydropyrazino[2,1-c][1,4]oxazine (461 mg, 1.8 mmol) to give (S)-4-(hexahydropyrazino[2,1-c][1,4]oxazin-8(1H)-yl)aniline as a white solid.

### Example 122

### Preparation of (S)-6-bromo-N-(4-(hexahydropyrazino[2,1-c][1,4]oxazin-8(1H)-yl)phenyl)imidazo[1,2-a]pyrazin-8-amine

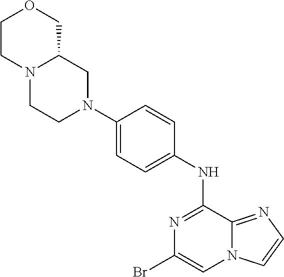 [SMILES: Brc1cn2ccnc2c(Nc2ccc(N3CCN4CCOC[C@@H]4C3)cc2)n1]

This compound was prepared utilizing procedure C in Example 3 above, using instead (S)-4-(hexahydropyrazino[2,1-c][1,4]oxazin-8(1H)-yl)aniline (233 mg, 1.0 mmol) as a starting material to give (S)-6-bromo-N-(4-(hexahydropyrazino[2,1-c][1,4]oxazin-8(1H)-yl)phenyl)imidazo[1,2-a]pyrazin-8-amine as a white solid.

### Example 123

### Preparation of (S)-6-(2,3-dihydro-1H-pyrido[2,3-b][1,4]oxazin-7-yl)-N-(4-(hexahydropyrazino[2,1-c][1,4]oxazin-8(1H)-yl)phenyl)imidazo[1,2-a]pyrazin-8-amine

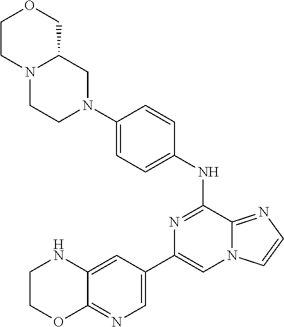 [SMILES: c1cn2cc(-c3cnc4c(c3)NCCO4)nc(Nc3ccc(N4CCN5CCOC[C@@H]5C4)cc3)c2n1]

This compound was synthesized according to procedure F in Example 5b above.

MS (ESI+) m/z 485.5 (M+H); NMR (δ, ppm): 9.44 (s, 1H), 8.40 (s, 1H), 7.96 (m, 4H), 7.60 (d, 1H), 7.44 (d, 1H), 6.98 (d, 2H), 6.26 (s, 1H), 4.30 (m, 2H), 3.76 (m, 2H), 3.50 (m, 3H), 3.19 (m, 2H), 2.71 (m, 3H), 2.26 (m, 5H).

### Example 124

### Preparation of 1-(2-cyclopropoxyethyl)-4-(2-methoxy-4-nitrophenyl)piperazine

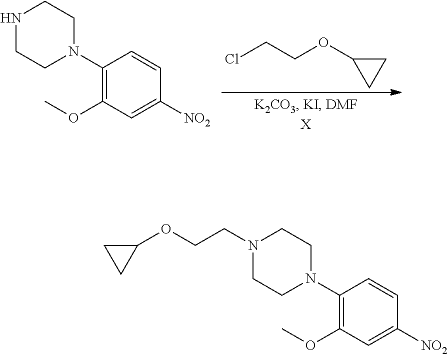 [SMILES: COc1cc([N+](=O)[O-])ccc1N1CCNCC1 ; ClCCOC1CC1 ; COc1cc([N+](=O)[O-])ccc1N1CCN(CCOC2CC2)CC1]

A mixture of 1-(2-methoxy-4-nitrophenyl)piperazine (490 mg, 2.0 mmol), (2-chloroethoxy)-cyclopropane (0.34 mL, 3.1 mmol), potassium carbonate (570 mg, 4.1 mmol), potassium iodide (33 mg, 0.2 mmol) in dimethylformamide (7 mL) was heated to 75° C. After 16 h, ethyl acetate (10 mL) and water (10 mL) were added. The aqueous layer was separated and extracted with ethyl acetate (2×10 mL). The combined organic extracts were washed with brine (10 mL), dried over sodium sulfate and concentrated in vacuo. The resulting residue was purified by column chromatography eluting with a gradient of 100% methylene chloride—100% 60:35:5 methylene chloride:diethylether:methanol to afford 1-(2-cyclopropoxyethyl)-4-(2-methoxy-4-nitrophenyl)piperazine.

### Example 125

### Preparation of 4-(4-(2-cyclopropoxyethyl)piperazin-1-yl)-3-methoxyaniline

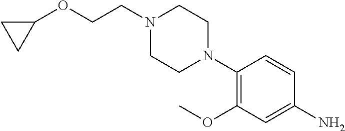 [SMILES: COc1cc(N)ccc1N1CCN(CCOC2CC2)CC1]

To a suspension of 1-(2-cyclopropoxyethyl)-4-(2-methoxy-4-nitrophenyl)piperazine (500 mg, 1.56 mmol) in ethanol (150 mL) was added 10% palladium on carbon (50% wet, 100 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, and washed with ethanol. The filtrate was concentrated in vacuo to give 4-(4-(2-cyclopropoxyethyl)piperazin-1-yl)-3-methoxyaniline.

### Example 126

### Preparation of 6-bromo-N-(4-(4-(2-cyclopropoxyethyl)piperazin-1-yl)-3-methoxyphenyl)imidazo[1,2-a]pyrazin-8-amine

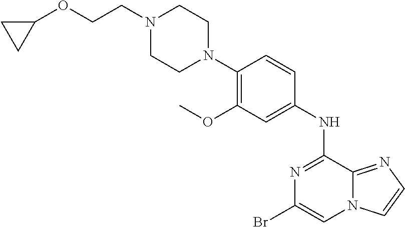 [SMILES: COc1cc(Nc2nc(Br)cn3ccnc23)ccc1N1CCN(CCOC2CC2)CC1]

A mixture of 4-(4-(2-cyclopropoxyethyl)piperazin-1-yl)-3-methoxyaniline (450 mg, 1.56 mmol), 6,8-dibromoimidazo[1,2-a]pyrazine (440 g, 1.6 mmol) and camphorsulfonic acid (370 mg, 1.6 mmol) in isopropanol (11 mL) was heated at reflux for 7 hours. After being cooled down to room temperature, methylene chloride (10 mL) and saturated aqueous sodium bicarbonate (10 mL) were added. The aqueous layer was separated and extracted with methylene chloride (2×10 mL). The combined organic extracts were washed with brine (10 mL), dried over sodium sulfate and concentrated in vacuo. The resulting residue was purified by column chromatography eluting with a gradient of 100% methylene chloride—100% 60:35:5 methylene chloride:diethylether:methanol to afford 6-bromo-N-(4-(4-(2-cyclopropoxyethyl)piperazin-1-yl)-3-methoxyphenyl)imidazo[1,2-a]pyrazin-8-amine.

### Example 127

### Preparation of N-(4-(4-(2-cyclopropoxyethyl)piperazin-1-yl)-3-methoxyphenyl)-6-(2,3-dihydro-1H-pyrido[2,3-b][1,4]oxazin-7-yl)imidazo[1,2-a]pyrazin-8-amine

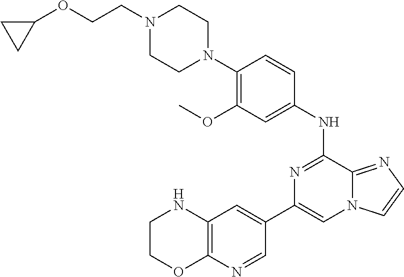 [SMILES: COc1cc(Nc2nc(-c3cnc4c(c3)NCCO4)cn3ccnc23)ccc1N1CCN(CCOC2CC2)CC1]

This compound was synthesized according to procedure F in Example 5b above.

¹H NMR (200 MHz, d₆-DMSO): δ 9.47 (s, 1H), 8.40 (s, 1H), 8.00 (d, 1H), 7.94 (d, 1H), 7.89 (d, 1H), 7.65 (dd, 1H), 7.60 (d, 1H), 7.44 (d, 1H), 6.89 (d, 1H), 5.70 (brs, 1H), 4.29 (t, 1H), 3.79 (s, 3H), 3.55 (dd, 2H), 3.29 (m, 5H), 2.92 (m, 4H), 2.51 (m, 4H), 0.44 (m, 4H). MS (ESI+) m/z 543.5 (M+H).

### Example 128

### Preparation of tert-butyl 4-(2-methoxy-4-nitrophenyl)-2,2-dimethylpiperazine-1-carboxylate

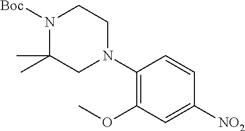 [SMILES: COc1cc([N+](=O)[O-])ccc1N1CCN(C(=O)OC(C)(C)C)C(C)(C)C1]

A mixture of 1-fluoro-2-methoxy-4-nitrobenzene (1.7 g, 10 mmol), 1-boc-2,2-dimethylpiperazine (2.6 g, 12 mmol) and potassium carbonate (2.9 g, 21.1 mmol) in dimethylformamide (12 mL) was stirred at 100° C. for 3 days. The reaction was cooled to room temperature, diluted with methylene chloride (10 mL) and water (10 mL). The aqueous layer was separated and extracted with methylene chloride (2×10 mL). The combined organic extracts were washed with water (4×10 mL) and brine (10 mL) and dried over sodium sulfate. After concentration, crude tert-butyl 4-(2-methoxy-4-nitrophenyl)-2,2-dimethylpiperazine-1-carboxylate was carried into the next step.

### Example 129

### Preparation of 1-(2-methoxy-4-nitrophenyl)-3,3-dimethylpiperazine

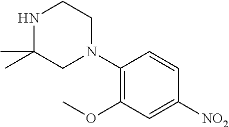 [SMILES: COc1cc([N+](=O)[O-])ccc1N1CCNC(C)(C)C1]

To a solution of tert-butyl 4-(2-methoxy-4-nitrophenyl)-2,2-dimethylpiperazine-1-carboxylate (4.3 g, 12 mmol) in methylene chloride (120 mL) was added trifluoroacetic acid (19 mL, 240 mmol). The mixture stirred at room temperature for 16 hours. After this time, sat aqueous sodium bicarbonate (50 mL) was carefully added. The aqueous layer was separated and extracted with methylene chloride (2×30 mL). The combined organic extracts were washed with brine (50 mL), dried over sodium sulfate and concentration in vacuo. The resulting residue was purified by column chromatography eluting with a gradient of 100% methylene chloride—100% 75:18:7 methylene chloride:diethylether:methanol to afford 1-(2-methoxy-4-nitrophenyl)-3,3-dimethylpiperazine.

### Example 130

### Preparation of 4-(2-methoxy-4-nitrophenyl)-2,2-dimethyl-1-(oxetan-3-yl)piperazine

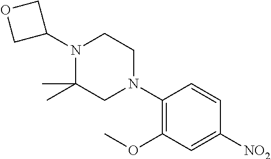 [SMILES: COc1cc([N+](=O)[O-])ccc1N1CCN(C2COC2)C(C)(C)C1]

To a solution of 1-(2-methoxy-4-nitrophenyl)-3,3-dimethylpiperazine (750 mg, 2.8 mmol), sodium cyanoborohydride (530 mg, 8.5 mmol), zinc chloride (580 mg, 4.2 mmol) in methanol (56 mL) was added oxetan-3-one (1.8 mL, 28.2 mmol). The mixture stirred at 75° C. for 16 h. After this time, the reaction mixture was concentrated. To the residue was added methylene chloride (25 mL) and 10% aqueous potassium carbonate (25 mL). The aqueous layer was extracted with methylene chloride (2×15 mL). The combined organic layers were washed with brine (30 mL), dried over sodium sulfate and concentrated under reduced pressure. The resulting residue was purified by column chromatography eluting with a gradient of 100% methylene chloride—100% 60:35:5 methylene chloride:diethylether:methanol to afford 4-(2-methoxy-4-nitrophenyl)-2,2-dimethyl-1-(oxetan-3-yl)piperazine.

### Example 131

### Preparation of 4-(3,3-dimethyl-4-(oxetan-3-yl)piperazin-1-yl)-3-methoxyaniline

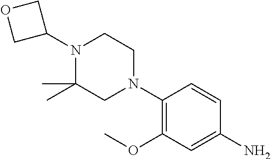 [SMILES: COc1cc(N)ccc1N1CCN(C2COC2)C(C)(C)C1]

To a suspension of 4-(2-methoxy-4-nitrophenyl)-2,2-dimethyl-1-(oxetan-3-yl)piperazine (350 mg, 1.1 mmol) in ethanol (70 mL) was added 10% Pd/C (50% wet, 70 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, and washed with ethanol. The filtrate was concentrated in vacuo to give 4-(3,3-dimethyl-4-(oxetan-3-yl)piperazin-1-yl)-3-methoxyaniline.

### Example 132

### Preparation of 6-bromo-N-(4-(3,3-dimethyl-4-(oxetan-3-yl)piperazin-1-yl)-3-methoxyphenyl)imidazo[1,2-a]pyrazin-8-amine

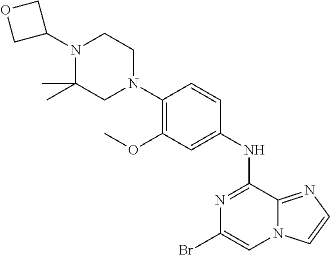 [SMILES: COc1cc(Nc2nc(Br)cn3ccnc23)ccc1N1CCN(C2COC2)C(C)(C)C1]

This compound was prepared by direct analogy to procedure C in Example 3, using 4-(3,3-dimethyl-4-(oxetan-3-yl)piperazin-1-yl)-3-methoxyaniline (320 mg, 1.1 mmol) as a starting material to afford 6-bromo-N-(4-(3,3-dimethyl-4-(oxetan-3-yl)piperazin-1-yl)-3-methoxyphenyl)imidazo[1,2-a]pyrazin-8-amine.

### Example 133

### Preparation of 6-(2,3-dihydro-1H-pyrido[2,3-b][1,4]oxazin-7-yl)-N-(4-(3,3-dimethyl-4-(oxetan-3-yl)piperazin-1-yl)-3-methoxyphenyl)imidazo[1,2-a]pyrazin-8-amine

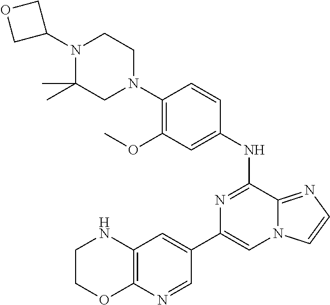 [SMILES: COc1cc(Nc2nc(-c3cnc4c(c3)NCCO4)cn3ccnc23)ccc1N1CCN(C2COC2)C(C)(C)C1]

This compound was synthesized according to procedure F in Example 5b above.

¹H NMR (200 MHz, d₆-DMSO): δ 9.45 (s, 1H), 8.40 (s, 1H), 7.99 (d, 1H), 7.94 (d, 1H), 7.89 (d, 1H), 7.65 (dd, 1H), 7.60 (d, 1H), 7.44 (d, 1H), 6.82 (d, 1H), 6.31 (brs, 1H), 4.59 (dd, 2H), 4.46 (dd, 2H), 4.39 (dd, 2H), 4.14 (m, 1H), 3.79 (s, 3H), 3.29 (m, 2H), 2.93 (m, 2H), 2.63 (m, 4H), 0.99 (s, 6H). MS (ESI+) m/z 543.6 (M+H).

### Example 134

### Preparation of tert-butyl 4-(4-(6-bromoimidazo[1,2-a]pyrazin-8-ylamino)-2-methoxyphenyl)piperazine-1-carboxylate

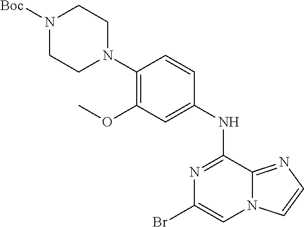 [SMILES: COc1cc(Nc2nc(Br)cn3ccnc23)ccc1N1CCN(C(=O)OC(C)(C)C)CC1]

This compound was prepared by direct analogy to procedure C in Example 3, using tert-butyl 4-(4-amino-2-methoxyphenyl)piperazine-1-carboxylate (3.2 g, 11.5 mmol) as a starting material to afford tert-butyl 4-(4-(6-bromoimidazo[1,2-a]pyrazin-8-ylamino)-2-methoxyphenyl)piperazine-1-carboxylate.

### Example 135

### Preparation of tert-butyl 4-(4-(6-(2,3-dihydro-1H-pyrido[2,3-b][1,4]oxazin-7-yl)imidazo[1,2-a]pyrazin-8-ylamino)-2-methoxyphenyl)piperazine-1-carboxylate

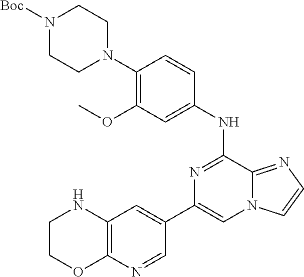 [SMILES: COc1cc(Nc2nc(-c3cnc4c(c3)NCCO4)cn3ccnc23)ccc1N1CCN(C(=O)OC(C)(C)C)CC1]

This compound was prepared by direct analogy to procedure G in Example 6 above, using tert-butyl-4-(4-(6-bromoimidazo[1,2-a]pyrazin-8-ylamino)-2-methoxyphenyl)piperazine-1-carboxylate (300 mg, 0.6 mmol) as a starting material to afford tert-butyl 4-(4-(6-(2,3-dihydro-1H-pyrido[2,3-b][1,4]oxazin-7-yl)imidazo[1,2-a]pyrazin-8-ylamino)-2-methoxyphenyl)piperazine-1-carboxylate.

### Example 136

### Preparation of 6-(2,3-dihydro-1H-pyrido[2,3-b][1,4]oxazin-7-yl)-N-(3-methoxy-4-(piperazin-1-yl)phenyl)imidazo[1,2-a]pyrazin-8-amine

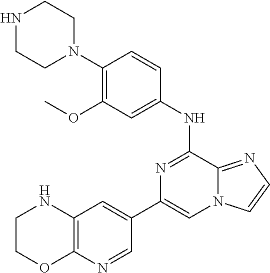 [SMILES: COc1cc(Nc2nc(-c3cnc4c(c3)NCCO4)cn3ccnc23)ccc1N1CCNCC1]

To a solution of tert-butyl 4-(4-(6-(2,3-dihydro-1H-pyrido[2,3-b][1,4]oxazin-7-yl)imidazo[1,2-a]pyrazin-8-ylamino)-2-methoxyphenyl)piperazine-1-carboxylate (226 mg, 0.4 mmol) in methylene chloride (10 mL) was added trifluoroacetic acid (0.8 mL, 10 mmol). The mixture stirred at room temperature for 16 hours. After this time, saturated aqueous sodium bicarbonate (10 mL) was carefully added. The aqueous layer was separated and extracted with methylene chloride (2×10 mL). The combined organic extracts were washed with brine (10 mL), dried over sodium sulfate and concentration in vacuo. The resulting residue was purified by column chromatography eluting with a gradient of 100% methylene chloride—100% 9:1 methylene chloride:methanol to afford 6-(2,3-dihydro-1H-pyrido[2,3-b][1,4]oxazin-7-yl)-N-(3-methoxy-4-(piperazin-1-yl)phenyl)imidazo[1,2-a]pyrazin-8-amine.

### Example 137

### Preparation of (S)-1-(4-(4-((6-(2,3-dihydro-1H-pyrido[2,3-b][1,4]oxazin-7-yl)imidazo[1,2-a]pyrazin-8-yl)amino)-2-methoxyphenyl)piperazin-1-yl)-2-hydroxypropan-1-one

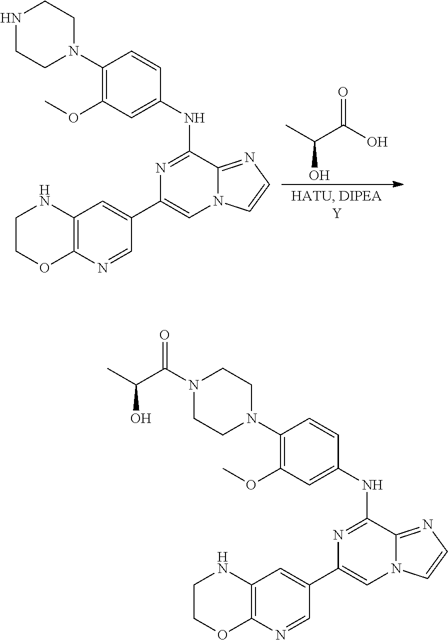

To a solution of 6-(2,3-dihydro-1H-pyrido[2,3-b][1,4]oxazin-7-yl)-N-(3-methoxy-4-(piperazin-1-yl)phenyl)imidazo[1,2-a]pyrazin-8-amine (107 mg, 0.23 mmol), L-(+)-lactic acid (21 mg, 0.23 mmol) and DIPEA (120 mL, 0.7 mmol) in dimethylformamide (1.2 mL) was added HATU (89 mg, 0.23 mmol). The mixture stirred at room temperature for 2 hours. After this time, 9:1 methylene chloride:methanol (5 mL) and 10% aqueous potassium carbonate (5 mL). The aqueous layer was extracted with methylene chloride (2×5 mL). The combined organic layers were washed with brine (10 mL), dried over sodium sulfate and concentrated under reduced pressure. The resulting residue was purified by column chromatography using a Biotage KPNH 12+M column eluting with a gradient of 9:1 hexanes:ethyl acetate—100% ethyl acetate to afford (S)-1-(4-(4-(6-(2,3-dihydro-1H-pyrido[2,3-b][1,4]oxazin-7-yl)imidazo[1,2-a]pyrazin-8-ylamino)-2-methoxyphenyl)piperazin-1-yl)-2-hydroxypropan-1-one.

¹H NMR (200 MHz, d₆-DMSO): δ 9.50 (s, 1H), 8.41 (s, 1H), 7.99 (d, 1H), 7.94 (d, 1H), 7.92 (d, 1H), 7.68 (dd, 1H), 7.60 (d, 1H), 7.44 (d, 1H), 6.90 (d, 1H), 6.20 (brs, 1H), 4.89 (d, 1H), 4.46 (ddd, 1H), 4.39 (dd, 2H), 3.81 (s, 3H), 3.71-349 (m, 4H), 3.33-3.25 (m, 2H), 2.98-2.82 (m, 4H), 1.20 (d, 3H). MS (ESI+) m/z 531.5 (M+H).

### Example 138

### Preparation of 4-(2-fluoro-4-nitrophenyl)-3,3-dimethylmorpholine

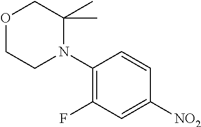 [SMILES: CC1(C)COCCN1c1ccc([N+](=O)[O-])cc1F]

A mixture of 3,4-difluoronitrobenzene (0.46 mL, 4.1 mmol), 3,3-dimethylmorpholine hydrochloric acid (1.3 g, 8.2 mmol) and potassium carbonate (1.6 g, 11.3 mmol) in dimethylamine (4.1 mL) was stirred at 125° C. for 16 hours and then at 150° C. for 24 hours. The reaction was cooled to room temperature, diluted with methylene chloride (10 mL) and water (10 mL). The aqueous layer was separated and extracted with methylene chloride (2×10 mL). The combined organic extracts were washed with water (4×10 mL) and brine (10 mL), dried over sodium sulfate and concentrated in vacuo. The resulting residue was purified by column chromatography eluting with a gradient of 100% hexanes—100% 3:1 hexanes:ethyl acetate to afford 4-(2-fluoro-4-nitrophenyl)-3,3-dimethylmorpholine.

### Example 139

### Preparation of 4-(2-methoxy-4-nitrophenyl)-3,3-dimethylmorpholine

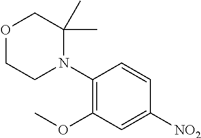 [SMILES: COc1cc([N+](=O)[O-])ccc1N1CCOCC1(C)C]

A mixture of 4-(2-fluoro-4-nitrophenyl)-3,3-dimethylmorpholine (690 mg, 2.7 mmol) and sodium methoxide (25 wt. % in methanol, 3.7 mL, 16.3 mmol) in dimethylformamide (5.5 mL) was stirred at 55° C. for 16 h. The reaction was cooled to room temperature, diluted with methylene chloride (10 mL) and water (10 mL). The aqueous layer was separated and extracted with methylene chloride (2×10 mL). The combined organic extracts were washed with water (2×10 mL) and brine (10 mL), dried over sodium sulfate and concentrated under reduced pressure and diethylether (10 mL) was added. The resulting residue was purified by column chromatography column eluting with a gradient of 100% hexanes—100% 4:1 hexanes:ethyl acetate to afford 4-(2-methoxy-4-nitrophenyl)-3,3-dimethylmorpholine.

### Example 140

### Preparation of 4-(3,3-dimethylmorpholinyl)-3-methoxyaniline

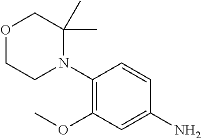 [SMILES: COc1cc(N)ccc1N1CCOCC1(C)C]

To a suspension of 4-(2-methoxy-4-nitrophenyl)-3,3-dimethylmorpholine (450 mg, 1.7 mmol) in ethanol (50 mL) was added 10% Pd/C (50% wet, 100 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, washed with ethanol and concentrated in vacuo. The resulting residue was purified by column chromatography eluting with a gradient of 100% methylene chloride—100% 60:35:5 methylene chloride:diethylether:methanol to afford 4-(3,3-dimethylmorpholinyl)-3-methoxyaniline.

### Example 141

### Preparation of 6-bromo-N-(4-(3,3-dimethylmorpholinyl)-3-methoxyphenyl)imidazo[1,2-a]pyrazin-8-amine

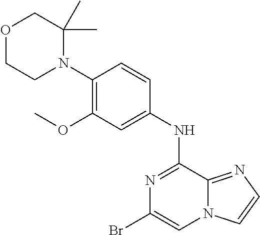 [SMILES: COc1cc(Nc2nc(Br)cn3ccnc23)ccc1N1CCOCC1(C)C]

This compound was prepared by direct analogy to procedure C In Example 3 above, using instead 4-(3,3-dimethylmorpholinyl)-3-methoxyaniline (205 mg, 0.9 mmol) as a starting material to afford 6-bromo-N-(4-(3,3-dimethylmorpholinyl)-3-methoxyphenyl)imidazo[1,2-a]pyrazin-8-amine.

### Example 142

### Preparation of 6-(2,3-dihydro-1H-pyrido[2,3-b][1,4]oxazin-7-yl)-N-(4-(3,3-dimethylmorpholinyl)-3-methoxyphenyl)imidazo[1,2-a]pyrazin-8-amine

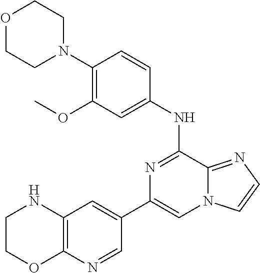 [SMILES: COc1cc(Nc2nc(-c3cnc4c(c3)NCCO4)cn3ccnc23)ccc1N1CCOCC1]

This compound was synthesized according to procedure F in Example 5b above.

¹H NMR (200 MHz, d₆-DMSO): δ 9.55 (s, 1H), 8.42 (s, 1H), 8.06-7.88 (m, 3H), 7.73-7.57 (m, 2H), 7.43 (s, 1H), 7.12 (d, 1H), 6.21 (brs, 1H), 4.37-4.21 (m, 2H), 3.84-3.57 (m, 5H), 3.42-3.20 (m, 5H), 3.11-2.85 (m, 2H), 0.95 (s, 6H). MS (ESI+) m/z 488.5 (M+H).

### Example 143

### Preparation of benzyl 4-benzyl 1-tert-butyl 2-(hydroxymethyl)piperazine-1,4-dicarboxylate

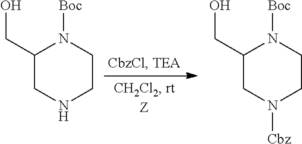 [SMILES: CC(C)(C)OC(=O)N1CCNCC1CO ; CC(C)(C)OC(=O)N1CCN(C(=O)OCc2ccccc2)CC1CO]

To a stirred mixture of tert-butyl 2-(hydroxymethyl)piperazine-1-carboxylate (14 g, 64.8 mmol) and triethylamine (21.7 mL, 155.5 mmol) in methylene chloride (574 ml), CbzCl (22.4 mL, 149.0 mmol) was added dropwise at 0° C. under nitrogen gas (N₂). The mixture was stirred at room temperature overnight. The reaction mixture was washed with water, dried over sodium sulfate and purified by silica gel on chromatography column (25-50% ethyl acetate/PE) to get 4-benzyl 1-tert-butyl 2-(hydroxymethyl)piperazine-1,4-dicarboxylate.

### Example 144

### Preparation of benzyl 3-(hydroxymethyl)piperazine-1-carboxylate

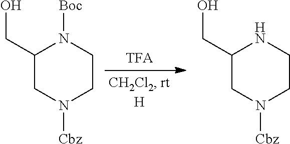 [SMILES: CC(C)(C)OC(=O)N1CCN(C(=O)OCc2ccccc2)CC1CO ; O=C(OCc1ccccc1)N1CCNC(CO)C1]

To a solution of 4-benzyl 1-tert-butyl 2-(hydroxymethyl)piperazine-1,4-dicarboxylate (20.4 g, 58.3 mmol) in methylene chloride (130 mL) was added trifluoroacetic acid (65 mL). The reaction mixture was stirred at room temperature under nitrogen gas for 3 hours. The solution was removed to give benzyl 3-(hydroxymethyl)piperazine-1-carboxylate which was used for next step without further purification.

### Example 145

### Preparation of benzyl 3-(hydroxymethyl)-4-methylpiperazine-1-carboxylate

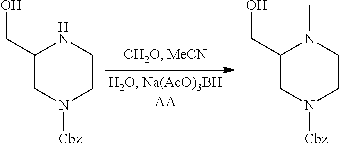 [SMILES: O=C(OCc1ccccc1)N1CCNC(CO)C1 ; CN1CCN(C(=O)OCc2ccccc2)CC1CO]

To a solution of benzyl 3-(hydroxymethyl)piperazine-1-carboxylate (17 g, 68 mmol) in MeCN (180 mL) and water (38 mL) was added CH₂O (16.6 g, 205 mmol, 37% in H₂O) followed by Na(OAc)₃BH (28.8 g, 141 mmol). The reaction mixture was stirred for 2 h at room temperature. The reaction mixture was then basified with sodium carbonate (aq), extracted with methylene chloride (1 L) and methanol (100 mL), and washed with saturated sodium bicarbonate. The organic layer was separated, dried over sodium sulfate, and concentrated in vacuo. The crude product was purified by silica gel chromatography column (0-10% methanol/methylene chloride) to afford benzyl 3-(hydroxymethyl)-4-methylpiperazine-1-carboxylate.

### Example 146

### Preparation of benzyl 3-(fluoromethyl)-4-methylpiperazine-1-carboxylate

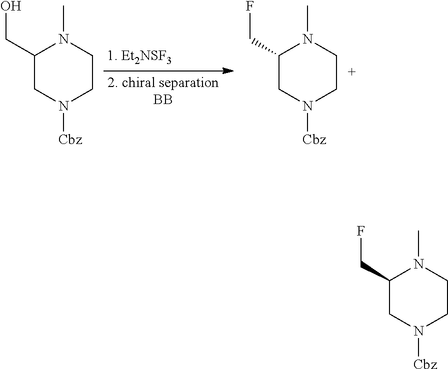 [SMILES: CN1CCN(C(=O)OCc2ccccc2)CC1CO ; "BB" ; CN1CCN(C(=O)OCc2ccccc2)C[C@@H]1CF ; CN1CCN(C(=O)OCc2ccccc2)C[C@H]1CF]

To a solution of benzyl 3-(hydroxymethyl)-4-methylpiperazine-1-carboxylate (1 g, 3.79 mmol) in dry methylene chloride (70 mL) was added dropwise a solution of DAST (3.66 g, 22.7 mmol) in dry methylene chloride (40 mL) at −65° C. under nitrogen gas. The solution was then allowed to warm slowly to room temperature and was stirred for 15 hours. On workup, the reaction solution was cooled to 5° C. and cold water was added dropwise while the temperature was maintained at 10° C. The pH of the aqueous phase was adjusted to 9.0 with aqueous sodium hydroxide. The organic phase was separated and the aqueous phase was extracted repeatedly with methylene chloride. The combined organic phases were washed with water, dried over sodium sulfate and concentrated. The crude product was purified by silica gel on chromatography column (0-10% methanol/methylene chloride) to afford benzyl 3-(fluoromethyl)-4-methylpiperazine-1-carboxylate. The enantiomers were separated using Thar 80 preparative SFC using ChiralPak AD-H column.

### Example 147

### Preparation of (R or S)-2-(fluoromethyl)-1-methylpiperazine

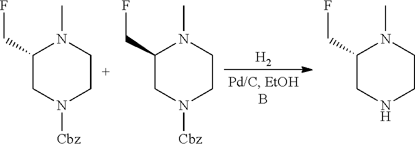

To a suspension of (R or S)-benzyl 3-(fluoromethyl)-4-methylpiperazine-1-carboxylate (824 mg, 3.1 mmol) in iso-propanol (100 mL) was added 10% Pd/C (50% wet, 165 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, washed with iso-propanol and concentrated in vacuo to afford (R or S)-2-(fluoromethyl)-1-methylpiperazine.

### Example 148

### Preparation of (R or S)-2-(fluoromethyl)-4-(2-methoxy-4-nitrophenyl)-1-methylpiperazine

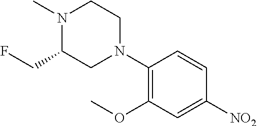 [SMILES: COc1cc([N+](=O)[O-])ccc1N1CCN(C)[C@@H](CF)C1]

A mixture of 1-fluoro-2-methoxy-4-nitrobenzene (410 mg, 2.4 mmol), (R or S)-2-(fluoromethyl)-1-methylpiperazine (410 mg, 1.3 mmol) and potassium carbonate (580 mg, 4.2 mmol) in dimethylformamide (10 mL) was stirred at 100° C. for 16 h. The reaction was cooled to room temperature, diluted with methylene chloride (10 mL) and water (10 mL). The aqueous layer was separated and extracted with methylene chloride (2×10 mL). The combined organic extracts were washed with water (4×10 mL) and brine (10 mL) and dried over sodium sulfate. After concentration, the resulting residue was purified by column chromatography eluting with a gradient of 100% methylene chloride—100% 75:17:8 methylene chloride:diethylether:methanol to afford (R or S)-2-(fluoromethyl)-4-(2-methoxy-4-nitrophenyl)-1-methylpiperazine.

### Example 149

### Preparation of (R or S)-4-(3-(fluoromethyl)-4-methylpiperazin-1-yl)-3-methoxyaniline

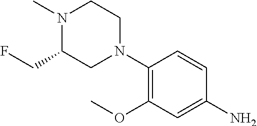 [SMILES: COc1cc(N)ccc1N1CCN(C)[C@@H](CF)C1]

To a suspension of (R or S)-2-(fluoromethyl)-4-(2-methoxy-4-nitrophenyl)-1-methylpiperazine (160 mg, 0.56 mmol) in ethanol (25 mL) was added 10% Pd/C (50% wet, 40 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, washed with ethanol and concentrated in vacuo. The filtrate was concentrated in vacuo to give (R or S)-4-(3-(fluoromethyl)-4-methylpiperazin-1-yl)-3-methoxyaniline.

### Example 150

### Preparation of (R or S)-6-bromo-N-(4-(3-(fluoromethyl)-4-methylpiperazin-1-yl)-3-methoxyphenyl)imidazo[1,2-a]pyrazin-8-amine

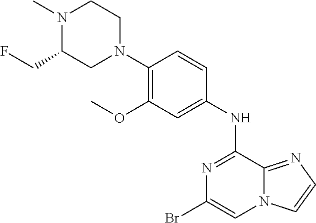 [SMILES: COc1cc(Nc2nc(Br)cn3ccnc23)ccc1N1CCN(C)[C@@H](CF)C1]

A mixture of (R or S)-4-(3-(fluoromethyl)-4-methylpiperazin-1-yl)-3-methoxyaniline (143 mg, 0.56 mmol) 6,8-dibromoimidazo[1,2-a]pyrazine (226 mg, 0.82 mmol) and CSA (190 mg, 0.82 mmol) in isopropanol (5.5 mL) was heated at reflux for 16 h. After being cooled down to room temperature, methylene chloride (10 mL) and saturated aqueous sodium bicarbonate (10 mL) were added. The aqueous layer was separated and extracted with methylene chloride (2×10 mL). The combined organic extracts were washed with brine (10 mL), dried over sodium sulfate and concentrated in vacuo. The resulting residue was purified by column chromatography eluting with a gradient of 100% methylene chloride—100% 60:35:5 methylene chloride:diethylether:methanol to afford (R or S)-6-bromo-N-(4-(3-(fluoromethyl)-4-methylpiperazin-1-yl)-3-methoxyphenyl)imidazo[1,2-a]pyrazin-8-amine.

### Example 151

### Preparation of (R or S)-6-(2,3-dihydro-1H-pyrido[2,3-b][1,4]oxazin-7-yl)-N-(4-(3-(fluoromethyl)-4-methylpiperazin-1-yl)-3-methoxyphenyl)imidazo[1,2-a]pyrazin-8-amine

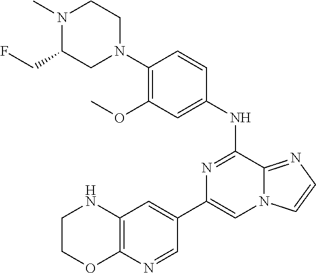 [SMILES: COc1cc(Nc2nc(-c3cnc4c(c3)NCCO4)cn3ccnc23)ccc1N1CCN(C)[C@@H](CF)C1]

This compound was synthesized according to procedure F in Example 5b above.

¹H NMR (200 MHz, d₆-DMSO): δ 9.48 (s, 1H), 8.41 (s, 1H), 8.01-7.92 (m, 2H), 7.88 (m, 1H), 7.69 (dd, 1H), 7.60 (s, 1H), 7.45 (d, 1H), 6.89 (d, 1H), 6.21 (brs, 1H), 4.69-4.38 (m, 2H), 4.32-4.25 (m, 2H), 3.80 (s, 3H), 3.31-3.10 (m, 3H), 2.81-2.63 (m, 2H), 2.62-2.48 (m, 2H), 2.47-2.20 (m, 6H). MS (ESI+) m/z 505.5 (M+H).

### Example 152

### Preparation of (S)-2-hydroxy-1-(4-(2-methoxy-4-nitrophenyl)piperazin-1-yl)propan-1-one

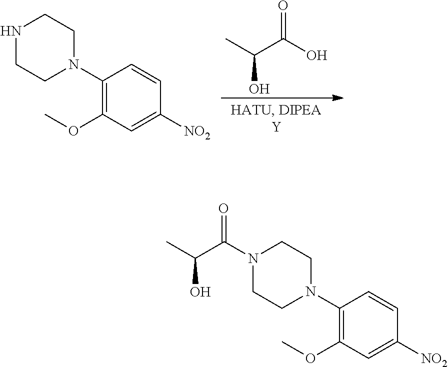

To a solution of 1-(2-methoxy-4-nitrophenyl)piperazine (1.9 g, 7.9 mmol), L-(+)-lactic acid (780 mg, 8.7 mmol) and DIPEA (4.1 mL, 23.6 mmol) in dimethylformamide (39 mL) was added HATU (3.3 g, 8.7 mmol). The mixture stirred at room temperature for 2 hours. After this time, ethyl acetate (50 mL) and water (50 mL) were added. The aqueous layer was extracted with ethyl acetate (2×20 mL). The combined organic layers were washed with water (4×50 mL) and brine (50 mL), dried over sodium sulfate and concentrated under reduced pressure. The resulting residue was purified by column chromatography eluting with a gradient of 1:1 hexanes:ethyl acetate—100% ethyl acetate to afford (S)-2-hydroxy-1-(4-(2-methoxy-4-nitrophenyl)piperazin-1-yl)propan-1-one.

### Example 153

### Preparation of (S)-1-(4-(2-methoxy-4-nitrophenyl)piperazin-1-yl)propan-2-ol

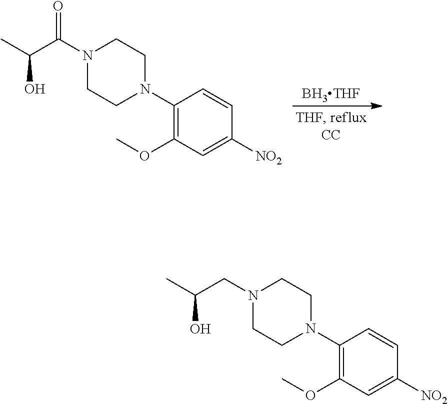 [SMILES: COc1cc([N+](=O)[O-])ccc1N1CCN(C(=O)[C@H](C)O)CC1 ; COc1cc([N+](=O)[O-])ccc1N1CCN(C[C@H](C)O)CC1 ; "BH3·THF"]

To a solution of (S)-2-hydroxy-1-(4-(2-methoxy-4-nitrophenyl)piperazin-1-yl)propan-1-one (3.4 g, 11.1 mmol) in tetrahydrofuran (25 mL) was added BH₃.THF (33 mL, 1 M in THF) dropwise. The mixture was then heated to reflux and stirred for 1 h. After this time, the mixture was cooled to 0° C. with an ice bath and methanol (10 mL) was carefully added. After stirring for 15 min, the mixture was concentrated in vacuo. To the resulting residue was added methanol (30 mL) and 1M aqueous HCl (30 mL). The mixture was then heated to reflux and stirred for 30 min. After this time, the mixture was cooled to room temperature and 1M aqueous NaOH was added until the pH >7. The mixture was diluted with methylene chloride (60 mL) and water (30 mL). The aqueous layer was separated and extracted with methylene chloride (2×30 mL). The combined organic extracts were washed with brine (30 mL), dried over sodium sulfate and concentrated in vacuo. The resulting residue was purified by column chromatography eluting with a gradient of 100% methylene chloride—100% 75:18:7 methylene chloride:diethylether:methanol to afford (S)-1-(4-(2-methoxy-4-nitrophenyl)piperazin-1-yl)propan-2-ol.

### Example 154

### Preparation of (S)-1-(4-(4-amino-2-methoxyphenyl)piperazin-1-yl)propapan-2-ol

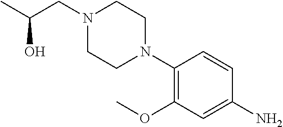 [SMILES: COc1cc(N)ccc1N1CCN(C[C@H](C)O)CC1]

To a suspension of (S)-1-(4-(2-methoxy-4-nitrophenyl)piperazin-1-yl)propan-2-ol (2.1 g, 7.2 mmol) in ethanol (150 mL) was added 10% Pd/C (50% wet, 400 mg dry weight) in a 500-mL Parr hydrogenation bottle. The bottle was evacuated, charged with hydrogen gas to a pressure of 50 psi and shaken at room temperature for 2 hours on a Parr hydrogenation apparatus. The reaction mixture was filtered, washed with ethanol and concentrated in vacuo. The filtrate was concentrated in vacuo to afford (S)-1-(4-(4-amino-2-methoxyphenyl)piperazin-1-yl)propan-2-ol.

### Example 155

### Preparation of (S)-1-(4-(4-(6-bromoimidazo[1,2-a]pyrazin-8-ylamino)-2-methoxyphenyl)piperazin-1-yl)propan-2-ol

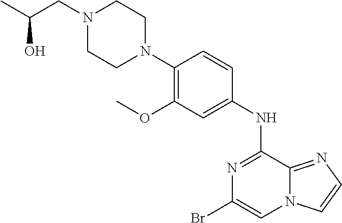 [SMILES: COc1cc(Nc2nc(Br)cn3ccnc23)ccc1N1CCN(C[C@H](C)O)CC1]

A mixture of (S)-1-(4-(4-amino-2-methoxyphenyl)piperazin-1-yl)propan-2-ol (1.9 g, 7.1 mmol) 6,8-dibromoimidazo[1,2-a]pyrazine (2.0 g, 7.1 mmol) and CSA (1.7 g, 7.1 mmol) in isopropanol (47 mL) was heated at reflux for 16 h. After being cooled down to room temperature, methylene chloride (50 mL) and saturated aqueous sodium bicarbonate (50 mL) were added. The aqueous layer was separated and extracted with methylene chloride (2×40 mL). The combined organic extracts were washed with brine (50 mL) and dried over sodium sulfate. Upon concentrating in vacuo to near dryness the crude product crashed out. The solid was filtered and washed with diethylether to afford (S)-1-(4-(4-(6-bromoimidazo[1,2-a]pyrazin-8-ylamino)-2-methoxyphenyl)piperazin-1-yl)propan-2-ol.

### Example 156

### Preparation of (S)-1-(4-(4-((6-(2,3-dihydro-1H-pyrido[2,3-b][1,4]oxazin-7-yl)imidazo[1,2-a]pyrazin-8-yl)amino)-2-methoxyphenyl)piperazin-1-yl)propan-2-ol

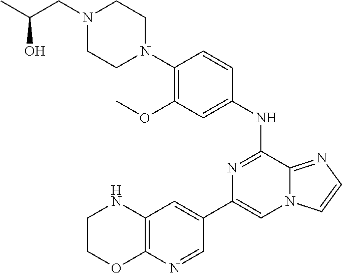 [SMILES: COc1cc(Nc2nc(-c3cnc4c(c3)NCCO4)cn3ccnc23)ccc1N1CCN(C[C@H](C)O)CC1]

This compound was synthesized according to procedure F in Example 5b above.

¹H NMR (200 MHz, d₆-DMSO): δ 9.46 (s, 1H), 8.40 (s, 1H), 7.99 (d, 1H), 7.95 (m, 1H), 7.89 (d, 1H), 7.65 (dd, 1H), 7.60 (s, 1H), 7.45 (d, 1H), 6.89 (d, 1H), 6.20 (brs, 1H), 4.39-4.23 (m, 3H), 3.82-3.67 (m, 4H), 3.00-2.82 (m, 4H), 2.69-2.51 (m, 4H), 2.47-2.13 (m, 4H), 1.05 (m, 3H). MS (ESI+) m/z 517.6 (M+H).

### Example 157

### Preparation of tert-butyl 4-(4-nitrophenyl)-5,6-dihydropyridine-1(2H)-carboxylate

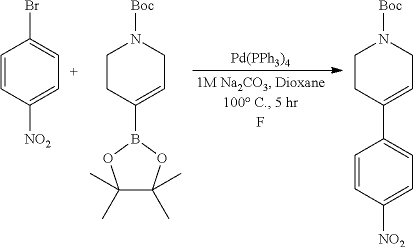

A 100 mL sealed tube with a magnetic stirrer was charged with 1-bromo-4-nitrobenzene (1.2 g, 5.9 mmol), tert-butyl 4-(4,4,5,5-tetramethyl-1,3,2-dioxaborolan-2-yl)-5,6-dihydropyridine-1(2H)-carboxylate (1.92 g, 6.2 mmol), 20 mL 1N sodium carbonate and 50 mL, and dioxane 50 mL. After degassed for 5 minutes, palladium tetrakis (0.29 g, 0.25 mmol) was added. The reaction mixture was heated at 100° C. for 5 hours. After this time, the mixture was cooled to room temperature, partitioned between ethyl acetate (50 mL) and water (30 mL). The organic phase was separated, and the aqueous layer was extracted with ethyl acetate (30 mL×3). The combined organic phases were washed with brine, dried over sodium sulfate and concentrated under reduced pressure. The residue was purified by flash column chromatography (silica, 0-80% ethyl acetate) to give the desired product as a pink-yellow solid.

### Example 158

### Preparation of tert-butyl 4-(4-aminophenyl)piperidine-1-carboxylate

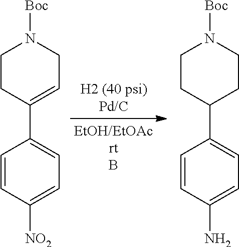 [SMILES: CC(C)(C)OC(=O)N1CC=C(c2ccc([N+](=O)[O-])cc2)CC1 ; CC(C)(C)OC(=O)N1CCC(c2ccc(N)cc2)CC1]

A Parr reactor bottle was purged with nitrogen and charged with 10% palladium on carbon (600 mg, 40% weight) and a solution of tert-butyl 4-(4-nitrophenyl)-5,6-dihydropyridine-1(2H)-carboxylate (1.5 g) in ethanol (50 mL) and Ethyl Acetate (20 mL). The bottle was attached to a Parr hydrogenator, evacuated, charged with hydrogen gas to a pressure of 40 psi and shaken for 3 h. After this time, the hydrogen was evacuated, and the reaction mixture was filtered through a pad of Celite 521. The filter cake was washed with ethanol (2×25 mL), and the combined filtrates were concentrated to dryness under reduced pressure to afford tert-butyl 4-(4-aminophenyl)piperidine-1-carboxylate as a brown solid.

### Example 159

### Preparation of tert-butyl 4-(4-(6-bromoimidazo[1,2-a]pyrazin-8-ylamino)phenyl)piperidine-1-carboxylate

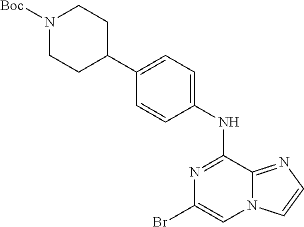 [SMILES: CC(C)(C)OC(=O)N1CCC(c2ccc(Nc3nc(Br)cn4ccnc34)cc2)CC1]

A mixture of tert-butyl 4-(4-aminophenyl)piperidine-1-carboxylate (1.18 g, 4.3 mmol), 6,8 dibromoimidazo[1,2-a]pyrazine (1.0 g, 3.6 mmol) and N,N-diisopropylethylamine (2.5 mL, 14.3 mmol) in isopropanol (18 mL) was heated at 110° C. for 16 h. After being cooled down to room temperature, the solid was filtered, washed with isopropanol (2×), and dried to give the title compound as a yellow solid.

### Example 160

### Preparation of 6-bromo-N-(4-(piperidin-4-yl)phenyl)imidazo[1,2-a]pyrazin-8-amine

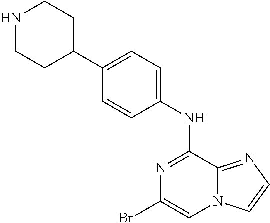 [SMILES: Brc1cn2ccnc2c(Nc2ccc(C3CCNCC3)cc2)n1]

Tert-butyl 4-(4-(6-bromoimidazo[1,2-a]pyrazin-8-ylamino)phenyl)piperidine-1-carboxylate (1.6 g, 3.3 mmol) was dissolved in methylene chloride (30 mL). Trifluoroacetic acid (5.2 mL, 68 mmol) was added, and the mixture was stirred at room temperature for 4 h. After this time, the mixture was basified by saturated sodium bicarbonate, and the aqueous layer was extracted with methylene chloride (2×20 mL). The combined organic layers were washed with brine and dried over sodium sulfate. The drying agent was removed by filtration. The filtrate was concentrated, and the resulting residue was used without purification in the next step.

### Example 161

### Preparation of 6-bromo-N-(4-(1-(oxetan-3-yl)piperidin-4-yl)phenyl)imidazo[1,2-a]pyrazin-8-amine

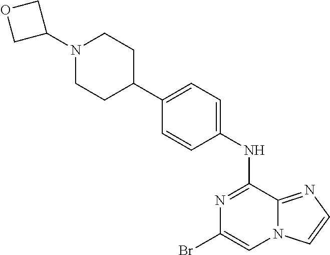 [SMILES: Brc1cn2ccnc2c(Nc2ccc(C3CCN(C4COC4)CC3)cc2)n1]

A mixture of sodium cyanoborohydride (340 mg, 5.4 mmol) and zinc chloride (373 mg, 2.7 mmol) in methanol (15 mL) was stir at room temperature for 15 min to generate a colorless clear solution. A 50-mL sealed tube with a magnetic stirrer was purged with nitrogen and charged with 6-bromo-N-(4-(piperidin-4-yl)phenyl)imidazo[1,2-a]pyrazin-8-amine (670 mg, 1.8 mmol), oxetan-3-one (648 mg, 9 mmol) and methanol (15 mL). The above clear solution of sodium cyanoborohydride and zinc chloride was added, and the reaction was stirred at room temperature for 12 hours. After this time, the reaction mixture was concentrated, and the residue was partitioned between methylene chloride/methanol (4:1) and 10% aqueous potassium carbonate. The aqueous layer was extracted with methylene chloride/methanol (4:1) for three times. The combined organic layers were washed with brine, dried over sodium sulfate and concentrated under reduced pressure to afford the titled compound as a light yellow solid.

### Example 162

### Preparation of 6-(2,3-dihydro-1H-pyrido[2,3-b][1,4]oxazin-7-yl)-N-(4-(1-(oxetan-3-yl)piperidin-4-yl)phenyl)imidazo[1,2-a]pyrazin-8-amine

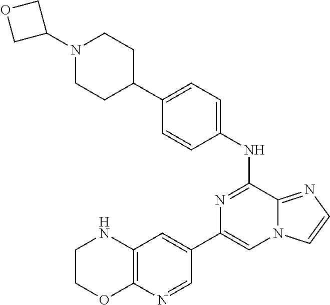 [SMILES: c1cn2cc(-c3cnc4c(c3)NCCO4)nc(Nc3ccc(C4CCN(C5COC5)CC4)cc3)c2n1]

This compound was synthesized according to procedure F in Example 5b above.

[M+H]=484.5 g/mol; NMR (δ, ppm): 9.58 (s, 1H), 8.42 (s, 1H), 8.02 (d, 2H), 7.88 (d, 2H), 7.60 (s, 1H), 7.42 (d, 1H), 7.21 (d, 2H), 6.22 (s, 1H), 4.52 (t, 2H), 4.42 (t, 2H), 4.30 (br, 2H), 2.80 (br, 2H), 2.50 (m, 2H), 1.78 (m, 7H).

### Example 163

### Preparation of 1-(2-methoxyethyl)-4-(4-nitrophenyl)piperazine

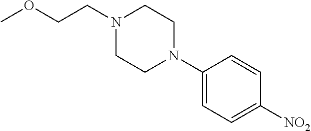 [SMILES: COCCN1CCN(c2ccc([N+](=O)[O-])cc2)CC1]

In a sealed tube, a mixture of 1-fluoro-4-nitrobenzene (3.0 g, 21 mmol), 1-(2-methoxyethyl)piperazine (3.0 g, 21 mmol) and potassium carbonate (3.8 g, 27 mmol) in dimethylformamide (15 mL) was stirred at 100° C. for 16 h. After this time, the mixture was cooled to room temperature, partitioned between methylene chloride (30 mL) and water (50 mL). The organic phase was separated, and the aqueous layer was extracted with methylene chloride (30 mL×3). The combined organic phases were washed with brine, dried over sodium sulfate and concentrated under reduced pressure to afford the titled compound as a yellow-orange solid.

### Example 164

### Preparation of 4-(4-(2-methoxyethyl)piperazin-1-yl)aniline

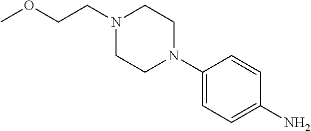 [SMILES: COCCN1CCN(c2ccc(N)cc2)CC1]

A Parr reactor bottle was purged with nitrogen and charged with 10% palladium on carbon (370 mg, 10% weight) and a solution of 1-(2-methoxyethyl)-4-(4-nitrophenyl)piperazine (3.7 g, 14 mmol) in ethanol (25 mL) and Ethyl Acetate (20 mL). The bottle was attached to a Parr hydrogenator, evacuated, charged with hydrogen gas to a pressure of 40 psi and shaken for 2 h. After this time, the hydrogen was evacuated, and the reaction mixture was filtered through a pad of Celite 521. The filter cake was washed with ethanol (2×25 mL), and the combined filtrates were concentrated to dryness under reduced pressure to afford the titled compound as a brown solid.

### Example 165

### Preparation of 6-bromo-N-(4-(4-(2-methoxyethyl)piperazin-1-yl)phenyl)imidazo[1,2-a]pyrazin-8-amine

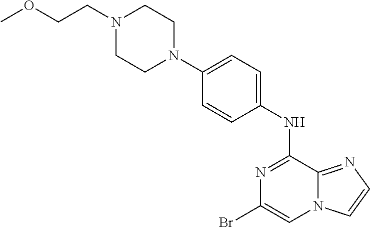 [SMILES: COCCN1CCN(c2ccc(Nc3nc(Br)cn4ccnc34)cc2)CC1]

A 50 ml sealed tube with a magnetic stirrer was charged with 4-(4-(2-methoxyethyl)piperazin-1-yl)aniline (1.3 g, 5.5 mmol), 6,8 dibromoimidazo[1,2-a]pyrazine (1.2 g, 4.3 mmol), cesium carbonate (2.8 g, 8.6 mmol) and 1,4-dioxane (20 mL). After degassed for 10 minutes, tris(dibenzylideneacetone)dipalladium(0) (0.2 g, 0.2 mmol) and Xantphos (0.18 g, 0.3 mmol) were added. The reaction mixture was heated at 100° C. for 16 hours. After this time, the mixture was cooled to room temperature, partitioned between methylene chloride (50 mL) and water (30 mL). The organic phase was separated, and the aqueous layer was extracted with methylene chloride (30 mL×3). The combined organic phases were washed with brine, dried over sodium sulfate and concentrated under reduced pressure. The residue was purified by flash column chromatography (silica, 3:1 methylene chloride/methanol) to give the titled compound as a brown solid.

### Example 166

### Preparation of 6-(2,3-dihydro-1H-pyrido[2,3-b][1,4]oxazin-7-yl)-N-(4-(4-(2-methoxyethyl)piperazin-1-yl)phenyl)imidazo[1,2-a]pyrazin-8-amine

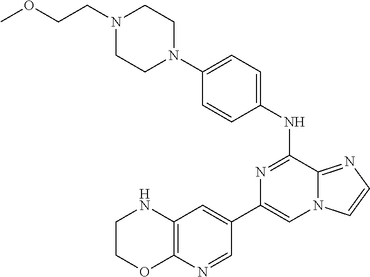 [SMILES: COCCN1CCN(c2ccc(Nc3nc(-c4cnc5c(c4)NCCO5)cn4ccnc34)cc2)CC1]

This compound was synthesized according to procedure F in Example 5b above.

[M+H]=487.5 g/mol; NMR (δ, ppm): 9.41 (s, 1H), 8.40 (s, 1H), 7.92 (m, 4H), 7.60 (s, 1H), 7.40 (s, 1H), 6.93 (d, 2H), 6.12 (s, 1H), 4.31 (br, 2H), 3.48 (t, 2H), 3.24 (s, 3H), 3.08 (br, 4H), 2.56 (br, 4H), 2.54 (m, 2H), 2.50 (m, 2H).

### Example 167

### Preparation of 2-(thiazol-2-yl)propan-2-ol

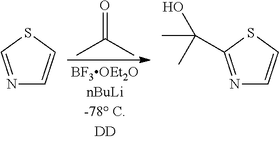

At −78° C. to mixture of thiazole (1 g, 11.7 mmol) and propan-2-one (1.02 g, 17.6 mmol) in tetrahydrofuran (10 mL) was added boron trifluoride etherate (BF₃.Et₂O) (1.7 mL, 14.2 mmol) dropwisely. The mixture was stirred at the same temperature for 30 minutes. Then it was followed up the addition of n-butyl lithium (2.5 N in hexane) (6 ml, 15.2 mmol) dropwisely, the reaction mixture was stirred at −78° C. for 1 hour. At −78° C. the reaction was quenched with 1N sodium hydroxide (10 mL) and diluted with ethyl acetate (20 mL), and then the mixture was warmed up to room temperature. Partioned the two layers and the organic phase was washed with water and brine, dried with sodium sulfate, purified by silicon gel column. The title compound was obtained; MS [M+H]=144.21.

### Example 168

### Preparation of 2-(5-(8-((3-methoxy-4-morpholinophenyl)amino)imidazo[1,2-a]pyrazin-6-yl)thiazol-2-yl)propan-2-ol

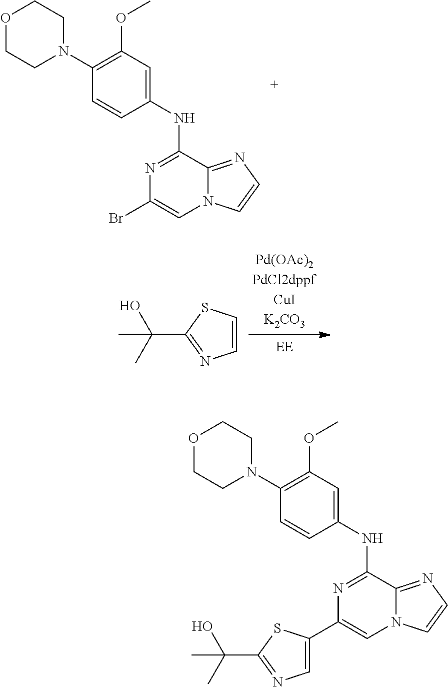

6-Bromo-N-(3-methoxy-4-morpholinophenyl)imidazo[1,2-a]pyrazin-8-amine (200 mg, 0.5 mmol) was dissolved in dioxane (3 mL) at room temperature and followed by the addition of 2-(thiazol-2-yl)propan-2-ol (0.14 g, 1 mmol), palladium(II) acetate (Pd(OAc)₂) (11.2 mg, 0.05 mmol), PCy₃.HBF₄ (37 mg, 0.1 mmol), Pd(dppf)₂Cl₂ (40 mg, 0.05 mmol), copper iodide (50 mg, 0.25 mmol), potassium carbonate (210 mg, 1.5 mmol), and picric acid (16 mg, 0.15 mmol). Then the reaction mixture was subjected to microwave at 120° C. for 90 minutes. The reaction was diluted with ethyl acetate (5 ml) and water (3 mL), black precipitate was filtered off. Two layers were portioned and organic phase was concentrate down and purified by HPLC. Title compound was obtained in solid form (60 mg, 0.12 mmol).

¹H-NMR (400 MHz, CD3OD): δ 8.34 (s, 1H), 8.05 (s, 1H), 7.99 (d, 1H), 7.96 (s, 1H), 7.80 (d, 1H), 7.56 (d, 1H), 7.24 (dd, 1H), 6.96 (d, 1H), 3.99 (s, 3H), 3.83 (dd, 4H), 3.02 (dd, 4H), 1.61 (s, 6H); MS [M+H]⁺=467.18.

### Example 169

### Preparation of 1,1,1-trifluoro-2-(thiazol-2-yl)propan-2-ol

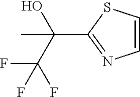

This compound was prepared according to procedure DD.

### Example 170

### Preparation of 1,1,1-trifluoro-2-(5-(8-((3-methoxy-4-morpholinophenyl)amino)imidazo[1,2-a]pyrazin-6-yl)thiazol-2-yl)propan-2-ol

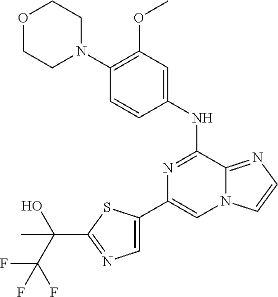 [SMILES: COc1cc(Nc2nc(-c3cnc(C(C)(O)C(F)(F)F)s3)cn3ccnc23)ccc1N1CCOCC1]

This compound was prepared according to procedure EE.

¹H-NMR (400 MHz, CD3OD): δ 8.58 (s, 1H) 8.40 (d, 1H), 8.21 (s, 1H), 7.92 (d, 1H), 7.67 (s, 1H), 7.56 (d, 1H), 7.44 (d, 1H), 4.17 (s, 3H), 4.04 (m, 4H), 3.64 (m, 4H), 1.83 (s, 6H). MS [M+H]⁺=521.12.

### Example 171

### Preparation of 3-(thiazol-2-yl)oxetan-3-ol

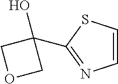

This compound was prepared according to procedure DD.

### Example 172

### 3-(5-(8-((3-methoxy-4-morpholinophenyl)amino)imidazo[1,2-a]pyrazin-6-yl)thiazol-2-yl)oxetan-3-ol

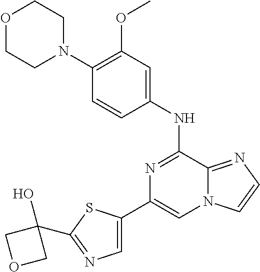 [SMILES: COc1cc(Nc2nc(-c3cnc(C4(O)COC4)s3)cn3ccnc23)ccc1N1CCOCC1]

This compound was prepared according to procedure EE.

¹H-NMR (400 MHz, CD3OD): δ 8.45 (s, 1H) 8.20 (s, 1H), 8.16 (s, 1H), 7.82 (d, 1H), 7.58 (s, 1H), 7.40 (m, 2H), 4.96 (d, 2H), 4.75 (d, 2H), 4.06 (s, 3H), 3.95 (m, 4H), 3.49 (m, 4H) MS [M+H]⁺=481.14.

### Example 173

Preparation of 2-(5-(8-((3-methoxy-4-(4-(oxetan-3-yl)piperazin-1-yl)phenyl)amino)imidazo[1,2-a]pyrazin-6-yl)thiazol-2-yl)propan-2-ol

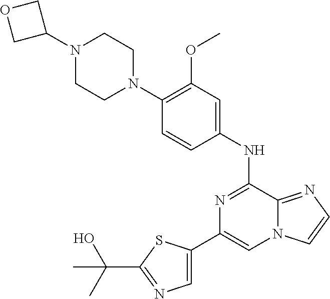 [SMILES: COc1cc(Nc2nc(-c3cnc(C(C)(C)O)s3)cn3ccnc23)ccc1N1CCN(C2COC2)CC1]

This compound was prepared according to procedure EE.

¹H-NMR (400 MHz, CD3OD): δ 8.44 (s, 1H) 8.10 (s, 1H), 8.06 (d, 1H), 7.89 (s, 1H), 7.33 (dd, 1H), 7.06 (d, 1H), 4.92-4.79 (m, 4H), 4.47 (m, 1H), 4.03 (s, 3H), 3.49-3.32 (m, 8H), 1.62 (s, 6H) MS [M+H]⁺=522.63.

### Example 174

### Preparation of 2-(5-(8-((4-(4-(oxetan-3-yl)piperazin-1-yl)phenyl)amino)imidazo[1,2-a]pyrazin-6-yl)thiazol-2-yl)propan-2-ol

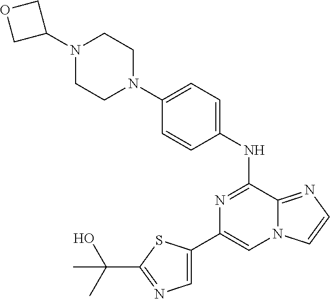 [SMILES: CC(C)(O)c1ncc(-c2cn3ccnc3c(Nc3ccc(N4CCN(C5COC5)CC4)cc3)n2)s1]

This compound was prepared according to procedure EE.

¹H-NMR (400 MHz, CD₃OD): δ 8.33 (s, 1H) 8.02 (s, 1H), 7.87 (m, 2H), 7.80 (s, 1H), 7.56 (s, 1H), 7.04 (m, 2H), 4.85-4.72 (m, 4H), 4.39 (m, 1H), 3.38-3.23 (m, 8H), 1.55 (m, 6H); MS [M+H]⁺=492.14

### Example 175

### Preparation of 2-(4-((6-(2,3-dihydro-1H-pyrido[2,3-b][1,4]oxazin-7-yl)imidazo[1,2-a]pyrazin-8-yl)amino)phenyl)propan-2-ol

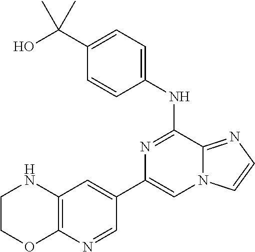 [SMILES: CC(C)(O)c1ccc(Nc2nc(-c3cnc4c(c3)NCCO4)cn3ccnc23)cc1]

This compound was synthesized according to procedure F in Example 5b above.

¹H-NMR (400 MHz, CD₃OD) δ 8.05 (s, 1H) 7.89 (d, 1H), 7.82 (m, 2H), 7.68 (s, 1H), 7.47 (m, 3H), 7.40 (d, 1H), 4.36 (m, 2H), 3.35 (m, 2H), 1.54 (m, 6H); MS [M+H]⁺=402.9

### Example 176

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)-3-methylazetidin-3-ol

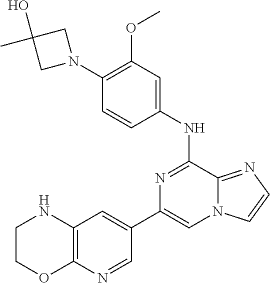 [SMILES: COc1cc(Nc2nc(-c3cnc4c(c3)NCCO4)cn3ccnc23)ccc1N1CC(C)(O)C1]

This compound was synthesized according to procedure F in Example 5b above.

¹H-NMR (400 MHz, DMSO) δ9.31 (s, 1H), 8.34 (s, 1H) 7.96 (s, 1H), 7.91 (s, 1H), 7.78 (s, 1H), 7.56 (s, 3H), 7.54 (s, 1H), 7.41 (s, 1H), 6.38 (d, 1H), 6.17 (s, 1H), 4.26 (m, 2H), 3.72 (m, 5H), 3.52 (m, 4H), 1.42 (m, 3H); MS [M+H]⁺=460.13.

### Example 177

### Preparation of 6-(2,3-dihydro-1H-pyrido[2,3-b][1,4]oxazin-7-yl)-N-(3-methoxy-4-(tetrahydro-1H-furo[3,4-c]pyrrol-5(3H)-yl)phenyl)imidazo[1,2-a]pyrazin-8-amine

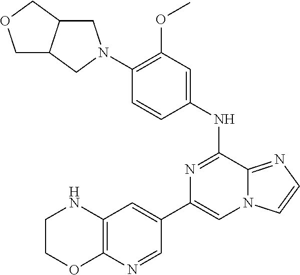 [SMILES: COc1cc(Nc2nc(-c3cnc4c(c3)NCCO4)cn3ccnc23)ccc1N1CC2COCC2C1]

This compound was synthesized according to procedure F in Example 5b above.

¹H NMR (400 MHz, CD₃OD): δ 8.46 (s, 1H), 8.28 (s, 1H), 8.07 (d, J=2.0 Hz, 1H), 7.95 (d, J=1.1 Hz, 1H), 7.76-7.68 (m, 2H), 7.59 (m, 2H), 4.55-4.53 (m, 2H), 4.08 (m, 5H), 3.96 (d, J=9.6 Hz, 2H), 3.67 (m, 4H), 3.64 (m, 4H).

### Example 178

### Preparation of (S)-(1-(4-((6-(2,3-dihydro-1H-pyrido[2,3-b][1,4]oxazin-7-yl)imidazo[1,2-a]pyrazin-8-yl)amino)-2-methoxyphenyl)piperidin-3-yl)methanol

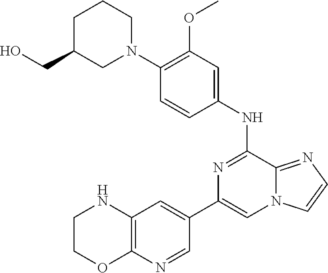 [SMILES: COc1cc(Nc2nc(-c3cnc4c(c3)NCCO4)cn3ccnc23)ccc1N1CCC[C@H](CO)C1]

This compound was synthesized according to procedure F in Example 5b above.

¹H NMR (400 MHz, CD₃OD): δ 8.46 (s, 1H), 8.34 (d, J=2.1 Hz, 1H), 8.07 (d, J=2.0 Hz, 1H), 7.95 (d, J=1.2 Hz, 1H), 7.71 (dd, J=7.6, 1.6 Hz, 2H), 7.65 (d, J=9.0 Hz, 2H), 7.58 (dd, J=9.0, 2.2 Hz, 2H), 4.53 (m, 2H), 4.09 (s, 3H), 3.73-3.48 (m, 10H), 2.27-1.91 (m, 4H), 1.48 (m, 1H); MS [M+H]⁺=488.24.

### Example 179

### Preparation of 1-(4-aminophenyl)cyclobutanol

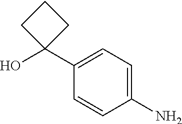 [SMILES: Nc1ccc(C2(O)CCC2)cc1]

To solution of 4-bromo-N,N-bis(trimethylsilyl)aniline (1 g, 3.16 mmol) in tetrahydrofuran (10 mL) was added n-butyl lithium (n-BuLi) (1.6 N in Hexane) (4 mL, 6.32 mmol) dropwisely at −78° C., then the resulting mixture was stirred at the same temperature for 1 h. cyclobutanone (0.2 g, 2.84 mmol) was added into the mixture slowly, then the reaction was warmed up to room temperate in 30 minutes. The reaction was quenched by sat. ammonium chloride at 0° C., and was diluted with ethyl acetate, two layers were portioned and aqueous phase was extracted with ethyl acetate (2×) and combined organic phase was washed with brine and dried with MgSO₄. Solvent was stripped off and the residue was purified by silicon gel. The title compound was obtained.

### Example 180

### Preparation of 1-(4-(6-bromoimidazo[1,2-a]pyrazin-8-ylamino)phenyl)cyclobutanol

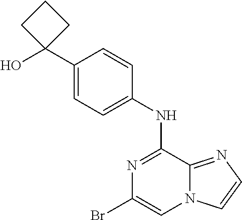 [SMILES: OC1(c2ccc(Nc3nc(Br)cn4ccnc34)cc2)CCC1]

This compound was prepared according to procedure C in Example 3 above.

### Example 181

### Preparation of 1-(4-((6-(2,3-dihydro-1H-pyrido[2,3-b][1,4]oxazin-7-yl)imidazo[1,2-a]pyrazin-8-yl)amino)phenyl)cyclobutanol

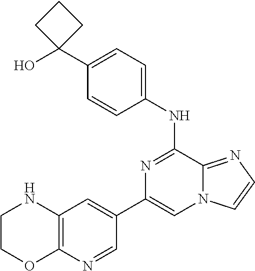 [SMILES: OC1(c2ccc(Nc3nc(-c4cnc5c(c4)NCCO5)cn4ccnc34)cc2)CCC1]

This compound was synthesized according to procedure F in Example 5b above.

¹H NMR (400 MHz, DMSO) δ 9.53 (s, 1H), 8.41 (s, 1H), 8.02 (d, 2H), 7.93 (d, 2H), 7.59-7.52 (m, 2H), 7.47 (m, 1H), 7.41 (m, 1H), 6.23 (s, 1H), 5.33 (s, 1H), 4.24 (m, 2H), 3.27 (m, 2H), 2.39-2.32 (m, 2H), 2.24-2.17 (m, 2H), 1.88-1.76 (m, 1H), 1.61-1.51 (m, 1H); MS [M+H]⁺=415.04.

### Example 182

### Preparation of 3-(4-((6-(2,3-dihydro-1H-pyrido[2,3-b][1,4]oxazin-7-yl)imidazo[1,2-a]pyrazin-8-yl)amino)phenyl)oxetan-3-ol

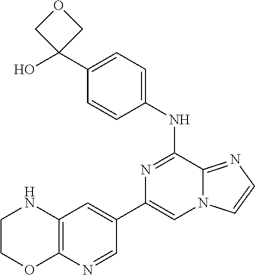 [SMILES: OC1(c2ccc(Nc3nc(-c4cnc5c(c4)NCCO5)cn4ccnc34)cc2)COC1]

This compound was synthesized according to procedure F in Example 5b above.

¹H NMR (400 MHz, DMSO): δ 9.62 (s, 1H), 8.42 (s, 1H), 8.08 (d, 2H), 7.92 (d, 2H), 7.8 (s, 1H), 7.52 (d, 1H), 7.41 (s, 1H), 6.23 (s, 1H), 6.19 (s, 1H), 4.68 (m, 4H), 4.24 (m, 2H), 3.24 (m, 2H); MS [M+H]⁺=417.12

### Example 183

### Preparation of 6-(2,3-dihydro-1H-pyrido[2,3-b][1,4]oxazin-7-yl)-N-(3-methoxy-4-(1,4-dioxa-8-azaspiro[4.5]decan-8-yl)phenyl)imidazo[1,2-a]pyrazin-8-amine

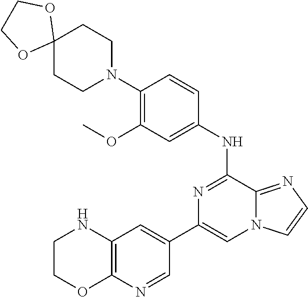 [SMILES: COc1cc(Nc2nc(-c3cnc4c(c3)NCCO4)cn3ccnc23)ccc1N1CCC2(CC1)OCCO2]

This compound was synthesized according to procedure F in Example 5b above.

¹H NMR (300 MHz, d6-DMSO): δ 8.13 (d, 1H), 7.93 (s, 1H), 7.83 (s, 1H), 7.75 (d, 1H), 7.53 (dd, 2H), 7.41 (d, 1H), 7.25 (dd, 1H), 6.96 (d, 1H), 4.44 (t, 2H), 3.99 (s, 3H), 3.97 (m, 2H), 3.91 (m, 2H), 3.44 (t, 2H), 3.12 (m, 4H), 1.92 (m, 4H); MS [M+H]+=516.13.

### Example 184

### Preparation 1-(4-((6-(2,3-dihydro-1H-pyrido[2,3-b][1,4]oxazin-7-yl)imidazo[1,2-a]pyrazin-8-yl)amino)-2-methoxyphenyl)piperidin-4-one

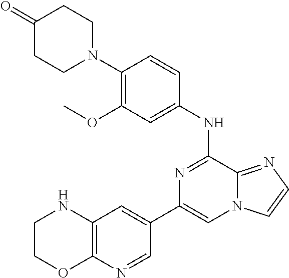 [SMILES: COc1cc(Nc2nc(-c3cnc4c(c3)NCCO4)cn3ccnc23)ccc1N1CCC(=O)CC1]

To a solution of 6-(2,3-dihydro-1H-pyrido[2,3-b][1,4]oxazin-7-yl)-N-(3-methoxy-4-(1,4-dioxa-8-azaspiro[4.5]decan-8-yl)phenyl)imidazo[1,2-a]pyrazin-8-amine (650 mg) in EtOH (10 mL) was added concentrated HCl (3 mL). After reflux for overnight, the reaction was neutralized with sodium bicarbonate and extracted with ethyl acetate. The organic phase was dried over sodium sulfate, filtered and the filtrate was concentrated under reduced pressure. The residue was purified by silica gel column to afford 1-(4-(6-(2,3-dihydro-1H-pyrido[2,3-b][1,4]oxazin-7-yl)imidazo[1,2-a]pyrazin-8-ylamino)-2-methoxyphenyl)piperidin-4-one.

¹H NMR (CDCl₃): δ 8.17 (d, 1H), 8.04 (br., 1H), 7.86 (d, 1H), 7.83 (d, 1H), 7.57 (dd, 2H), 7.41 (d, 1H), 7.31 (dd, 1H), 6.97 (d, 1H), 4.45 (t, 2H), 3.96 (s, 3H), 3.46 (t, 2H), 3.34 (t, 4H), 2.64 (t, 4H); MS [M+H]+=472.22.

### Example 185

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)piperidin-4-one oxime

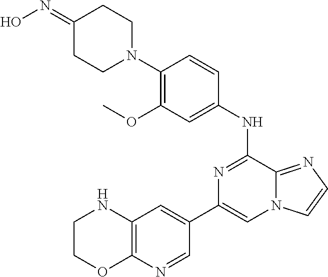 [SMILES: COc1cc(Nc2nc(-c3cnc4c(c3)NCCO4)cn3ccnc23)ccc1N1CCC(=NO)CC1]

To a solution of 1-(4-(6-(2,3-dihydro-1H-pyrido[2,3-b][1,4]oxazin-7-yl)imidazo[1,2-a]pyrazin-8-ylamino)-2-methoxyphenyl)piperidin-4-one (180 mg) in EtOH (20 mL) was added hydroxylamine hydrochloride (36 mg) and Sodium acetate (82 mg). After stirred for overnight at 50° C., the reaction mixture was extracted with ethyl acetate. The organic phase was dried over sodium sulfate, filtered and the filtrate was concentrated under reduced pressure. The residue was purified by silica gel column to afford 1-(4-(6-(2,3-dihydro-1H-pyrido[2,3-b][1,4]oxazin-7-yl)imidazo[1,2-a]pyrazin-8-ylamino)-2-methoxyphenyl)piperidin-4-one oxime.

¹H NMR (300 MHz, d6-DMSO): δ 10.35 (s, 1H), 9.48 (s, 1H), 8.42 (s, 1H), 7.96 (s, 1H), 7.89 (s, 1H), 7.64 (dd, 1H), 7.59 (s, 1H), 7.44 (d, 1H), 6.9 (d, 1H), 6.29 (s, 1H), 4.26 (m, 2H), 3.798 (s, 3H), 3.28 (m, 2H), 3.0 (t, 2H), 2.94 (t, 2H), 2.57 (t, 2H), 2.32 (t, 2H); MS [M+H]+=487.15.

### Example 186

### Preparation of 1-(4-((6-(2,3-dihydro-1H-pyrido[2,3-b][1,4]oxazin-7-yl)imidazo[1,2-a]pyrazin-8-yl)amino)-2-methoxyphenyl)piperidin-4-one O-methyl oxime

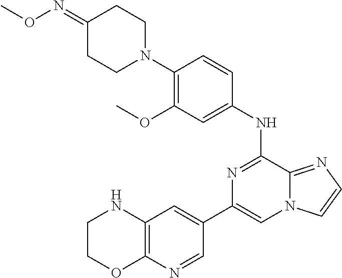 [SMILES: CON=C1CCN(c2ccc(Nc3nc(-c4cnc5c(c4)NCCO5)cn4ccnc34)cc2OC)CC1]

To a solution of 1-(4-(6-(2,3-dihydro-1H-pyrido[2,3-b][1,4]oxazin-7-yl)imidazo[1,2-a]pyrazin-8-ylamino)-2-methoxyphenyl)piperidin-4-one (260 mg) in methanol (10 mL) was added methoxyamine hydrochloride (84 mg) and triethylamine (202 mg). After stirred for overnight at 50° C., the reaction mixture was extracted with ethyl acetate. The organic phase was washed with brine, dried over sodium sulfate, filtered and the filtrate was concentrated under reduced pressure. The residue was purified by silica gel column to afford 1-(4-(6-(2,3-dihydro-1H-pyrido[2,3-b][1,4]oxazin-7-yl)imidazo[1,2-a]pyrazin-8-ylamino)-2-methoxyphenyl)piperidin-4-one-O-methyl oxime.

¹H NMR (DMSO-d): δ 9.496 (s, 1H), 8.4 (s, 1H), 7.97 (d, 1H), 7.94 (s, 1H), 7.91 (d, 1H), 7.65 (dd, 1H), 7.59 (s, 1H), 7.43 (d, 1H), 6.89 (d, 1H), 6.23 (s, 1H), 4.27 (t, 2H), 3.798 (s, 3H), 3.72 (s, 3H), 3.28 (m, 2H), 3.03 (t, 2H), 2.96 (t, 2H), 2.57 (t, 2H), 2.34 (t, 2H); MS [M+H]+=501.11.

### Example 187

### Preparation of E and Z 1-(4-aminophenyl)ethanone O-methyl oxime

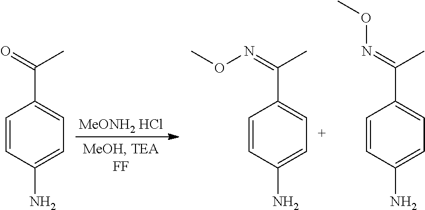

To a solution of 4-acetoaniline (1.35 g) in methanol (50 mL) was added methoxyamine hydrochloride (840 mg) and triethylamine (2 mL). After stirred for overnight at 50° C., the reaction mixture was extracted with ethyl acetate. The organic phase was washed with brine, dried over sodium sulfate, filtered and the filtrate was concentrated under reduced pressure. The residue was purified by silica gel column to afford (Z)-1-(4-aminophenyl)ethanone O-methyl oxime and (E)-1-(4-aminophenyl)ethanone O-methyl oxime.

### Example 188

### Preparation of (E)-1-(4-((6-(2,3-dihydro-1H-pyrido[2,3-b][1,4]oxazin-7-yl)imidazo[1,2-a]pyrazin-8-yl)amino)phenyl)ethanone O-methyl oxime

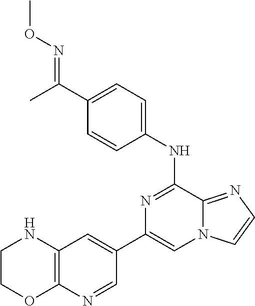 [SMILES: CO/N=C(\C)c1ccc(Nc2nc(-c3cnc4c(c3)NCCO4)cn3ccnc23)cc1]

This compound was synthesized according to procedure F in Example 5b above.

¹H NMR (DMSO-d): δ 9.81 (s, 1H), 8.49 (s, 1H), 8.18 (s, 1H), 8.15 (s, 1H), 7.96 (d, 1H), 7.95 (d, 1H), 7.67 (s, 1H), 7.65 (s, 1H), 7.63 (d, 1H), 7.45 (d, 1H), 6.29 (s, 1H), 4.28 (t, 2H), 3.88 (s, 3H), 3.3 (m, 2H), 2.15 (s, 3H); MS [M+H]+=416.12.

### Example 189

### Preparation of (Z)-1-(4-((6-(2,3-dihydro-1H-pyrido[2,3-b][1,4]oxazin-7-yl)imidazo[1,2-a]pyrazin-8-yl)amino)phenyl)ethanone O-methyl oxime

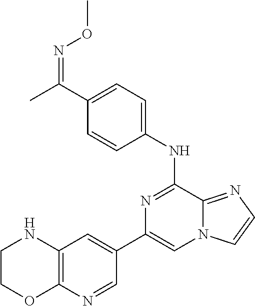 [SMILES: CO/N=C(/C)c1ccc(Nc2nc(-c3cnc4c(c3)NCCO4)cn3ccnc23)cc1]

This compound was synthesized according to procedure F in Example 5b above.

¹H NMR (DMSO-d): δ 9.82 (s, 1H), 8.487 (s, 1H), 8.17 (s, 1H), 8.148 (s, 1H), 7.97 (d, 1H), 7.95 (d, 1H), 7.63 (d, 1H), 7.603 (s, 1H), 7.43 (d, 1H), 6.26 (s, 1H), 4.28 (t, 2H), 3.74 (s, 3H), 3.3 (m, 2H), 2.13 (s, 3H); MS [M+H]+=416.08.

### Example 190

### Preparation of 4-(4-fluoropiperidin-1-yl)-3-methoxyaniline

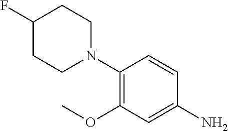 [SMILES: COc1cc(N)ccc1N1CCC(F)CC1]

In a parr bottle, a suspension of 4-fluoro-1-(2-methoxy-4-nitrophenyl)piperidine (1.11 g, 4.3 mmol) in methanol/methylene chloride (8:2, 30 mL) was added Pd/C (0.2 g). The mixture was hydrogenated at 50 psi for 1 hour. The reaction mixture was filtered, and washed with methanol. The filtrate was concentrated in vacuo to give 4-(4-fluoropiperidin-1-yl)-3-methoxyaniline as a brown solid.

### Example 191

### Preparation of 6-bromo-N-(4-(4-fluoropiperidin-1-yl)phenyl)imidazo[1,2-a]pyrazin-8-amine

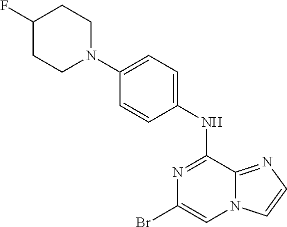 [SMILES: FC1CCN(c2ccc(Nc3nc(Br)cn4ccnc34)cc2)CC1]

A mixture of 4-(4-fluoropiperidin-1-yl)-3-methoxyaniline (0.98 g, 4.3 mmol), 6,8 dibromoimidazo[1,2-a]pyrazine (1 g, 3.6 mmol) in dimethylamine (3 mL) was heated at 130° C. for 30 min. After being cooled down to room temperature, 2M potassium carbonate (30 mL) was added. The mixture was stirred at room temperature for 30 minutes the solid was filtered and washed with water followed by isopropanol (2×), dried to give 6-bromo-N-(4-(4-fluoropiperidin-1-yl)phenyl)imidazo[1,2-a]pyrazin-8-amine.

### Example 192

### Preparation of 6-(2,3-dihydro-1H-pyrido[2,3-b][1,4]oxazin-7-yl)-N-(4-(4-fluoropiperidin-1-yl)-3-methoxyphenyl)imidazo[1,2-a]pyrazin-8-amine

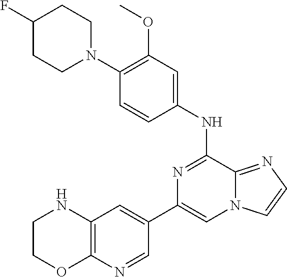 [SMILES: COc1cc(Nc2nc(-c3cnc4c(c3)NCCO4)cn3ccnc23)ccc1N1CCC(F)CC1]

This compound was synthesized according to procedure F in Example 5b above.

[M+H]=476.5; NMR (δ, ppm): 9.42 (s, 1H), 8.43 (s, 1H), 8.01 (d, 1H), 7.96 (d, 1H), 7.91 (d, 1H), 7.69-7.65 (dd, 1H), 7.62 (d, 1H), 7.47 (d, 1H), 6.94-6.91 (d, 1H), 6.21 (s, 1H), 4.88-4.69 (m, 1H), 4.32-4.29 (m, 2H), 3.82 (s, 3H), 3.34-3.30 (m, 2H), 3.08 (m, 2H), 2.90-2.83 (m, 2H), 2.07-1.85 (m, 4H).

### Example 193

### Preparation of 6-(2,3-dihydro-1H-pyrido[2,3-b][1,4]oxazin-7-yl)-N-(3,4-dimethoxyphenyl)imidazo[1,2-a]pyrazin-8-amine

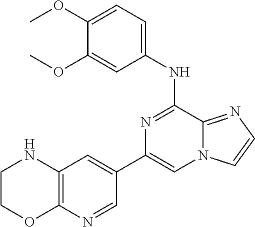 [SMILES: COc1ccc(Nc2nc(-c3cnc4c(c3)NCCO4)cn3ccnc23)cc1OC]

This compound was synthesized according to procedure F in Example 5b above.

[M+H]=405.6 g/mol; NMR (δ, ppm): 9.49 (s, 1H), 8.42 (s, 1H), 8.00 (d, 1H), 7.96 (d, 1H), 7.94 (d, 1H), 7.69-7.66 (dd, 1H), 7.62 (d, 1H), 7.46 (d, 1H), 6.98-6.95 (d, 1H), 6.21 (s, 1H), 4.32-4.29 (m, 2H), 3.79 (s, 3H), 3.75 (s, 3H), 3.33-3.30 (m, 2H).

### Example B1

### Ramos Cell pBLNK(Y96) Assay

A standard cellular Syk Kinase Assay was used to test certain compounds disclosed herein is as follows.

Ramos cells were serum-starved at 2×10⁶ cells/mL in serum-free RPM I for 1 hour in an upright T175 Falcon TC flask. Cells were centrifuged (11 00 rpm×5 min) and incubated at a density of 0.5×10⁷ cells/ml in the presence of a test compound or DMSO controls for 1 hour at 37° C. Cells were then stimulated by incubating with 10 μg/ml anti-human IgM F(ab)₂ for 5 minutes at 37° C. Cells were pelleted, lysed in 40 μL cell lysis buffer, and mixed with Invitrogen SDS-PAGE loading buffer. 20 μL of cell lysate for each sample were subject to SDS-PAGE and western blotting with antiphosphoBLNK(Tyr96) antibody (Cell Signaling Technology #3601) to assess Syk activity and anti-Syk antibody (BD Transduction Labs #611116) to control for total protein load in each lysate. The images were detected using fluorescent secondary detection systems and the LiCor Odyssey software.

### Example B2

### High Throughput Syk Assay

The following high throughput Syk assay was performed on the compounds of Table 1 as follows.

Syk activity was measured by detecting phosphorylated peptide substrate formation using antibody against phosphorylated peptide substrate. This is a time-resolved fluorescence resonance energy transfer (TR-FRET) immunoassay, based on the KinEASE Assay (Cisbio). The assay was designed as a simple two-step, endpoint assay (a 5 μL enzyme reaction followed by 5 μL Stop and Detect Solution) performed in Perkin Elmer ProxiPlate-384 Plus plates. K252a, a non selective kinase inhibitor was used as a positive control. Test compounds (in DMSO) were spotted into 384 well plates using a Labcyte® Echo 550 Liquid Handling System prior to addition of Syk enzyme and peptide substrate. Reaction solutions were delivered using a Multi-Flo (Bio-Tek Instruments). The enzyme and peptide solution was incubated with compound for 60 minutes at room temp before the reaction was initiated by the addition of ATP. The standard 5 uL reaction mixture contained 3 μM ATP, 0.16 μM peptide, 0.5 nM of Syk in reaction buffer (50 mM Hepes, pH 7.0, 0.02% NaN₃, 0.01% BSA, 0.1 mM Orthovanadate, 5 mM MgCl₂, 1 mM DTT). After 60 min of incubation at room temperature, 5 μL of Stop and Detect Solution (1:200 Cryptate labeled anti-phosphorylated peptide antibody solution and 14 nM Tracer in a 50 mM Hepes pH 7.0 detection buffer containing sufficient EDTA) was added. The plate was then further incubated for 60 minutes at room temperature and read on Envision 2103 Multilabeled reader from PerkinElmer. The europium donor was excited using a 340-nm excitation filter with a 30-nm band pass. Energy transfer to the tracer was measured using a filter centered at 665 nm with a 10 nm bandpass. This signal was then referenced to the emission from europium peak, using a 615 nm, 10-nm bandpass filter. The “emission ratio” was calculated as the 665 nm signal divided by the 615 nm signal. Percentage of inhibition was calculated as below:

% Inhibition=100×(Ratio_{Sample}−Ratio_{0% Inhibition})/(Ratio_{100% Inhibition}−Ratio_{0% Inhibition})

The 0% inhibition value comes from a control well lacking inhibitor. The 100% inhibition value comes from control wells containing a saturating amount of known inhibitor K252a.

### Example B3

### Kinetic Solubility Study

The compounds tested (as listed in Table 2 below) were dissolved in dimethyl sulfoxide (DMSO) at a 10 mM concentration. A 3 μL sample aliquot of the DMSO solution is then added to 297 μL of desired aqueous media (pH 2, pH 5.4, and pH 7.4). The sample is then incubated for 24 hrs at 37° C. with shaking. After 24 hrs the samples are centrifuged, an aliquot of the supernatant liquid is removed and analyzed by HPLC. The compound concentration in the sample is determined by reference to a sample of the compound with known standard concentration.

TABLE 2

| | | Solubility (uM) | | |
|---|---|---|---|---|
| Compound No. | Compound Structure | pH 7.4 | pH 5.4 | pH 2 |
| 90x | 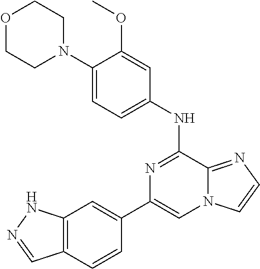 [SMILES: COc1cc(Nc2nc(-c3ccc4cn[nH]c4c3)cn3ccnc23)ccc1N1CCOCC1] | 3 | 3 | 94 |
| 14x | 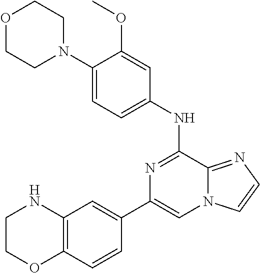 [SMILES: COc1cc(Nc2nc(-c3ccc4c(c3)NCCO4)cn3ccnc23)ccc1N1CCOCC1] | 8 | 10 | 87 |
| 28x | 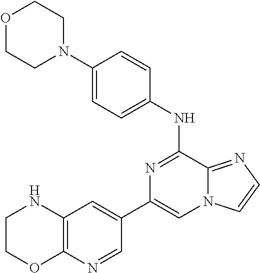 [SMILES: c1cn2cc(-c3cnc4c(c3)NCCO4)nc(Nc3ccc(N4CCOCC4)cc3)c2n1] | 9 | 9 | 90 |
| 1 EX 5 | 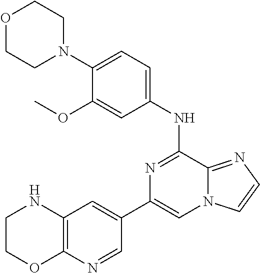 [SMILES: COc1cc(Nc2nc(-c3cnc4c(c3)NCCO4)cn3ccnc23)ccc1N1CCOCC1] | 95 | 95 | 98 |
| 4 EX 12 | 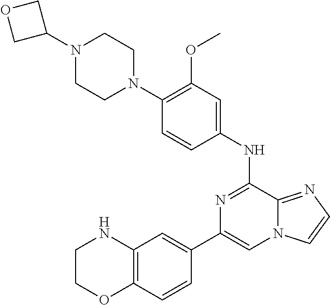 [SMILES: COc1cc(Nc2nc(-c3ccc4c(c3)NCCO4)cn3ccnc23)ccc1N1CCN(C2COC2)CC1] | 57 | 94 | 94 |
| 3 EX 8 | 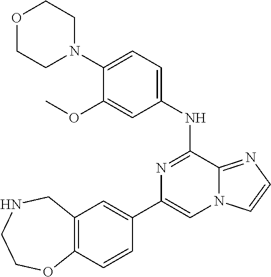 [SMILES: COc1cc(Nc2nc(-c3ccc4c(c3)CNCCO4)cn3ccnc23)ccc1N1CCOCC1] | 100 | 100 | 100 |
| 24 EX 32 | 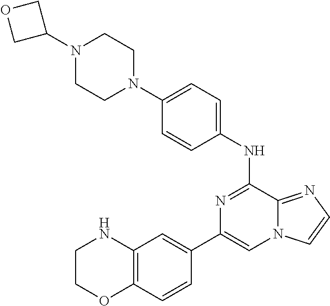 [SMILES: c1cn2cc(-c3ccc4c(c3)NCCO4)nc(Nc3ccc(N4CCN(C5COC5)CC4)cc3)c2n1] | 33 | 61 | 100 |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

What is claimed is:

1. A compound having the structure of formula (Ib): 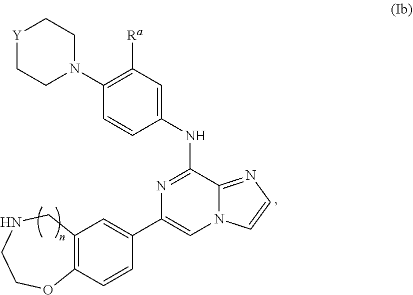
or a pharmaceutically acceptable salt thereof, wherein:
X is N or CH;
n is 0 or 1;
R^{a} is unsubstituted alkoxy; and
Y is O or NR^{2a}, wherein R^{2a} is selected from the group consisting of unsubstituted alkyl, substituted alkyl, unsubstituted heterocycloalkyl, and substituted heterocycloalkyl,
provided that the compound is other than Compound No. 14x, 34x, 77x, 78x or 79x.

2. A compound having the structure of formula (Ia): 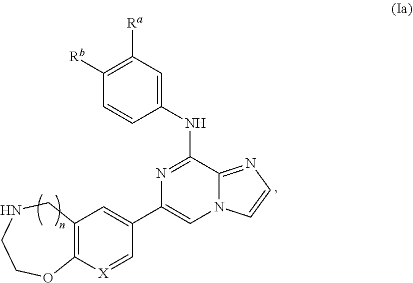
or a pharmaceutically acceptable salt thereof, wherein:
X is N or CH;
n is 0 or 1;
R^{a} is unsubstituted alkoxy; and
R^{b} is selected from the group consisting of unsubstituted morpholinyl, substituted morpholinyl, unsubstituted piperazinyl, and substituted piperazinyl,
provided that the compound is other than Compound No. 14x, 34x, 77x, 78x or 79x.

3. The compound of claim 2, or a pharmaceutically acceptable salt thereof, wherein R^{b} is:
unsubstituted morpholinyl,
substituted morpholinyl with one, two or three substituents independently selected from the group consisting of unsubstituted alkyl and substituted alkyl;
unsubstituted piperazinyl; or
substituted piperazinyl with one, two or three substituents independently selected from the group consisting of unsubstituted alkyl, substituted alkyl, unsubstituted heterocycloalkyl, and substituted heterocycloalkyl.

4. The compound of claim 2, or a pharmaceutically acceptable salt thereof, wherein R^{b} is selected from the group consisting of:

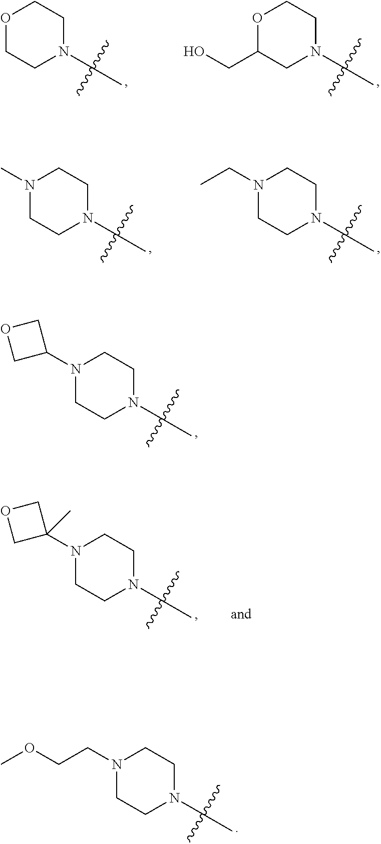

5. The compound of claim 2, or a pharmaceutically acceptable salt thereof, wherein:
X is N or CH;
R^{a} is methoxy;
R^{b} is unsubstituted morpholinyl, substituted morpholinyl, unsubstituted piperazinyl or substituted piperazinyl; and
n is 0 or 1.

6. A compound having the structure of formula (Ic): 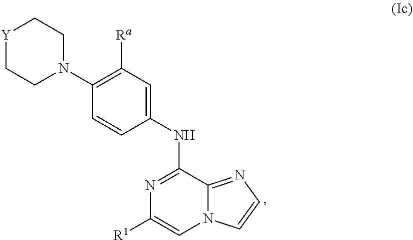
or a pharmaceutically acceptable salt thereof, wherein:
R¹ is 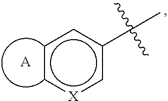
wherein A is selected from the group consisting of unsubstituted morpholinyl, substituted morpholinyl, unsubstituted homomorpholinyl, and substituted homomorpholinyl;
X is N or CR^{x}, wherein R^{x} is hydrogen or C₁₋₆ alkyl;
R^{a} is unsubstituted alkoxy; and
Y is O or NR^{2a}, and wherein R^{2a} is selected from the group consisting of unsubstituted alkyl, substituted alkyl, unsubstituted heterocycloalkyl, and substituted heterocycloalkyl,
provided that the compound is other than Compound No. 14x, 34x, 77x, 78x or 79x.

7. The compound of claim 6, or a pharmaceutically acceptable salt thereof, wherein R¹ is selected from the group consisting of:

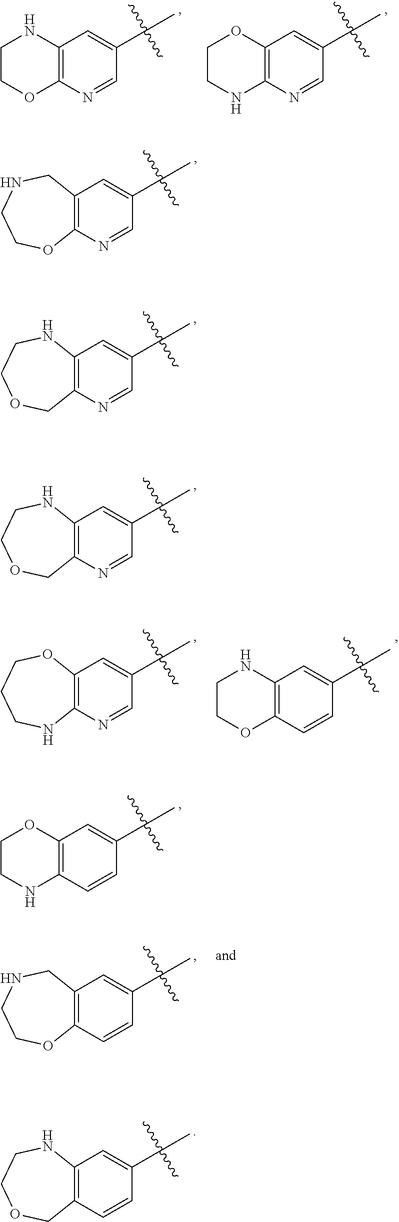

8. A compound having the structure of formula (Id): 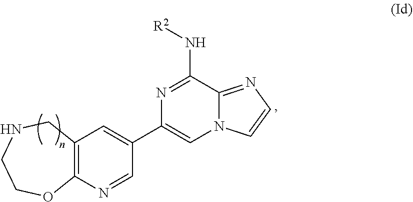
or a pharmaceutically acceptable salt thereof, wherein:
n is 0 or 1;
R² is selected from the group consisting of unsubstituted phenyl, substituted phenyl, unsubstituted pyridinyl, substituted pyridinyl, unsubstituted pyrazolyl, substituted pyrazolyl, unsubstituted thiazolyl, and substituted thiazolyl,
provided that the compound is other than Compound No. 28x or 37x.

9. The compound of claim 8, or a pharmaceutically acceptable salt thereof, wherein R² is:
unsubstituted phenyl;
substituted phenyl with one or two substituents independently selected from the group consisting of unsubstituted alkyl, substituted alkyl, unsubstituted cycloalkyl, substituted cycloalkyl, unsubstituted heterocycloalkyl, substituted heterocycloalkyl, unsubstituted aryl, substituted aryl, unsubstituted heteroaryl, substituted heteroaryl, unsubstituted alkoxy, substituted alkoxy, unsubstituted cycloalkyloxy, substituted cycloalkyloxy, unsubstituted heterocycloalkyloxy, substituted heterocycloalkyloxy, unsubstituted amino, substituted amino, unsubstituted sulfonyl, substituted sulfonyl, and oxime;
unsubstituted pyridinyl;
substituted pyridinyl with one or two substituents independently selected from the group consisting of unsubstituted morpholinyl and substituted morpholinyl;
unsubstituted pyrazolyl;
substituted pyrazolyl with one or two substituents independently selected from the group consisting of unsubstituted alkyl and substituted alkyl;
unsubstituted thiazolyl; or
substituted thiazolyl with one or two substituents independently selected from the group consisting of unsubstituted alkyl and substituted alkyl.

10. A compound having the structure of formula (Ie): 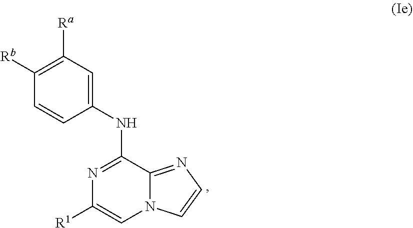
or a pharmaceutically acceptable salt thereof, wherein:
R¹ is substituted thiazolyl;
R^{a} is hydrogen, halo or unsubstituted alkoxy; and
R^{b} is selected from the group consisting of unsubstituted alkyl, substituted alkyl, unsubstituted alkoxy, substituted alkoxy, unsubstituted sulfonyl, substituted sulfonyl, unsubstituted morpholinyl, substituted morpholinyl, unsubstituted homomorpholinyl, substituted homomorpholinyl, unsubstituted piperazinyl, substituted piperazinyl, unsubstituted piperidinyl, substituted piperidinyl, unsubstituted pyrrolidinyl, substituted pyrrolidinyl, unsubstituted azetidinyl, and substituted azetidinyl.

11. The compound of claim 10, or a pharmaceutically acceptable salt thereof, wherein R^{b} is unsubstituted morpholinyl.

12. The compound of claim 10, or a pharmaceutically acceptable salt thereof, wherein R¹ is substituted thiazolyl with one or two substituents selected from the group consisting of unsubstituted alkyl, substituted alkyl, unsubstituted cycloalkyl, substituted cycloalkyl, unsubstituted heterocycloalkyl, and substituted heterocycloalkyl.

13. A compound selected from the group consisting of: 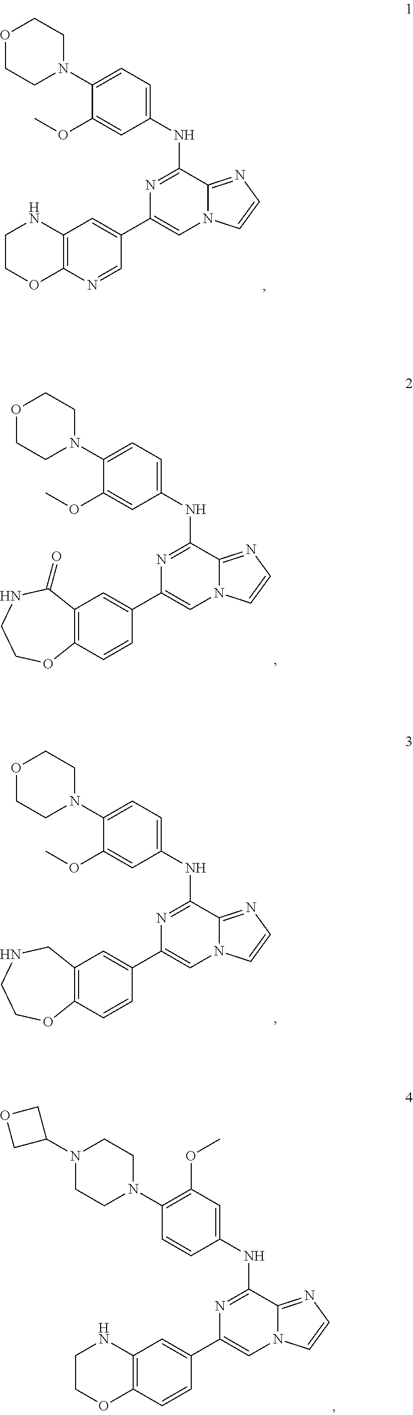 [SMILES: COc1cc(Nc2nc(-c3cnc4c(c3)NCCO4)cn3ccnc23)ccc1N1CCOCC1 ; COc1cc(Nc2nc(-c3ccc4c(c3)C(=O)NCCO4)cn3ccnc23)ccc1N1CCOCC1 ; COc1cc(Nc2nc(-c3ccc4c(c3)CNCCO4)cn3ccnc23)ccc1N1CCOCC1 ; COc1cc(Nc2nc(-c3ccc4c(c3)NCCO4)cn3ccnc23)ccc1N1CCN(C2COC2)CC1] 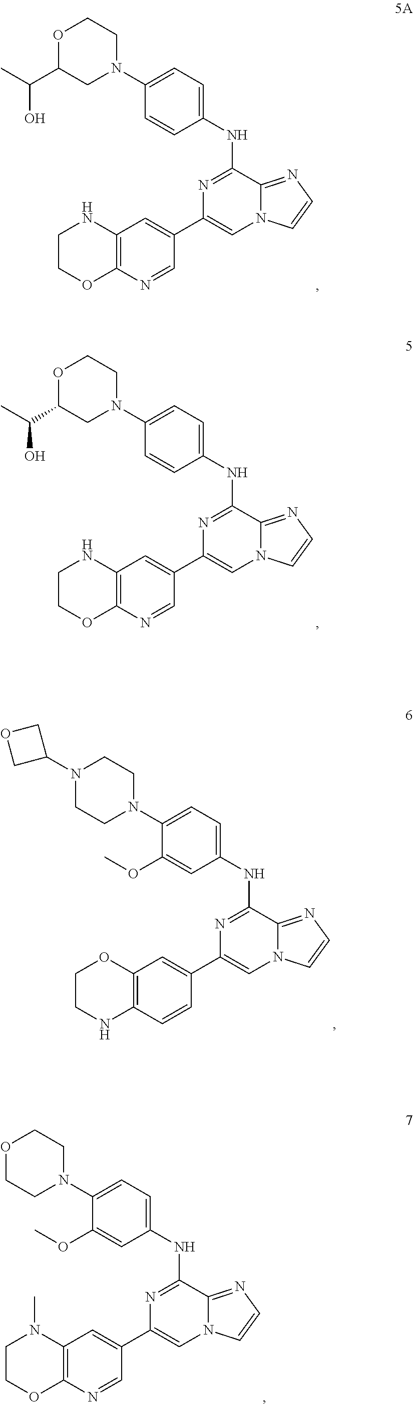 [SMILES: CC(O)C1CN(c2ccc(Nc3nc(-c4cnc5c(c4)NCCO5)cn4ccnc34)cc2)CCO1 ; C[C@H](O)[C@H]1CN(c2ccc(Nc3nc(-c4cnc5c(c4)NCCO5)cn4ccnc34)cc2)CCO1 ; COc1cc(Nc2nc(-c3ccc4c(c3)OCCN4)cn3ccnc23)ccc1N1CCN(C2COC2)CC1 ; COc1cc(Nc2nc(-c3cnc4c(c3)N(C)CCO4)cn3ccnc23)ccc1N1CCOCC1] 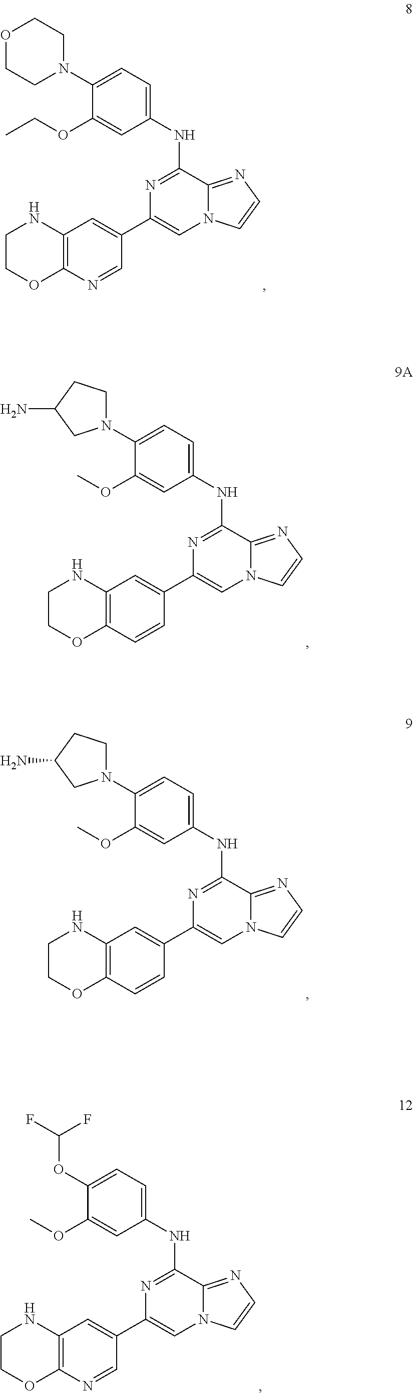 [SMILES: CCOc1cc(Nc2nc(-c3cnc4c(c3)NCCO4)cn3ccnc23)ccc1N1CCOCC1 ; COc1cc(Nc2nc(-c3ccc4c(c3)NCCO4)cn3ccnc23)ccc1N1CCC(N)C1 ; COc1cc(Nc2nc(-c3ccc4c(c3)NCCO4)cn3ccnc23)ccc1N1CC[C@@H](N)C1 ; COc1cc(Nc2nc(-c3cnc4c(c3)NCCO4)cn3ccnc23)ccc1OC(F)F] 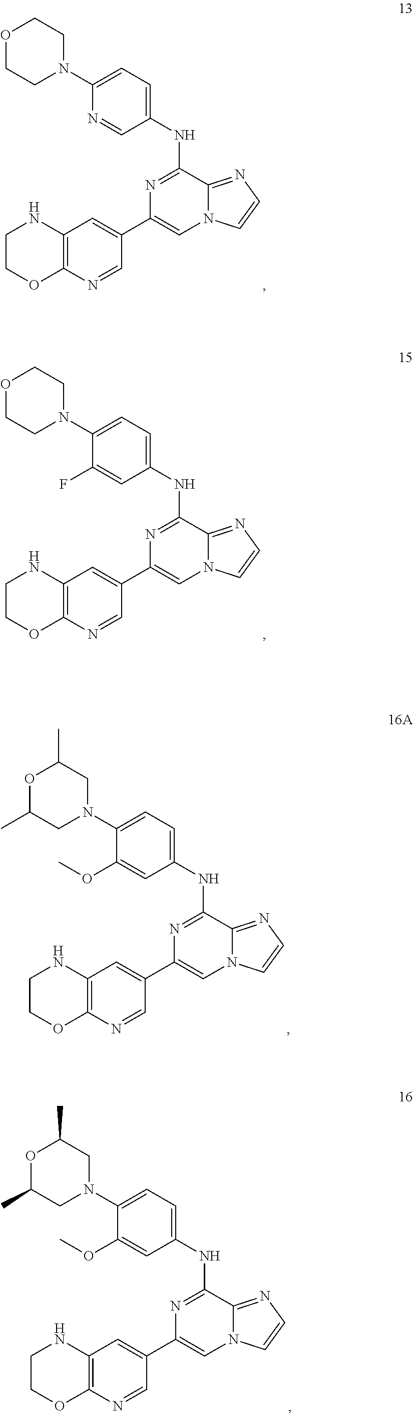 [SMILES: c1cn2cc(-c3cnc4c(c3)NCCO4)nc(Nc3ccc(N4CCOCC4)nc3)c2n1 ; Fc1cc(Nc2nc(-c3cnc4c(c3)NCCO4)cn3ccnc23)ccc1N1CCOCC1 ; COc1cc(Nc2nc(-c3cnc4c(c3)NCCO4)cn3ccnc23)ccc1N1CC(C)OC(C)C1 ; COc1cc(Nc2nc(-c3cnc4c(c3)NCCO4)cn3ccnc23)ccc1N1C[C@@H](C)O[C@@H](C)C1] 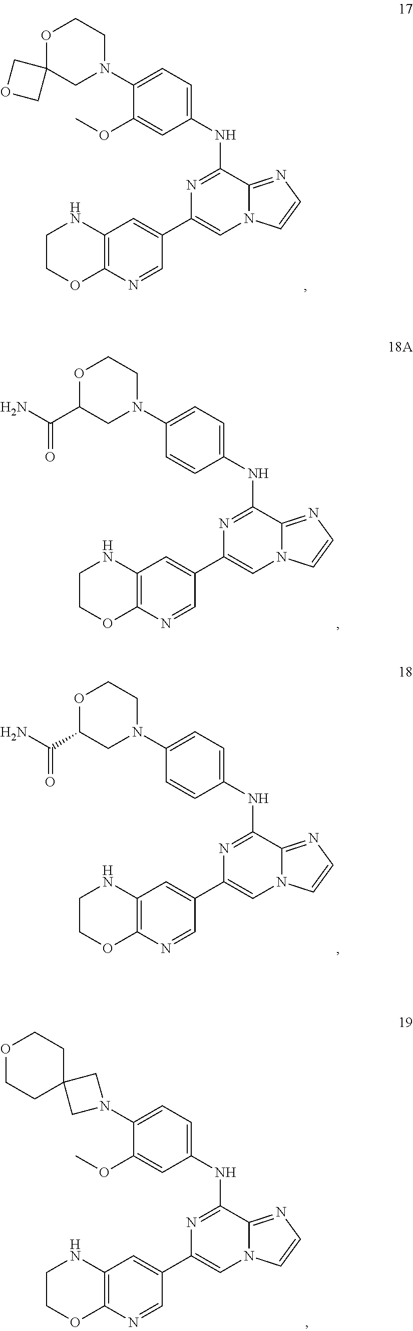 [SMILES: COc1cc(Nc2nc(-c3cnc4c(c3)NCCO4)cn3ccnc23)ccc1N1CCOC2(COC2)C1 ; NC(=O)C1CN(c2ccc(Nc3nc(-c4cnc5c(c4)NCCO5)cn4ccnc34)cc2)CCO1 ; NC(=O)[C@H]1CN(c2ccc(Nc3nc(-c4cnc5c(c4)NCCO5)cn4ccnc34)cc2)CCO1 ; COc1cc(Nc2nc(-c3cnc4c(c3)NCCO4)cn3ccnc23)ccc1N1CC2(CCOCC2)C1] 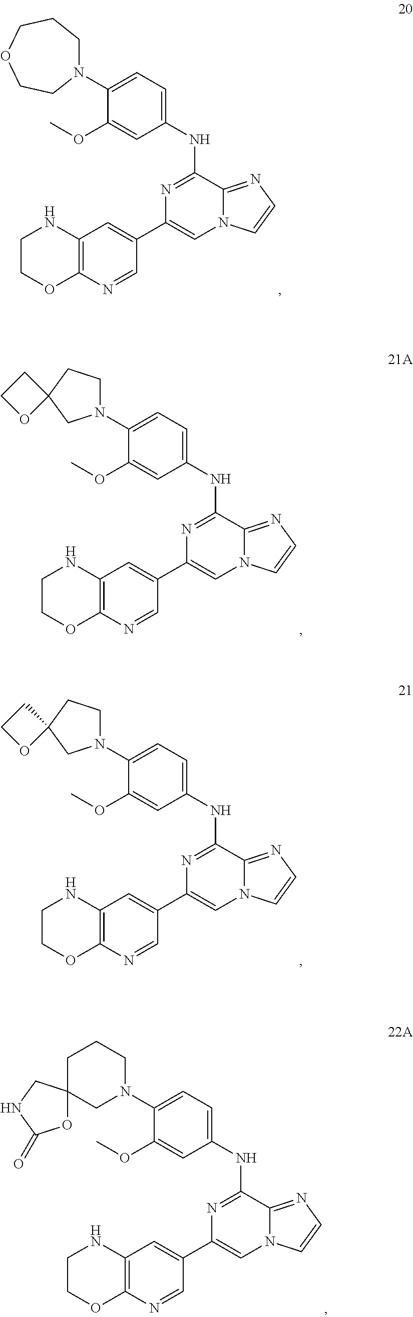 [SMILES: COc1cc(Nc2nc(-c3cnc4c(c3)NCCO4)cn3ccnc23)ccc1N1CCCOCC1 ; COc1cc(Nc2nc(-c3cnc4c(c3)NCCO4)cn3ccnc23)ccc1N1CCC2(CCO2)C1 ; COc1cc(Nc2nc(-c3cnc4c(c3)NCCO4)cn3ccnc23)ccc1N1CC[C@@]2(CCO2)C1 ; COc1cc(Nc2nc(-c3cnc4c(c3)NCCO4)cn3ccnc23)ccc1N1CCCC2(CNC(=O)O2)C1] 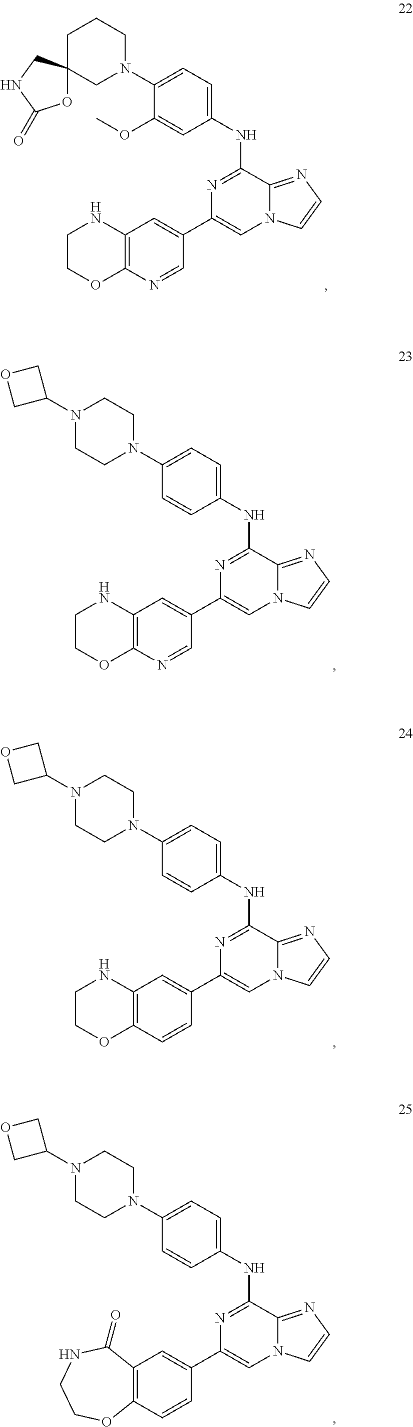 [SMILES: COc1cc(Nc2nc(-c3cnc4c(c3)NCCO4)cn3ccnc23)ccc1N1CCC[C@]2(CNC(=O)O2)C1 ; c1cn2cc(-c3cnc4c(c3)NCCO4)nc(Nc3ccc(N4CCN(C5COC5)CC4)cc3)c2n1 ; c1cn2cc(-c3ccc4c(c3)NCCO4)nc(Nc3ccc(N4CCN(C5COC5)CC4)cc3)c2n1 ; O=C1NCCOc2ccc(-c3cn4ccnc4c(Nc4ccc(N5CCN(C6COC6)CC5)cc4)n3)cc21] 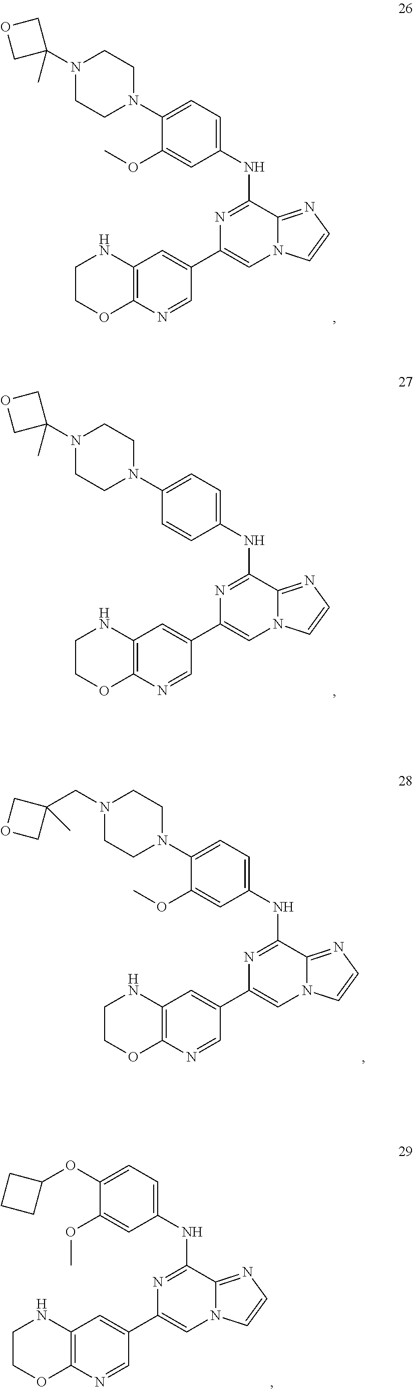 [SMILES: COc1cc(Nc2nc(-c3cnc4c(c3)NCCO4)cn3ccnc23)ccc1N1CCN(C2(C)COC2)CC1 ; CC1(N2CCN(c3ccc(Nc4nc(-c5cnc6c(c5)NCCO6)cn5ccnc45)cc3)CC2)COC1 ; COc1cc(Nc2nc(-c3cnc4c(c3)NCCO4)cn3ccnc23)ccc1N1CCN(CC2(C)COC2)CC1 ; COc1cc(Nc2nc(-c3cnc4c(c3)NCCO4)cn3ccnc23)ccc1OC1CCC1] 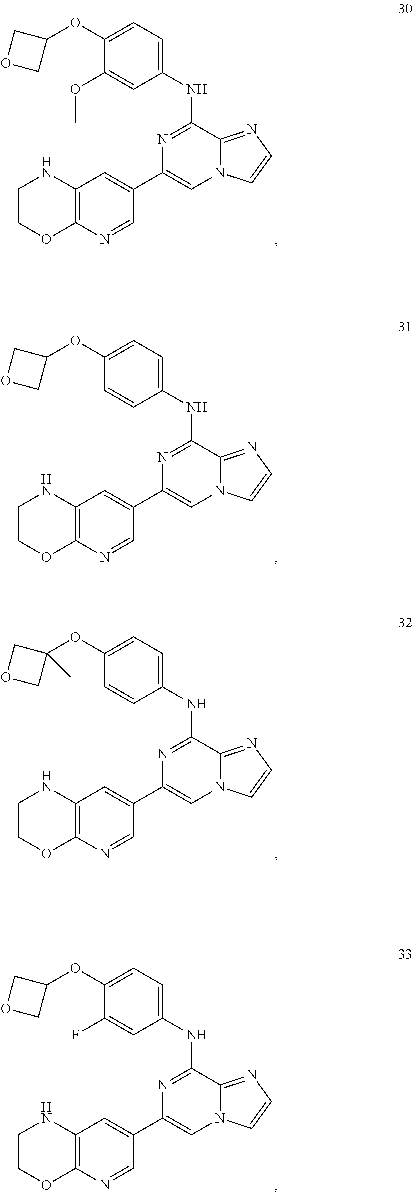 [SMILES: COc1cc(Nc2nc(-c3cnc4c(c3)NCCO4)cn3ccnc23)ccc1OC1COC1 ; c1cn2cc(-c3cnc4c(c3)NCCO4)nc(Nc3ccc(OC4COC4)cc3)c2n1 ; CC1(Oc2ccc(Nc3nc(-c4cnc5c(c4)NCCO5)cn4ccnc34)cc2)COC1 ; Fc1cc(Nc2nc(-c3cnc4c(c3)NCCO4)cn3ccnc23)ccc1OC1COC1] 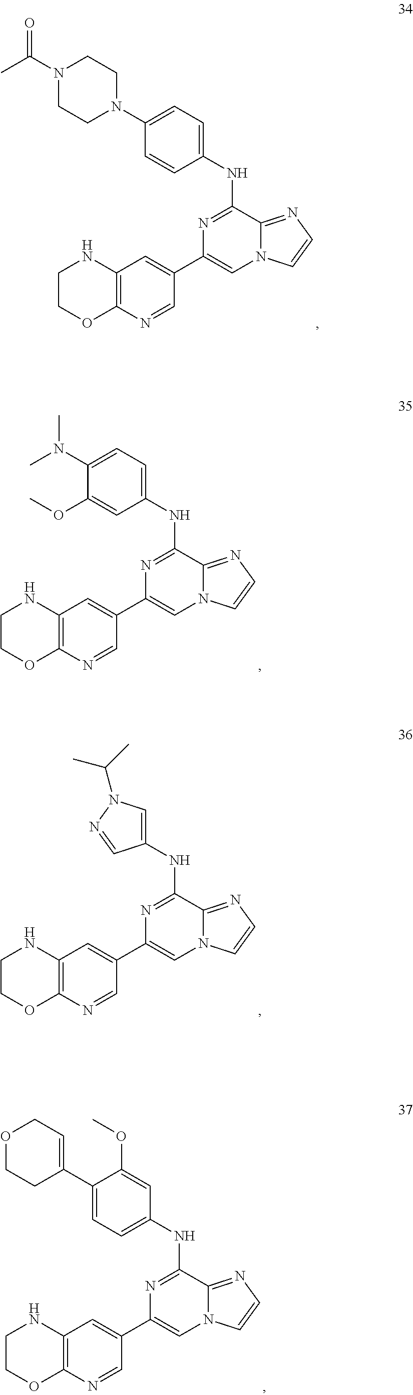 [SMILES: CC(=O)N1CCN(c2ccc(Nc3nc(-c4cnc5c(c4)NCCO5)cn4ccnc34)cc2)CC1 ; COc1cc(Nc2nc(-c3cnc4c(c3)NCCO4)cn3ccnc23)ccc1N(C)C ; CC(C)n1cc(Nc2nc(-c3cnc4c(c3)NCCO4)cn3ccnc23)cn1 ; COc1cc(Nc2nc(-c3cnc4c(c3)NCCO4)cn3ccnc23)ccc1C1=CCOCC1] 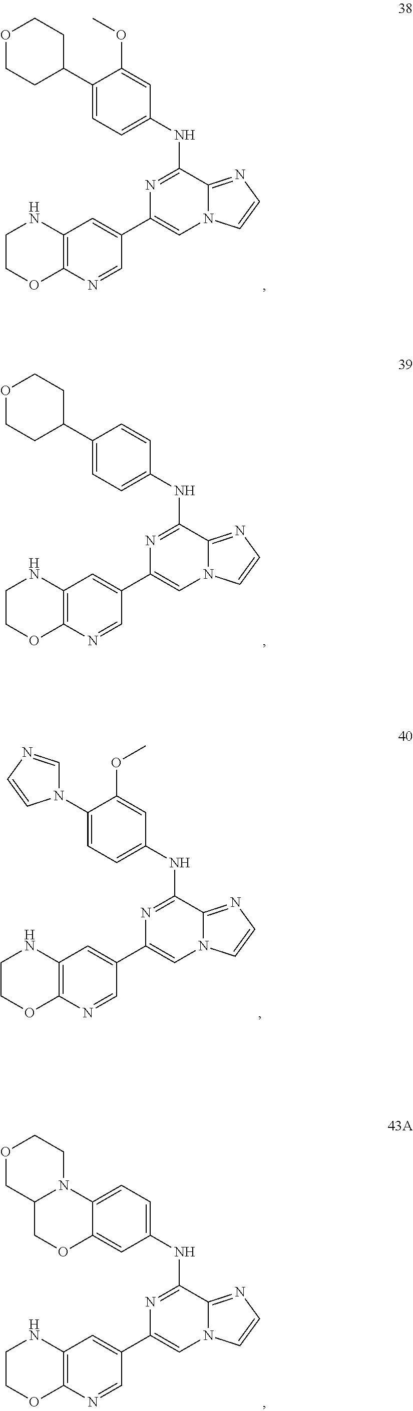 [SMILES: COc1cc(Nc2nc(-c3cnc4c(c3)NCCO4)cn3ccnc23)ccc1C1CCOCC1 ; c1cn2cc(-c3cnc4c(c3)NCCO4)nc(Nc3ccc(C4CCOCC4)cc3)c2n1 ; COc1cc(Nc2nc(-c3cnc4c(c3)NCCO4)cn3ccnc23)ccc1-n1ccnc1 ; c1cn2cc(-c3cnc4c(c3)NCCO4)nc(Nc3ccc4c(c3)OCC3COCCN43)c2n1] 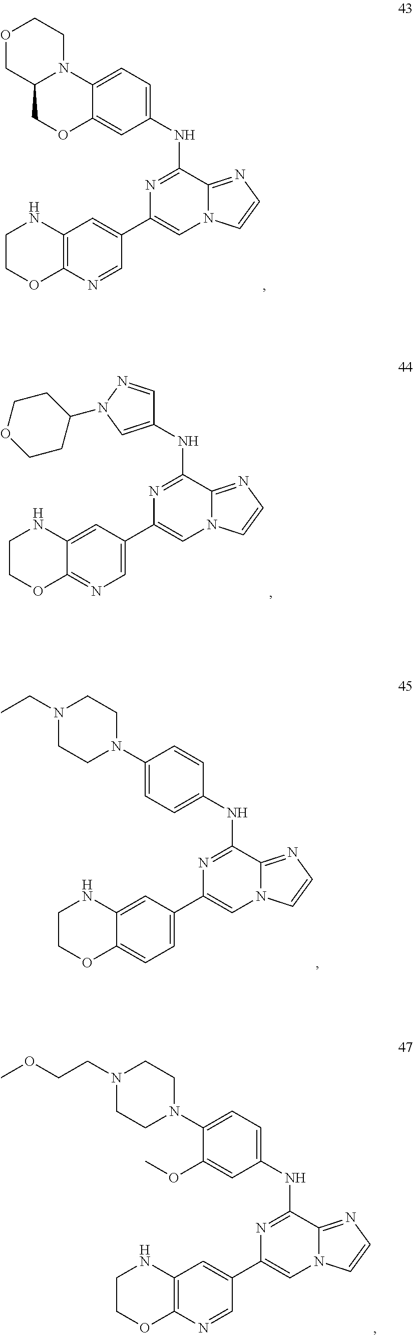 [SMILES: c1cn2cc(-c3cnc4c(c3)NCCO4)nc(Nc3ccc4c(c3)OC[C@H]3COCCN43)c2n1 ; c1cn2cc(-c3cnc4c(c3)NCCO4)nc(Nc3cnn(C4CCOCC4)c3)c2n1 ; CCN1CCN(c2ccc(Nc3nc(-c4ccc5c(c4)NCCO5)cn4ccnc34)cc2)CC1 ; COCCN1CCN(c2ccc(Nc3nc(-c4cnc5c(c4)NCCO5)cn4ccnc34)cc2OC)CC1] 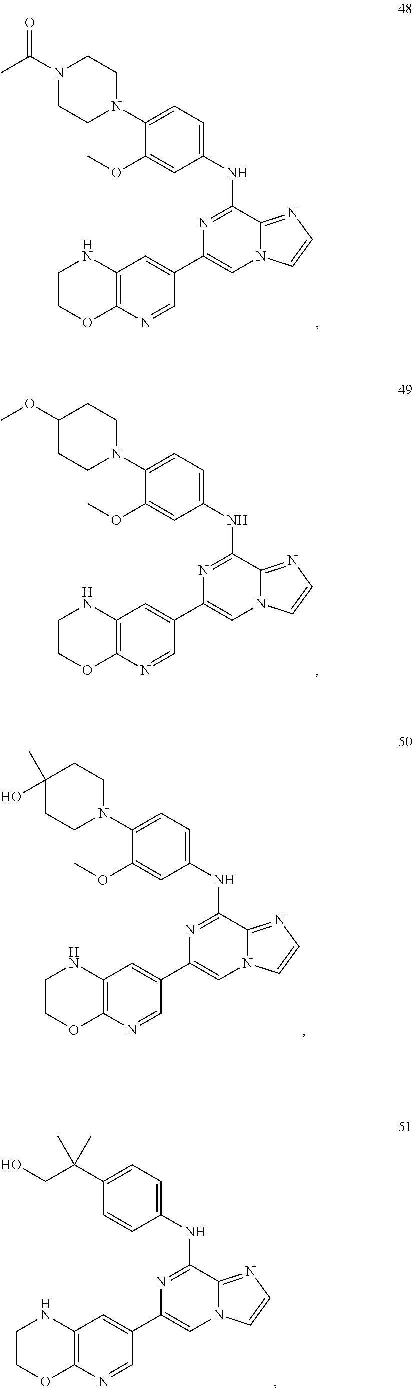 [SMILES: COc1cc(Nc2nc(-c3cnc4c(c3)NCCO4)cn3ccnc23)ccc1N1CCN(C(C)=O)CC1 ; COc1cc(Nc2nc(-c3cnc4c(c3)NCCO4)cn3ccnc23)ccc1N1CCC(OC)CC1 ; COc1cc(Nc2nc(-c3cnc4c(c3)NCCO4)cn3ccnc23)ccc1N1CCC(C)(O)CC1 ; CC(C)(CO)c1ccc(Nc2nc(-c3cnc4c(c3)NCCO4)cn3ccnc23)cc1] 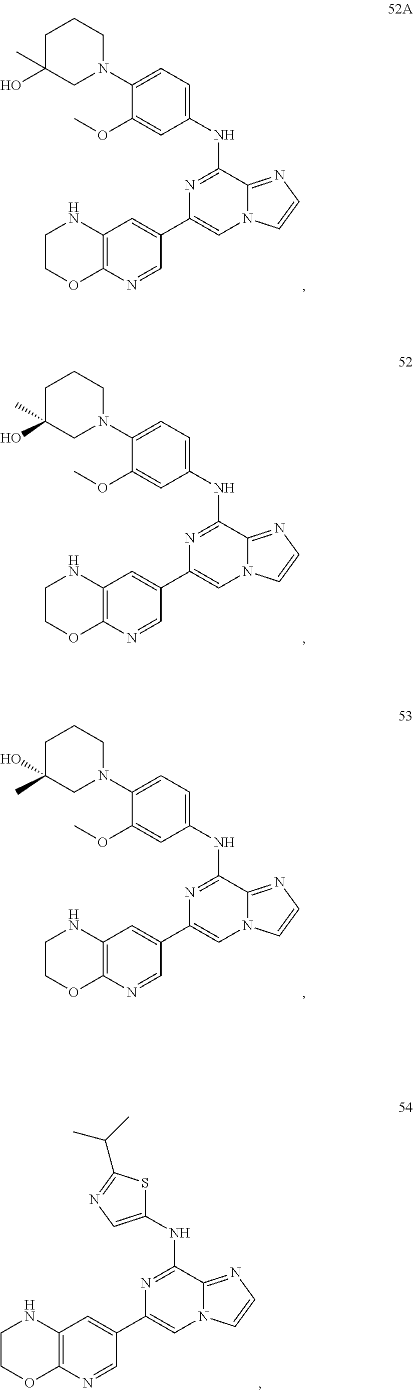 [SMILES: COc1cc(Nc2nc(-c3cnc4c(c3)NCCO4)cn3ccnc23)ccc1N1CCCC(C)(O)C1 ; COc1cc(Nc2nc(-c3cnc4c(c3)NCCO4)cn3ccnc23)ccc1N1CCC[C@](C)(O)C1 ; COc1cc(Nc2nc(-c3cnc4c(c3)NCCO4)cn3ccnc23)ccc1N1CCC[C@@](C)(O)C1 ; CC(C)c1ncc(Nc2nc(-c3cnc4c(c3)NCCO4)cn3ccnc23)s1] 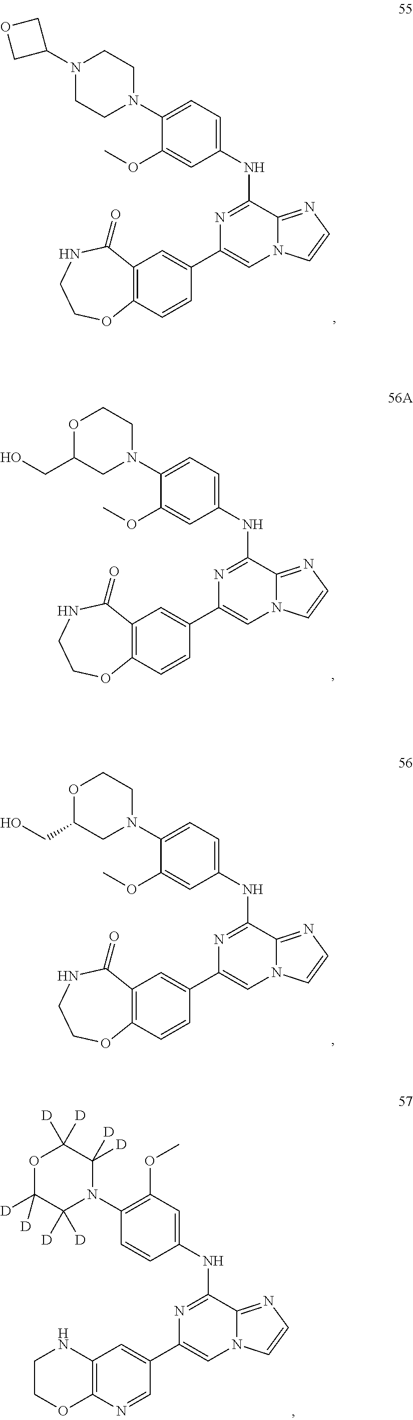 [SMILES: COc1cc(Nc2nc(-c3ccc4c(c3)C(=O)NCCO4)cn3ccnc23)ccc1N1CCN(C2COC2)CC1 ; COc1cc(Nc2nc(-c3ccc4c(c3)C(=O)NCCO4)cn3ccnc23)ccc1N1CCOC(CO)C1 ; COc1cc(Nc2nc(-c3ccc4c(c3)C(=O)NCCO4)cn3ccnc23)ccc1N1CCO[C@@H](CO)C1 ; [2H]C1([2H])OC([2H])([2H])C([2H])([2H])N(c2ccc(Nc3nc(-c4cnc5c(c4)NCCO5)cn4ccnc34)cc2OC)C1([2H])[2H]] 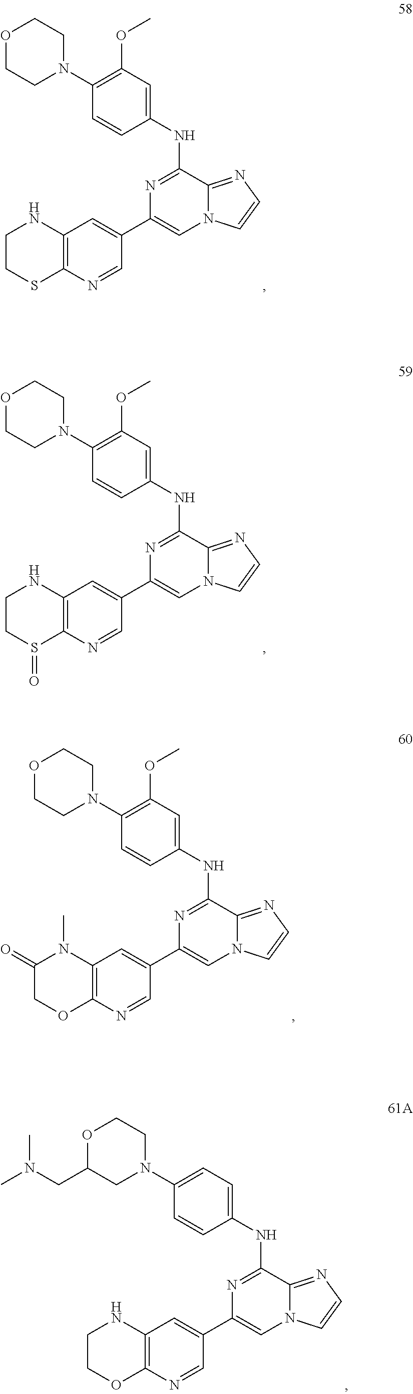 [SMILES: COc1cc(Nc2nc(-c3cnc4c(c3)NCCS4)cn3ccnc23)ccc1N1CCOCC1 ; COc1cc(Nc2nc(-c3cnc4c(c3)NCCS4=O)cn3ccnc23)ccc1N1CCOCC1 ; COc1cc(Nc2nc(-c3cnc4c(c3)N(C)C(=O)CO4)cn3ccnc23)ccc1N1CCOCC1 ; CN(C)CC1CN(c2ccc(Nc3nc(-c4cnc5c(c4)NCCO5)cn4ccnc34)cc2)CCO1] 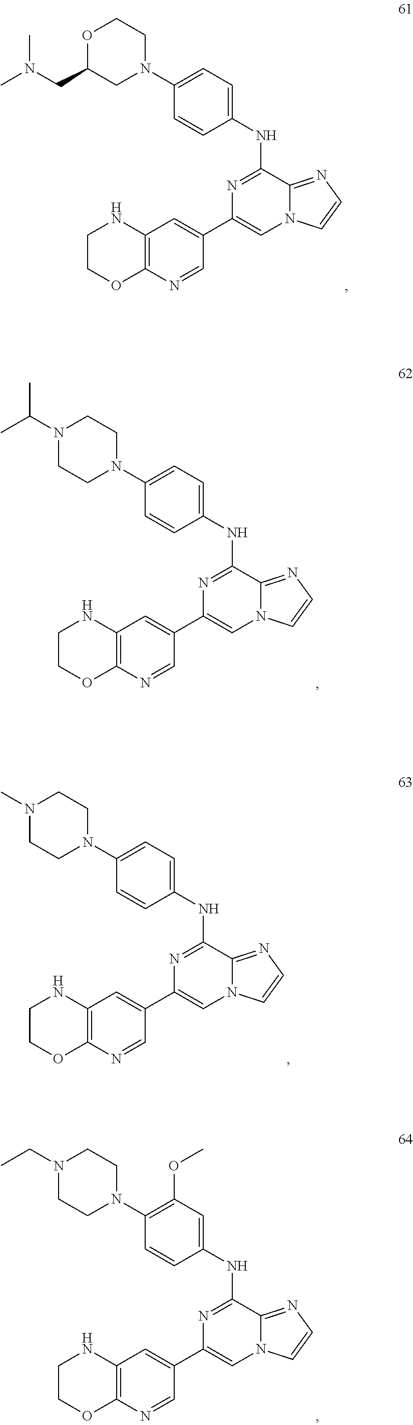 [SMILES: CN(C)C[C@@H]1CN(c2ccc(Nc3nc(-c4cnc5c(c4)NCCO5)cn4ccnc34)cc2)CCO1 ; CC(C)N1CCN(c2ccc(Nc3nc(-c4cnc5c(c4)NCCO5)cn4ccnc34)cc2)CC1 ; CN1CCN(c2ccc(Nc3nc(-c4cnc5c(c4)NCCO5)cn4ccnc34)cc2)CC1 ; CCN1CCN(c2ccc(Nc3nc(-c4cnc5c(c4)NCCO5)cn4ccnc34)cc2OC)CC1] 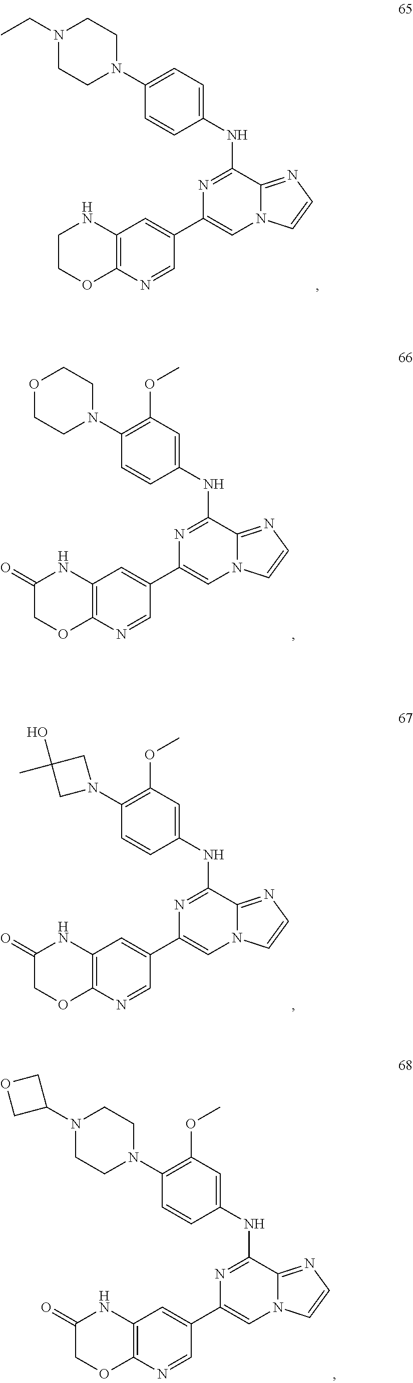 [SMILES: CCN1CCN(c2ccc(Nc3nc(-c4cnc5c(c4)NCCO5)cn4ccnc34)cc2)CC1 ; COc1cc(Nc2nc(-c3cnc4c(c3)NC(=O)CO4)cn3ccnc23)ccc1N1CCOCC1 ; COc1cc(Nc2nc(-c3cnc4c(c3)NC(=O)CO4)cn3ccnc23)ccc1N1CC(C)(O)C1 ; COc1cc(Nc2nc(-c3cnc4c(c3)NC(=O)CO4)cn3ccnc23)ccc1N1CCN(C2COC2)CC1] 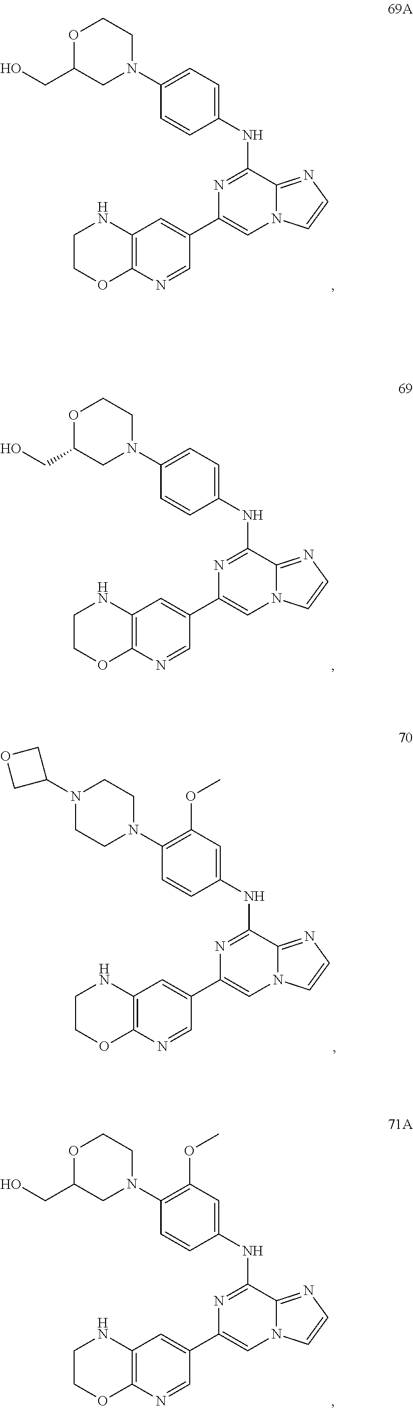 [SMILES: OCC1CN(c2ccc(Nc3nc(-c4cnc5c(c4)NCCO5)cn4ccnc34)cc2)CCO1 ; OC[C@H]1CN(c2ccc(Nc3nc(-c4cnc5c(c4)NCCO5)cn4ccnc34)cc2)CCO1 ; COc1cc(Nc2nc(-c3cnc4c(c3)NCCO4)cn3ccnc23)ccc1N1CCN(C2COC2)CC1 ; COc1cc(Nc2nc(-c3cnc4c(c3)NCCO4)cn3ccnc23)ccc1N1CCOC(CO)C1] 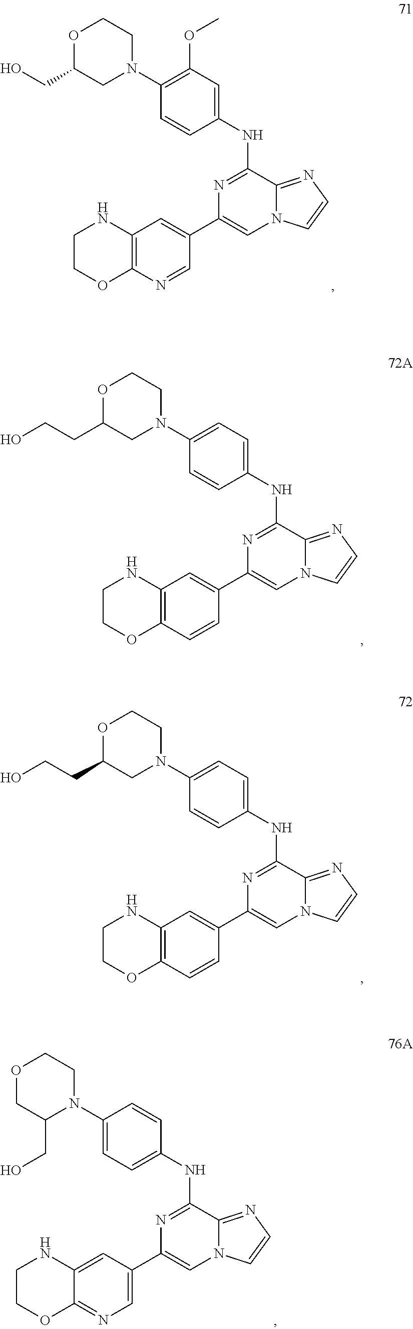 [SMILES: COc1cc(Nc2nc(-c3cnc4c(c3)NCCO4)cn3ccnc23)ccc1N1CCO[C@@H](CO)C1 ; OCCC1CN(c2ccc(Nc3nc(-c4ccc5c(c4)NCCO5)cn4ccnc34)cc2)CCO1 ; OCC[C@@H]1CN(c2ccc(Nc3nc(-c4ccc5c(c4)NCCO5)cn4ccnc34)cc2)CCO1 ; OCC1COCCN1c1ccc(Nc2nc(-c3cnc4c(c3)NCCO4)cn3ccnc23)cc1] 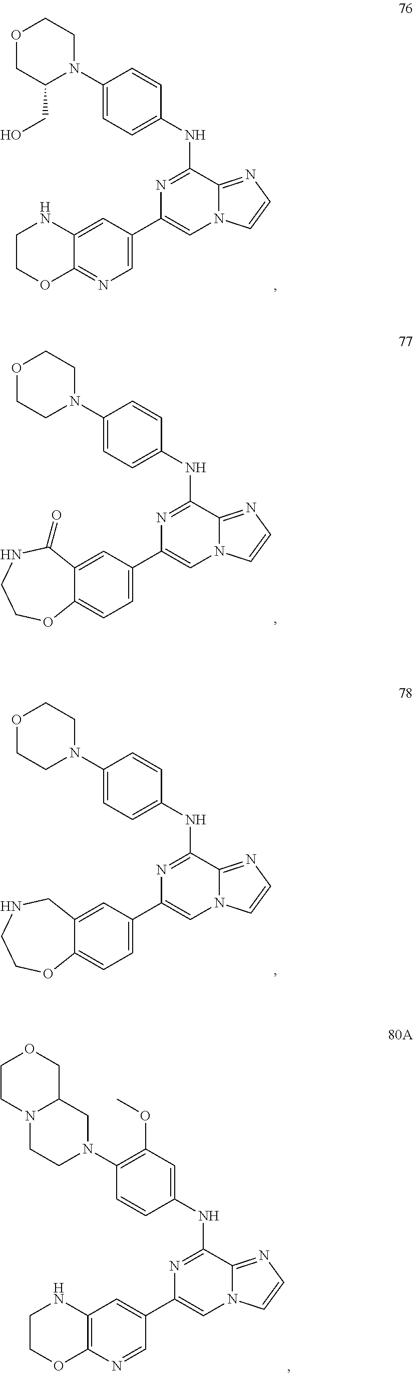 [SMILES: OC[C@@H]1COCCN1c1ccc(Nc2nc(-c3cnc4c(c3)NCCO4)cn3ccnc23)cc1 ; O=C1NCCOc2ccc(-c3cn4ccnc4c(Nc4ccc(N5CCOCC5)cc4)n3)cc21 ; c1cn2cc(-c3ccc4c(c3)CNCCO4)nc(Nc3ccc(N4CCOCC4)cc3)c2n1 ; COc1cc(Nc2nc(-c3cnc4c(c3)NCCO4)cn3ccnc23)ccc1N1CCN2CCOCC2C1] 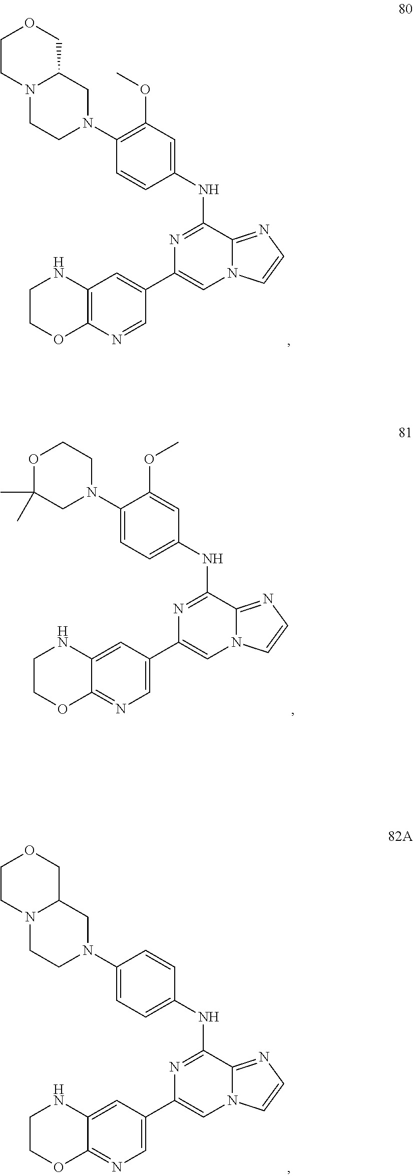 [SMILES: COc1cc(Nc2nc(-c3cnc4c(c3)NCCO4)cn3ccnc23)ccc1N1CCN2CCOC[C@@H]2C1 ; COc1cc(Nc2nc(-c3cnc4c(c3)NCCO4)cn3ccnc23)ccc1N1CCOC(C)(C)C1 ; c1cn2cc(-c3cnc4c(c3)NCCO4)nc(Nc3ccc(N4CCN5CCOCC5C4)cc3)c2n1] 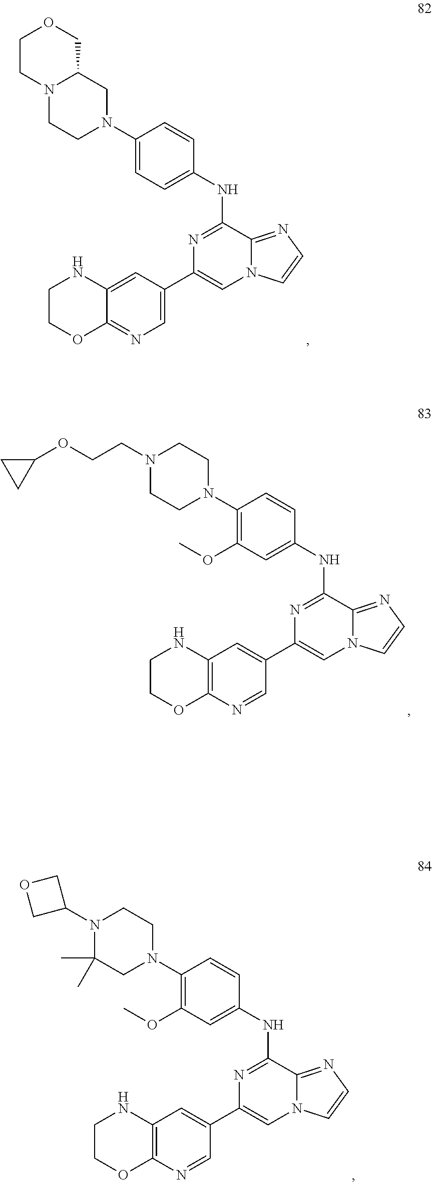 [SMILES: c1cn2cc(-c3cnc4c(c3)NCCO4)nc(Nc3ccc(N4CCN5CCOC[C@@H]5C4)cc3)c2n1 ; COc1cc(Nc2nc(-c3cnc4c(c3)NCCO4)cn3ccnc23)ccc1N1CCN(CCOC2CC2)CC1 ; COc1cc(Nc2nc(-c3cnc4c(c3)NCCO4)cn3ccnc23)ccc1N1CCN(C2COC2)C(C)(C)C1] 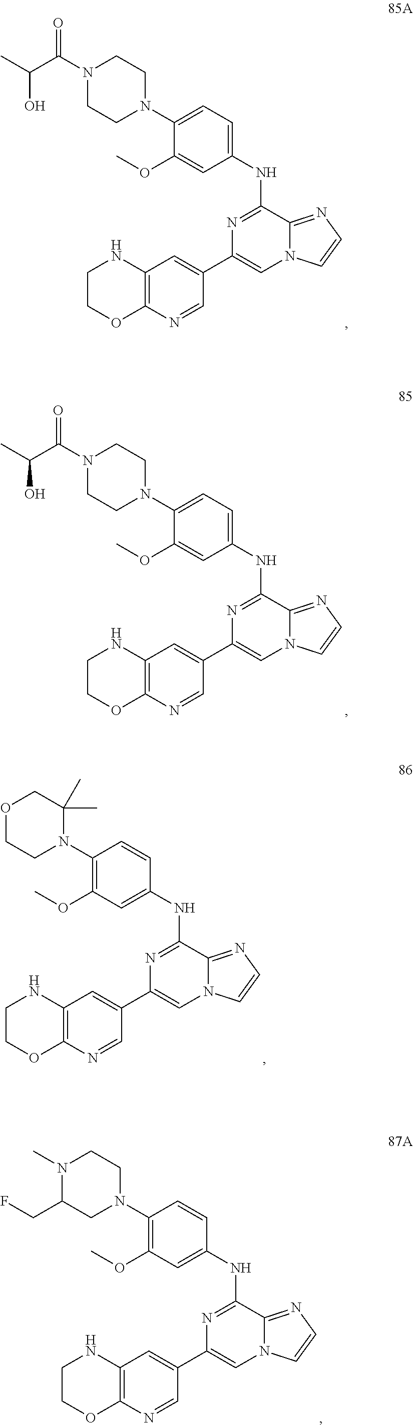 [SMILES: COc1cc(Nc2nc(-c3cnc4c(c3)NCCO4)cn3ccnc23)ccc1N1CCN(C(=O)C(C)O)CC1 ; COc1cc(Nc2nc(-c3cnc4c(c3)NCCO4)cn3ccnc23)ccc1N1CCN(C(=O)[C@H](C)O)CC1 ; COc1cc(Nc2nc(-c3cnc4c(c3)NCCO4)cn3ccnc23)ccc1N1CCOCC1(C)C ; COc1cc(Nc2nc(-c3cnc4c(c3)NCCO4)cn3ccnc23)ccc1N1CCN(C)C(CF)C1] 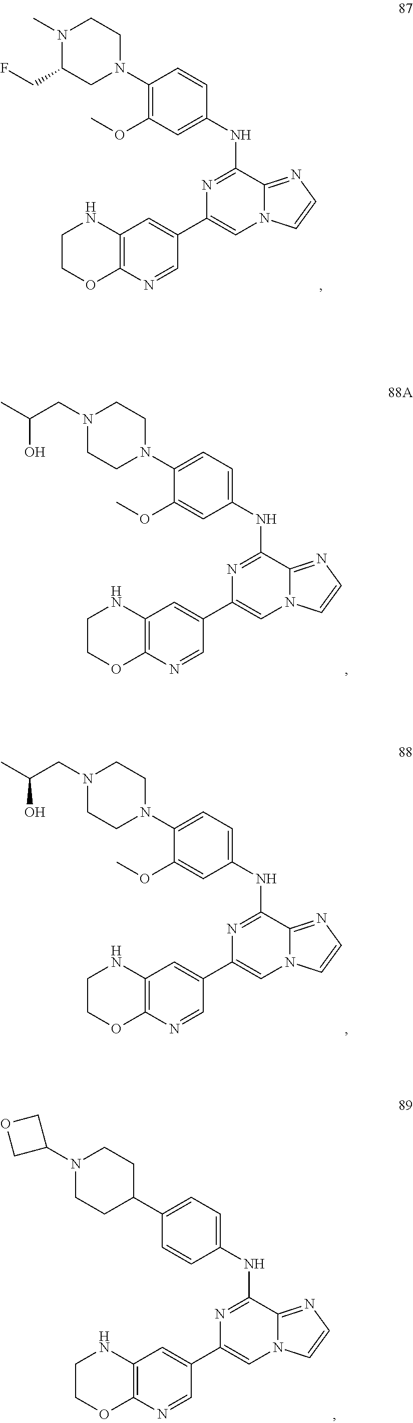 [SMILES: COc1cc(Nc2nc(-c3cnc4c(c3)NCCO4)cn3ccnc23)ccc1N1CCN(C)[C@@H](CF)C1 ; COc1cc(Nc2nc(-c3cnc4c(c3)NCCO4)cn3ccnc23)ccc1N1CCN(CC(C)O)CC1 ; COc1cc(Nc2nc(-c3cnc4c(c3)NCCO4)cn3ccnc23)ccc1N1CCN(C[C@H](C)O)CC1 ; c1cn2cc(-c3cnc4c(c3)NCCO4)nc(Nc3ccc(C4CCN(C5COC5)CC4)cc3)c2n1] 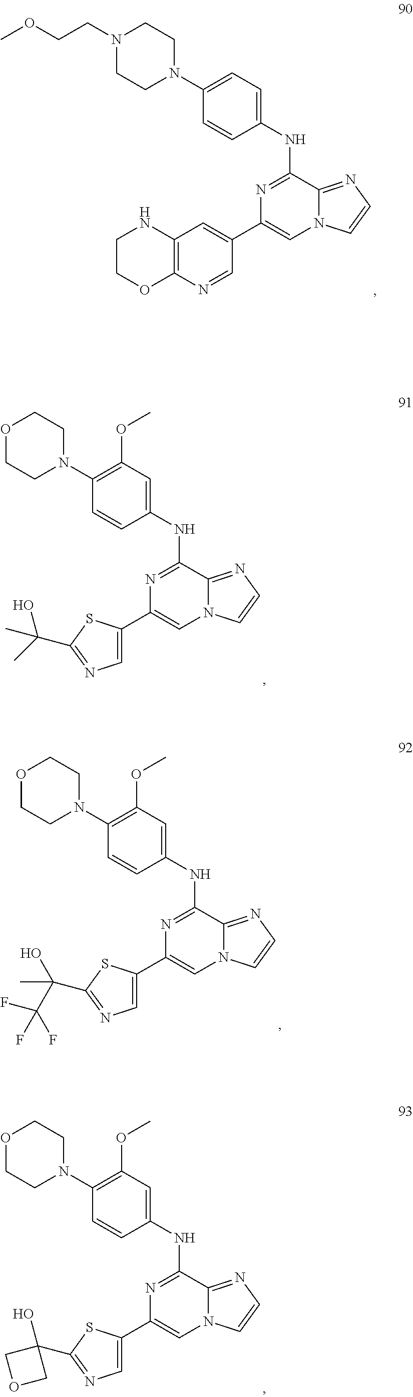 [SMILES: COCCN1CCN(c2ccc(Nc3nc(-c4cnc5c(c4)NCCO5)cn4ccnc34)cc2)CC1 ; COc1cc(Nc2nc(-c3cnc(C(C)(C)O)s3)cn3ccnc23)ccc1N1CCOCC1 ; COc1cc(Nc2nc(-c3cnc(C(C)(O)C(F)(F)F)s3)cn3ccnc23)ccc1N1CCOCC1 ; COc1cc(Nc2nc(-c3cnc(C4(O)COC4)s3)cn3ccnc23)ccc1N1CCOCC1] 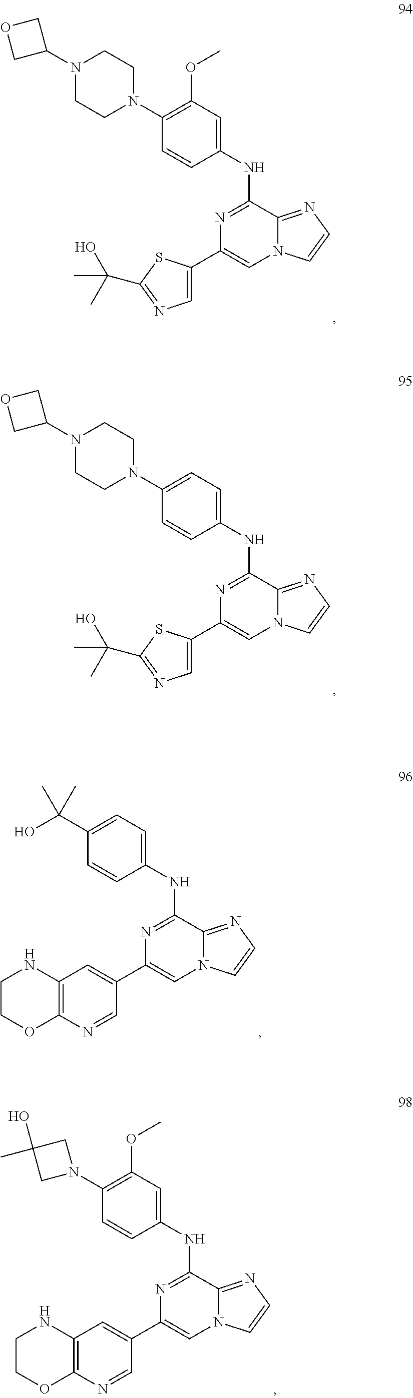 [SMILES: COc1cc(Nc2nc(-c3cnc(C(C)(C)O)s3)cn3ccnc23)ccc1N1CCN(C2COC2)CC1 ; CC(C)(O)c1ncc(-c2cn3ccnc3c(Nc3ccc(N4CCN(C5COC5)CC4)cc3)n2)s1 ; CC(C)(O)c1ccc(Nc2nc(-c3cnc4c(c3)NCCO4)cn3ccnc23)cc1 ; COc1cc(Nc2nc(-c3cnc4c(c3)NCCO4)cn3ccnc23)ccc1N1CC(C)(O)C1] 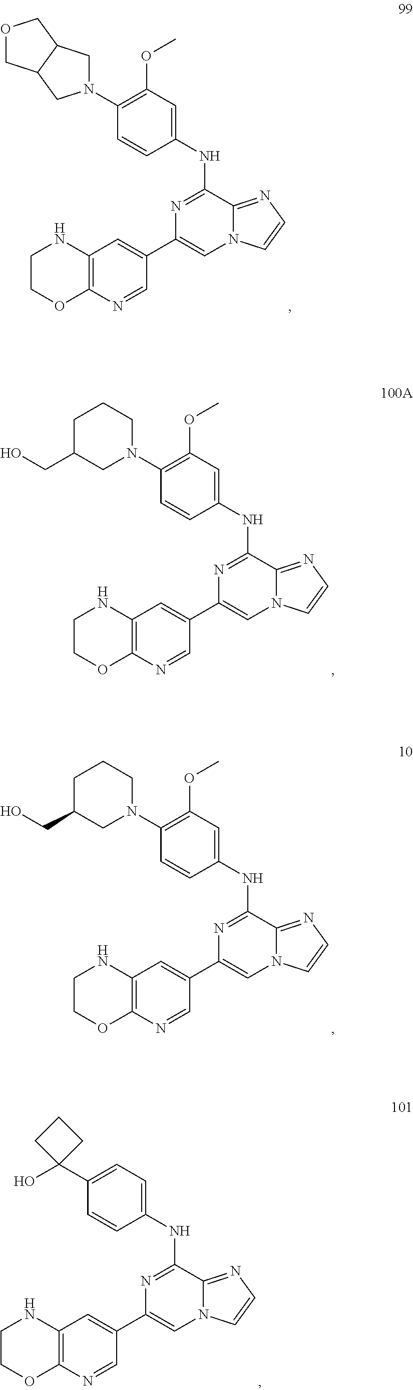 [SMILES: COc1cc(Nc2nc(-c3cnc4c(c3)NCCO4)cn3ccnc23)ccc1N1CC2COCC2C1 ; COc1cc(Nc2nc(-c3cnc4c(c3)NCCO4)cn3ccnc23)ccc1N1CCCC(CO)C1 ; COc1cc(Nc2nc(-c3cnc4c(c3)NCCO4)cn3ccnc23)ccc1N1CCC[C@H](CO)C1 ; OC1(c2ccc(Nc3nc(-c4cnc5c(c4)NCCO5)cn4ccnc34)cc2)CCC1] 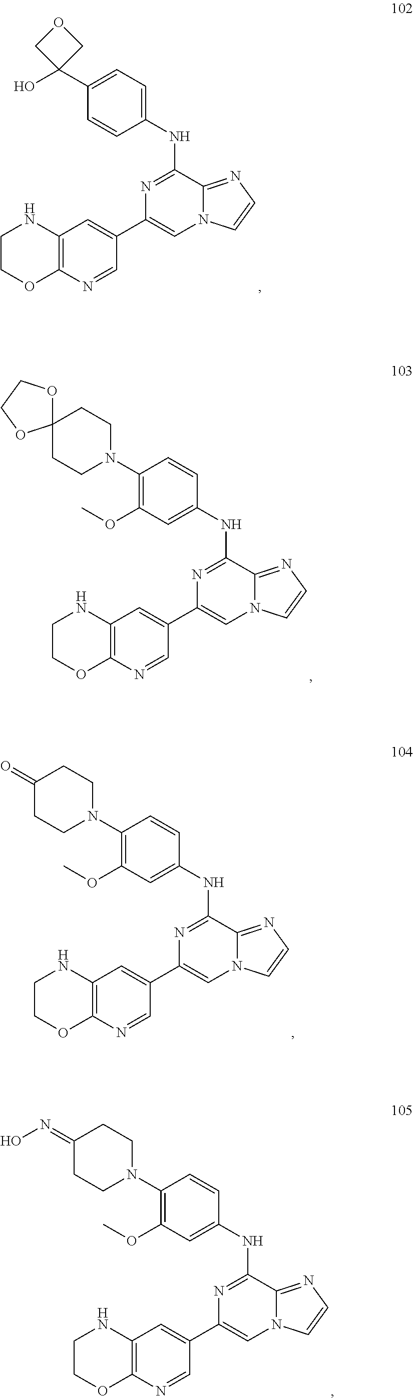 [SMILES: OC1(c2ccc(Nc3nc(-c4cnc5c(c4)NCCO5)cn4ccnc34)cc2)COC1 ; COc1cc(Nc2nc(-c3cnc4c(c3)NCCO4)cn3ccnc23)ccc1N1CCC2(CC1)OCCO2 ; COc1cc(Nc2nc(-c3cnc4c(c3)NCCO4)cn3ccnc23)ccc1N1CCC(=O)CC1 ; COc1cc(Nc2nc(-c3cnc4c(c3)NCCO4)cn3ccnc23)ccc1N1CCC(=NO)CC1] 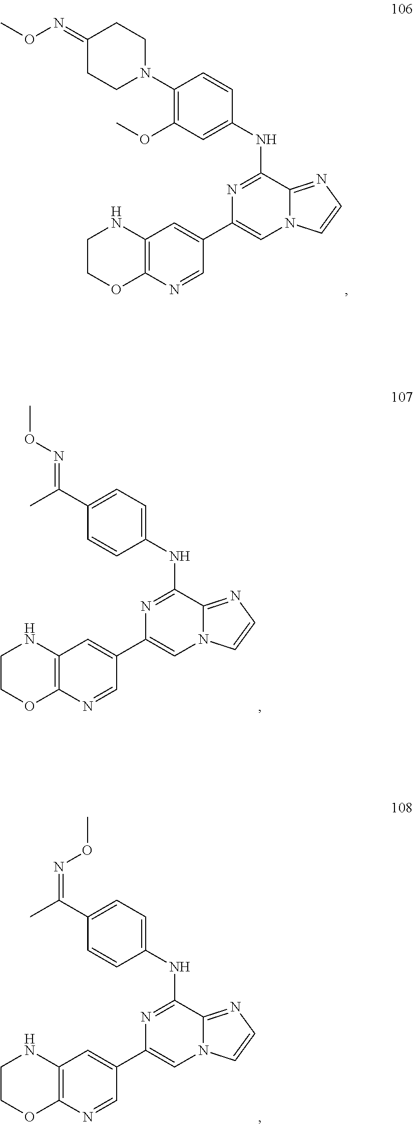 [SMILES: CON=C1CCN(c2ccc(Nc3nc(-c4cnc5c(c4)NCCO5)cn4ccnc34)cc2OC)CC1 ; CO/N=C(\C)c1ccc(Nc2nc(-c3cnc4c(c3)NCCO4)cn3ccnc23)cc1 ; CO/N=C(/C)c1ccc(Nc2nc(-c3cnc4c(c3)NCCO4)cn3ccnc23)cc1] 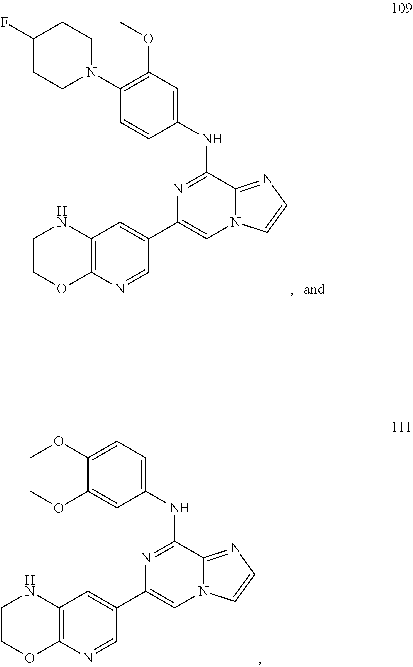 [SMILES: COc1cc(Nc2nc(-c3cnc4c(c3)NCCO4)cn3ccnc23)ccc1N1CCC(F)CC1 ; COc1ccc(Nc2nc(-c3cnc4c(c3)NCCO4)cn3ccnc23)cc1OC]
or a pharmaceutically acceptable salt thereof.

14. A pharmaceutical composition comprising:
at least one compound of claim 13; and
at least one pharmaceutically acceptable vehicle selected from the group consisting of carriers, adjuvants, and excipients.

15. A method for treating a patient having a disease responsive to the inhibition of Syk activity, comprising administering to the patient a compound of claim 13.

16. The method of claim 15, wherein the disease is selected from the group consisting of cancer, an allergic disorder, an inflammatory disease, an autoimmune disease, and an acute inflammatory reaction.

17. The method of claim 15, wherein the disease is lymphoma or leukemia.

18. The method of claim 17, wherein the disease is selected from the group consisting of B-cell lymphoma, Hodgkin's lymphoma, non-Hodgkin's lymphoma, hairy cell leukemia, multiple myeloma, chronic myelogenous leukemia, acute myelogenous leukemia, chronic lymphocytic leukemia, acute lymphocytic leukemia, rheumatoid arthritis, allergic rhinitis, chronic obstructive pulmonary disease (COPD), adult respiratory distress syndrome (ARDS), multiple sclerosis, inflammatory bowel disease, Crohn's disease, ulcerative colitis, systemic lupus erythematosus, ovarian cancer, and polycystic kidney disease.

19. The method of claim 15, wherein the patient is a human.

20. The method of claim 15, wherein the compound is administered to the patient intravenously, intramuscularly, parenterally, nasally or orally.
